(19) United States
(12) Patent Application Publication
(10) Pub. No.: US 2023/0017786 A1
(43) Pub. Date: Jan. 19, 2023

(54) DEXTROMETHADONE AS A DISEASE-MODIFYING TREATMENT FOR NEUROPSYCHIATRIC DISORDERS AND DISEASES

(71) Applicants: Paolo L. MANFREDI, Miami Beach, FL (US); Charles E. Inturrisi, New York, NY (US); University of Padova, Padua (IT); Institute for Research in Biomedicine, Bellinzona (CH)

(72) Inventors: Paolo L. Manfredi, Miami Beach, FL (US); Charles E. Inturrisi, New York, NY (US); Sara De Martin, Vigonza (IT); Andrea Mattarei, Perarolo di Vigonza (IT); Jacopo Sgrignani, Almenno San Salvatore (IT); Andrea Cavalli, Zurich (CH)

(21) Appl. No.: 17/787,608

(22) PCT Filed: Dec. 30, 2020
(86) PCT No.: PCT/US2020/067498

§ 371 Date: Jun. 21, 2022

Related U.S. Application Data
(60) Provisional application No. 63/031,785, filed on May 29, 2020.
(60) Provisional application No. 63/010,391, filed on Apr. 15, 2020.
(60) Provisional application No. 62/993,188, filed on Mar. 23, 2020.
(60) Provisional application No. 62/963,874, filed on Jan. 21, 2020.
(60) Provisional application No. 62/956,839, filed on Jan. 3, 2020.

(51) Int. Cl. A61K 31/137; A61K 33/06; A61K 33/30; A61P 25/00

(52) U.S. Cl. CPC A61K 31/137; A61K 33/06; A61K 33/30; A61P 25/00

(57) ABSTRACT

Methods and compositions for modifying the course and severity of neuropsychiatric disorders. The method includes administering a composition to a subject suffering from a neuropsychiatric disorder, wherein the composition includes a substance selected from dextromethadone, dextromethadone metabolites, d-methadol, d-alpha-acetylmethadol, d-alpha-normethadol, l-alpha-normethadol, and pharmaceutically acceptable salts thereof.

### CROSS-REFERENCE TO RELATED APPLICATIONS

This application claims the benefit of the filing dates of U.S. Patent Application Ser. No. 63/031,785 filed May 29, 2020, U.S. Patent Application Ser. No. 63/010,391 filed Apr. 15, 2020, U.S. Patent Application Ser. No. 62/993,188 filed Mar. 23, 2020, U.S. Patent Application Ser. No. 62/963,874 filed Jan. 21, 2020, and U.S. Patent Application Ser. No. 62/956,839 filed Jan. 3, 2020, the disclosures of all of which are incorporated by reference herein in their entireties.

### FIELD OF THE INVENTION

The present invention relates to the treatment of various disorders and diseases, and to compounds and/or compositions for such treatment.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Many neuropsychiatric disorders are significant clinical conditions that negatively affect various aspects of an individual's life. For example, major depressive disorder (MDD) is a significant clinical condition that impacts mood, behavior, cognition, motivation, energy, ability to socialize and work, and basic functions, such as appetite, sexual activity, and sleep. It is a mental disorder generally characterized by at least two weeks of low mood that is present across most situations. It is often accompanied by low self-esteem, loss of interest in normally enjoyable activities, including eating and sexual activity, decreased cognitive functions, low energy, and pain and/or suffering without a clear cause. MDD can negatively affect an individual's personal family and social life, work life, and/or education—as well as sleeping, eating, sexual habits, and general health—and can result in suicide.

MDD is believed to be caused by a combination of genetic and environmental factors. Risk factors include a family history of the condition, major life changes, health problems, certain medical conditions, certain medications, and substance abuse. A substantial amount of the risk is considered to be related to genetics. The diagnosis of MDD is based on the person's reported experiences and examination by a trained health care provider. Testing may be done to rule out physical conditions that can cause similar symptoms. MDD is more severe and lasts longer than the isolated symptom of depression (a depressed mood), which is a sad or depressed feeling that may be self-contained and short-lived, does not generally affect cognitive functions and energy levels, and does not substantially impair the ability to work or socialize.

The most widely used criteria for diagnosing depressive disorders and diseases are found in the American Psychiatric Association's Diagnostic and Statistical Manual of Mental Disorders (DSM-5), which is typically used in the United States and non-European countries, and the World Health Organization's International Statistical Classification of Diseases and Related Health Problems (ICD-10), which is typically used in European countries.

MDD is classified as a mood disorder in DSM-5. The diagnosis hinges on the presence of single or recurrent major depressive episodes. Further qualifiers are used to classify both the episode itself and the course of the disorder. The ICD-10 system lists similar criteria for the diagnosis of a depressive episode (mild, moderate, or severe).

More specifically, to be diagnosed with MDD under DSM-5, a subject must have 5 or more of the following symptoms, and experience them at least once a day for a period of more than 2 weeks: (1) feeling sad or irritable most of the day, nearly every day; (2) being less interested in most activities that were once enjoyed; (3) sudden weight gain or loss, or change in appetite; (4) trouble falling asleep or wanting to sleep more than usual; (5) feelings of restlessness; (6) unusually tired or lack of energy; (7) worthless or guilty feelings, often about things that wouldn't normally make the subject feel that way; (8) difficulty concentrating, thinking, or making decisions; and (9) thoughts of harming oneself or committing suicide.

One emerging characteristic of MDD, and other neuropsychiatric disorders, is a dysfunction in molecular functions of certain brain cells (e.g., neurons and astrocytes) resulting in dysfunction of neuronal circuits (i.e., the multiple neurons interconnected by synapses, e.g., cells that are part of the endorphin system). This neuronal circuit dysfunction in light of the present application can be particularly characterized or caused by a dysfunction of ion channels [e.g., ion channels integral to the N-methyl-D-aspartate receptor (“NMDAR”)].

Patients with MDD are typically treated with standard antidepressant medications and/or counseling, with the initial step taken by primary care providers often being the prescription of antidepressant medications. Such medications include selective serotonin reuptake inhibitors (SSRIs) [which include well-known drugs such as fluoxetine (Prozac) and citalopram (Celexa)], serotonin and norepinephrine reuptake inhibitors (SNRIs), and bupropion. Serotonin is a brain chemical that is believed to be central to regulation of mood. Patients with MDD have been thought to have low levels of serotonin. Therefore, increasing the amount of available serotonin is widely considered to be useful in the treatment of these patients.

While the exact mechanism of action of SSRIs and SNRIs is unknown, the postulated mechanism is the inhibition of inward transporters with increase in select neurotransmitters at synaptic junctions (serotonin and/or norepinephrine). The effectiveness of these drugs, acutely and chronically, is highly unpredictable. The same unpredictability in response is shared by atypical antidepressants acting on different receptors and/or pathways. In the case of MDD, the effect size of these treatments tends to be low (around 0.3) and in the case of SSRIs (the current standard treatment for MDD), the therapeutic effect is usually delayed by 4-8 weeks when present (over 50% of patients do not respond to first line antidepressants), and generally requires prolonged treatment over months. In summary, attempts to direct modulation of neurotransmitter receptors and pathways in MDD—as well as in chronic disorders such as chronic pain disorders, anxiety disorders, and other neuropsychiatric disorders (including schizophrenia)—have been disappointing, and current treatments have been largely unsuccessful and are based on a symptomatic approach (drugs that result in an increase in serotonin, a chemical thought to control mood).

For example, while some mental symptoms may be temporarily improved by modulating the neurotransmitter pathway of choice for a particular symptom or symptoms (e.g., modulation of the serotonin pathway by a SSRI drug for depression), this modulation is also likely to interfere with the function of other neurons in other circuits or areas of the brain (or even in other tissues, e.g., extra CNS tissues) that also function at least partly with the same neurotransmitter pathway, but may not have been dysfunctional. Additionally, pharmacologically-induced acute changes in neurotransmitter concentrations in the synaptic cleft are likely to trigger compensatory biofeedback mechanisms with unpredictable longer term consequences. And so, these currently-used drugs, especially when used chronically, are likely to result in poor and unpredictable long-term outcomes because of molecular feedback mechanisms. Because of the non-selectiveness implicit in their mechanism of action, some neurotransmitter pathway modulating drugs (e.g., SSRIs) will also have effects on pathways outside of the nervous system and cause additional side effects, such as sexual dysfunction and metabolic side effects such as weight gain, impaired glucose tolerance, diabetes, and lipid metabolism dysfunction.

Further, with a multiplicity of different endogenous neurotransmitter/receptor systems, the manipulation of one neurotransmitter system (or a handful of neurotransmitter systems) may modulate the function of a dysfunctional circuit in a manner that may improve select target symptoms, but does not act (or is unlikely to act) on the primary cause of the dysfunction (e.g., NMDAR hyperactivity) for that circuit. Thus, such a drug is unlikely to restore physiological cellular and circuit functions. As a result, the dysfunctional cell that triggered and maintained the disorder will continue to be dysfunctional despite (and often because of) pharmacologically-induced changes in surrounding levels of neurotransmitters. Fluoxetine and other drugs categorized as SSRIs for MDD are an example of such a neurotransmitter pathway modulating drug for the serotonin/5-HT receptor system. In clinical trials, they have typically shown a weak effect size and delayed, unpredictable, and often un-sustained efficacy.

Furthermore, upon discontinuation of SSRIs, patients are likely to experience withdrawal symptoms, as happens with most drugs that influence neurotransmitters and their pathways. And the abrupt discontinuation of symptomatic drugs may even result in a phenomenon of augmentation of symptoms (worsening of symptoms compared to pre-treatment baseline). In some instances, after a certain amount of time, augmentation may be seen even when the symptomatic drug is continued rather than discontinued (e.g. in the case of dopamine agonists).

Though these drawbacks of current drug treatments are well known, clinicians continue to use these drugs because they have few (if any) effective alternative options for managing inadequate response to antidepressant therapy. Furthermore, hitherto the understanding of the molecular mechanism underlying MDD and related neuropsychiatric disorders has been limited. And so, when first line antidepressants are not successful in alleviating the manifestations of MDD, clinicians may maximize doses of the initial standard antidepressant, change to a different antidepressant, resort to electroconvulsive therapy, or augment treatment with off-label medications—even in view of all of the drawbacks associated with these therapies. While some patients experience symptom improvement with subsequent or augmented treatment approaches, the likelihood of remission decreases with additional treatment steps and those who undergo more treatment steps before becoming symptom-free are more likely to relapse. The greatest patient benefit is realized when the first or second treatment approaches are successful, but such success is often not obtained with current treatment approaches.

Additionally, the slow onset of action and the side effects of currently available treatments also contribute to poor patient adherence. To date, the US Food and Drug Administration (FDA) has approved only 3 drugs as adjunctive therapy to antidepressants for the treatment of MDD. All three are second-generation atypical antipsychotics (aripiprazole, quetiapine extended release, and brexpiprazole) and carry an increased risk for neuroleptic malignant syndrome, tardive dyskinesia, and metabolic side effects including diabetes mellitus, dyslipidemia, and weight gain. Further, the delayed onset of action of standard antidepressants is linked to suicidal risk.

An additional problem with current methods and compositions for treating MDD (and other disorders) is that certain individuals may be resistant to treatments. Treatment-resistant depression (TRD) is a term used in clinical psychiatry to describe a condition that affects people with MDD (and other similar disorders) who do not respond adequately to a course of appropriate antidepressant medication within a certain time. Standard definitions of TRD vary. For regulatory purposes (FDA), TRD is currently defined as failure to respond to at least two adequate trials with standard antidepressants in the current major depressive episode. Inadequate response has traditionally been defined as no clinical response whatsoever (e.g. no improvement in depressive symptoms). However, many clinicians consider a response inadequate if the person does not achieve full remission of symptoms. People with TRD who do not adequately respond to antidepressant treatment are sometimes referred to as pseudoresistant. Some factors that contribute to inadequate treatment are: early discontinuation of treatment, insufficient dosage of medication, patient noncompliance, misdiagnosis, and concurrent neuropsychiatric disorders. Cases of TRD may also be categorized based on the medications to which patients are resistant (e.g.: SSRI-resistant). In TRD, the clinical benefits and quality of life improvement achieved by adding further treatments such as psychotherapy, lithium, or atypical antipsychotics is weakly supported as of 2020.

Thus, to date, treatments for disorders such as MDD and TRD (and other disorders similar to MDD, such as Persistent Depressive Disorder, Postpartum Depression Disorder, and Social Anxiety Disorder, among others) are suboptimal. Recently, treatments (other than those described above) have been proposed for treating isolated symptoms affecting mood (such as the isolated symptom of depression).

For example, the present inventors have previously disclosed that dextromethadone can be used to treat the symptoms of pain and addiction (see U.S. Pat. No. 6,008,258) and can be used to treat select isolated psychological and/or psychiatric symptoms (see U.S. Pat. No. 9,468,611), in that select enantiomers of molecules presently included in the opioid class and their derivatives modulate NMDARs at doses and or concentrations that do not have clinically meaningful opioid receptor effects and that these select enantiomers may be therapeutic for pain and isolated psychiatric symptoms.

However, MDD is a defined disorder that is more complex and grave, as a pathological entity, than an isolated psychiatric symptom (such as the isolated symptom of depression). As noted above, there is agreement among experts that isolated psychiatric symptoms do not define neuropsychiatric disorders, and that the treatment of isolated symptoms does not translate to affecting the course of clinical neuropsychiatric disorders. Treatments for isolated symptoms of depression (such as those in U.S. Pat. No. 9,468,611) are thus not viewed as translatable to treating MDD, and so have not been used to treat MDD. Furthermore, the improvement of mood in the absence of an improvement in the disorder may not affect improvements in motivation, cognition, social and work abilities, or sleep.

In that regard, DSM-5 defines a neuropsychiatric disorder as “a syndrome characterized by clinically significant disturbance in an individual's cognition, emotion regulation, or behavior that reflects a dysfunction in the psychological, biological, or developmental processes underlying mental functioning.” The final draft of ICD-11 (the subsequent version to ICD-10) contains a very similar definition. There is agreement among experts that isolated psychiatric symptoms do not define neuropsychiatric disorders as defined by DSM5 and ICD-11. Psychiatric symptoms, for example, could be isolated traits of the individual rather than an actual part of diseases or disorders. Furthermore, psychiatric symptoms could be due to other primary disorders, e.g., fatigue in patients with cancer or anemia, or anxiety in patients with pheochromocytoma, or depressed mood in patients with hypothyroidism. Additionally, the treatment of isolated symptoms is not necessarily expected to impact on the course of neuropsychiatric disorders. As such, to date, treatments for isolated psychiatric symptoms (e.g., treatments for the isolated symptom of depression) have never been seen as translatable to neuropsychiatric disorders (e.g., MDD) because, while such treatments can alleviate a symptom (such as a symptom of depression), they are not seen as having a therapeutic effect on the course of a defined neuropsychiatric disorder. To date there is no treatment for MDD that has shown to have a therapeutic effect on its course.

As mentioned above, MDD is believed to be caused by a combination of genetic and environmental factors. The genetic+environmental paradigm (G+E) is becoming increasingly complex for neuropsychiatric disorders. To date, over 100 independent genetic variants have been linked to an increased risk for developing MDD [Howard D M, Adams M J, Clarke T K, Hafferty J D, Gibson J, Shirali M, et al. (March 2019), “Genome-wide meta-analysis of depression identifies 102 independent variants and highlights the importance of the prefrontal brain regions”, Nature Neuroscience, 22 (3): 343-352.]. Some of these variants may include genetic abnormalities in ion channels, including NMDARs. MDD has been linked to (1) neuronal loss and atrophy in select brain areas, including the mesial prefrontal cortex (mPFC) and the hippocampus [Kempton M J, Salvador Z, Munafò MR, Geddes J R, Simmons A, Frangou S, Williams S C (2011), “Structural neuroimaging studies in major depressive disorder. Meta-analysis and comparison with bipolar disorder”, Archives of General Psychiatry, 68 (7): 675-690], and (2) altered neuronal circuits (Korgaonkar M S, Goldstein-Piekarski A N, Fornito A, Williams L M. Intrinsic connectomes are a predictive biomarker of remission in major depressive disorder, Mol Psychiatry, 2019 Nov. 6). Furthermore, MDD is associated with increased cardiovascular risk, cancer and obesity (Howard et al., 2019). These associated and/or linked diseases, the laboratory indicators of systemic inflammation, and the imaging suggesting structural brain changes (neuronal atrophy and apoptosis) cited above, are part of a disorder that goes well beyond individual symptoms, and this disorder is unlikely to improve substantially with a purely symptomatic treatment. Available treatments, including SSRIs, SNRI, bupropion, atypical antipsychotics, have not been shown to influence disease course. SSRIs, SNRI, bupropion, and atypical antipsychotics have shown similar effects when administered earlier or later in the course of the disease, and this is a characteristic indicative of symptomatic treatments (whereas a treatment with the potential for favorably altering the course of a disease by remediating its pathogenetic mechanism—a disease-modifying treatment—is instead more effective when administered early in the course of the disease).

Thus, MDD and TRD and other neuropsychiatric disorders are not defined solely by the presence of symptoms such as depression, anxiety, fatigue, and mood instability. While the symptoms of depression, anxiety, fatigue, and mood instability may be integral to the diagnosis of MDD and TRD, depressed mood alone is not sufficient for the diagnosis of MDD. And so, a drug that symptomatically improves depressed mood, and has no other effect, may not impact significantly on the course of MDD, TRD, or other neuropsychiatric disorders. Effective disease-modifying treatment of neuropsychiatric disorders, including MDD and other diseases and disorders requires a drug that has effects that go beyond symptomatic treatment of one or more psychiatric symptoms. Such a disease-modifying treatment would be highly desirable, but to date such a treatment is unknown. Even for the recently approved drug esketamine, which is limited to TRD due to cognitive and other side effects, a disease modifying effect has not been demonstrated.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

As described above, current treatments for MDD and other neuropsychiatric disorders are inadequate. The effectiveness of current drug regimens is highly unpredictable, and attempts to direct modulation of neurotransmitter receptors and pathways in MDD—as well as in other chronic disorders such as chronic pain disorders, anxiety disorders, and other neuropsychiatric disorders including schizophrenia—have been disappointing. Among the issues noted are (1) that current drugs used to target neuronal circuit dysfunction may trigger feedback molecular actions that cause or aggravate neuropsychiatric symptoms and disorders; (2) these drugs may also interfere with non-dysfunctional neuronal circuits within the same neurotransmitter pathway; (3) that the non-selectiveness of action of current drugs results in effects on tissues outside the nervous system, causing additional side effects; (4) that current drugs may alter the function of a dysfunctional circuit in a way that improves symptoms, but does not act on the primary cause of dysfunction; (5) that patients may experience withdrawal upon discontinuation of currently used drugs; and (6) that patients may actually experience a worsening of symptoms upon discontinuation of currently used drugs.

Further, as described above, while there are treatments for individual symptoms (such as the isolated symptom of depression), such treatments (e.g., compounds and/or compositions for symptomatic treatment) are not considered useful for treatment of disorders such as MDD. For example, while certain drugs that have positive effect on the isolated symptom of depression have been shown to have favorable safety, tolerability and pharmacokinetic profiles (see Bernstein G, Davis K, Mills C, Wang L, McDonnell M, Oldenhof J, et al. Characterization of the safety and pharmacokinetic profile of D-methadone, a novel N-methyl-D-aspartate receptor antagonist in healthy, opioid-naive subjects: results of two phase 1 studies. J Clin Psychopharmacol. 2019; 39:226-37), there has been no teaching or suggestion of efficacy of such drugs for MDD, or any neuropsychiatric disorder, and no teaching or suggestion about efficacy for MDD in the absence of cognitive side effects

And while yet further studies have shown that, in animal models of depressive-like behavior, a drug like dextromethadone induces rapid antidepressant actions through mTORC1-mediated synaptic plasticity in the mPFC similar to ketamine (see e.g., Fogaça MV, Fukumoto K, Franklin T, et al. N-Methyl-D-aspartate receptor antagonist d-methadone produces rapid, mTORC1-dependent antidepressant effects. Neuropsychopharmacology. 2019; 44(13):2230-2238), these findings are limited to an attempt to explain improvements in experimentally induced depressive-like behavior in murine models. But this has never been seen as translatable to neuropsychiatric disorders like MDD because these murine models of depressive-like behavior are used to determine the potential for chemicals to exert behavioral improvement that could potentially translate into antidepressant effects in humans; and that would only be indicative of a drug that is useful for isolated symptoms of depression (which as noted above is separate from the clinical disorder of MDD, and treatments are not seen as translatable between the two).

Aspects of the present invention, however, reduce and/or eliminate issues with present treatments for MDD and other such disorders. In general, an overarching aspect of the present invention provides a disease-modifying treatment for MDD and other disorders. A “disease-modifying” treatment, or a treatment with “disease-modifying” potential, as used herein, includes a drug treatment with the potential for favorably altering the course of an illness by remediating its pathogenetic mechanism. A disease-modifying treatment is therefore potentially curative. In contrast, symptomatic treatments are generally only palliative—they alleviate symptoms, but do not directly address the molecular cause of the disease.

Herein, in discussing the novel disease-modifying treatment developed by the present inventors, both the terms “disease” and “disorder” may be used. In general, a “disease” has a defined (or better defined) pathophysiology, whereas in a “disorder” an explanation of pathophysiology is deficient or lacking. MDD (and other disorders discussed herein) are defined by those skilled in the art as a “disorder” or “disorders” because a clear explanation of pathophysiology is lacking. However, the work of the present inventors (disclosed herein) has for the first time elucidated the pathophysiology of MDD (in general—that excessive Ca2+ influx via NMDARs (e.g., tonically active NMDARs containing GluN2C and GluN2D subunits) in neurons that are part of certain circuits (e.g., the endorphin circuit), and that this excessive influx directly impairs neural plasticity (e.g., production of synaptic proteins such as the GluN1 subunit and other NMDAR subunits) necessary to form neuronal connections (e.g., “healthy” emotional memory that can replace pathological emotional memory). With the elucidation of this pathophysiology by the present inventors, though the Examples presented in this application, MDD (and other disorders that share a similar pathophysiology) could now be considered a disease rather than a disorder. And so, both terms “disease” and “disorder” may be used interchangeably herein when discussing these maladies.

And so, one aspect of the present invention is directed to a method of treating a neuropsychiatric disorder, the method including administering a composition to a subject suffering from a neuropsychiatric disorder, wherein the composition includes a substance to treat the disorder (in a manner that exhibits disease-modifying effects). In this aspect, the substance may be selected from dextromethadone, dextromethadone metabolites, d-methadol, d-alpha-acetylmethadol, d-alpha-normethadol, I-alpha-normethadol, and pharmaceutically acceptable salts thereof. The neuropsychiatric disorder to be treated may be selected from (but is not limited to) Major Depressive Disorder, Persistent Depressive Disorder, Disruptive Mood Dysregulation Disorder, Premenstrual Dysphoric Disorder, Postpartum Depression Disorder, Bipolar Disorder, Hypomania and Mania disorder, Generalized Anxiety Disorder, Social Anxiety Disorder, Somatic Symptom Disorder, Bereavement Depressive Disorder, Adjustment Depressive Disorder, Post-traumatic Stress Disorder, Obsessive Compulsive Disorder, Chronic Pain Disorder, Substance Use Disorder and Overactive Bladder Disorder.

Another aspect of the present invention is directed to a method for treating a neuropsychiatric disorder, the method including (1) diagnosing an individual with a neuropsychiatric disorder, (2) developing a course of treating the neuropsychiatric disorder of the individual, and (3) administering a substance to the individual as at least part of said course of treating the neuropsychiatric disorder of the individual. In this aspect, the substance may be chosen from dextromethadone, dextromethadone metabolites, d-methadol, d-alpha-acetylmethadol, d-alpha-normethadol, I-alpha-normethadol, and pharmaceutically acceptable salts thereof. The neuropsychiatric disorder to be treated may be selected from (but is not limited to) Major Depressive Disorder, Persistent Depressive Disorder, Disruptive Mood Dysregulation Disorder, Premenstrual Dysphoric Disorder, Postpartum Depression Disorder, Bipolar Disorder, Hypomania and Mania disorder, Generalized Anxiety Disorder, Social Anxiety Disorder, Somatic Symptom Disorder, Bereavement Depressive Disorder, Adjustment Depressive Disorder, Post-traumatic Stress Disorder, Obsessive Compulsive Disorder, Chronic Pain Disorder, Substance Use Disorder and Overactive Bladder Disorder.

One embodiment of this aspect of the invention may include a method for treating MDD including (1) diagnosing an individual with MDD, (2) developing a course of treating the MDD of the individual, and (3) administering dextromethadone to the individual as at least part of the course of treating the MDD of the individual.

Another aspect of the present invention is directed to a method of treating a neuropsychiatric disorder, the method including inducing the synthesis and the membrane expression in a subject of NMDAR subunits, AMPAR subunits, or other synaptic proteins that contribute to neuronal plasticity and assembled NMDAR channels. The subject, in this aspect, suffers from a neuropsychiatric disorder (examples of such neuropsychiatric disorders include Major Depressive Disorder, Persistent Depressive Disorder, Disruptive Mood Dysregulation Disorder, Premenstrual Dysphoric Disorder, Postpartum Depression Disorder, Bipolar Disorder, Hypomania and Mania disorder, Generalized Anxiety Disorder, Social Anxiety Disorder, Somatic Symptom Disorder, Bereavement Depressive Disorder, Adjustment Depressive Disorder, Post-traumatic Stress Disorder, Obsessive Compulsive Disorder, Chronic Pain Disorder, Substance Use Disorder and Overactive Bladder Disorder). In this aspect of the invention, inducing the synthesis of NMDAR subunits, AMPAR subunits, or other synaptic proteins that contribute to neuronal plasticity is accomplished by administering to the subject a substance selected from d-methadone, d-methadone metabolites, d-methadol, d-alpha-acetylmethadol, d-alpha-normethadol, I-alpha-normethadol, and pharmaceutically acceptable salts thereof.

Another aspect of the present invention is directed to a method for treating a disease or disorder characterized by a dysfunction of ion channels, the method including (1) diagnosing an individual with a disease or disorder characterized by a dysfunction of ion channels, (2) developing a course of treating the disease or disorder of the individual, wherein the course of treating the disease or disorder involves resolution of the dysfunction of ion channels, and (3) administering a substance to the individual as at least part of the course of resolving the dysfunction of ion channels. The substance used may be chosen from dextromethadone, dextromethadone metabolites, d-methadol, d-alpha-acetylmethadol, d-alpha-normethadol, I-alpha-normethadol, and pharmaceutically acceptable salts thereof.

Another aspect of the present invention is directed to a method for diagnosing a disorder as a disease caused, worsened, or maintained by pathologically hyperactive NMDAR channels. The method of this aspect includes administering a composition to a subject that has been diagnosed with at least one disorder of unclear pathophysiology chosen from neurological disorders, neuropsychiatric disorders, ophthalmic disorders, otologic disorders, metabolic disorders, osteoporosis, urogenital disorders, renal impairment, infertility, premature ovarian failure, liver disorders, immunological disorders, oncological disorders, cardiovascular disorders. The composition includes a substance selected from dextromethadone, dextromethadone metabolites, d-methadol, d-alpha-acetylmethadol, d-alpha-normethadol, l-alpha-normethadol, and pharmaceutically acceptable salts thereof. One then determines the effectiveness of the composition in the at least one disorder by measuring endpoints specific for each disorder before and after the administration of the composition, and diagnoses the subject with a disorder caused, worsened, or maintained by pathologically hyperactive NMDAR channels if the subject exhibits improvement of specific endpoints. As the endpoints may be specific to a particular disorder, the measurement of the endpoints following administration of the composition allows one to determine the particular disorder to be diagnosed.

Based on the determination described above, it is possible to diagnose a disorder as caused by excessive Ca²⁺ influx via NMDARs in certain brain cells. The disorder may be chosen from neurological disorders, neuropsychiatric disorders, ophthalmic disorders, otologic disorders, metabolic disorders, osteoporosis, urogenital disorders, including overactive bladder disorder, renal impairment, infertility, premature ovarian failure, liver disorders, immunological disorders, oncological disorders, cardiovascular disorders, including arrhythmias, heart failure and angina, inflammatory disorders and other disease and disorders triggered, maintained or worsened by pathologically hyperactivated NMDARs.

In support of these and other aspects of the present invention, the present inventors now disclose for the first time that dextromethadone has rapid, robust, sustained, and statistically significant efficacy, with a large effect size, for MDD (and thus potentially for other neuropsychiatric disorders and TRD), without cognitive side effects at MDD-effective doses. Discussion and data demonstrating this is shown below in the Examples, (and particularly in Example 3), and only the data in the Examples of this application allow for the conclusion that dextromethadone could have disease-modifying effects on neuropsychiatric disorders such as MDD. The present inventors have also determined that dextromethadone induces this sustained therapeutic response without side effects and without evidence of withdrawal or rebound, signaling a previously unrecognized specific disease-modifying mechanism of action.

Regarding this novel discovery and disclosure of the present inventors that dextromethadone has rapid, robust, sustained, and statistically significant efficacy with a large effect size for patients with a diagnosis of MDD and/or TRD: As will be described in greater detail below, the inventors disclose a double-blind, placebo-controlled, prospective, randomized, clinical trial that shows that dextromethadone can induce remission of disease in over 30% of patients who had failed on prior antidepressant treatments, compared to a remission rate of 5% in patients randomized to placebo (disease remission defined as a MADRS score of 10 or less; the MADRS rating scale measures not only depressed mood but also provides measures for motivation, cognition-ability to concentrate, sleep, appetite, social abilities, and suicidal risk). Further, this remission occurred within the first week of treatment, with improvements seen as early as day two and with statistical significance reached by day four. Notably, the remission persisted for at least one week after discontinuation of treatment, and likely longer for some patients. No withdrawal or even rebound signs or symptoms were present, as accurately measured with ad hoc scales described in Example 3.

As a general rule (as described above), the effects of symptomatic drugs for chronic conditions will rapidly decrease or abruptly cease after discontinuation of the drug (especially after abrupt discontinuation); and the abrupt discontinuation of symptomatic drugs may even result in the phenomenon of withdrawal symptoms and signs, and even augmentation of symptoms (i.e., worsening of symptoms compared to pre-treatment baseline). Contrary to this, the present inventors have now discovered that improvements from dextromethadone persisted upon completion of the treatment cycle, signaling for the first time disease-modifying effects of dextromethadone. The fact that the remission induced by dextromethadone in patients with MDD persists after discontinuation of treatment signals that the action of dextromethadone is not purely symptomatic, i.e., dextromethadone does not simply lift the mood of patients, an effect that would cease upon discontinuation of the drug (as happens, for example, with the use of opioids or alcohol, and even with the use of all presently approved standard antidepressant treatments). Thus, this persistence of disease remission suggests a previously unrecognized disease-modifying mechanism of action for dextromethadone (e.g., a primary effect on modulation of neuroplasticity, which persists beyond discontinuation of treatment), rather than a mere symptomatic treatment.

This discovery by the inventors creates aspects of the present invention directed to the use of dextromethadone for the therapeutic disease-modifying treatment of MDD, as well as for other neuropsychiatric diseases (as opposed to symptomatic treatment). As described above, the treatment of isolated symptoms is not necessarily expected to impact on the course of neuropsychiatric disorders. The genetic+environmental paradigm (G+E) is becoming increasingly complex for neuropsychiatric disorders. Insofar, over 100 independent genetic variants have been linked to an increased risk for developing MDD (Howard D M et al., 2019). Some of these variants may include genetic abnormalities in ion channels, including NMDARs. Furthermore, MDD and TRD have been found to be linked to inflammatory states [Milenkovic V M, Stanton E H, Nothdurfter C, Rupprecht R, Wetzel C H, The Role of Chemokines in the Pathophysiology of Major Depressive Disorder, Int J Mol Sci. 2019; 20(9):2283; Ho et al., 2017]. By modulating inflammation, dextromethadone may impact on the course of the disorder (i.e., exhibit disease/disorder-modifying effects now elucidated for the first time by the present inventors).

MDD has been linked to neuronal loss and atrophy in select brain areas, including the mesial prefrontal cortex (mPFC) and the hippocampus (Kempton et al. 2011), and has been linked to altered neuronal circuits (Korgaonkar et al., 2019). Furthermore, MDD is associated with increased cardiovascular risk, cancer, and obesity (Howard et al., 2019). These associated and/or linked diseases, the laboratory indicators of systemic inflammation, and the imaging suggesting structural brain changes (neuronal atrophy and apoptosis) cited above, are unlikely to improve with a purely symptomatic treatment. All of the above, including linked diseases, immunological abnormalities, and structural CNS deficits (both at the level of reversible neuronal circuitry failure or at the level of irreversible neuronal apoptosis) could instead be improved or cured by a disease-modifying treatment like dextromethadone, as now strongly signaled by the data shown in the Examples below (and particularly in the data shown and discussed in Example 3).

Furthermore, with a multiplicity of different endogenous neurotransmitter/receptor systems, the manipulation of one neurotransmitter system (or even of a handful of neurotransmitter systems) may modulate the function of a dysfunctional circuit and this modulation may improve target symptoms as is postulated for some of the drugs currently in clinical use. However, the drug is unlikely to act on the primary cause of the dysfunction for that circuit (e.g., NMDAR hyperactivity), and is thus unlikely to restore physiological cellular and circuit functions. In other words, the dysfunctional cell that triggered and maintained the disorder will continue to be dysfunctional, despite changes in surrounding levels of neurotransmitters (this is due to biofeedback mechanisms triggered by increased neurotransmitter levels; and so, these symptomatic treatments, while initially apparently helpful, may instead ultimately worsen the disease or disorder they were supposed to improve). As described above, fluoxetine and other drugs categorized as SSRIs for MDD are examples of such neurotransmitter pathway modulating drugs for the serotonin/5-HT receptor system. In clinical trials, they have typically shown a weak effect size and delayed and often incomplete and/or un-sustained efficacy (furthermore, upon discontinuation of SSRIs, patients are likely to experience withdrawal symptoms, as happens with most drugs that directly influence neurotransmitter concentrations and the pathways modulated by these neurotransmitters). And so, as has been described, these current treatments do not exhibit disease-modifying effects. Yet, to date, those skilled in the art continue to use such drugs because no more effective treatments have been discovered or disclosed.

However, based on the new data disclosed herein, the present inventors are able to now disclose the potential curative effects of dextromethadone both as adjunctive treatment or as monotherapy. In that regard, the present inventors disclose that the effects of dextromethadone were very robust in patients with MDD and concurrent antidepressant treatment, signaling the potentially curative actions of dextromethadone not only for the CNS abnormalities associated with MDD but also for CNS abnormalities possibly associated with MDD treatments. In other words, the down-regulation exerted by dextromethadone on excessive Ca²⁺ influx in select neurons with pathologically hyperactive NMDARs is likely to occur with or without concurrent neuropharmacological treatment and in disorders or diseases where the hyperactivity of NMDARs is primary or secondary to a variety of triggers, including treatment with antidepressants.

In light of the results of the present inventors' studies, which are presented in the Examples below, the present inventors disclose that dextromethadone can be used as a disease-modifying treatment for MDD in patients receiving antidepressant treatments (and having inadequate response to those treatments), and also disclose that the selective regulatory actions of dextromethadone on excessive Ca²⁺ influx may be useful for patients who have not yet received treatments that potentially may alter CNS neurotransmitter pathways (dextromethadone as the initial disease-modifying therapeutic agent, i.e., dextromethadone monotherapy for neuropsychiatric disorders). Furthermore, the inventors disclose that dextromethadone and behavioral psychotherapy may be successfully combined in the treatment of MDD and related disorders: e.g., certain patients may be receptive to psychotherapy only after downregulation of excessive NMDAR activity (i.e., after downregulation of pathologically open NMDAR channels with excessive Ca²⁺ influx).

The present inventors' uncovering of the full potential of dextromethadone therapy as an NMDAR ion channel modulator represents a paradigm shift in the molecular understanding of a multiplicity of neuropsychiatric diseases and disorders, including MDD, and thus for the treatment of a multiplicity of disorders and diseases, extending the therapeutic preventive and diagnostic clinical and research armamentarium beyond presently available symptomatic neuropsychiatric drugs to disease modifying drugs addressing the molecular pathophysiology. Downregulation of excessive Ca²⁺ influx in cells (neurons or other cells) that are part of a select CNS circuitry (or extra CNS tissue) will allow cells to return to function and to autoregulate amounts of neurotransmitter synthesis (and other synaptic and extrasynaptic proteins) and their membrane expression (including synaptic scaffolding and framework) and/or release (e.g., NGF, including BDNF).

This fine regulation is virtually impossible when neurotransmitters or agonist/antagonist drugs for select receptors (e.g., drugs agonist at dopamine, GABA, opioid receptors) are directly modulated by drugs. While drugs directly targeting receptors may be very effective for acute treatment of many symptoms (e.g., opioids for acute pain, benzodiazepines for panic attacks and dopamine blockers for psychotic events), and while their short term side effects are well-understood and accepted, these same drugs are less effective and their long-term effects are less understood and less predictable and thus their uses can not only fail to cure the disease but also be detrimental when the treatments are chronic. The chronic treatment with opioids for chronic pain, or with benzodiazepines for chronic disorders (e.g., GAD, PTSD, OCD) where anxiety is prominent, or dopamine blockers for chronic management of psychotic conditions, generally results in severe and sometimes irreversible side effects, including worsening of the primary disorder. The new data regarding dextromethadone disclosed by the inventors herein, as well as the newly revealed mechanism of action of dextromethadone herein, allow for a better targeted treatment of disorders such as MDD, MDD related disorders, other neuropsychiatric diseases, and even extra CNS diseases.

These and other advantages of the application will be apparent to those of skill in the art with reference to the drawings and the detailed description below.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a graph showing L-glutamic acid CRC in the presence of 10 μM glycine for cell lines GluN2A, GluN2B, GluN2C, and GluN2C. Data are reported as mean±SEM, n=5.

FIG. 2A is a graph showing the 100 nm L-Glutamate Effect on GluN2A.

FIG. 2B is a graph showing the 100 nm L-Glutamate Effect on GluN2B.

FIG. 2C is a graph showing the 100 nm L-Glutamate Effect on GluN2C.

FIG. 2D is a graph showing the 100 nm L-Glutamate Effect on GluN2D.

FIG. 2E is a graph showing the 100 nm L-Glutamate Effect on GluN2C (cells with low expression level).

FIG. 3A is a graph showing the effect of dextromethadone on L-glutamate concentration response curve (CRC) in receptor type GluN1-GluN2A.

FIG. 3B is a graph showing the effect of dextromethadone on L-glutamate CRC in receptor type GluN1-GluN2B.

FIG. 3C is a graph showing the effect of dextromethadone on L-glutamate CRC in receptor type GluN1-GluN2C.

FIG. 3D is a graph showing the effect of dextromethadone on L-glutamate CRC in receptor type GluN1-GluN2D.

FIG. 4A is a graph showing the effect of memantine on L-glutamate CRC in receptor type GluN1-GluN2A.

FIG. 4B is a graph showing the effect of memantine on L-glutamate CRC in receptor type GluN1-GluN2B.

FIG. 4C is a graph showing the effect of memantine on L-glutamate CRC in receptor type GluN1-GluN2C.

FIG. 4D is a graph showing the effect of memantine on L-glutamate CRC in receptor type GluN1-GluN2D.

FIG. 5A is a graph showing the effect of (±)-ketamine on L-glutamate CRC in receptor type GluN1-GluN2A.

FIG. 5B is a graph showing the effect of (±)-ketamine on L-glutamate CRC in receptor type GluN1-GluN2B.

FIG. 5C is a graph showing the effect of (±)-ketamine on L-glutamate CRC in receptor type GluN1-GluN2C.

FIG. 5D is a graph showing the effect of (±)-ketamine on L-glutamate CRC in receptor type GluN1-GluN2D.

FIG. 6A is a graph showing the effect of (±)-MK 801 on L-glutamate CRC in receptor type GluN1-GluN2A.

FIG. 6B is a graph showing the effect of (±)-MK 801 on L-glutamate CRC in receptor type GluN1-GluN2B.

FIG. 6C is a graph showing the effect of (±)-MK 801 on L-glutamate CRC in receptor type GluN1-GluN2C.

FIG. 6D is a graph showing the effect of (±)-MK 801 on L-glutamate CRC in receptor type GluN1-GluN2D.

FIG. 7A is a graph showing the effect of dextromethorphan on L-glutamate CRC in receptor type GluN1-GluN2A.

FIG. 7B is a graph showing the effect of dextromethorphan on L-glutamate CRC in receptor type GluN1-GluN2B.

FIG. 7C is a graph showing the effect of dextromethorphan on L-glutamate CRC in receptor type GluN1-GluN2C.

FIG. 7D is a graph showing the effect of dextromethorphan on L-glutamate CRC in receptor type GluN1-GluN2D.

FIG. 8A is a graph showing the % effect of dextromethadone on 4.6 nM L-glutamate for receptor subtypes GluN2A, GluN2B, GluN2C, and GluN2D.

FIG. 8B is a graph showing the % effect of dextromethadone on 14 nM L-glutamate for receptor subtypes GluN2A, GluN2B, GluN2C, and GluN2D.

FIG. 8C is a graph showing the % effect of dextromethadone on 41 nM L-glutamate for receptor subtypes GluN2A, GluN2B, GluN2C, and GluN2D.

FIG. 8D is a graph showing the % effect of dextromethadone on 123 nM L-glutamate for receptor subtypes GluN2A, GluN2B, GluN2C, and GluN2D.

FIG. 8E is a graph showing the % effect of dextromethadone on 370 nM L-glutamate for receptor subtypes GluN2A, GluN2B, GluN2C, and GluN2D.

FIG. 8F is a graph showing the % effect of dextromethadone on 1.1 μM L-glutamate for receptor subtypes GluN2A, GluN2B, GluN2C, and GluN2D.

FIG. 8G is a graph showing the % effect of dextromethadone on 3.3 μM L-glutamate for receptor subtypes GluN2A, GluN2B, GluN2C, and GluN2D.

FIG. 8H is a graph showing the % effect of dextromethadone on 10 μM L-glutamate for receptor subtypes GluN2A, GluN2B, GluN2C, and GluN2D.

FIG. 8I is a graph showing the % effect of dextromethadone on 100 μM L-glutamate for receptor subtypes GluN2A, GluN2B, GluN2C, and GluN2D.

FIG. 8J is a graph showing the % effect of dextromethadone on 1 mM L-glutamate for receptor subtypes GluN2A, GluN2B, GluN2C, and GluN2D.

FIG. 9A is a graph showing the % effect of (±)-ketamine on 4.6 nM L-glutamate for receptor subtypes GluN2A, GluN2B, GluN2C, and GluN2D.

FIG. 9B is a graph showing the % effect of (±)-ketamine on 14 nM L-glutamate for receptor subtypes GluN2A, GluN2B, GluN2C, and GluN2D.

FIG. 9C is a graph showing the % effect of (±)-ketamine on 41 nM L-glutamate for receptor subtypes GluN2A, GluN2B, GluN2C, and GluN2D.

FIG. 9D is a graph showing the % effect of (±)-ketamine on 123 nM L-glutamate for receptor subtypes GluN2A, GluN2B, GluN2C, and GluN2D.

FIG. 9E is a graph showing the % effect of (±)-ketamine on 370 nM L-glutamate for receptor subtypes GluN2A, GluN2B, GluN2C, and GluN2D.

FIG. 9F is a graph showing the % effect of (±)-ketamine on 1.1 μM L-glutamate for receptor subtypes GluN2A, GluN2B, GluN2C, and GluN2D.

FIG. 9G is a graph showing the % effect of (±)-ketamine on 3.3 μM L-glutamate for receptor subtypes GluN2A, GluN2B, GluN2C, and GluN2D.

FIG. 9H is a graph showing the % effect of (±)-ketamine on 10 μM L-glutamate for receptor subtypes GluN2A, GluN2B, GluN2C, and GluN2D.

FIG. 9I is a graph showing the % effect of (±)-ketamine on 100 μM L-glutamate for receptor subtypes GluN2A, GluN2B, GluN2C, and GluN2D.

FIG. 9J is a graph showing the % effect of (±)-ketamine on 1 mM L-glutamate for receptor subtypes GluN2A, GluN2B, GluN2C, and GluN2D.

FIG. 10A is a graph showing the % effect of memantine on 14 nM L-glutamate for receptor subtypes GluN2A, GluN2B, GluN2C, and GluN2D.

FIG. 10B is a graph showing the % effect of memantine on 41 nM L-glutamate for receptor subtypes GluN2A, GluN2B, GluN2C, and GluN2D.

FIG. 10C is a graph showing the % effect of memantine on 123 nM L-glutamate for receptor subtypes GluN2A, GluN2B, GluN2C, and GluN2D.

FIG. 10D is a graph showing the % effect of memantine on 370 nM L-glutamate for receptor subtypes GluN2A, GluN2B, GluN2C, and GluN2D.

FIG. 10E is a graph showing the % effect of memantine on 1.1 μM L-glutamate for receptor subtypes GluN2A, GluN2B, GluN2C, and GluN2D.

FIG. 10F is a graph showing the % effect of memantine on 3.3 μM L-glutamate for receptor subtypes GluN2A, GluN2B, GluN2C, and GluN2D.

FIG. 10G is a graph showing the % effect of memantine on 10 μM L-glutamate for receptor subtypes GluN2A, GluN2B, GluN2C, and GluN2D.

FIG. 10H is a graph showing the % effect of memantine on 100 μM L-glutamate for receptor subtypes GluN2A, GluN2B, GluN2C, and GluN2D.

FIG. 10I is a graph showing the % effect of memantine on 1 mM L-glutamate for receptor subtypes GluN2A, GluN2B, GluN2C, and GluN2D.

FIG. 11A is a graph showing the % effect of dextromethorphan on 4.6 nM L-glutamate for receptor subtypes GluN2A, GluN2B, GluN2C, and GluN2D.

FIG. 11B is a graph showing the % effect of dextromethorphan on 14 nM L-glutamate for receptor subtypes GluN2A, GluN2B, GluN2C, and GluN2D.

FIG. 11C is a graph showing the % effect of dextromethorphan on 41 nM L-glutamate for receptor subtypes GluN2A, GluN2B, GluN2C, and GluN2D.

FIG. 11D is a graph showing the % effect of dextromethorphan on 123 nM L-glutamate for receptor subtypes GluN2A, GluN2B, GluN2C, and GluN2D.

FIG. 11E is a graph showing the % effect of dextromethorphan on 370 nM L-glutamate for receptor subtypes GluN2A, GluN2B, GluN2C, and GluN2D.

FIG. 11F is a graph showing the % effect of dextromethorphan on 1.1 μM L-glutamate for receptor subtypes GluN2A, GluN2B, GluN2C, and GluN2D.

FIG. 11G is a graph showing the % effect of dextromethorphan on 3.3 μM L-glutamate for receptor subtypes GluN2A, GluN2B, GluN2C, and GluN2D.

FIG. 11H is a graph showing the % effect of dextromethorphan on 10 μM L-glutamate for receptor subtypes GluN2A, GluN2B, GluN2C, and GluN2D.

FIG. 11I is a graph showing the % effect of dextromethorphan on 100 μM L-glutamate for receptor subtypes GluN2A, GluN2B, GluN2C, and GluN2D.

FIG. 11J is a graph showing the % effect of dextromethorphan on 1 mM L-glutamate for receptor subtypes GluN2A, GluN2B, GluN2C, and GluN2D.

FIG. 12A is a graph showing the % effect of (±)-MK801 on 4.6 nM L-glutamate for receptor subtypes GluN2A, GluN2B, GluN2C, and GluN2D.

FIG. 12B is a graph showing the % effect of (±)-MK801 on 14 nM L-glutamate for receptor subtypes GluN2A, GluN2B, GluN2C, and GluN2D.

FIG. 12C is a graph showing the % effect of (±)-MK801 on 41 nM L-glutamate for receptor subtypes GluN2A, GluN2B, GluN2C, and GluN2D.

FIG. 12D is a graph showing the % effect of (±)-MK801 on 123 nM L-glutamate for receptor subtypes GluN2A, GluN2B, GluN2C, and GluN2D.

FIG. 12E is a graph showing the % effect of (±)-MK801 on 370 nM L-glutamate for receptor subtypes GluN2A, GluN2B, GluN2C, and GluN2D.

FIG. 12F is a graph showing the % effect of (±)-MK801 on 1.1 μM L-glutamate for receptor subtypes GluN2A, GluN2B, GluN2C, and GluN2D.

FIG. 12G is a graph showing the % effect of (±)-MK801 on 3.3 μM L-glutamate for receptor subtypes GluN2A, GluN2B, GluN2C, and GluN2D.

FIG. 12H is a graph showing the % effect of (±)-MK801 on 10 μM L-glutamate for receptor subtypes GluN2A, GluN2B, GluN2C, and GluN2D.

FIG. 12I is a graph showing the % effect of (±)-MK801 on 100 μM L-glutamate for receptor subtypes GluN2A, GluN2B, GluN2C, and GluN2D.

FIG. 12J is a graph showing the % effect of (±)-MK801 on 1 mM L-glutamate for receptor subtypes GluN2A, GluN2B, GluN2C, and GluN2D.

FIG. 13A is a photograph showing expression of the NMDAR1 subunit in ARPE-19 cells.

FIG. 13B is a photograph showing expression of the NMDAR2A subunit in ARPE-19 cells.

FIG. 13C is a photograph showing expression of the NMDAR2B subunit in ARPE-19 cells.

FIG. 14 is a graph showing cell viability of ARPE-19 cells after treatment with the NMDAR agonist L-glutamate alone (10 mM L-Glu) or in combination with dextromethadone. ***P<0.001 versus control cells treated with vehicle (one-way ANOVA followed by Tukey's post hoc test).

FIG. 15A is a graph showing protein expression of the NMDAR1 subunit (control=untreated cells; acute=24-hour treatment; chronic=6-day treatment). Data are expressed as mean±SEM.

FIG. 15B is a graph showing protein expression of the NMDAR2A subunit (control=untreated cells; acute=24-hour treatment; chronic=6-day treatment). Data are expressed as mean±SEM.

FIG. 15C is a graph showing protein expression of the NMDAR2B subunit (control=untreated cells; acute=24-hour treatment; chronic=6-day treatment). Data are expressed as mean±SEM.

FIG. 16 is a graph showing hypothetic values for NR1 subunits at various glutamate concentrations.

FIG. 17 is a schematic showing the screening and dosing schedule for patients in a Phase 2 study of two doses of dextromethadone in patients with MDD.

FIG. 18 is a table of treatment-emergent adverse events—overall summary safety population.

FIGS. 19A and 19B combined provide a table of treatment-emergent adverse events by system organ class and preferred term safety population.

FIG. 20 is a table of adverse events of special interest (AESI) by system organ class and preferred term safety population.

FIG. 21 is a table of clinician administered dissociative states scale scores.

FIG. 22 is a graph showing plasma concentrations of dextromethadone by dose level (25 mg and 50 mg) at Day 1.

FIG. 23 is a graph showing trough plasma concentration levels of dextromethadone by dose level (25 mg and 50 mg).

FIG. 24 is a graph showing that MADRS scores in the treatment groups of the Phase 2 study achieved statistically significant difference versus placebo from Day 4 through Day 14.

FIG. 25 is a graph showing the percentage of remitters, with MADRS<10 points.

FIG. 26 is a graph showing the percentage of responders with MADRS>50% reduction from baseline.

FIG. 27A is a graph showing the effect of 10 μM gentamicin on 0.04 μM L-glutamate for a cell line expressing diheteromeric recombinant human NMDAR containing GluN1 plus GluN2A.

FIG. 27B is a graph showing the effect of 10 μM gentamicin on 0.04 μM L-glutamate for a cell line expressing diheteromeric recombinant human NMDAR containing GluN1 plus GluN2B.

FIG. 27C is a graph showing the effect of 10 μM gentamicin on 0.04 μM L-glutamate for a cell line expressing diheteromeric recombinant human NMDAR containing GluN1 plus GluN2C.

FIG. 27D is a graph showing the effect of 10 μM gentamicin on 0.04 μM L-glutamate for a cell line expressing diheteromeric recombinant human NMDAR containing GluN1 plus GluN2D.

FIG. 28A is a graph showing the effect of 10 μM gentamicin on 0.2 μM L-glutamate for a cell line expressing diheteromeric recombinant human NMDAR containing GluN1 plus GluN2A.

FIG. 28B is a graph showing the effect of 10 μM gentamicin on 0.2 μM L-glutamate for a cell line expressing diheteromeric recombinant human NMDAR containing GluN1 plus GluN2B.

FIG. 28C is a graph showing the effect of 10 μM gentamicin on 0.2 μM L-glutamate for a cell line expressing diheteromeric recombinant human NMDAR containing GluN1 plus GluN2C.

FIG. 28D is a graph showing the effect of 10 μM gentamicin on 0.2 μM L-glutamate for a cell line expressing diheteromeric recombinant human NMDAR containing GluN1 plus GluN2D.

FIG. 29A is a graph showing the effect of 10 μM gentamicin on 10 μM L-glutamate for a cell line expressing diheteromeric recombinant human NMDAR containing GluN1 plus GluN2A.

FIG. 29B is a graph showing the effect of 10 μM gentamicin on 10 μM L-glutamate for a cell line expressing diheteromeric recombinant human NMDAR containing GluN1 plus GluN2B.

FIG. 29C is a graph showing the effect of 10 μM gentamicin on 10 μM L-glutamate for a cell line expressing diheteromeric recombinant human NMDAR containing GluN1 plus GluN2C.

FIG. 29D is a graph showing the effect of 10 μM gentamicin on 10 μM L-glutamate for a cell line expressing diheteromeric recombinant human NMDAR containing GluN1 plus GluN2D.

FIG. 30 is a graph showing a quinolinic acid CRC plot for each of the four NMDA receptor subtypes (GluN2A, GluN2B, GluN2C, and GluN2D).

FIG. 31 is a graph showing a gentamicin CRC plot for each of the four NMDA receptor subtypes (GluN2A, GluN2B, GluN2C, and GluN2D).

FIG. 32A is a graph showing the effect of 100 μM-1,000 μM of quinolinic acid, and quinolinic acid with the addition of 10 μM dextromethadone, in the presence of 10 μM glycine, using GluN2A.

FIG. 32B is a graph showing the effect of 100 μM-1,000 μM of quinolinic acid, and quinolinic acid with the addition of 10 μM dextromethadone, in the presence of 10 μM glycine, using GluN2B.

FIG. 32C is a graph showing the effect of 100 μM-1,000 μM of quinolinic acid, and quinolinic acid with the addition of 10 μM dextromethadone, in the presence of 10 μM glycine, using GluN2C.

FIG. 32D is a graph showing the effect of 100 μM-1,000 μM of quinolinic acid, and quinolinic acid with the addition of 10 μM dextromethadone, in the presence of 10 μM glycine, using GluN2D.

FIG. 33A is a graph showing the effect of 40 nM L-glutamate, and L-glutamate with the addition of 100 μM quinolinic acid and/or 10 μM dextromethadone, in the presence of 10 μM glycine, using GluN2A.

FIG. 33B is a graph showing the effect of 40 nM L-glutamate, and L-glutamate with the addition of 100 μM quinolinic acid and/or 10 μM dextromethadone, in the presence of 10 μM glycine, using GluN2B.

FIG. 33C is a graph showing the effect of 40 nM L-glutamate, and L-glutamate with the addition of 100 μM quinolinic acid and/or 10 μM dextromethadone, in the presence of 10 μM glycine, using GluN2C.

FIG. 33D a graph showing the effect of 40 nM L-glutamate, and L-glutamate with the addition of 100 μM quinolinic acid and/or 10 μM dextromethadone, in the presence of 10 μM glycine, using GluN2D.

FIG. 34A is a graph showing the effect of 40 nM L-glutamate, and L-glutamate with the addition of 1,000 μM quinolinic acid and/or 10 μM dextromethadone, in the presence of 10 μM glycine, using GluN2A.

FIG. 34B is a graph showing the effect of 40 nM L-glutamate, and L-glutamate with the addition of 1,000 μM quinolinic acid and/or 10 μM dextromethadone, in the presence of 10 μM glycine, using GluN2B.

FIG. 34C is a graph showing the effect of 40 nM L-glutamate, and L-glutamate with the addition of 1,000 μM quinolinic acid and/or 10 μM dextromethadone, in the presence of 10 μM glycine, using GluN2C.

FIG. 34D is a graph showing the effect of 40 nM L-glutamate, and L-glutamate with the addition of 1,000 μM quinolinic acid and/or 10 μM dextromethadone, in the presence of 10 μM glycine, using GluN2D.

FIG. 35A is a graph showing the effect of 200 nM L-glutamate, and L-glutamate with the addition of 100 μM quinolinic acid and/or 10 μM dextromethadone, in the presence of 10 μM glycine, using GluN2A.

FIG. 35B is a graph showing the effect of 200 nM L-glutamate, and L-glutamate with the addition of 100 μM quinolinic acid and/or 10 μM dextromethadone, in the presence of 10 μM glycine, using GluN2B.

FIG. 35C is a graph showing the effect of 200 nM L-glutamate, and L-glutamate with the addition of 100 μM quinolinic acid and/or 10 μM dextromethadone, in the presence of 10 μM glycine, using GluN2C.

FIG. 35D is a graph showing the effect of 200 nM L-glutamate, and L-glutamate with the addition of 100 μM quinolinic acid and/or 10 μM dextromethadone, in the presence of 10 μM glycine, using GluN2D.

FIG. 36A is a graph showing the effect of 200 nM L-glutamate, and L-glutamate with the addition of 1,000 μM quinolinic acid and/or 10 μM dextromethadone, in the presence of 10 μM glycine, using GluN2A.

FIG. 36B is a graph showing the effect of 200 nM L-glutamate, and L-glutamate with the addition of 1,000 μM quinolinic acid and/or 10 μM dextromethadone, in the presence of 10 μM glycine, using GluN2B.

FIG. 36C is a graph showing the effect of 200 nM L-glutamate, and L-glutamate with the addition of 1,000 μM quinolinic acid and/or 10 μM dextromethadone, in the presence of 10 μM glycine, using GluN2C.

FIG. 36D is a graph showing the effect of 200 nM L-glutamate, and L-glutamate with the addition of 1,000 μM quinolinic acid and/or 10 μM dextromethadone, in the presence of 10 μM glycine, using GluN2D.

FIG. 37A is a graph showing the effect of 1,000 μM quinolinic acid, and quinolinic acid with the addition of 10 g/ml gentamicin and/or 10 μM dextromethadone, in the presence of 10 μM glycine, using GluN2A.

FIG. 37B is a graph showing the effect of 1,000 μM quinolinic acid, and quinolinic acid with the addition of 10 g/ml gentamicin and/or 10 μM dextromethadone, in the presence of 10 μM glycine, using GluN2B.

FIG. 37C is a graph showing the effect of 1,000 μM quinolinic acid, and quinolinic acid with the addition of 10 g/ml gentamicin and/or 10 μM dextromethadone, in the presence of 10 μM glycine, using GluN2C.

FIG. 37D is a graph showing the effect of 1,000 μM quinolinic acid, and quinolinic acid with the addition of 10 g/ml gentamicin and/or 10 μM dextromethadone, in the presence of 10 μM glycine, using GluN2D.

FIGS. 38A-H are scatter dot plots of MDARS CFB, with FIGS. 38A-D being scatter dot plots of MDARS CFB at day 7 and 14 of patients treated with placebo or 25 mg of dextromethadone (REL-1017) (horizontal bars indicate median); and with FIGS. 38E-H being scatter dot plots of MDARS CFB at day 7 and 14 of patients treated with placebo or 50 mg of dextromethadone (REL-1017) (horizontal bars indicate median).

FIG. 39 is a chart showing a test item application protocol diagram.

FIG. 40 is a graph showing the effect of test items on L-Glutamate/Glycine elicited current through hGluN1/hGluN2C NMDAR.

FIG. 41 shows sample currents recorded in hGluN1/hGluN2C-CHO cells, showing representative current traces recorded from two different cells, added with 10/10 μM L-glutamate/glycine in the absence or in the presence of 10 μM dextromethadone (left) or 1 μM (±)-ketamine (right).

FIG. 42 includes graphs showing sample traces of test item onset and offset kinetic experiments for 10 μM dextromethadone treated cell (left), or 1 μM (±)-ketamine treated cell (right).

FIG. 43 is a graph showing a summary of test item onset kinetic experiments, where traces represent % current recorded for 10 μM dextromethadone (middle line; grey shading), 10 μM (±)-ketamine (bottom line; black shading), and 1 μM (±)-ketamine (top line; light grey shading), while internal black lines are relative fittings.

FIG. 44 is a graph showing a comparison of the tau-on of 10 μM dextromethadone (left column) and 1 μM (±)-ketamine (right column) experiments of Example 6, Part I.

FIG. 45 is a graph showing a summary of test item offset kinetic experiments, where traces represent % current recorded for 10 μM dextromethadone (grey shading), 1 μM (±)-ketamine (black shading) and 10 μM (±)-ketamine (light grey shading), while internal black lines are relative fittings.

FIG. 46 is a graph showing a comparison of the tau-off of 10 μM dextromethadone (left column) and 1 μM (±)-ketamine (right column) experiments.

FIG. 47 is a graph demonstrating that intracellular dextromethadone did not modify 10/10 μM L-glutamate/glycine induced current.

FIG. 48 is a graph demonstrating that intracellular dextromethadone did not increase current block by extracellular dextromethadone.

FIG. 49 is a chart showing a test item application protocol diagram.

FIG. 50 is a chart showing the effect of test item sample traces in a trapping assay.

FIGS. 51A-51C are graphs showing Block (FIG. 51A), Residual Block (FIG. 51B) and Block Trapped (FIG. 51C) produced by 10 μM dextromethadone (left columns in 51A-C) or 1 μM (±)-ketamine (right columns in 51A-C). Values are reported as mean±sem (n=13 for dextromethadone and n=11 for (±)-ketamine). Unpaired t-test was performed.

FIGS. 52A-52C are graphs showing gene expression of cytokines [IL-6 (FIG. 52A), IL-10 (FIG. 52B), and CCL2 (FIG. 52C)] involved in inflammation as measured by qRT-PCR in rat livers via standard diet, Western diet, and Western diet+d-methadone. **p<0.01, ***p<0.001 and ****p<0.0001; one-way ANOVA followed by Tukey's post hoc test.

FIGS. 53A-53C are photographs resulting from a histological analysis of liver tissue by hematoxylin-eosin staining of paraffine-embedded liver slices, demonstrating that rats fed with Standard diet show a normal liver architecture (FIG. 53A), whereas lipid accumulation leading to hepatic steatosis with the typical ballooning was observed in rats fed with Western diet (FIG. 53B, arrow), while a reduction of steatosis could be observed in the rats treated with d-methadone (FIG. 53C). Photographs at 10× magnification.

FIGS. 54A-54B are graphs showing expression of two genes [GPAT4 (FIG. 54A) and SREPB2 (FIG. 54B)] involved in lipid metabolism by qRT-PCR, and demonstrating that gene expression of both GPAT4 and SREPB2 was significantly increased by Western Diet administration, and d-methadone treatment was able to cause a significant drop of their expression. *p<0.05, **p<0.01, ***p<0.001 and ****p<0.0001; one-way ANOVA followed by Tukey's post hoc test.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As used herein, the terms dextromethadone; esmethadone; REL-1017; S-methadone; d-methadone; and (+)-methadone define the same chemical molecule and are interchangeable.

A “disease-modifying” treatment or a treatment with “disease-modifying” potential, as used herein, includes a drug treatment with the potential for favorably altering the course of an illness by remediating its pathogenetic molecular mechanism. A disease-modifying treatment is therefore potentially curative. In contrast, symptomatic treatments are generally only palliative, they alleviate symptoms but do not address the molecular cause of the disease.

In the case of dextromethadone and MDD, it is hypothesized by the present inventors that, at least for a subset of patients, MDD is caused by excessive Ca²⁺ influx via NMDARs in certain CNS cells, e.g., neurons or astrocytes that are part of the endorphin pathway. This excessive Ca²⁺ influx in these CNS cells activates the intracellular downstream signal that impairs the production of various synaptic proteins. The unavailability of these synaptic proteins then impedes the formation of neuronal connections (e.g., neuronal connections necessary for the formation of emotional memory) and causes the phenotype of depression in humans with MDD. This excessive Ca²⁺ entry is preferentially via NMDAR channels that contain NR2c and NR2D subunits during resting membrane potential (tonically and pathologically hyperactive NMDARs containing GluN2c and GluN2D subunits).

Dextromethadone, as disclosed by the inventors' carries a positive charge which renders it similar to Mg²⁺ in its voltage dependent NMDAR channel block, inserts itself in the pore of the NMDAR and (similarly to Mg²⁺) and down-regulates the excess Ca²⁺ influx. The reduction of previously excessive Ca²⁺ influx to physiological amounts activates downstream signaling that results in production of adequate amounts of synaptic proteins for constructing new “healthy” emotional memory in select brain circuitry. Thus, MDD is relieved through curative molecular mechanisms and not by relieving symptoms by simply acting directly for example on opioid receptors or even serotonin receptors as previously hypothesized for most drugs with effects on the isolated symptom of depression.

And so, dextromethadone is potentially curative, and thus disease-modifying, for MDD and related disorders, e.g., disorders caused by excessive Ca²⁺ in select CNS cell populations, including cells part of select circuits. In the case of MDD, the inventors disclose that the endorphin circuit is relevant and that the opioid affinity of dextromethadone may direct the molecule towards opioid receptors structurally associated with NMDARs (dual receptors, heteroreceptors) expressed by neurons part of the endorphin circuits. This binding to opioid receptors, disclosed by the inventors, does not result in typical opioid effects as has been believed to date by those of ordinary skill in the art. This lack of typical opioid effects at MDD-effective doses, previously unknown, is related to the structural association of these opioid receptors with NMDARs as detailed in the Examples below.

As used herein, “memory” includes cognitive memory, emotional memory, social memory, and motor memory. The terms “memory,” “learning,” (LTP)+(LTD),” “neural plasticity,” (“spine enlargement”+“spinogenesis”+“synaptic strengthening”+“neurite growth”+synaptic pruning) and “connectome” may be used interchangeably herein. Individuality and self-awareness are forms of memory. MDD and related disorders can be viewed as manifestations of pathological emotional memory.

As used herein, “synaptic framework” may include all elements present at neuronal synapses, including all receptors, including excitatory and inhibitory receptors, including ionotropic and metabotropic receptors. And including synaptic vesicles in presynaptic neurons. And including all elements of the post-synaptic density. And including synaptic cleft molecules, including adhesion proteins.

As used herein, “NMDAR framework” may include all elements of the glutamateregic system, including NMDAR subtype relative and absolute density, and location. It includes the framework of the synaptic “hotspot” (a 100-200 nanomolar diameter area on the membrane of the glutamate receiving cell, closest to the releasing glutamate area of the glutamate releasing cell). NMDAR subtypes may include NR1-2A-D di-heteromers and tri-heteromers including NR1-NR2A-D (e.g., NR1-2A-2B) and tri-heteromers NR1-2A-D-3 A-B (e.g., NR1-2D-3A or NR1-NR3A-NR2C) and di-heteromers NR1-NR3A-B. NMDAR membrane location may include synaptic (presynaptic and postsynaptic), perisynaptic, extrasynaptic, and on non-neuronal membranes, e.g., on astrocytes or extra CNS cell populations. Location may refer to specific areas within the brain and or specific neuronal circuits, including microcircuits, and or specific receptor systems (e.g., endorphin system). In some respects, the NMDAR framework is intended to include other glutamate receptors (e.g., AMPARs and Kainate receptors and metabotropic NMDARs).

As used herein, “Positive Allosteric Modulators (PAMs)” and “Negative Allosteric Modulators (NAMs)” refer to endogenous and exogenous ions and molecules (including endogenous and exogenous toxins, peptides, steroids (including hormones), and drugs and physical and chemical stimuli, that are capable of influencing the opening of ion channels including, in particular, the opening and closing of NMDARs. Gentamicin is included among allosteric modulators of the NMDAR. PAMs and NAMs can be noncompetitive when binding in proximity but not at the agonist site. Or, they can be uncompetitive when binding at a site distant from the agonist site, as is the case for dextromethadone and other channel pore blockers described herein.

As used herein, “agonist substances” refers to endogenous and exogenous molecules capable of influencing the opening of ion channels, including the opening and closing of NMDARs, by binding to the agonist sites of the NMDAR (including the NMDA site). Such molecules include toxins and drugs, and endogenous substances such as quinolinic acid.

As used herein, “epigenetic code” refers to a code for epigenetic instructions (some of which may be mediated via Cam-CaMKII, CREB, and m-ToR pathways) represented by differential patterns of precisely regulated Ca²⁺ influx via NMDARs that in turn regulate cellular select translation, synthesis, assembly of proteins and differentiation, migration, and neuronal plasticity, including the constant reshaping of the neuronal connectome, including regulation of the NMDAR framework itself (regulation of the regulator, in a real time constant self-learning paradigm). This epigenetic code consisting of precise and ever changing (subsequent stimuli determine a different pattern of Ca2+ influx) amounts of Ca²⁺ influx via NMDARs is shared by all species with NMDARs and NMDAR framework. These differential patterns of Ca²⁺ influx regulate and in turn are regulated by the NMDAR framework. The code (i.e., the differential patterns of Ca²⁺ influx) is shared within species with the same NMDAR subunits GluN1, GluN2A-D, and GluN3A-B, and related isoforms and potential subtypes. GluN3A-B subunits may function as a brake to LTP by not allowing glutamate binding and by forming NMDAR subtypes impermeable or relatively impermeable to Ca²⁺. When part of the synaptic framework these subtypes function as down-regulators of Ca²⁺ influx. Cell (neuronal and non-neuronal cells) activity is thus regulated by net Ca²⁺ influx across the different ion channels, including in particular NMDAR channels.

NMDAR mediated Ca²⁺ entry activates down-stream signaling pathways such as: (1) Cam-CaMKII-GIT1βPIX-RAC1-PAK1, (actin remodeling pathway), (2) RAS-MEK-ERK1-2-CREB (cyclic AMP-responsive element-binding protein (CREB)-mediated transcription gene expression pathway), (3) PI3K-AKT-REHB-mTOR [mechanistic target of rapamycin (mTOR)-dependent mRNA translation of plasticity-related proteins (PRPs)], and (4) PRP pathway. Activation of one or more of these pathways, among other downstream effects, mediates synapse modulation including synapse maintenance and spine enlargement and memory consolidation.

As described above, while treatment of isolated psychiatric symptoms (such as the isolated psychiatric symptom of depression) has been previously described, as of yet there is no effective disease-modifying treatment for neuropsychiatric disorders (such as MDD and related disorders). Disease-modifying treatments require a drug or drugs that go beyond symptomatic treatment of one or more psychiatric symptoms. The present inventors have now resolved the issues described in the Background. In that regard, the inventors now disclose that dextromethadone unexpectedly induces rapid, robust, and sustained potentially curative therapeutic effects in patients with MDD. Furthermore, these effects are achieved at doses devoid of cognitive side effects. This signals a previously unrecognized specific disease-modifying mechanism of action, rather than symptomatic treatment of psychiatric symptoms.

And so, aspects of the present invention reduce and/or eliminate issues with present treatments for MDD and other such disorders. In general, an overarching aspect of the present invention provides a disease-modifying treatment for MDD and other disorders. A “disease-modifying” treatment, or a treatment with “disease-modifying” potential, as used herein, includes a drug treatment with the potential for favorably altering the course of an illness by remediating its pathogenetic mechanism. A disease-modifying treatment is therefore potentially curative. In contrast, symptomatic treatments are generally only palliative—they alleviate symptoms, but do not address the molecular cause of the disease.

And so, one aspect of the present invention is directed to a method of treating a neuropsychiatric disorder, the method including administering a composition to a subject suffering from a neuropsychiatric disorder, wherein the composition includes a substance selected from d-methadone, d-methadone metabolites, d-methadol, d-alpha-acetylmethadol, d-alpha-normethadol, l-alpha-normethadol, and pharmaceutically acceptable salts thereof. The neuropsychiatric disorder may be selected from (but is not limited to) Major Depressive Disorder, Persistent Depressive Disorder, Disruptive Mood Dysregulation Disorder, Premenstrual Dysphoric Disorder, Postpartum Depression Disorder, Bipolar Disorder, Hypomania and Mania disorder, Generalized Anxiety Disorder, Social Anxiety Disorder, Somatic Symptom Disorder, Bereavement Depressive Disorder, Adjustment Depressive Disorder, Post-traumatic Stress Disorder, Obsessive Compulsive Disorder, Chronic Pain Disorder, Substance Use Disorder, Overactive Bladder Disorder.

Another aspect of the present invention is directed to a method for treating a neuropsychiatric disorder, the method including (1) diagnosing an individual with a neuropsychiatric disorder, (2) developing a course of treating the neuropsychiatric disorder of the individual, and (3) administering a substance to the individual as at least part of said course of treating the neuropsychiatric disorder of the individual. In this aspect, the substance may be chosen from dextromethadone, dextromethadone metabolites, d-methadol, d-alpha-acetylmethadol, d-alpha-normethadol, I-alpha-normethadol, and pharmaceutically acceptable salts thereof. The neuropsychiatric disorder to be treated may be selected from (but is not limited to) Major Depressive Disorder, Persistent Depressive Disorder, Disruptive Mood Dysregulation Disorder, Premenstrual Dysphoric Disorder, Postpartum Depression Disorder, Bipolar Disorder, Hypomania and Mania disorder, Generalized Anxiety Disorder, Social Anxiety Disorder, Somatic Symptom Disorder, Bereavement Depressive Disorder, Adjustment Depressive Disorder, Post-traumatic Stress Disorder, Obsessive Compulsive Disorder, Chronic Pain Disorder, Substance Use Disorder and Overactive Bladder Disorder.

One embodiment of this aspect of the invention may include a method for treating MDD including (1) diagnosing an individual with MDD, (2) developing a course of treating the MDD of the individual, and (3) administering dextromethadone to the individual as at least part of the course of treating the MDD of the individual.

Another aspect of the present invention is directed to a method of treating a neuropsychiatric disorder, the method including inducing the synthesis in a subject of NMDAR subunits, AMPAR subunits, or other synaptic proteins that contribute to neuronal plasticity and assembled and expressed NMDAR channels. The subject, in this aspect, suffers from a neuropsychiatric disorder (examples of such neuropsychiatric disorders include Major Depressive Disorder, Persistent Depressive Disorder, Disruptive Mood Dysregulation Disorder, Premenstrual Dysphoric Disorder, Postpartum Depression Disorder, Bipolar Disorder, Hypomania and Mania disorder, Generalized Anxiety Disorder, Social Anxiety Disorder, Somatic Symptom Disorder, Bereavement Depressive Disorder, Adjustment Depressive Disorder, Post-traumatic Stress Disorder, Obsessive Compulsive Disorder, Chronic Pain Disorder, Substance Use Disorder and Overactive Bladder Disorder). In this aspect of the invention, inducing the synthesis of NMDAR subunits, AMPAR subunits, or other synaptic proteins that contribute to neuronal plasticity is accomplished by administering to the subject a substance selected from d-methadone, d-methadone metabolites, d-methadol, d-alpha-acetylmethadol, d-alpha-normethadol, l-alpha-normethadol, and pharmaceutically acceptable salts thereof.

Another aspect of the present invention is directed to a method for treating a disease or disorder characterized by a dysfunction of ion channels, the method including (1) diagnosing an individual with a disease or disorder characterized by a dysfunction of ion channels, (2) developing a course of treating the disease or disorder of the individual, wherein the course of treating the disease or disorder involves resolution of the dysfunction of ion channels, and (3) administering a substance to the individual as at least part of the course of resolving the dysfunction of ion channels. The substance used may be chosen from dextromethadone, dextromethadone metabolites, d-methadol, d-alpha-acetylmethadol, d-alpha-normethadol, I-alpha-normethadol, and pharmaceutically acceptable salts thereof. In certain embodiments, the ion channels are integral to one or more NMDARs. In certain embodiments, the ion channels are integral to NMDARs comprising the Glun2C subunit. In certain embodiments, the ion channels are integral to NMDARs comprising the Glun2D subunit. In certain embodiments, the ion channels are integral to NMDARs comprising the Glun2B subunit. In certain embodiments, the ion channels are integral to NMDARs comprising the Glun2A subunit. In certain embodiments, the ion channels are integral to NMDARs comprising the Glun3A subunits.

Another aspect of the present invention is directed to a method for diagnosing a disorder as a disorder caused, worsened, or maintained by pathologically hyperactive NMDAR channels. The method of this aspect includes administering a composition to a subject that has been diagnosed with at least one disorder of unclear pathophysiology chosen from neurological disorders, neuropsychiatric disorders, ophthalmic disorders, otologic disorders, metabolic disorders, osteoporosis, urogenital disorders, renal impairment, infertility, premature ovarian failure, liver disorders, immunological disorders, oncological disorders, cardiovascular disorders. The composition includes a substance selected from dextromethadone, dextromethadone metabolites, d-methadol, d-alpha-acetylmethadol, d-alpha-normethadol, l-alpha-normethadol, and pharmaceutically acceptable salts thereof. One then determines the effectiveness of the composition in the at least one disorder by measuring endpoints specific for each disorder before and after the administration of the composition, and diagnoses the subject with a disorder caused, worsened, or maintained by pathologically hyperactive NMDAR channels if the subject exhibits improvement of specific endpoints. As the endpoints may be specific to a particular disorder, the measurement of the endpoints following administration of the composition allows one to determine the particular disorder to be diagnosed.

In certain embodiments, based on aspects of the invention recited above, the substance is the sole active agent in the composition for treating said neuropsychiatric disorder.

In certain embodiments, based on aspects of the invention recited above, the substance is isolated from its enantiomer or synthesized de novo.

In certain embodiments, based on aspects of the invention recited above, the administering of the composition occurs under conditions effective for the substance to bind to an NMDA receptor of the subject and cause relief to the subject by modifying the course and severity of said neuropsychiatric disorder. In certain embodiments, relief is chosen from cure of said neuropsychiatric disorder, prevention of said neuropsychiatric disorder, reduction in severity of said neuropsychiatric disorder, and reduction in duration of said neuropsychiatric disorder.

In certain embodiments, based on aspects of the invention recited above, the administering of the composition occurs as monotherapy.

In certain embodiments, based on aspects of the invention recited above, the administering of the composition occurs as part of adjunctive treatment to a second substance.

In certain embodiments, based on aspects of the invention recited above, the administering of the composition occurs under conditions effective for an action at a ion channel, neurotransmitter systems, neurotransmitter pathway, or receptor selected from an ionotropic glutamate receptor, a 5-HT2A receptor, a 5-HT2C receptor, an opioid receptor, an AChR, a SERT, a NET, a sigma 1 receptor, a K channel, a Na channel, and a Ca channel. In certain embodiments, the receptor is an opioid receptor and is chosen from MOR, KOR, and DOR. In other embodiments, the administering of the composition occurs under conditions effective for an action at an ionotropic glutamate receptor, and wherein the ionotropic glutamate receptor is an NMDAR. In other embodiments, the action at the ionotropic glutamate receptor includes voltage dependent channel block of NMDARs expressed by the membrane of a cell. In other embodiments, the action at the ionotropic glutamate receptor includes voltage dependent channel block of NMDARs expressed by the membrane of a cell with a preferential effect on NMDAR containing NR2C and NR2D subunits. And, in other embodiments, the action at the ionotropic glutamate receptor includes the induction of synthesis of NMDAR subunits or other synaptic proteins that contribute to neuronal plasticity and contributes to the membrane expression of said synaptic proteins.

In certain embodiments, based on aspects of the invention recited above, the subject is a vertebrate. And, in certain embodiments, the vertebrate is a human.

In certain embodiments, based on aspects of the invention recited above, the substance is dextromethadone. In certain embodiments, the dextromethadone is in the form of a pharmaceutically acceptable salt. In certain embodiments, the dextromethadone is delivered at a total daily dosage of 0.1 mg to 5,000 mg.

In certain embodiments, based on aspects of the invention recited above, the administering of the composition modifies the course and severity of said neuropsychiatric disorder in a subject, and wherein the relief begins within a period of time chosen from two weeks or less after the initial administration of the substance, seven days or less after the initial administration of the substance, four days or less after the initial administration of the substance, and two days or less after the initial administration of the substance.

In certain embodiments, based on aspects of the invention recited above, a therapeutic effect of dextromethadone resulting from administering the composition reaches an effect size greater than or equal to 0.3 in phase 2 clinical trials or an effect size greater than or equal to 0.5 in phase 2 clinical trials, or an effect size greater than or equal to 0.7 in phase 2 clinical trials. In certain embodiments, the therapeutic effect is sustained for at least one week after the discontinuation of treatment. In certain embodiments, the duration of the therapeutic effect after the discontinuation of treatment is equal to or greater than the duration of the treatment.

In certain embodiments, based on aspects of the invention recited above, the administering of the composition occurs in addition to or in combination with the administration of one or more antidepressant medications to the subject.

In certain embodiments, based on aspects of the invention recited above, the administering of the composition occurs in addition to or in combination with the administration of one or more of magnesium, zinc, or lithium to the subject.

In certain embodiments, based on aspects of the invention recited above, the subject has a body mass index equal or less than 35.

In certain embodiments, based on aspects of the invention recited above, administering the composition is used to improve cognitive function, improve social function, improve sleep, improve sexual function, improve ability to perform at work, or improve motivation for social activities.

In certain embodiments, based on aspects of the invention recited above, the administering of the composition is performed orally, buccally, sublingually, rectally, vaginally, nasally, via aerosol, transdermally, parenterally, intravenously, subcutaneously, epidurally, intrathecally, intra-auricularly, intraocularly, or topically.

In certain embodiments, based on aspects of the invention recited above, the administering of the composition occurs at a dose of 0.01-1000 mg per day.

In certain embodiments, based on aspects of the invention recited above, the administering of the composition occurs at a dose of 25 mg per day. In certain embodiments, based on aspects of the invention recited above, the administering of the composition occurs at a dose of 50 mg per day.

In certain embodiments, based on aspects of the invention recited above, the administration of the composition includes administering a loading dose of the composition followed by administration of a daily dose of the composition.

In certain embodiments, based on aspects of the invention recited above, the loading dose of the composition includes an amount of the substance that is greater than the amount of the substance present in each daily dose of the composition.

In certain embodiments, based on aspects of the invention recited above, plasma levels at or higher than steady state are reached on the first day of administration of the composition. In certain embodiments, plasma levels at or higher than steady state are reached within 4 hours of administration of the composition.

In certain embodiments, based on aspects of the invention recited above, following administering of the composition, total plasma levels of the substance in the subject are in a range of 5 ng/ml to 3000 ng/ml.

In certain embodiments, based on aspects of the invention recited above, following administration of the composition, unbound levels of the substance in the subject are 0.5 nM to 1,500 nM.

In certain embodiments, based on aspects of the invention recited above, following administering of the composition, unbound levels of the substance in the subject are in a range of 0.1 nM to 1,500 nM.

In certain embodiments, based on aspects of the invention recited above, the administering of the composition occurs as an intermittent treatment schedule selected from every other day, once every three days, once weekly, every other week, every other two weeks, one week per month, every other month, every other 2 months, every other three months, one week per year, and one month per year.

In certain embodiments, based on aspects of the invention recited above, the administration of the composition is alternated with a placebo in the selected intermittent treatment schedule.

In certain embodiments, based on aspects of the invention recited above, instead of or in addition to placebo the method includes one or more of magnesium, zinc, or lithium.

In certain embodiments, aspects of the invention may be further associated with a digital application to monitor the course of the disorder including the digital monitoring of symptoms and signs and functional and disability outcomes.

Additionally, the inventors also disclose for the first time in this application that dextromethadone decreases NAFLD and potentially NASH and modulates inflammatory markers in rats on “Western Diet” (as shown below in Example 11). The inventors also disclose for the first time in this application that dextromethadone has the potential to modulate biomarkers associated with MDD and TRD in patients (as shown below in Example 7).

Regarding the inventors' discovery (disclosed herein) that dextromethadone has rapid, robust, sustained, and statistically significant efficacy with a large effect size for patients with a diagnosis of MDD and/or TRD: As will be described in greater detail below, the inventors disclose a double-blind, placebo-controlled, prospective, randomized clinical trial that shows that dextromethadone can induce remission of disease (defined as a MADRS score of 10 or less) in over 30% of patients compared to a remission rate of 5% in patients randomized to placebo, within the first week of treatment. Notably, the remission persisted for at least one week after discontinuation of treatment, and longer for some patients. The MADRS rating scale measures not only depressed mood but also provides measures for motivation, cognition-ability to concentrate, sleep, appetite, social abilities, and suicidal risk.

As a general rule (as described above), the effects of symptomatic drugs for chronic conditions after discontinuation of the drug (especially after abrupt discontinuation, as in the case of the clinical trial disclosed by the inventors) tend to rapidly decrease or abruptly cease; and the abrupt discontinuation of symptomatic drugs may even result in the phenomenon of augmentation of symptoms (worsening of symptoms compared to pre-treatment baseline), as well as withdrawal symptoms. Contrary to this, the present inventors have now discovered that improvements resulting from disease-modifying treatments (such as those disclosed herein) tend to persist upon completion of the treatment cycle. The fact that the remission induced by dextromethadone in patients with MDD persists after discontinuation of treatment signals that the action of dextromethadone is not purely symptomatic (i.e., dextromethadone does not simply lift the mood of patients, an effect that would cease upon discontinuation of the drug, as may happen for example with the use of opioids or alcohol for MDD). Thus, this persistence of disease remission suggests a previously unrecognized disease-modifying mechanism of action for dextromethadone (e.g., modulation of neuroplasticity which persists beyond discontinuation of treatment), rather than a mere symptomatic treatment (as was previously thought).

Furthermore, the inventors disclose novel molecular mechanisms that explain these disease-modifying effects of dextromethadone. These mechanisms are described in greater detail in Examples 1-11, below.

The present inventors have described differential block of NMDAR subtypes containing two different subunits: 2A and 2B. The present inventors have now determined (1) that the differential NMDAR block extends to all tested NMDAR subtypes (subtypes A, B, C, and D) and, in particular, to subtypes C and D, and (2) that the block is dependent on the concentration of glutamate and is active even at very low concentrations of glutamate (the concentration of glutamate in the synaptic area is influenced by several variables, including intensity and timing of stimuli; glutamate clearance; et cetera). Even very low concentrations of glutamate may exert downstream consequences, especially if present in the extracellular space for prolonged periods of time (tonic ambient glutamate). The inventors' work in this regard is detailed in Example 1, below.

Example 1 also discloses that, among all tested compounds with known NMDAR blocking activity (tested components included other NMDAR channel blockers approved by the FDA and experimental drugs, such as MK-801), dextromethadone has the lowest potency and the least subtype preference, characteristics that the present inventors believe may explain its effectiveness without side effects. Furthermore, the inventors noted a preference for GluN2C for all tested compounds in clinical use, with the exception of MK-801 (a higher affinity NMDAR blocker with no clinical uses due to its severe cognitive side effects). This GluN2C preference shared by select NMDAR uncompetitive channel blockers, and previously undisclosed for dextromethadone, now provides for an understanding of the downstream effects of differential patterns of Ca²⁺ influx and the potential therapeutic effects of this new class of drugs in pathological states.

Example 2 (below) demonstrates that dextromethadone induces GluN1 mRNA in ARPE-19 retinal pigment cells, and also discloses that dextromethadone induces the synthesis and expression of select protein subunits that form NMDARs (including GluN1, which is necessary for membrane expression of NMDARs). Furthermore, dextromethadone is now shown (by the present inventors) to also influence transcription of GluN2C and 2D mRNA and synthesis of the related proteins, subunits 2C and 2D.

The work of the present inventors detailed in Example 2 now also demonstrates that dextromethadone differentially modulates the synthesis of NMDAR subunits (e.g., it modulates that synthesis of GluN2A subunits but not GluN2B subunits). This selectivity, exhibited in the tested cell line (ARPE-19) in Example 2, not only signals the regulatory effect of dextromethadone (and thus the regulatory effect of differential patterns of Ca²⁺ influx modulated by dextromethadone), but also signals subunit-selective effects on the synthesis of proteins that form NMDARs. These findings of the present inventors reveal novel aspects at the basis for physiologic and pathologic memory formation, including its relation to MDD (and other disorders of similar pathophysiological basis).

In that regard, NMDARs have been recognized as central and essential for memory formation in vertebrates, and the four different subtypes (GluN2A-D) have been present across all vertebrate species for over 500 million years. This underscores the evolutionary importance of widening coding capability offered by NMDAR differentiation in subtypes (fine tuning of the differential Ca²⁺ influx patterns that form the epigenetic code). The NMDAR blocking effect of dextromethadone and the resulting downregulation of Ca²⁺ influx resulting in modulation of protein transcription and synthesis in ARPE-19 cells (1) includes NMDAR proteins, and (2) is selective for NMDAR subtypes, e.g., GluN1 and GluN2A subunits versus Glun2B subunits, and thus is selective for NMDAR subtype assembly and expression in this cell line (as outlined in Example 2). These mechanisms result in the induction of synthesis of new NMDAR select subunits (and thus assembly and expression of new NMDAR select subtypes) and signal the potential for synapse modulating/strengthening effects (e.g., modulation of post-synaptic NMDARs) for dextromethadone.

These newly recognized mechanisms (disclosed by the present inventors) are separate from and in addition to the effects on production of BDNF in human subjects (BDNF is capable of retrograde pre-synaptic strengthening and neurite growth effects) disclosed by the inventors [De Martin S, Vitolo O, Bernstein G, Alimonti A, Traversa S, Inturrisi C E, Manfredi P L, The NMDAR Antagonist Dextromethadone Increases Plasma BDNF Levels in Healthy Volunteers Undergoing a 14-Day In-Patient Phase 1 Study, ACNP 57th Annual Meeting: Poster Session II. ACNP 57th Annual Meeting: Poster Session II. Neuropsychopharmacol. 43, 228-382 (2018)]. While that study may have shown enhancement of BDNF plasma levels from dextromethadone in healthy volunteers, the subjects did not have a diagnosis of MDD, and so there has been no teaching or suggestion of treatment of MDD with dextromethadone. In fact, the enhancement of BDNF in patients with MDD has not been shown to be consistently present with dextromethadone, and so the teachings of studies such as De Martin has never been applied to MDD (as described above in the Background, treatments using dextromethadone have been limited to treatment of isolated symptoms, and that treatment has never been seen as translatable to neuropsychiatric disorders such as MDD). The disclosure of Example 2 (post-synaptic NMDAR modulation by dextromethadone, revealed by the induction of synthesis of select NMDAR subunits), however, provides a complementary mechanism for dextromethadone-induced neural plasticity from BDNF and adds new levels of understanding to the mechanism of neuronal transcription, production and release of BDNF.

In Example 3, the inventors also disclose the unexpected results of a Phase 2a trial of dextromethadone in patients with MDD. The molecular mechanisms for synaptic strengthening disclosed by the work of the present inventors (and described throughout the Examples) potentially explain the unexpected disease-modifying effects of dextromethadone in patients with MDD and support the novel disclosures in this application of uses of dextromethadone as a disease-modifying treatment for MDD and related disorders, including TRD, as well as a multiplicity of neuropsychiatric disorders and other disorders.

The disclosure herein of previously unknown molecular effects and mechanisms of action for dextromethadone additionally signals its potential efficacy for a multiplicity of neuropsychiatric, metabolic, and cardiovascular diseases and disorders. The present inventors are now able to disclose that (in certain subsets of patients) diseases and disorders are triggered or maintained by excessive Ca²⁺ influx through pathologically hyperactive NMDARs. Prior to the work of the present inventors disclosed herein, it was believed by those of ordinary skill in the art that the main mode of action of dextromethadone was the block of hyperactive NMDAR channels at the PCP site of the intramembrane domain of NMDARs, and that receptor occupancy by dextromethadone was therapeutic only for the symptomatic treatment of isolated psychological symptoms (such as isolated symptoms of pain, addiction, depression, and anxiety). The work and discoveries of the inventors outlined in Examples 1-11, however, signal that dextromethadone can be therapeutic (as a disease-modifying agent) for a multiplicity of diseases and disorders, including MDD and related disorders, sleep disorders, anxiety disorders, and cognitive disorders, well beyond receptor occupancy (because of persistent neural plasticity effects) and thus, not be merely a symptomatic agent as previously thought.

As is now disclosed, dextromethadone exerts its disease-modifying therapeutic effects by modulating the production and membrane expression of novel and functional NMDARs, thereby potentially re-equilibrating the functionality (e.g., production of synaptic strength, and thus production of memory) of certain cells and re-instituting their role (e.g., connectivity) within circuits and tissues. The GluN1 subunit is essential for receptor expression. Therefore, dextromethadone may not only modulate pathologically hyperactive NMDAR, but may also induce the synthesis and expression of new functional NMDARs, which then allow for proper functioning of certain neuronal cells that are part of certain circuits (i.e., pre- and post-synaptic strengthening of synapses, and memory formation, including emotional memory formation and modulation). Dextromethadone, and potentially other NMDAR blocking agents, not only changes the pattern of Ca²⁺ entry by blocking the pore channel of the NMDAR (an action that potentially explains symptomatic effects) but also changes the NMDAR expression on cell membranes (a novel mechanism of action disclosed by the present inventors that explains its unexpected disease-modifying robust, rapid, sustained effect demonstrated by the clinical study results illustrated particularly in Example 3, below).

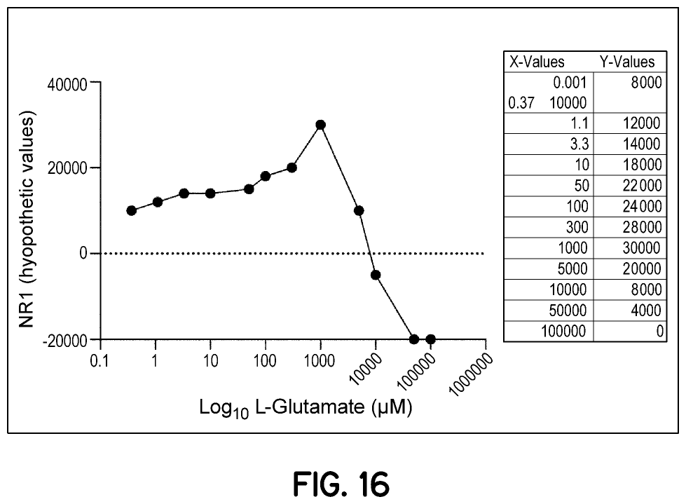

As described above, the inventors show (in Example 2) that dextromethadone not only induces the mRNA for GluN1 but also modulates the production of the GluN1 protein subunit and other GluN2A protein subunits. The present inventors also found that these effects were more evident in cells exposed to low concentrations of dextromethadone for one week (matching the clinical protocol of Example 3, where patients were treated with a relatively low drug dose for one week). While not being bound to any theory, the present inventors believe that NMDARs expressed on the membrane of ARPE-19 cells exposed to excessive stimulation (by high concentration glutamate or for example by excessive light) open pathologically (i.e., excessively) and that excessive Ca²⁺ influx causes a shutdown of cellular activity (see FIG. 16, and Example 2), including shutdown of genes for production of synaptic proteins, including production of NMDAR subunits, and including NMDAR1 and differential modulation of NMDAR2A-D.

When cells impaired by excessive stimulation and/or Ca²⁺ influx are exposed to dextromethadone, the excessive Ca²⁺ entry is downregulated and the production of synaptic proteins resumes. In the case of ARPE-19 cells, NMDAR1 subunits (necessary for membrane expression of the NMDAR) and, for example GluN2A subunits (but not GluN2B subunits) are induced. This selectivity is likely not casual but is potentially related to the functionality/specialization of the ARPE-19 cell line when exposed to a given amount of stimulation, e.g. light. This selective modulation of NMDAR subunits will differ when the stimulation is applied to a different cell line with a different functionality and with a different framework of membrane expression of NMDARs, and part of a different circuitry or different tissue, or even in the same cell line when differential stimulations are applied (different glutamate concentrations or different intensity or quality of light exposure: different experimental settings).

In addition to the above, the present inventors (in Example 5) also demonstrate herein the downregulation of Ca²⁺ influx by dextromethadone in cells exposed to a gentamicin, shown herein by the inventors to be a Positive Allosteric Modulator (PAM) of the NMDAR. Gentamicin is toxic for otologic hair cells, the cells that transduce sound into electrochemical signaling. To that end, Example 5 describes the potential disease-modifying effects of dextromethadone not only when excitotoxicity from excessive Ca²⁺ inflow is caused by excessive presynaptic glutamate release (e.g., during prolonged psychological stress), but also at very low glutamate concentrations (even physiological concentrations) when excessive Ca²⁺ influx is caused by a toxic PAM.

Toxic PAMs may be one of a multiplicity of different chemical entities and may act via two main mechanisms: (1) increasing the maximal response to glutamate (aPAM) and/or (2) shifting the ED50 of glutamate to the left (bPAM). In Example 5, gentamicin appears to act as an aPAM via mechanism (1) on GluN2B, and as a bPAM via mechanism (2) on GluN2A, GluN2C, and GluN2D. The bPAM mechanism on GluNC and GluND subunit containing NMDAR subtypes is of relevance to this disclosure because of the disclosed mechanism of action of dextromethadone. As suggested by Example 1 (preference for GluN1-GluN2C and activity at GluN2D subtypes), and by Examples 2, 5, and 6, dextromethadone may preferentially (selectively) block Ca²⁺ influx via tonically Ca²⁺ permeable GluN1-GluN2C and GluN1-GluN2D subtypes (and subtypes containing GluN3 subunits).

Dextromethadone, due to its mechanisms of action (block of excessive inward Ca²⁺ currents) with selectivity for NMDARs tonically and pathologically hyperactive GluN1-GluN2C (and GluN1-GluN2D subtypes and possibly subtypes containing GluN3 subunits), regardless of the cause (excessive glutamate or anyone of a multiplicity of molecules, acting at agonist sites or as PAMs, including exogenous and endogenous chemicals, including antibodies), is thus now determined by the present inventors to be potentially preventive, therapeutic, and/or diagnostic for a multiplicity of diseases triggered or maintained by pathologically and tonically excessively Ca²⁺ permeable NMDARs. In the case of MDD, NMDAR agonists (such as quinolinic acid) may also increase extracellular glutamate by different mechanisms [Guillemin G J, Quinolinic acid: neurotoxicity, FEBS J. 2012; 279(8):1355], thus further hyperactivating NMDARs. Dextromethadone also counteracts the additive neurotoxic effects of quinolinic acid, as seen in Example 5. Thus, the results of Examples 1 and 2, and the results for the NMDAR PAM gentamicin and the agonist quinolinic acid of Example 5, and the Phase 2 results in MDD patients, showing rapid, robust, and sustained efficacy detailed in Example 3, and the results and disclosure detailed in Examples 6-11, strongly signal disease-modifying effects of dextromethadone for patients with MDD and other diseases characterized by hyperactivation of NMDAR. Therefore, MDD related disorders, e.g., PPD (Maes M, et al. Depressive and anxiety symptoms in the early puerperium are related to increased degradation of tryptophan into kynurenine, a phenomenon which is related to immune activation. Life Sci. 2002; 71:1837-1848) and inflammatory states [Capuron L, et al. Interferon-alpha-induced changes in tryptophan metabolism: relationship to depression and paroxetine treatment, Biol. Psychiatry. 2003, 54:906-914; Raison C L, et al. CSF concentrations of brain tryptophan and kynurenines during immune stimulation with IFN-alpha: relationship to CNS immune responses and depression, Mol. Psychiatry. 2010, 15:393-403; Du J, Li X H, Li Y J. Glutamate in peripheral organs: Biology and pharmacology, Eur J Pharmacol. 2016; 784:42-48], may also be candidates for treatment with dextromethadone.

Patients with CNS disorders, including encephalopathy, associated with increased quinolinic acid levels in serum and/or CSF, as exemplified by patients with Lyme disease [Halperin J J, Heyes M P. Neuroactive kynurenines in Lyme borreliosis, Neurology. 1992; 42(1):43-50], are likely to improve with dextromethadone. Additionally, the immunological response to infection, causing alterations in the hypothalamic-pituitary-adrenal axis (as signaled by the lowering BP effects of dextromethadone in the present inventors' Phase 1 MAD study) and depression could all be positively influenced by dextromethadone and its downregulating excessive Ca²⁺ influx via hyper-stimulated NMDARs, e.g., by quinolinic acid [Ramirez L A, Perez-Padilla E A, Garcia-Oscos F, Salgado H, Atzori M, Pineda J C. A new theory of depression based on the serotonin/kynurenine relationship and the hypothalamic-pituitary-adrenal axis, Biomedica. 2018; 38(3):437-450. Published 2018 Sep. 1]. Modulation of the hypothalamic-pituitary-adrenal axis is also signaled by the lowering BP effects of dextromethadone in the present inventors' Phase 1 MAD study.

During normal (physiological) brain activity, stimulation and depolarization of the presynaptic neuron results in release of glutamate by its axon in the synaptic cleft, with opening of AMPARs (with Na⁺ influx, postsynaptic depolarization and release of NMDAR voltage dependent Mg²⁺ block) and with opening of NMDARs and Ca²⁺ influx. Ca²⁺ influx, at physiologic amounts, promotes neural plasticity via CaMKII activation at the post-synaptic level [induction of synthesis of synaptic proteins and strengthening of the synapse in the post-synaptic cell and also at the postsynaptic and presynaptic levels, via synthesis and release of BDNF in the extracellular space with synaptic strengthening and trophic (spine production and growth) and tropic (direction of growth) effects on neuritis]. Direct activation of NMDARs on the pre-synaptic cell may also contribute to neural plasticity (Berretta N, Jones R S. Tonic facilitation of glutamate release by presynaptic N-methyl-D-aspartate autoreceptors in the entorhinal cortex. Neuroscience 1996; 75:339-344) at the pre-synaptic level, e.g., by modulating glutamate stores.

The present inventors' experimental results shown in Examples 1-11 suggest that when the Ca²⁺ influx via NMDARs is excessive, cells halt the production of synaptic proteins and neurotrophic factors (a first step in excitotoxicity that can potentially progress to apoptosis). Dextromethadone, by downregulating excessive Ca²⁺ influx restores the neural plasticity machinery (production of synaptic proteins and neurotrophic factors, including BDNF). This potentially prevents progression of cellular dysfunction and apoptosis, and thus exerts disease-modifying treatment for MDD [as well as for MDD related disorders and potentially for a multiplicity of diseases triggered, maintained or worsened by excessive Ca²⁺ influx via NMDARs in select cells part of select cellular populations, tissues, circuits in the CNS and extra CNS (Du et al., 2016)].

The downstream effects of Ca²⁺ on the LTP machinery follow an inverted U curve: Ca²⁺ influx favors LTP up to a certain amount of Ca²⁺ influx and then, when Ca²⁺ influx becomes excessive, the cell becomes dysfunctional (excitotoxicity) and LTP is inhibited. If this excessive Ca²⁺ influx progresses the cell may be permanently damaged. When the neurons with hyper-stimulated NMDARs (where LTP is interrupted because of excitotoxicity) are part of one (or more) of a multiplicity of functional circuits or tissues, disorders and diseases specific for the impaired circuit or tissue may result.

Thus, the molecular effects of dextromethadone presented in the Examples provide a potential mechanism for the results seen in Example 3 with respect to MDD: i.e., the unexpectedly strongly positive (highly statistically significant p values with large effect size), rapid (the first signals of efficacy unexpectedly started on day two for the 25 mg dose and were statistically significant for both doses—25 mg and 50 mg—on day 4) and sustained/long lasting/persistent (statistically significant clinically meaningful therapeutic effects and large effect size persisted for at least one week after abrupt discontinuation of 1-week treatment course) efficacy results seen in the Phase 2a study detailed in Example 3. These neuroplasticity effects—which include NMDAR-mediated LTP—may also explain the unexpected signal for better efficacy seen in patients randomized to the 25 mg dose (with corresponding lower dextromethadone plasma concentration, around 300 nM) compared to patients receiving the 50 mg dose (with corresponding higher dextromethadone plasma concentration, around 600 nM) (seen in Example 3). The therapeutic effects of dextromethadone potentially follow an inverted U curve, similarly to what has been described for other NMDAR open channel blockers, such as ketamine. Finally, while the safety window for dextromethadone may be wide (Example 3), the therapeutic window, at least for MDD, may be tailored to daily doses between 5 and 100 mg, and/or 12.5-75 mg, and plasma concentrations between 50-900 ng/ml and/or free levels of 5-90 (see Example 3). This aspect is detailed below when BMI is taken into consideration in a sub-analysis of the Phase 2a study results.

From these robust efficacy results (including the sustained efficacy after discontinuation of the drug), it is now evident for the first time that dextromethadone does not simply improve isolated symptoms. Rather, dextromethadone shows a strong signal for exerting disease/disorder-modifying effects for patients with MDD, MDD related disorders, and potentially for patients suffering from other neuropsychiatric and metabolic disorders, and other disorders that are potentially associated with NMDAR hyperactivation (including disorders of the hypothalamic-pituitary axis, such as hypertension, and potentially cardiovascular and metabolic disorders and other disorders described by Du et al., 2016, which are incorporated by reference herein) and excessive Ca²⁺ influx in select cells.

These unexpectedly strongly positive and sustained effects are unprecedented in trials for MDD with drugs that do not cause psychotomimetic side effects. Furthermore, as detailed below, the extreme tolerability and safety of dextromethadone (with an adverse event profile similar to placebo at the very effective 25 mg oral daily dose) signals that the activity of dextromethadone for pathologically hyperactive channels (hyperactivated NMDARs) is highly selective (with select sparing of physiologically working channels). Therefore, the efficacy of dextromethadone can be potentially extended to a multiplicity of diseases and disorders triggered or maintained by cell/circuitry dysfunction due to hyperactivated NMDARs (e.g., NMDAR hyperstimulation by glutamate or other agonists or PAMs).

And so, while dextromethadone has been useful for the treatment of isolated symptoms, such as pain and depression (disclosed by the inventors in U.S. Pat. Nos. 6,008,258 and 9,468,611), the present inventors have now determined for the first time that it is capable of exhibiting disease-modifying effects, and so is also useful as a disease-modifying treatment for a multiplicity of diseases and disorders triggered, maintained or worsened by a halting of physiological neural plasticity and or a halting of other physiological cell functions caused by excessive Ca²⁺ influx in select cells, part of select subpopulations, tissues and/or circuits (this had not been recognized previously).

When hyperactivated NMDARs are expressed at select sites on the membrane of select cells part of specific structural and functional circuits, NMDARs allow excessive Ca²⁺ influx, causing cellular dysfunction (also called excitotoxicity) in select cells and cell lines and populations and tissues and circuits. In the Nervous System (NS) the dysfunction of CNS cells (including neurons, astrocytes, oligodendrocytes and other glial cells, including microglia), depending on temporospatial factors (developmental age and location within the NS) and NS cell subtype, causes altered brain connectivity in select circuits. Patients may manifest this circuit impairment as a syndrome, a disorder, or a disease, e.g., one of a multiplicity of neuropsychiatric disorders.

Such syndromes, disorders, or diseases may include MDD (listed in DMS5 and ICD11) or one or more of: Alzheimer's disease; presenile dementia; senile dementia; vascular dementia; Lewy body dementia; cognitive impairment [including mild cognitive impairment (MCI) associated with aging and with chronic disease and its treatment], Parkinson's disease and Parkinsonian related disorders, including but not limited to Parkinson dementia; disorders associated with accumulation of beta amyloid protein (including but not limited to cerebrovascular or disruption of tau protein and its metabolites including but not limited to frontotemporal dementia and its variants, frontal variant, primary progressive aphasias (semantic dementia and progressive non fluent aphasia), corticobasal degeneration, supranuclear palsy; epilepsy; NS trauma; NS infections; NS inflammation [including inflammation from autoimmune disorders (such as NMDAR encephalitis), and cytopathology from toxins (including microbial toxins, heavy metals, pesticides, etc.)]; stroke; multiple sclerosis; Huntington's disease; mitochondria! disorders; Fragile X syndrome; Angelman syndrome; hereditary ataxias; neuro-otological and eye movement disorders; neurodegenerative diseases of the retina like glaucoma, diabetic retinopathy, and age-related macular degeneration; amyotrophic lateral sclerosis; tardive dyskinesias; hyperkinetic disorders; attention deficit hyperactivity disorder (“ADHD”) and attention deficit disorders; restless leg syndrome; Tourette's syndrome; schizophrenia; autism spectrum disorders; tuberous sclerosis; Rett syndrome; Prader Willi syndrome; cerebral palsy; disorders of the reward system including but not limited to eating disorders [including anorexia nervosa (“AN”), bulimia nervosa (“BN”), and binge eating disorder (“BED”)], trichotillomania; dermotillomania; nail biting; substance and alcohol abuse and dependence; migraine; fibromyalgia; and peripheral neuropathy of any etiology.

The present inventors view the subsets of patients diagnosed with a neuropsychiatric disorder listed in DMS5 and ICD11, just as MDD patients described in Example 3, as suffering from disorders triggered and/or maintained by hyperactivated NMDARs. A drug like dextromethadone, with molecular actions disclosed in Examples 1-7 and clinical effects (efficacy and safety) presented in Example 3, is potentially safe and effective for select patients diagnosed with neuropsychiatric disorders listed in DMS5 and ICD11, including for NMDAR encephalitis and other immunological disorders affecting NMDARs and for diseases and disorders described by Du et al., 2016 (those diseases and disorders described in Du et al. being incorporated by reference herein).

Dextromethadone can thus be used not only as a preventive and/or therapeutic drug, but also as a safe and effective diagnostic tool for selecting patients diagnosed with neuropsychiatric disorder listed in DMS5 and ICD11 that may suffer from disorders triggered and/or maintained by hyperactive NMDARs. The present inventors thus also disclose dextromethadone not only as a preventive or therapeutic drug but also as a diagnostic tool for diagnosis of NMDAR dysfunction in a multiplicity of diseases and disorders, including neurological, neuropsychiatric, ophthalmic (including visual impairment), otologic (including hearing impairment, balance impairment, vertigo, tinnitus), metabolic (including impaired glucose tolerance and diabetes, liver disorders including NAFLD and NASH, osteoporosis), immunologic, oncologic and cardiovascular (including CAD, CHF, HTN) and other diseases and disorders such as those listed above and those described by Du et al., 2016. Dextromethadone administration by any of the routes disclosed herein will aid in the diagnosis of diseases and disorders triggered or maintained by hyperactive NMDARs in vertebrates, mammals and humans.

Based on the new experimental data disclosed herein, the present inventors also disclose that dextromethadone may selectively target certain pathologically hyperactive NMDARs (e.g., a subset of tonically hyperactive NMDARs, e.g., subtype NR1-GluN2C and/or NR1-GluN2D and or subtypes containing 3A and/or 3B subunits), and down-regulate the excessive Ca²⁺ influx only in hyperactive NMDAR channels that had been functionally and structurally impairing the cell. As shown by the FLIPR experiments of Example 1, the actions of dextromethadone at NMDAR are differential according to the intensity of the presynaptic stimulation (the blocking action of dextromethadone increases with increasing glutamate stimulation) and are differential based on the NMDAR subtype. This experiment does not include Mg²⁺ and therefore it is similar to a setting where AMPAR depolarization induced by pre-synaptic glutamate release has already released Mg²⁺ from the NMDAR into the synaptic cleft. The presence of Mg²⁺ in vivo is likely to make dextromethadone less relevant (i.e., dextromethadone is unlikely to have blocking effect on deactivated, Mg²⁺ blocked channels, because they are already blocked and inactive, e.g., subtypes GluN2A and B, impermeable to Ca²⁺ while blocked by Mg²⁺). These differential actions at receptor subtypes A-D by dextromethadone are however important for elucidating its actions selective for tonically and pathologically hyperactive channels, e.g., NR1-NR2C (and NR1-NR2D subtypes or 3A-B subunit containing subtypes). The downregulation of Ca²⁺ influx through the open pore channel afforded by dextromethadone modulates neural plasticity activity, including the induction of production of synaptic proteins, including NR1, NR2A-D and NR3A-B subunits (Example 2), and production of other synaptic proteins and neurotrophic factors in humans. Neurotrophic factors are known to act on both post-synaptic and pre-synaptic neural plasticity.

The present inventors disclose herein that the uncompetitive open channel blocker dextromethadone acts directly and selectively at pathologically hyperactive channels to regulate Ca⁺ influx and thus re-activate physiological neural plasticity pre- and post-synaptically in select cells. The block of pathologically hyperactive channels regulates excessive Ca²⁺ influx with positive downstream consequences, including gene activation for synthesis of key factors for neural plasticity, such as synaptic proteins, including GLUN1 and 2A subunits (Example 2), and neurotrophic factors, including BDNF. This activation of the synthetic neural plasticity activity of neurons signals the correction of an abnormality, excessive Ca²⁺ entry, that had caused the cell to stop its production of neural plasticity peptides and thus results in the resumption of physiological neural plasticity.

In support of the present inventors' disclosed mechanism of action, this re-activation of cellular function (selective for cells impaired by excessive Ca⁺ influx) and thus re-activation of impaired CNS circuitry, is manifested clinically by the present inventors' unexpected findings of rapid onset, robust, and sustained effects (after discontinuation of treatment) in patients with MDD. This finding (see Example 3) supports not only that NMDAR hyperactivation (and excessive Ca²⁺ influx in select neurons) was the culprit (trigger and/or maintaining factor) for MDD enrolled in the present inventors' trial (a novel pathogenetic mechanism for MDD and related disorders), but signals that dextromethadone is also potentially curative for MDD, for disorders related to MDD and for other neuro-psychiatric disorders, including disorders of the hypothalamus-pituitary axis that are triggered and/or maintained by pathologically hyperactive NMDARs and excessive Ca²⁺ influx and inhibition of neural plasticity or impairment of other cell functions, (e.g., see Example 5 with gentamicin acting as a PAM, and thus for diseases and disorders described by Du et al., 2016).

In the case of CNS disorders, excessive Ca²⁺ entry in select neurons, before the onset of excitotoxicity, may also result in excessive inhibitory activity, e.g., inhibitory interneurons projecting to medial prefrontal cortical (mPFC) neurons. By blocking pathologically hyperactive NMDAR channels, e.g., select tonically hyperactive NMDARs, dextromethadone may reduce or halt excessive inhibitory activity by interneurons, relieving the excessive inhibition of mPFC neurons. The control of inhibitory activity by means of opposite actions 1) GABAaR dispersion or 2) GABAaR clustering is a result of stimulus induced NMDAR activity [Bannai H, Niwa F, Sherwood M W, Shrivastava A N, Arizono M, Miyamoto A, Sugiura K, Levi S, Triller A, Mikoshiba K. Bidirectional control of synaptic GABAAR clustering by glutamate and calcium. Cell reports. 2015 Dec. 29; 13(12):2768-80]. Thus, the inhibitory activity, present for the homeostatic rhythms of brain networks, is controlled by NMDAR determined Ca²⁺ influx. When excessive, these Ca²⁺ inward currents can be potentially modulated by dextromethadone. Thus, not only excitatory activity but also inhibitory activity is regulated by NMDARs and Ca²⁺ signaling. The NMDAR framework is therefore not only in control of excitatory actions but also inhibitory actions by regulating, via Ca²⁺ signaling, the framework of all other receptors, including inhibitory receptors, such as GABAaRs.

The NMDAR assumes therefore a central regulatory position that receives environmental input and translates this input in finely regulated neuronal plasticity by controlling and modulating, via Ca²⁺ signaling and its downstream effects all synaptic frameworks. Such downstream effects include NGF and synaptic protein transcription, synthesis, transport and assembly, including transcription of receptor subunits for AMPAR, NMDARs, GABAaRs and virtually all other CNS receptors. The NMDAR thus controls the lifetime evolution of synaptic frameworks, which include NMDARs, as it is shaped by environmental stimuli.

Thus, diseases and disorders can be triggered, maintained or worsened by excessive activation of one or more NMDAR subtypes expressed by select neurons, integral to one of a multiplicity of different circuits, (e.g., activation triggered by glutamate mediated stimulation, including by life-stressors, or by other stimuli, or by endogenous or exogenous agonists and/or endogenous or exogenous PAMs, including toxins). This excessive NMDAR activation results in excessive Ca²⁺ influx via NMDARs into the post-synaptic neuron. Pre-synaptic glutamate receptors also have a role in neural plasticity (Baretta and Jones, 1996; Bouvier G, Bidoret C, Casado M, Paoletti P. Presynaptic NMDA receptors: Roles and rules. Neuroscience. 2015; 311:322-340) and thus may be regulated by dextromethadone. When Ca²⁺ influx in a select neuron is excessive it downregulates neural plasticity activity and reduces or interrupts its connectivity, altering (decreased synaptic machinery and strength) functionality (excessive Ca²⁺ influx may even affect vital structures and functions of the neuron, if excitotoxicity progresses towards cellular apoptosis) of its neuronal circuit. A drug like dextromethadone, with its unique molecular actions as an NMDAR blocker (Examples 1 and 5), downregulates excessive Ca²⁺ cellular influx in pathologically hyperactive NMDARs without effects on physiologically functioning NMDARs (this was demonstrated for the first time in the Phase 2a trial showing a lack of cognitive side effects at therapeutic doses, Example 3). Thus, cells (previously impaired by excitotoxicity) resume neural plasticity functions and restore NS circuits with resolution of circuitry failure (resolution not only of neuropsychiatric symptoms but also resolution of the neuro-psychiatric disorder: this disease-modifying effect is due to neural plasticity and not merely due to receptor occupancy and temporary effects from downregulation of Ca²⁺ influx, as shown by the sustained efficacy results shown in Example 3 after abrupt discontinuation of treatment and with decreasing plasma concentration of dextromethadone and consequential decrease receptor occupancy.

A drug like dextromethadone, which is well tolerated at disease-modifying effective doses, as confirmed for the first time in patients by the Phase 2a results presented in this application (Example 3), with disclosed differential Ca²⁺ downregulating actions for differential concentrations of glutamate stimulation (including for very low levels of glutamate), including in the presence of PAMs and other agonists (Example 5) and differential and unique actions at NMDAR subtypes (Examples 1, 5), unique “on”-“off” NMDAR kinetics (Example 6, Part I) and “trapping” profile (Example 6, Part II) and unique effects in the presence of physiological concentrations of Mg²⁺ at resting membrane potential (Example 6, Part III), is a potentially disease-modifying treatment for a multiplicity of diseases and disorders. Importantly, the blocking activity of dextromethadone at NMDAR channels does not interfere with physiological activity at effective doses (as demonstrated by lack of side effects at therapeutic doses, Example 3), as signaled by the results disclosed in Examples 1-11. Dextromethadone is thus a novel tool to explore brain functionality, both during physiological operations and under pathological circumstances. Additionally, the researcher and the practitioner will be armed with a novel diagnostic tool to select subsets of patients with NMDAR hyperfunction causing or maintaining or worsening one of a multiplicity of diseases and disorders.

Based on experimental findings with dextromethadone, in vitro and in vivo in healthy subjects and in patients with MDD, the inventors are now able to postulate that the shared epigenetic code, at the basis of the G+E paradigm, is determined by stimulus (environmental stimuli reaching cells) induced [presynaptic release of glutamate, integrated by agonists, PAMs and NAMs (e.g., activation of the polyamine site of NMDARs, or other allosteric or agonist sites by other NMDAR modulators, or toxins) determining differential patterns of Ca²⁺ cellular influx, with kinetics determined by the NMDAR framework. These differential patterns of Ca²⁺ influx determine, in health and in disease, in the brain (there will be other effects in other cells/tissues), postsynaptic and presynaptic neural plasticity modulation: e.g., excessive Ca²⁺ influx downregulates neural plasticity and a reduction of excessive Ca²⁺ influx, e.g., by the uncompetitive channel blocker dextromethadone, potentially results in resumption of physiological neural plasticity, as seen in experimental studies presented throughout the application. The shared code for brain activity—differential patterns of Ca²⁺ influx—has been shown by the inventors to regulate NMDAR expression (NMDAR framework) (Example 2). The pattern of postsynaptic Ca²⁺ influx after presynaptic release of glutamate is regulated by post-synaptic AMPAR and NMDAR expression (and pre-synaptic NMDAR expression, as shown by Berretta and Jones, 1996), and this post-synaptic AMPAR and NMDAR receptor expression (and pre-synaptic glutamate release) is in turn regulated by Ca²⁺ influx. Thus, NMDARs are both regulators and regulated by Ca²⁺ influx. This regulation of NMDAR expression (NMDAR framework) by stimulation-triggered differential patterns of Ca²⁺ influx that flow across NMDARs is the basis of neural plasticity and is the basis for the unique connectome of each individual. Each environmental interaction with an individual will thus affect a different NMDAR framework and result in a different amount of Ca²⁺ influx with different downstream consequences. Dextromethadone can correct excessive (pathological) Ca²⁺ influx via NMDARs.

### EXAMPLES

### Example 1—Mode of Action Fluorescence Imaging Plate Reader (FLIPR) Calcium Assay on Human NMDA Receptors Using GluN1-GluN2A, -2B, -2C, -2D Cell Lines

The following is a list of abbreviations used in this Example, and in the present application.

| Abbreviation | Definition or Expanded Term |
|---|---|
| AUC | Area under the curve |
| CHO | Chinese hamster ovary |
| CRC | Concentration response curve |
| DMSO | Dimethyl sulfoxide |
| FLIPR | Fluorescence imaging plate reader |
| Gly | Glycine |
| GLP | Good laboratory practice |
| K_{B} | Estimated test item equilibrium dissociation |
| | constant |
| Log | Base 10 logarithm |
| L-glu | L-glutamate |
| MW | Molecular weight |
| NA | Not available |
| NMDA | N-methyl-D-aspartate |
| NMDAR | N-methyl-D-aspartate receptor |
| QC | Quality control |
| SEM | Standard error of the mean |
| SOP | Standard operating procedure |
| α | Estimated test item cooperativity term |
| τ | Agonist efficacy value |

A. Introduction

This Example 1 demonstrates the mechanism of action of dextromethadone at the NMDAR subtypes and the relative potency at each channel subtype, and compares to other channel blockers. It also informs on the ability of dextromethadone to influence Ca²⁺ influx triggered by very low ambient glutamate. Together with other evidence disclosed herein, this corroborates the novel pathophysiology of MDD (excessive Ca²⁺ influx via tonically and pathologically activated NMDARs) disclosed by the inventors.

The mode of action FLIPR-calcium assay described herein was designed to establish test item effect, at 6 selected concentrations, on L-glutamate concentration response curve fitting parameters, in four human recombinant NMDA receptor types: GluN1-GluN2A, GluN1-GluN2B, GluN1-GluN2C, GluN1-GluN2D.

B. Test and Control Items

Five test items were selected for this study: dextromethadone hydrochloride (CAS #15284-15-8, supplied by Padova University); memantine hydrochloride (CAS #41100-52-1, supplied by Bio-Techne Tocris); (±)-ketamine hydrochloride (CAS #1867-669, supplied by Merck Sigma-Aldrich); (+)-MK 801 maleate (CAS #77086-22-7, supplied by Bio-Techne Tocris); and dextromethorphan hydrobromide monohydrate (CAS #6700-34-1, supplied by Merck Sigma-Aldrich).

The vehicle used was DMSO (CAS #67-68-5; supplied by Merck Sigma-Aldrich).

The test item formulation is shown in Table 1 below.

TABLE 1

| Nature of Formulation | DMSO solution | |
|---|---|---|
| Concentrations (400x | 20 | mM |
| in DMSO) | 5 | mM |
| | 1.25 | mM |
| | 312 | μM |
| | 78 | μM |
| | 19.5 | μM |
| Storage Conditions | | |
| Before dissolution | −20° C. for dextromethadone hydrochloride; | |
| (as solid): | ambient temperature/protected from light | |
| | for remaining test items | |
| After dissolution: | −20° | C. |

C. Test System

Test items were evaluated in FLIPR for their ability to modulate L-glutamate and glycine induced calcium entry in four CHO cell lines expressing diheteromeric human NMDA receptor (NMDAR): GluN-/GluN2A-CHO, GluN1-GluN2B-CHO, GluN1-GluN2C-CHO, GluN1-GluN2D-CHO.

D. Experimental Design

The study aimed to monitor the effect of the five test items on L-glutamate CRC, in presence of fixed 10 μM glycine concentration.

6 concentrations were tested for each test item: 50 μM, 12.5 μM, 3.13 μM, 0.781 μM, 0.195 μM, and 0.049 μM.

L-glutamate 11 point CRC included the following final concentrations: 100 mM, 1 mM, 100 μM, 10 μM, 3.3 μM, 1.1 μM, 370 nM, 123 nM, 41 nM, 13.7 nM, and 4.6 nM.

FLIPR determination of intracellular calcium level was used as a read-out for NMDAR activation.

E. Methods and Procedures

400× compound plates were prepared by Echo Labcyte system, containing in every well: 300 nl/well of 400× L-glutamate/glycine solution in H₂O and 300 nl/well of 400×test item solution in DMSO. 400× compound plate was stored at −20° C. until FLIPR experimental day.

4× compound plate was generated from 400× compound plate by addition of up to 30 μl/well of compound buffer on FLIPR experimental day. 4× L-glutamate solution was directly prepared only for 400 mM concentration, and dispensed in columns 1 and 12 of 4× compound plate.

A FLIPR system was used to monitor intracellular calcium level in NMDAR cell lines, pre-loaded for 1 hour with Fluo-4, and then washed with assay buffer. Intracellular calcium level was monitored for 10 seconds before and 5 minutes after test item addition, in presence of L-glutamate and glycine.

F. Data Handling and Analysis

AUC values of fluorescence were measured by ScreenWorks 4.1 (Molecular Devices) FLIPR software, to monitor calcium level during the 5 minutes after test item addition. Then, data were normalized by Excel 2013 (Microsoft Office) software, using wells added with 10 μM L-glutamate plus 10 μM glycine (column 23) as high control, and wells added with assay buffer only (column 24) as low control.

To assess plate quality, Z′ calculations were performed in Excel. Z′ was calculated according to the following equation:

Z′=1−3(σₕ+σₗ)/|μₕ−μₗ|

where μ and σ are the means and the standard deviations of the means of high (h) and low (l) controls, respectively.

A four parameter logistic equation was used in Prism 8 (GraphPad) software to calculate L-glutamate EC₅₀ and maximal effect, in the different experimental conditions:

Y=Bottom+(Top−Bottom)/(1+10\hat{ }((LogEC₅₀−Log[A])*HillSlope))

where Y is % effect of L-glutamate and [A] is L-glutamate molar concentration.

An operational equation for allosteric modulators (Leach K, Sexton P M and Christopoulos A, Allosteric GPCR modulators: taking advantage of permissive receptor pharmacology, Trends Pharmacol. Sci. 28: 382-389, 2007; Kenakin T P, Overview of receptor interaction of agonists and antagonists, Curr. Protoc. Pharmacol. Chapter 4: Unit 4.1, 2008, Kenakin T P, Biased signalling and allosteric machines: new vistas and challenges for drug discovery, Br. J. Pharmacol. 165: 1659-1669, 2012) was created in Prism 8 (GraphPad) software to estimate KB and a parameters for every test item, with the assumption that, as a pore blocker, every test item would be able to produce a compete blockade of agonist response, at sufficiently high concentration:

$$Y = {E}_{MAX} \frac{\frac{τ [ A ]}{E {C}_{50} ( τ + 1 )}}{( ( ( \frac{[ A ]}{E {C}_{50} ( τ + 1 )} ) + ( \frac{τ [ A ]}{E {C}_{50} ( τ + 1 )} ) ) ⋆ ( 1 + \frac{α [ B ]}{{K}_{B}} ) ) + \frac{[ B ]}{{K}_{B}} + 1}$$

where Y is % effect of L-glutamate; [A] is L-glutamate molar concentration; E_{MAX} is maximal possible L-glutamate effect, estimated from four parameter logistic equation; EC₅₀ is half maximal effective L-glutamate concentration, estimated from four parameter logistic equation; τ is an arbitrary L-glutamate efficacy value at NMDAR (set τ=100 for all receptors, in absence of consistent values for L-glutamate dissociation equilibrium constant in human dihetromeric NMDAR, which would be required to estimate T from EC₅₀); [B] is test item molar concentration; K_{B} is the estimated test item equilibrium dissociation constant; and a is the estimated cooperativity term, which indicates the effect of test item on L-glutamate dissociation equilibrium constant for the receptor (i.e., a is the estimated ratio between L-glutamate equilibrium dissociation constant in absence and in presence of test item, and it is expected to be 0<α≤1 for a negative allosteric modulator affecting agonist equilibrium dissociation constant).

The % affinity ratio was computed from estimated affinities, which are the reciprocal of K_{B}, and considering the highest affinity for a NMDAR subtype as 100%.

G. Protocol Deviations

The preparation of 400× concentrated solutions of L-glutamate and glycine occurred in H₂O, rather than in DMSO, due to poor L-glutamate solubility in DMSO. This protocol deviation neither affected the overall interpretation nor compromised the integrity of the study.

H. Results

1 Plate Z′ Values

5 cell plates for every cell line (GluN1-GluN2A, GluN1-GluN2B, GluN1-GluN2C, GluN1-GluN2D) were tested with the same compound plate, containing all test items.

All cell plates resulted with Z′ values >0.4, and were accepted.

Z′ values for GluN1-GluN2A for plates 1 to 5 were: 0.82, 0.80, 0.83, 0.83, 0.83;
Z′ values for GluN1-GluN2B for plates 1 to 5 were: 0.80, 0.77, 0.77, 0.81, 0.83;
Z′ values for GluN1-GluN2C for plates 1 to 5 were: 0.73, 0.53, 0.74, 0.71, 0.76; and
Z′ values for GluN1-GluN2D for plates 1 to 5 were: 0.70, 0.74, 0.65, 0.44, 0.64.

An additional 5 cell plates with GluN1-GluN2C cells were discarded, for low fluorescence values due to low receptor expression in that batch of cells.

2 L-Glutamate CRC

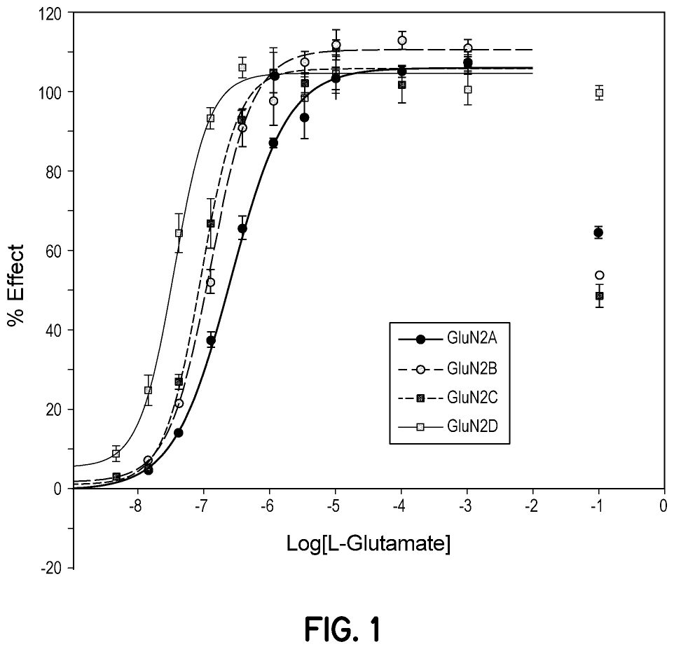

L-glutamic acid CRC in presence of 10 μM glycine was obtained for every cell line, and relative GraphPad Prism plot is shown in FIG. 1. Data are reported as mean±SEM, n=5.

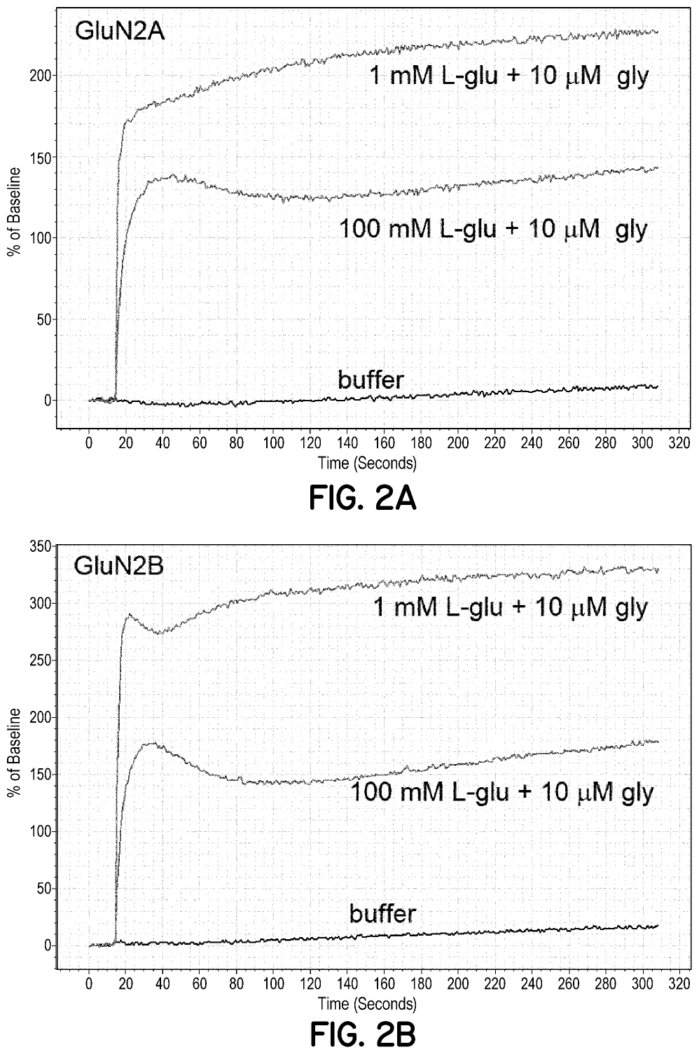

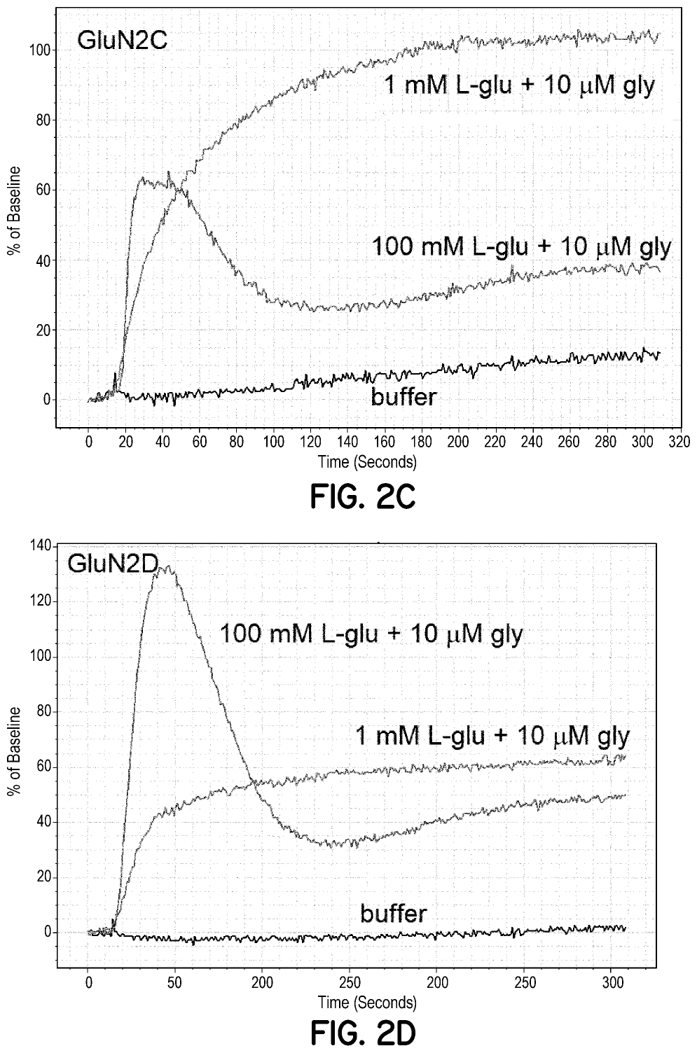

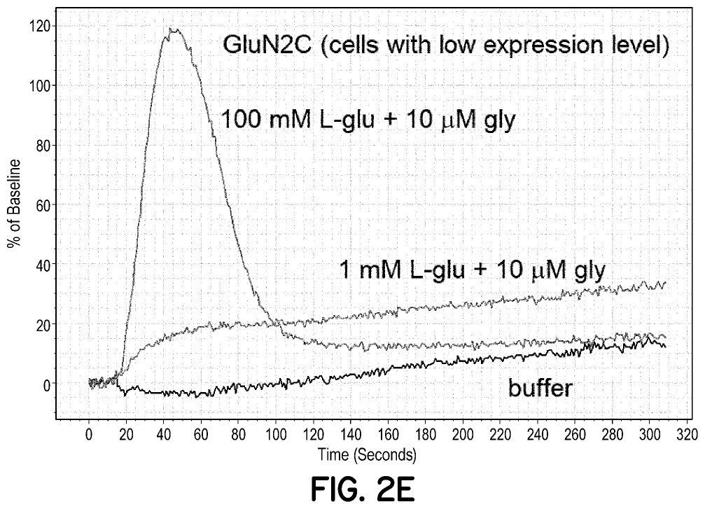

At 100 mM L-glutamate, % fluorescence values resulted sensibly lower, for all cell lines except GluN2D, and time-course of fluorescence resulted different from all other concentrations, with an initial transient peak lasting about 90 seconds. This transient peak was visible in all cell lines and especially in GluN2C and GluN2D cell lines, possibly due to lower expression levels of NMDAR in those cells, and even more in a GluN2C batch of cells expressing low levels of NMDAR (see traces in FIGS. 2A-2E). Therefore, 100 mM L-glutamate were reported in graphs but removed from data analysis.

Best-fit values for the 4 cell lines resulted as follows in Table 2:

TABLE 2

| | GluN2A | GluN2B | GluN2C | GluN2D |
|---|---|---|---|---|
| LogEC₅₀ | −6.6 | −6.9 | −7.1 | −7.5 |
| EC₅₀ (M) | 2.5e−007 | 1.3e−007 | 8.7e−008 | 3.4e−008 |
| HillSlope | 1.0 | 1.3 | 1.5 | 1.6 |
| Bottom | −0.62 | 1.7 | 0.88 | 5.3 |
| Top | 106 | 111 | 106 | 105 |
| Span | 107 | 109 | 105 | 99 |

3 Dextromethadone

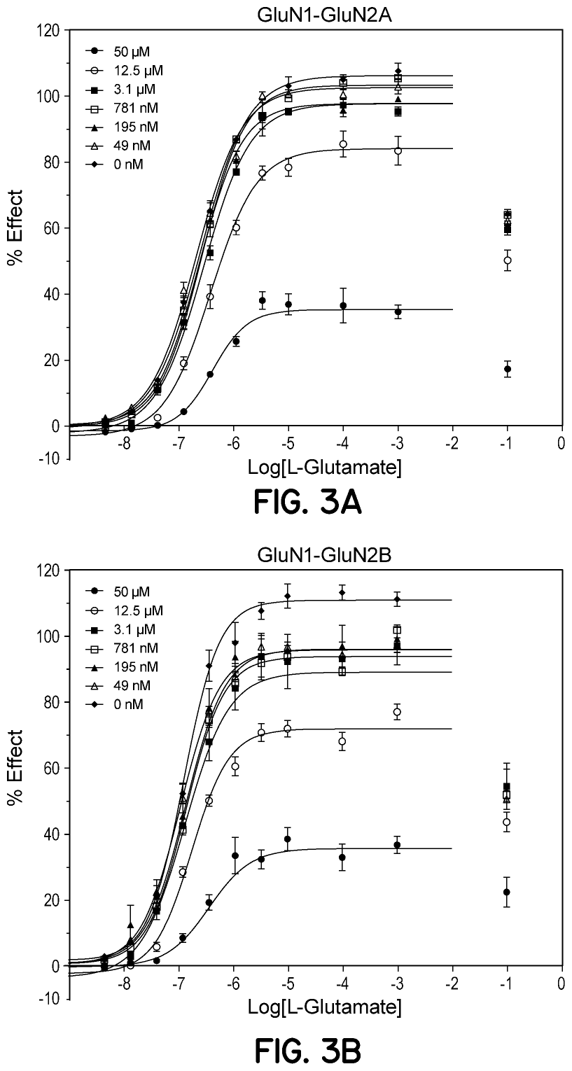

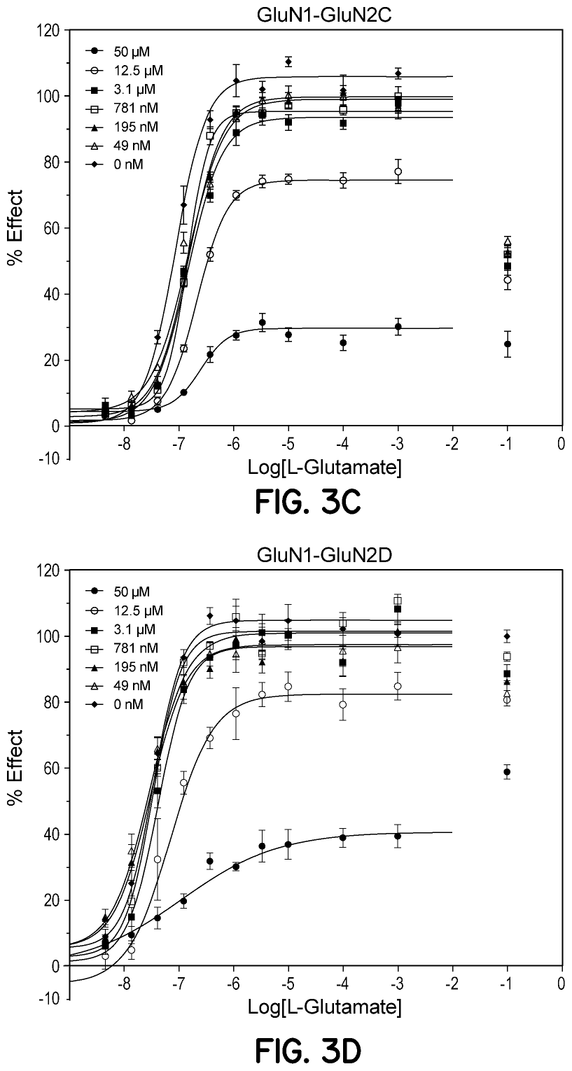

Dextromethadone effect on L-glutamate CRC in 4 NMDA receptor types is shown in FIGS. 3A-3D. 100 mM L-glutamate values were not used for the fittings. Data are reported as mean±SEM, n=5.

Dextromethadone four parameter logistic equation best-fit values resulted in GraphPad Prism data analysis as shown in Tables 3-6 below:

TABLE 3

| GluN2A | 50 μM | 12.5 μM | 3.1 μM | 781 nM | 195 nM | 49 nM | 0 nM |
|---|---|---|---|---|---|---|---|
| Bottom | −1.4 | −3.1 | −2.0 | −0.087 | 0.37 | −0.13 | −0.62 |
| Top | 35 | 84 | 98 | 103 | 98 | 103 | 106 |
| LogEC₅₀ | −6.4 | −6.4 | −6.6 | −6.6 | −6.6 | −6.7 | −6.6 |
| HillSlope | 1.4 | 1.0 | 1.0 | 1.1 | 1.1 | 1.0 | 1.0 |
| EC₅₀ (M) | 4.1e−7 | 3.8e−7 | 2.8e−7 | 2.6e−7 | 2.3e−7 | 2.1e−7 | 2.5e−7 |

TABLE 4

| GluN2B | 50 μM | 12.5 μM | 3.1 μM | 781 nM | 195 nM | 49 nM | 0 nM |
|---|---|---|---|---|---|---|---|
| Bottom | −0.34 | −2.3 | −3.6 | 0.52 | 0.68 | 0.43 | 1.7 |
| Top | 35 | 72 | 89 | 93 | 96 | 96 | 111 |
| LogEC₅₀ | −6.4 | −6.7 | −6.9 | −6.9 | −6.9 | −7.0 | −6.9 |
| HillSlope | 1.1 | 1.3 | 1.1 | 1.2 | 1.2 | 1.2 | 1.3 |
| EC₅₀ (M) | 3.7e−7 | 1.8e−7 | 1.3e−7 | 1.4e−7 | 1.4e−7 | 1.1e−7 | 1.3e−7 |

TABLE 5

| GluN2C | 50 μM | 12.5 μM | 3.1 μM | 781 nM | 195 nM | 49 nM | 0 nM |
|---|---|---|---|---|---|---|---|
| Bottom | 4.5 | 1.7 | 1.2 | 5.3 | 2.9 | 4.3 | 0.88 |
| Top | 30 | 75 | 94 | 95 | 100 | 99 | 106 |
| LogEC₅₀ | −6.6 | −6.7 | −6.8 | −6.8 | −6.8 | −6.8 | −7.1 |
| HillSlope | 1.7 | 1.5 | 1.4 | 2.2 | 1.4 | 1.3 | 1.5 |
| EC₅₀ (M) | 2.5e−7 | 2.1e−7 | 1.5e−7 | 1.4e−7 | 1.5e−7 | 1.5e−7 | 8.7e−8 |

TABLE 6

| GluN2D | 50 μM | 12.5 μM | 3.1 μM | 781 nM | 195 nM | 49 nM | 0 nM |
|---|---|---|---|---|---|---|---|
| Bottom | −0.55 | −5.6 | 1.1 | 2.6 | 5.3 | 5.1 | 5.3 |
| Top | 41 | 82 | 97 | 101 | 97 | 101 | 105 |
| LogEC₅₀ | −6.9 | −7.1 | −7.4 | −7.5 | −7.5 | −7.5 | −7.5 |
| HillSlope | 0.49 | 1.1 | 1.5 | 1.7 | 1.3 | 1.3 | 1.6 |
| EC₅₀ (M) | 1.1e−7 | 7.1e−8 | 4.2e−8 | 3.4e−8 | 3.0e−8 | 2.9e−8 | 3.4e−8 |

Operational analysis for allosteric modulators resulted in the K_{B}, % affinity ratio and α values shown in Table 7:

TABLE 7

| Cell line | K_{B} (M) | % affinity ratio | α | |
|---|---|---|---|---|
| GluN2A | 8.9e−6 | 51 | | 0.22 |
| GluN2B | 6.1e−6 | 74 | | 0.26 |
| GluN2C | 4.5e−6 | 100 | | 0.17 |
| GluN2D | 7.8e−6 | 58 | | 0.22 |

4 Memantine

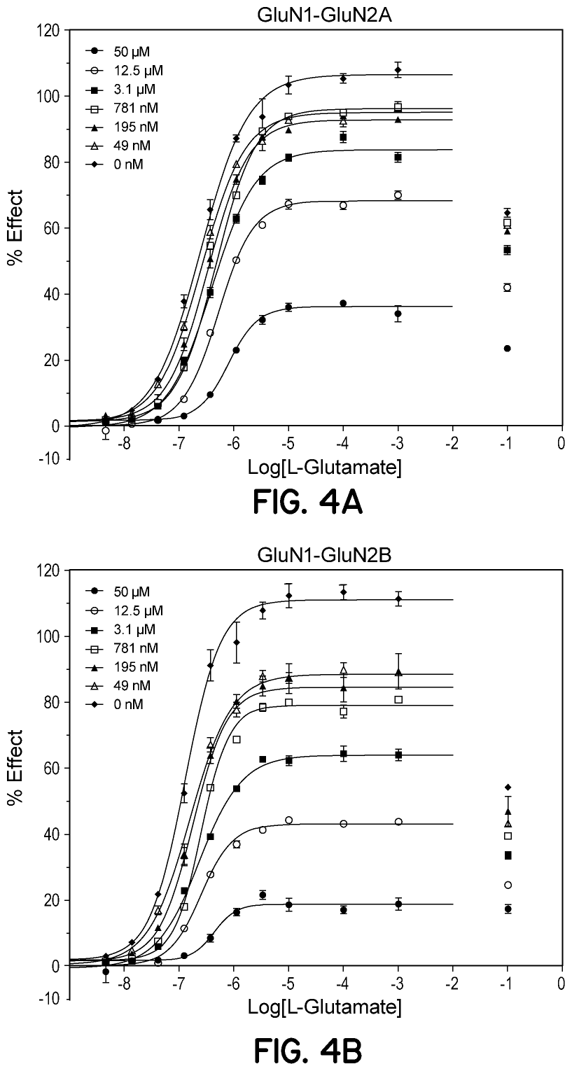

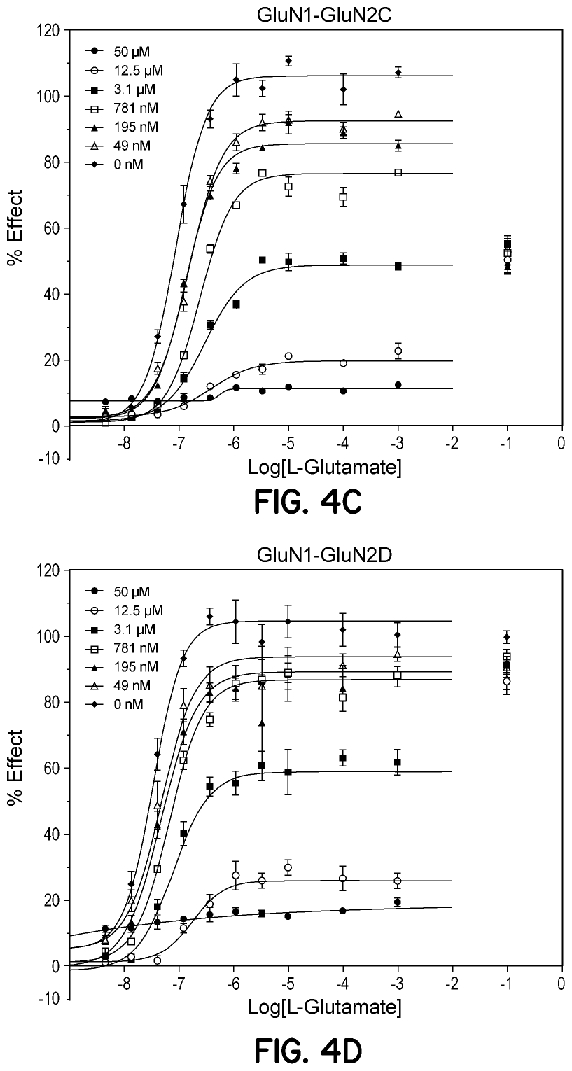

Memantine effect on L-glutamate CRC in 4 NMDA receptor types is shown in FIGS. 4A-4D. 100 mM L-glutamate values were not used for the fittings. Data are reported as mean±SEM, n=5.

Memantine four parameter logistic equation best-fit values resulted in GraphPad Prism data analysis as shown below in Tables 8-11 (values which are not considered a reliable fit are typed in boldface and underlined):

TABLE 8

| GluN2A | 50 μM | 12.5 μM | 3.1 μM | 781 nM | 195 nM | 49 nM | 0 nM |
|---|---|---|---|---|---|---|---|
| Bottom | 1.5 | −0.14 | −0.58 | 1.1 | 1.3 | 0.84 | −0.62 |
| Top | 36 | 68 | 83 | 96 | 92 | 95 | 106 |
| LogEC₅₀ | −6.1 | −6.3 | −6.4 | −6.3 | −6.5 | −6.6 | −6.6 |
| HillSlope | 1.6 | 1.3 | 1.1 | 1.2 | 1.2 | 1.1 | 1.0 |
| EC₅₀ (M) | 8.0e−7 | 5.2e−7 | 4.0e−7 | 4.7e−7 | 3.4e−7 | 2.6e−7 | 2.5e−7 |

TABLE 9

| GluN2B | 50 μM | 12.5 μM | 3.1 μM | 781 nM | 195 nM | 49 nM | 0 nM |
|---|---|---|---|---|---|---|---|
| Bottom | 1.5 | −0.073 | −0.85 | 1.6 | 1.3 | 0.24 | 1.7 |
| Top | 19 | 43 | 64 | 79 | 84 | 88 | 111 |
| LogEC₅₀ | −6.4 | −6.6 | −6.6 | −6.6 | −6.7 | −6.8 | −6.9 |
| HillSlope | 2.1 | 1.5 | 1.1 | 1.7 | 1.4 | 1.1 | 1.3 |
| EC₅₀ (M) | 4.3e−7 | 2.5e−7 | 2.3e−7 | 2.5e−7 | 1.8e−7 | 1.6e−7 | 1.3e−7 |

TABLE 10

| GluN2C | 50 μM | 12.5 μM | 3.1 μM | 781 nM | 195 nM | 49 nM | 0 nM |
|---|---|---|---|---|---|---|---|
| Bottom | 7.4 | 2.5 | 1.3 | 0.92 | 2.0 | 2.2 | 0.88 |
| Top | 11 | 20 | 49 | 76 | 85 | 92 | 106 |
| LogEC₅₀ | −6.3 | −6.4 | −6.5 | −6.6 | −6.9 | −6.8 | −7.1 |
| HillSlope | 6.1 | 1.1 | 1.2 | 1.4 | 1.5 | 1.4 | 1.5 |
| EC₅₀ (M) | 5.5e−7 | 3.8e−7 | 3.0e−7 | 2.4e−7 | 1.3e−7 | 1.5e−7 | 8.7e−8 |

TABLE 11

| GluN2D | 50 μM | 12.5 μM | 3.1 μM | 781 nM | 195 nM | 49 nM | 0 nM |
|---|---|---|---|---|---|---|---|
| Bottom | −97133 | 1.4 | −1.3 | 1.1 | −0.19 | 5.1 | 5.3 |
| Top | 19 | 26 | 59 | 87 | 89 | 94 | 105 |
| LogEC₅₀ | −37 | −6.7 | −7.1 | −7.2 | −7.3 | −7.3 | −7.5 |
| HillSlope | 0.14 | 1.5 | 1.3 | 1.4 | 1.3 | 1.4 | 1.6 |
| EC₅₀ (M) | 1.6e−37 | 1.8e−7 | 8.0e−8 | 6.8e−8 | 4.8e−8 | 4.8e−8 | 3.4e−8 |

Operational analysis for allosteric modulators resulted in the following K_{B}, % affinity ratio and α values shown in Table 12:

TABLE 12

| Cell line | K_{B} (M) | % affinity ratio | α | |
|---|---|---|---|---|
| GluN2A | 3.6e−6 | 8 | | 0.15 |
| GluN2B | 5.8e−7 | 48 | | 0.094 |
| GluN2C | 2.8e−7 | 100 | | 0.10 |
| GluN2D | 5.9e−7 | 47 | | 0.13 |

5 (±)-Ketamine

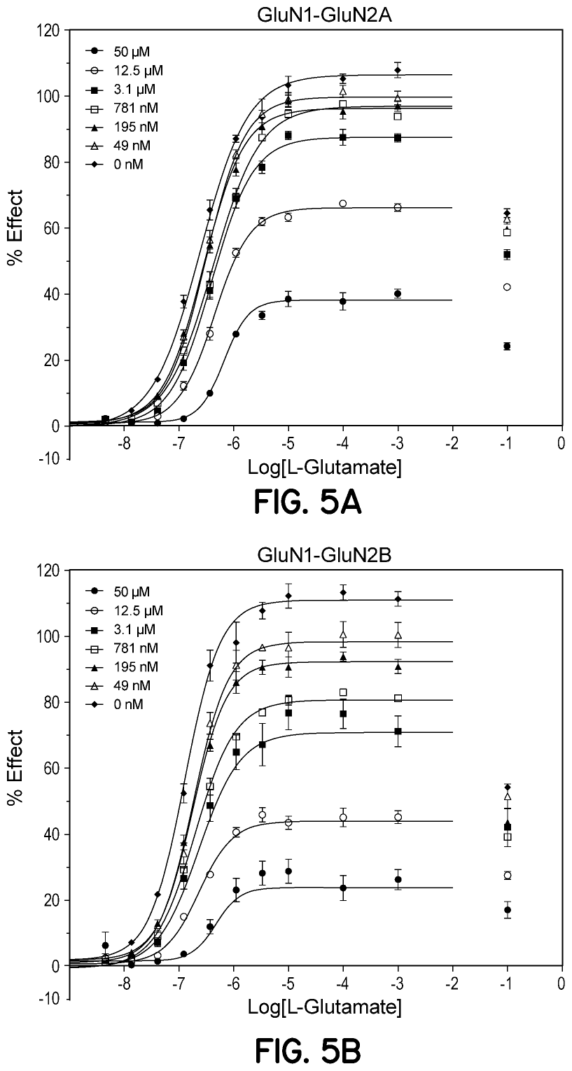

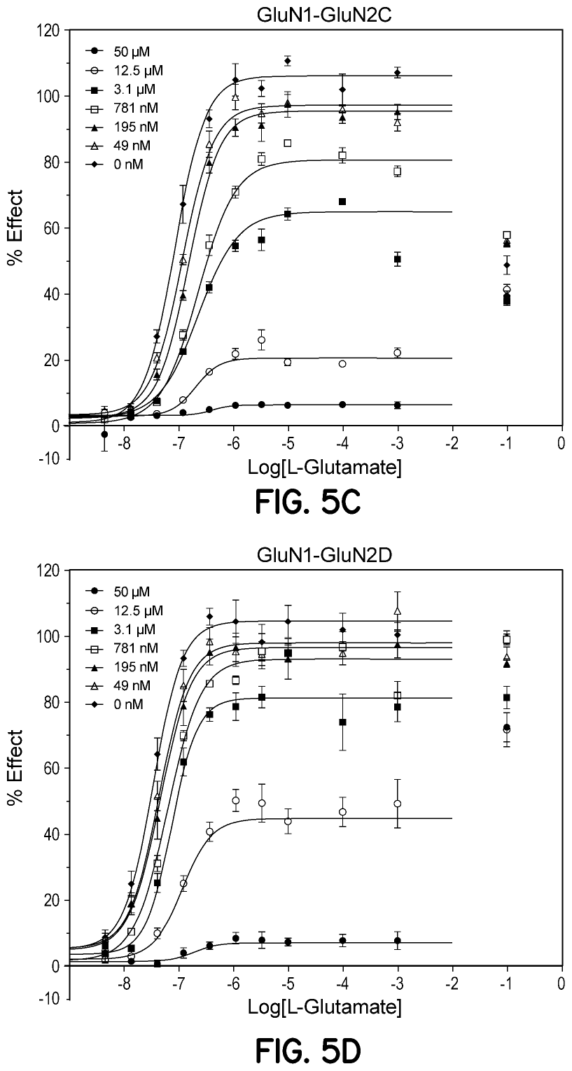

(±)-Ketamine effect on L-glutamate CRC in 4 NMDA receptor types is shown in FIGS. 5A-5D. 100 mM L-glutamate values were not used for the fittings. Data are reported as mean±SEM, n=5.

(±)-Ketamine four parameter logistic equation best-fit values resulted in GraphPad Prism data analysis as shown below in Tables 13-16.

TABLE 13

| GluN2A | 50 μM | 12.5 μM | 3.1 μM | 781 nM | 195 nM | 49 nM | 0 nM |
|---|---|---|---|---|---|---|---|
| Bottom | 0.99 | 0.48 | −0.090 | −0.20 | 0.62 | 0.98 | −0.62 |
| Top | 38 | 66 | 87 | 97 | 96 | 100 | 106 |
| LogEC₅₀ | −6.2 | −6.4 | −6.4 | −6.4 | −6.5 | −6.5 | −6.6 |
| HillSlope | 1.9 | 1.3 | 1.1 | 1.0 | 1.2 | 1.2 | 1.0 |
| EC₅₀ (M) | 6.7e−7 | 4.4e−7 | 4.0e−7 | 4.2e−7 | 2.8e−7 | 3.1e−7 | 2.5e−7 |

TABLE 14

| GluN2B | 50 μM | 12.5 μM | 3.1 μM | 781 nM | 195 nM | 49 nM | 0 nM |
|---|---|---|---|---|---|---|---|
| Bottom | 1.4 | 0.50 | −0.64 | −0.79 | 1.7 | 0.90 | 1.7 |
| Top | 24 | 44 | 70 | 80 | 92 | 98 | 111 |
| LogEC₅₀ | −6.3 | −6.6 | −6.6 | −6.7 | −6.8 | −6.7 | −6.9 |
| HillSlope | 2.0 | 1.4 | 1.1 | 1.2 | 1.4 | 1.4 | 1.3 |
| EC₅₀ (M) | 4.7e−7 | 2.3e−7 | 2.3e−7 | 2.0e−7 | 1.8e−7 | 1.8e−7 | 1.3e−7 |

TABLE 15

| GluN2C | 50 μM | 12.5 μM | 3.1 μM | 781 nM | 195 nM | 49 nM | 0 nM |
|---|---|---|---|---|---|---|---|
| Bottom | 3.0 | 2.8 | 2.1 | 0.59 | 2.5 | 3.2 | 0.88 |
| Top | 6.2 | 20 | 65 | 80 | 95 | 97 | 106 |
| LogEC₅₀ | −6.4 | −6.7 | −6.6 | −6.6 | −6.8 | −6.9 | −7.1 |
| HillSlope | 2.5 | 2.0 | 1.2 | 1.3 | 1.5 | 1.5 | 1.5 |
| EC₅₀ (M) | 4.1e−7 | 2.1e−7 | 2.3e−7 | 2.3e−7 | 1.6e−7 | 1.2e−7 | 8.7e−8 |

TABLE 16

| GluN2D | 50 μM | 12.5 μM | 3.1 μM | 781 nM | 195 nM | 49 nM | 0 nM |
|---|---|---|---|---|---|---|---|
| Bottom | 1.5 | 2.1 | 3.6 | 1.7 | 4.9 | 5.4 | 5.3 |
| Top | 7.1 | 45 | 81 | 93 | 97 | 98 | 105 |
| LogEC₅₀ | −6.7 | −6.9 | −7.1 | −7.2 | −7.3 | −7.4 | −7.5 |
| HillSlope | 2.0 | 1.6 | 1.8 | 1.4 | 1.5 | 1.6 | 1.6 |
| EC₅₀ (M) | 1.9e−7 | 1.2e−7 | 7.5e−8 | 6.3e−8 | 4.7e−8 | 4.4e−8 | 3.4e−8 |

Operational analysis for allosteric modulators resulted in the following K_{B}, % affinity ratio and α values shown in Table 17:

TABLE 17

| Cell line | K_{B} (M) | % affinity ratio | α |
|---|---|---|---|
| GluN2A | 4.3e−6 | 11 | 0.17 |
| GluN2B | 1.1e−6 | 42 | 0.14 |
| GluN2C | 4.6e−7 | 100 | 0.13 |
| GluN2D | 1.4e−6 | 33 | 0.15 |

6 (+)-MK 801

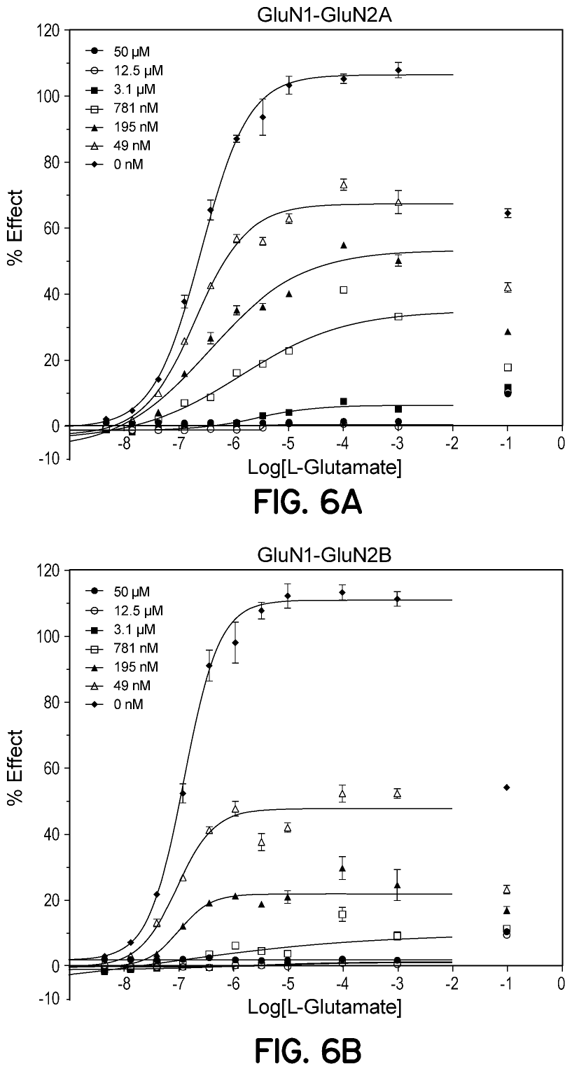

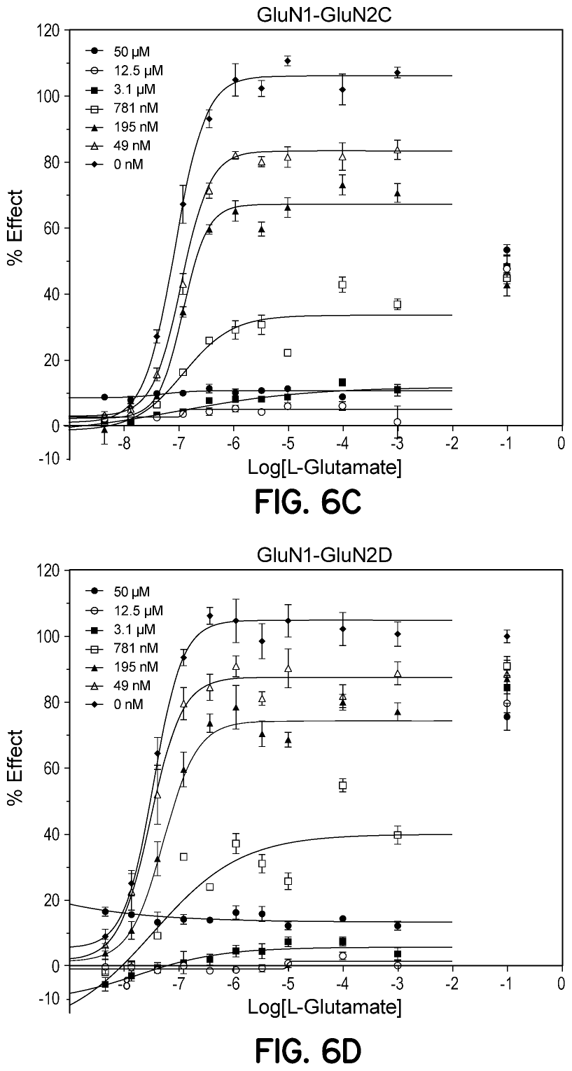

(+)-MK 801 effect on L-glutamate CRC in 4 NMDA receptor types is shown in FIGS. 6A-6D. 100 mM L-glutamate values were not used for the fittings. Data are reported as mean±SEM, n=5.

(+)-MK 801 four parameter logistic equation best-fit values resulted in GraphPad Prism data analysis as shown below in Tables 18-21 (values which are not considered a reliable fit are typed in boldface and underlined):

TABLE 18

| GluN2A | 50 μM | 12.5 μM | 3.1 μM | 781 nM | 195 nM | 49 nM | 0 nM |
|---|---|---|---|---|---|---|---|
| Bottom | N.A. | −1.3 | −1.5 | −4.5 | −7.4 | −3.0 | −0.62 |
| Top | N.A. | 0.21 | 6.1 | 35 | 53 | 67 | 106 |
| LogEC₅₀ | N.A. | −5.5 | −5.6 | −5.9 | −6.4 | −6.7 | −6.6 |
| HillSlope | N.A. | 30 | 0.81 | 0.46 | 0.52 | 0.91 | 1.0 |
| EC₅₀ (M) | N.A. | 3.4e−6 | 2.6e−6 | 1.3e−6 | 3.6e−7 | 2.0e−7 | 2.5e−7 |

TABLE 19

| GluN2B | 50 μM | 12.5 μM | 3.1 μM | 781 nM | 195 nM | 49 nM | 0 nM |
|---|---|---|---|---|---|---|---|
| Bottom | 1.7 | −0.37 | −1.3 | −7.4 | −0.47 | −0.35 | 1.7 |
| Top | 1.6 | 0.88 | 1.1 | 9.5 | 22 | 47 | 111 |
| LogEC₅₀ | 44 | −4.9 | −5.8 | −7.2 | −7.0 | −7.0 | −6.9 |
| HillSlope | 805 | 0.94 | 0.48 | 0.24 | 1.5 | 1.3 | 1.3 |
| EC₅₀ (M) | 1.3e+44 | 1.2e−5 | 1.7e−6 | 6.6e−8 | 1.0e−7 | 9.5e−8 | 1.3e−7 |

TABLE 20

| GluN2C | 50 μM | 12.5 μM | 3.1 μM | 781 nM | 195 nM | 49 nM | 0 nM |
|---|---|---|---|---|---|---|---|
| Bottom | 8.4 | 4.9 | −2.9 | −1.6 | 2.2 | 2.8 | 0.88 |
| Top | 11 | 2.5 | 12 | 33 | 67 | 83 | 106 |
| LogEC₅₀ | −7.3 | −6.9 | −7.0 | −6.9 | −6.9 | −6.9 | −7.1 |
| HillSlope | 1.7 | −18 | 0.36 | 0.97 | 1.9 | 1.6 | 1.5 |
| EC₅₀ (M) | 5.0e−8 | 1.2e−7 | 1.0e−7 | 1.3e−7 | 1.2e−7 | 1.1e−7 | 8.7e−8 |

TABLE 21

| GluN2D | 50 μM | 12.5 μM | 3.1 μM | 781 nM | 195 nM | 49 nM | 0 nM |
|---|---|---|---|---|---|---|---|
| Bottom | 13 | −1.0 | −11 | −20 | 1.1 | 1.5 | 5.3 |
| Top | 116593 | 1.3 | 5.5 | 40 | 74 | 87 | 105 |
| LogEC₅₀ | −23 | −5.0 | −7.7 | −7.5 | −7.3 | −7.5 | −7.5 |
| HillSlope | −0.30 | 30 | 0.56 | 0.54 | 1.4 | 1.5 | 1.6 |
| EC₅₀ (M) | 4.8e−24 | 9.6e−6 | 1.8e−8 | 3.4e−8 | 5.3e−8 | 3.1e−8 | 3.4e−8 |

Operational analysis for allosteric modulators resulted in the following K_{B}, % affinity ratio and α values shown in Table 22:

TABLE 22

| | | | |
|---|---|---|---|
| Cell line | K_{B} (M) | % affinity ratio | α |

| | | | |
|---|---|---|---|
| GluN2A | 1.1e−7 | 44 | 0.87 |
| GluN2B | 4.8e−8 | 100 | 1.0 |
| GluN2C | 1.4e−7 | 34 | 0.39 |
| GluN2D | 1.5e−7 | 32 | 0.36 |

7 Dextromethorphan

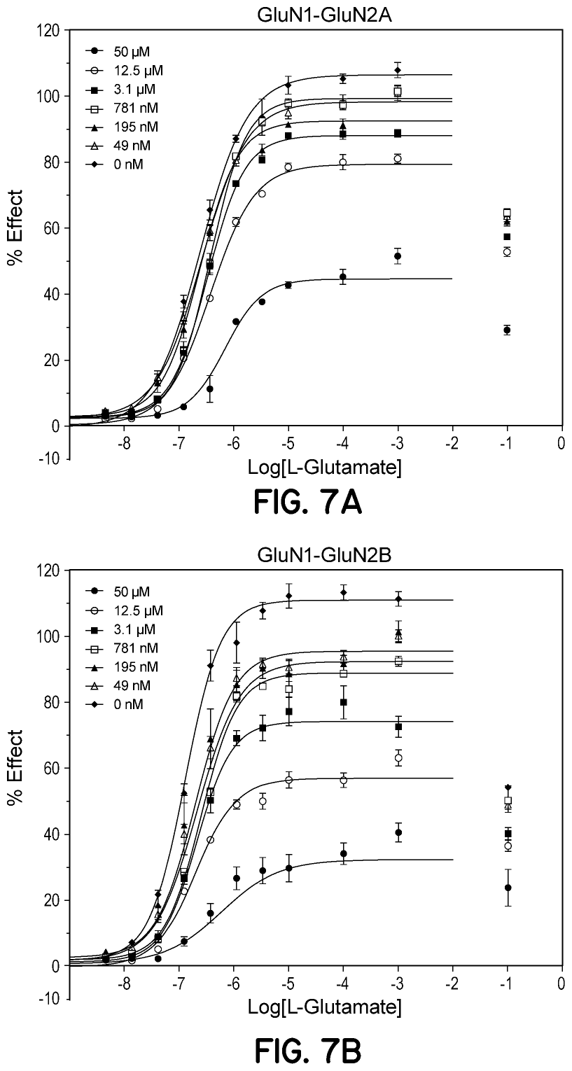

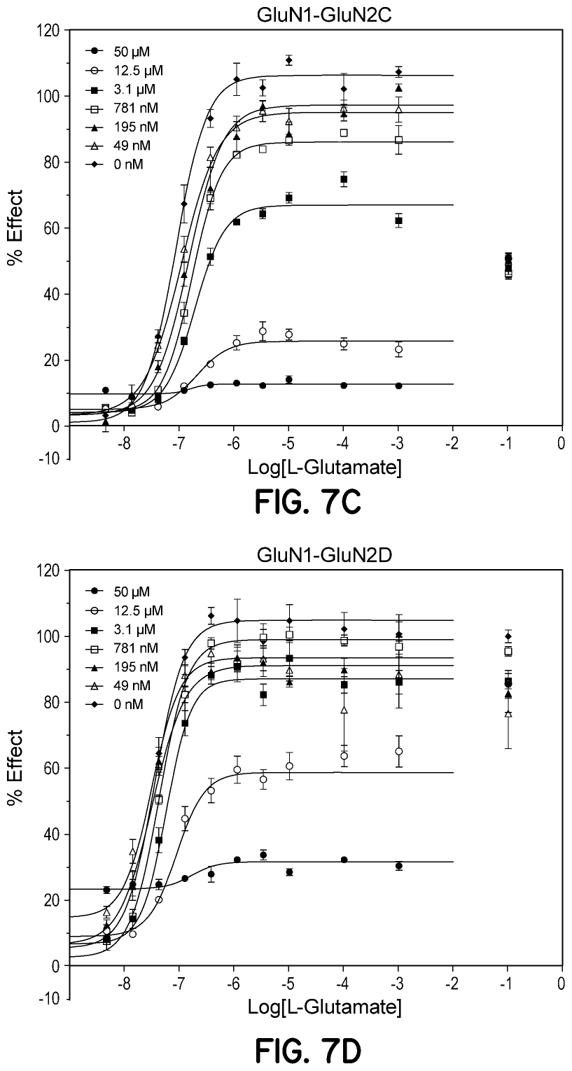

Dextromethorphan effect on L-glutamate CRC in 4 NMDA receptor types is shown in FIGS. 7A-7D. 100 mM L-glutamate values were not used for the fittings. Data are reported as mean±SEM, n=5.

Dextromethorphan four parameter logistic equation best-fit values resulted in GraphPad Prism data analysis as shown below in Tables 23-26 (values which are not considered a reliable fit are typed in boldface and underlined):

TABLE 23

| GluN2A | 50 μM | 12.5 μM | 3.1 μM | 781 nM | 195 nM | 49 nM | 0 nM |
|---|---|---|---|---|---|---|---|
| Bottom | 2.2 | 0.20 | 2.5 | 2.4 | 2.7 | 2.3 | −0.62 |
| Top | 44 | 79 | 88 | 99 | 92 | 98 | 106 |
| LogEC₅₀ | −6.2 | −6.4 | −6.5 | −6.4 | −6.6 | −6.6 | −6.6 |
| HillSlope | 1.3 | 1.1 | 1.3 | 1.3 | 1.2 | 1.0 | 1.0 |
| EC₅₀ (M) | 7.0e−7 | 3.8e−7 | 3.4e−7 | 3.8e−7 | 2.4e−7 | 2.6e−7 | 2.5e−7 |

TABLE 24

| GluN2B | 50 μM | 12.5 μM | 3.1 μM | 781 nM | 195 nM | 49 nM | 0 nM |
|---|---|---|---|---|---|---|---|
| Bottom | 0.49 | −0.38 | 1.1 | 1.7 | 1.5 | 2.4 | 1.7 |
| Top | 32 | 57 | 74 | 88 | 92 | 95 | 111 |
| LogEC₅₀ | −6.2 | −6.7 | −6.7 | −6.6 | −6.7 | −6.7 | −6.9 |
| HillSlope | 0.83 | 1.2 | 1.3 | 1.3 | 1.1 | 1.2 | 1.3 |
| EC₅₀ (M) | 5.9e−7 | 2.0e−7 | 2.1e−7 | 2.5e−7 | 2.1e−7 | 2.0e−7 | 1.3e−7 |

TABLE 25

| GluN2C | 50 μM | 12.5 μM | 3.1 μM | 781 nM | 195 nM | 49 nM | 0 nM |
|---|---|---|---|---|---|---|---|
| Bottom | 9.6 | 4.9 | 3.9 | 3.9 | 3.1 | 3.2 | 0.88 |
| Top | 13 | 26 | 67 | 86 | 97 | 95 | 106 |
| LogEC₅₀ | −6.8 | −6.7 | −6.7 | −6.8 | −6.8 | −7.0 | −7.1 |
| HillSlope | 3.0 | 1.5 | 1.6 | 1.6 | 1.4 | 1.3 | 1.5 |
| EC₅₀ (M) | 1.6e−7 | 2.1e−7 | 1.9e−7 | 1.7e−7 | 1.5e−7 | 1.1e−7 | 8.7e−8 |

TABLE 26

| GluN2D | 50 μM | 12.5 μM | 3.1 μM | 781 nM | 195 nM | 49 nM | 0 nM |
|---|---|---|---|---|---|---|---|
| Bottom | 23 | 8.8 | 6.6 | 2.4 | 6.3 | 15 | 5.3 |
| Top | 31 | 59 | 87 | 99 | 91 | 93 | 105 |
| LogEC₅₀ | −6.8 | −7.1 | −7.3 | −7.4 | −7.5 | −7.5 | −7.5 |
| HillSlope | 1.9 | 1.7 | 1.9 | 1.7 | 1.4 | 1.7 | 1.6 |
| EC₅₀ (M) | 1.6e−7 | 8.6e−8 | 5.6e−8 | 4.2e−8 | 3.1e−8 | 3.1e−8 | 3.4e−8 |

Operational analysis for allosteric modulators resulted in the following K_{B}, % affinity ratio and α values shown in Table 27:

TABLE 27

| Cell line | K_{B} (M) | % affinity ratio | α |
|---|---|---|---|
| GluN2A | 9.6e−6 | 13 | 0.25 |
| GluN2B | 1.9e−6 | 63 | 0.13 |
| GluN2C | 1.2e−6 | 100 | 0.24 |
| GluN2D | 6.7e−6 | 18 | 0.34 |

I. Discussion

L-glutamate effect on calcium mobilization showed differential activation of NMDAR heterodimeric receptors, the EC₅₀ rank order being GluN2A >GluN2B GluN2C>GluN2D, with EC₅₀ values of 2.5e-7, 1.3e-7, 8.7e-8, and 3.4e-8, respectively. The obtained potency rank order is in line with that described in literature with various methodologies (Paoletti P, Bellone C and Zhou Q, NMDA receptor subunit diversity: impact on receptor properties, synaptic plasticity and disease, Nat. Rev. Neurosci, 14: 383-400, 2013).

100 mM L-glutamate showed a calcium transient peak lasting about 90 seconds in all cell lines, more evident in a GluN2C batch of cells expressing low levels of NMDAR. It may be hypothesized that 100 mM L-glutamate effect on intracellular calcium levels might not be mediated by NMDAR, but rather by an osmotic cell reaction to such high concentration of a metabolite. The pathway involved in 100 mM L-glutamate induced intracellular calcium increase remains to be investigated.

5 test items were investigated for their effect, at 6 selected concentrations, on L-glutamate CRC: dextromethadone, memantine, (±)-ketamine, (+)-MK 801, and dextromethorphan. All 5 test items showed an insurmountable profile, typical of NMDAR pore blockers in FLIPR calcium assay. (+)-MK 801 resulted with highest estimated affinity for all NMDAR subtypes, compared to other test items, being able to reduce % effect of L-glutamate to less than 50% with all NMDAR subtypes already at 781 nM. (+)-MK 801 estimated K_{B} resulted 50 nM with any of the NMDAR subtypes. Memantine and (±)-ketamine resulted with K_{B} in the micromolar range, being sub-micromolar for memantine on GluN2B, GluN2C, GluN2D and for (±)-ketamine on GluN2C. Dextromethadone and dextromethorphan resulted with estimated K_{B} in the micromolar range with any of the NMDAR subtypes.

None of the compounds was selective for NMDAR containing a specific GluN2 subunit, although they mostly showed some GluN2 subunit preference. Among all tested compounds, dextromethadone showed the least subtype preference. All compounds except (+)-MK 801 showed preference for GluN2C containing subtypes compared to the other subtypes containing the subunits GluN2A, B, or D. (e.g., considering 100% the estimated % affinity for GluN2C containing NMDAR, then estimated % affinity for GluN2A containing NMDAR resulted: 51, 13, 11, and 8% for dextromethadone, dextromethorphan, (±)-ketamine, memantine, respectively. Only (+)-MK 801 showed slight preference for GluN2B containing NMDAR.

TABLE 28
K_{B} Table

| | K_{B} (μM) | | | |
|---|---|---|---|---|
| Test Item | GluN1/2A | GluN1/2B | GluN1/2C | GluN1/2D |
| Dextromethadone | 8.9 | 6.1 | 4.5 | 7.8 |
| Dextromethorphan | 9.6 | 1.9 | 1.2 | 6.7 |
| (±)-Ketamine | 4.3 | 1.1 | 0.46 | 1.4 |
| Memantine | 3.6 | 0.58 | 0.28 | 0.59 |
| (+)-MK 801 K_{B} | 0.11 | 0.048 | 0.14 | 0.15 |

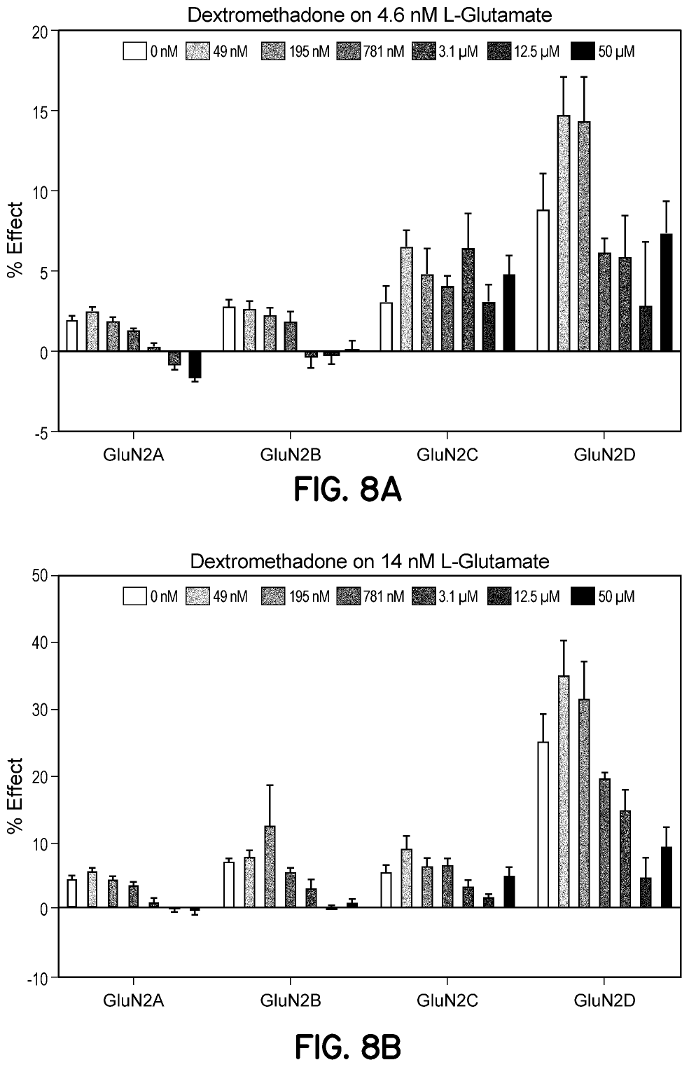

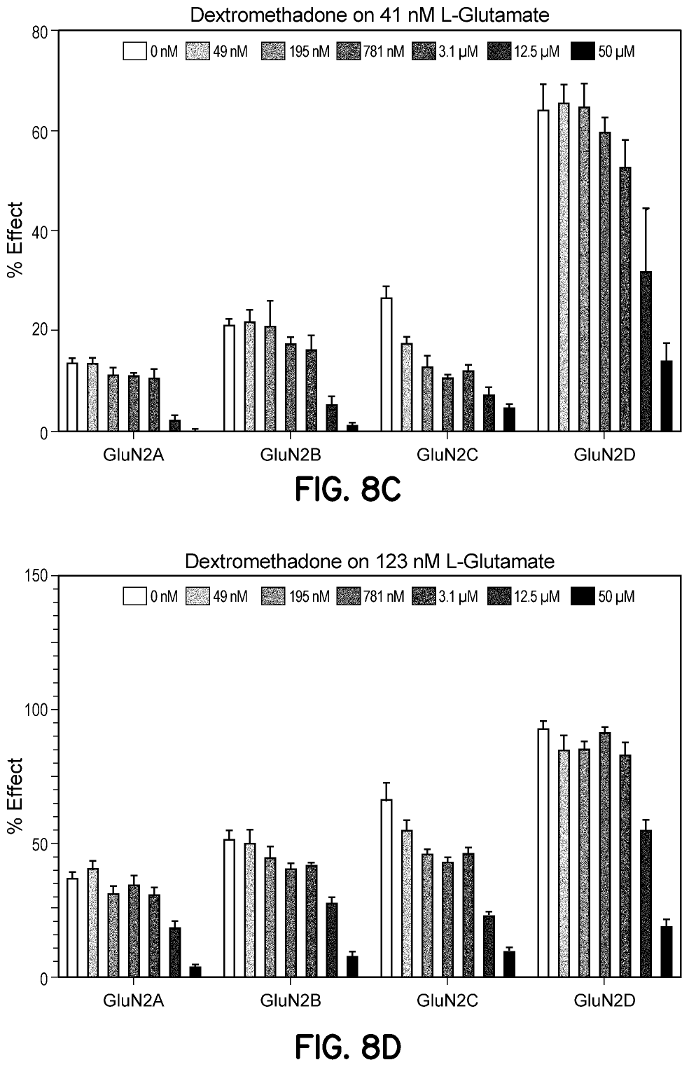

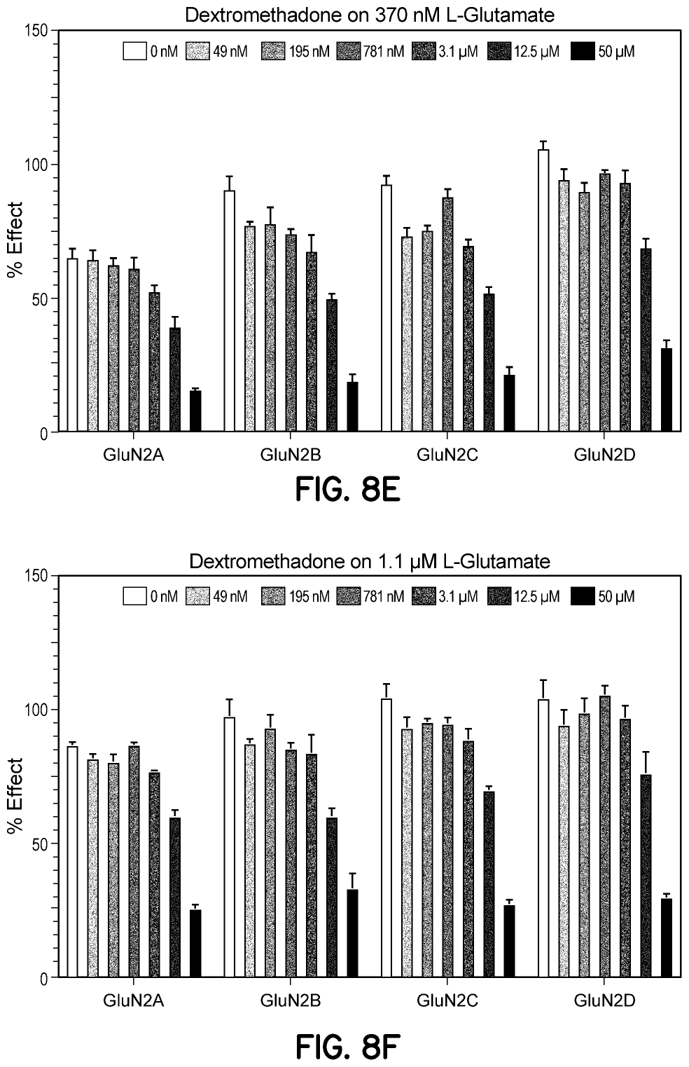

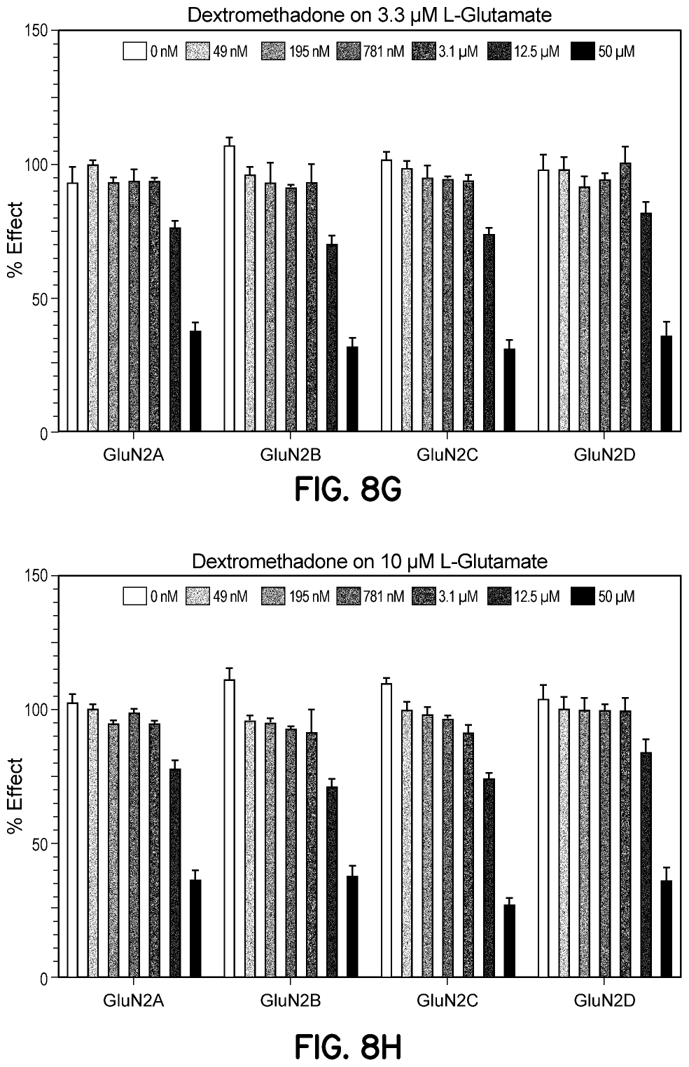

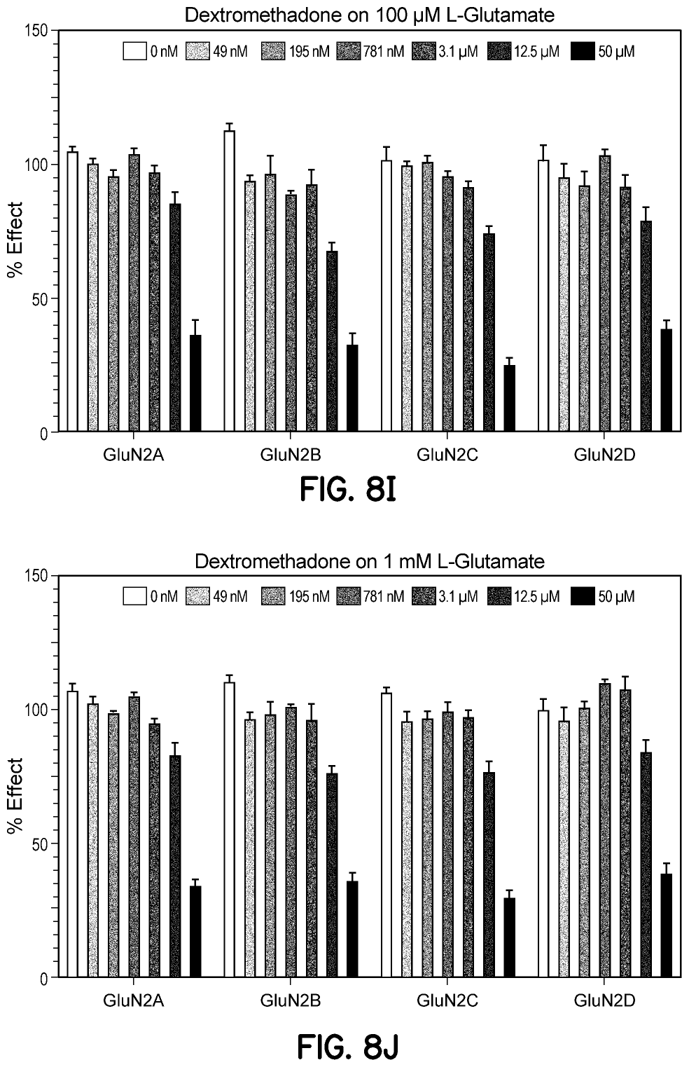

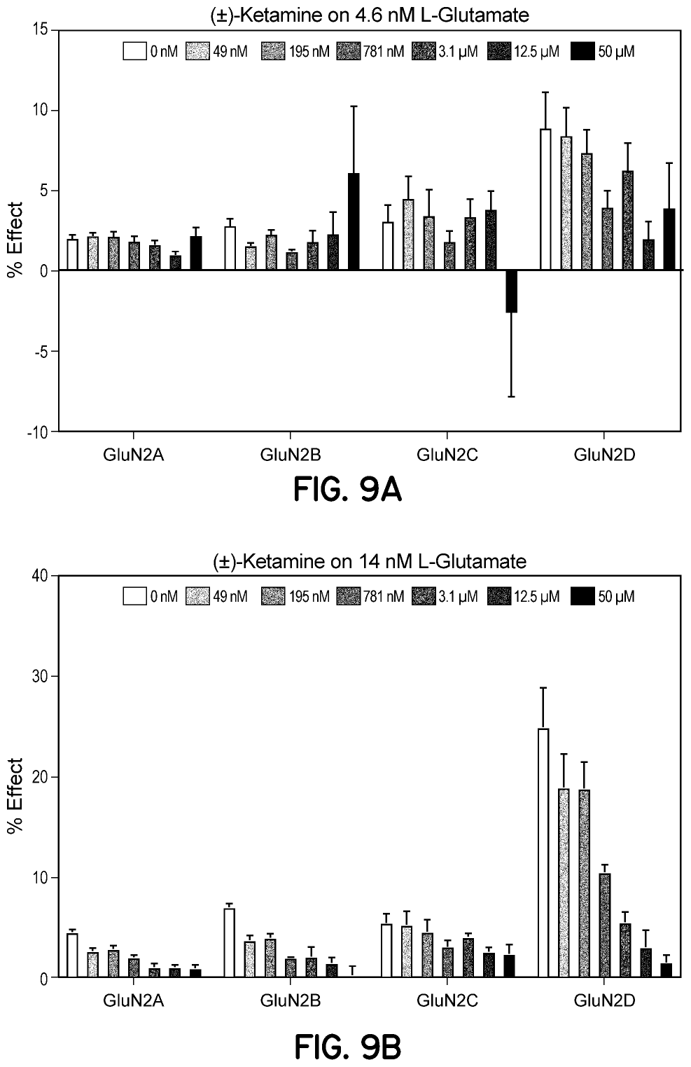

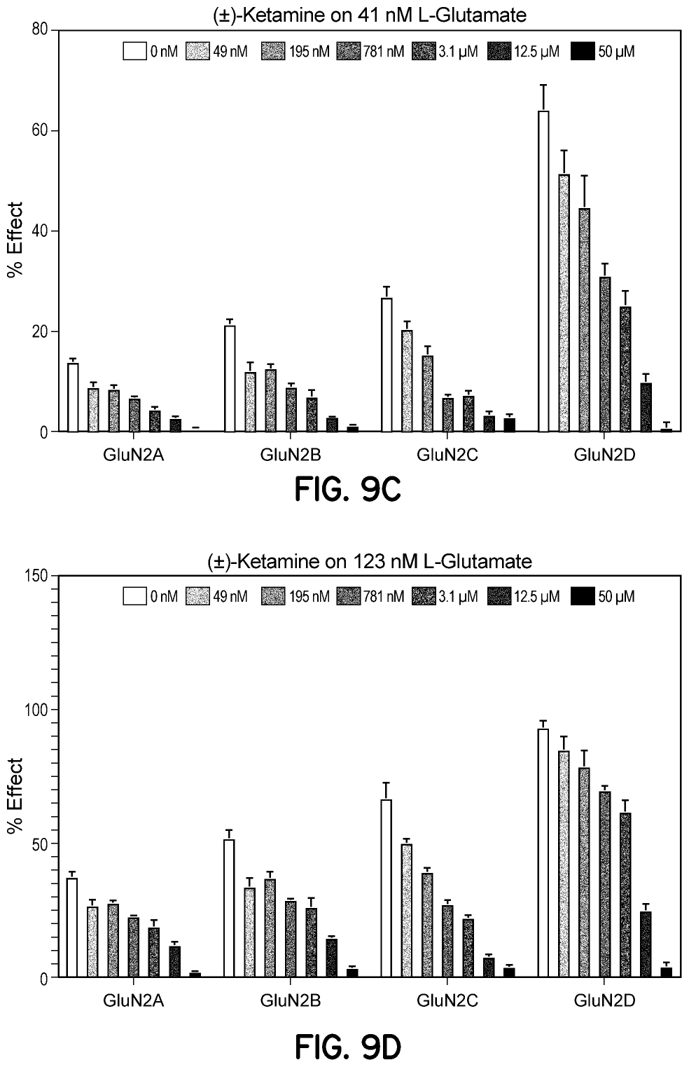

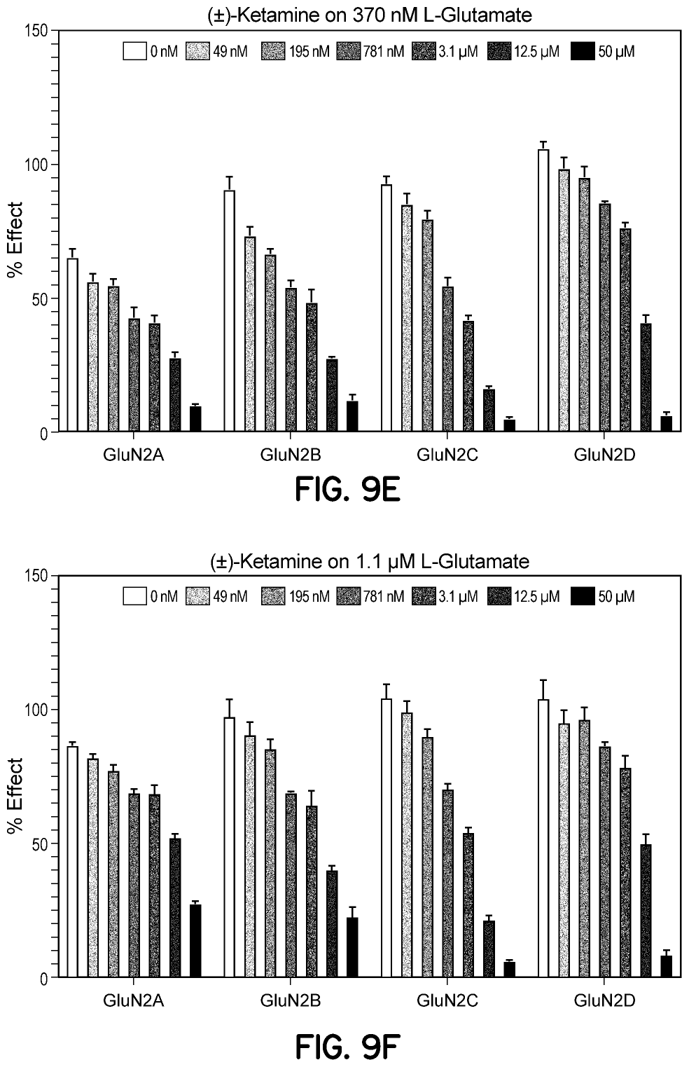

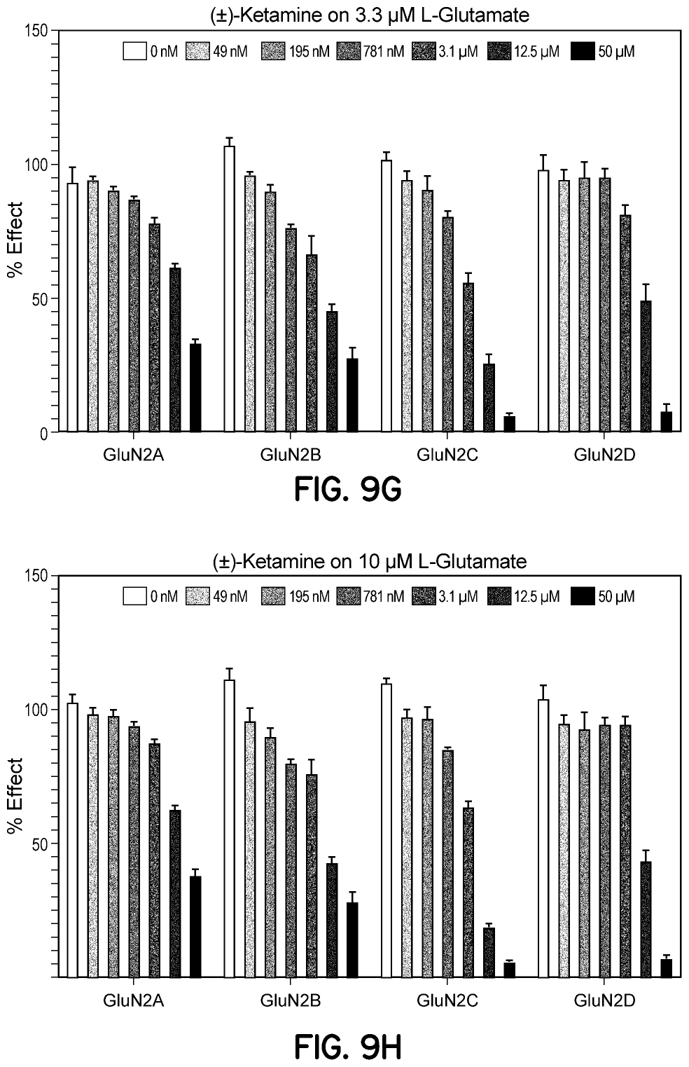

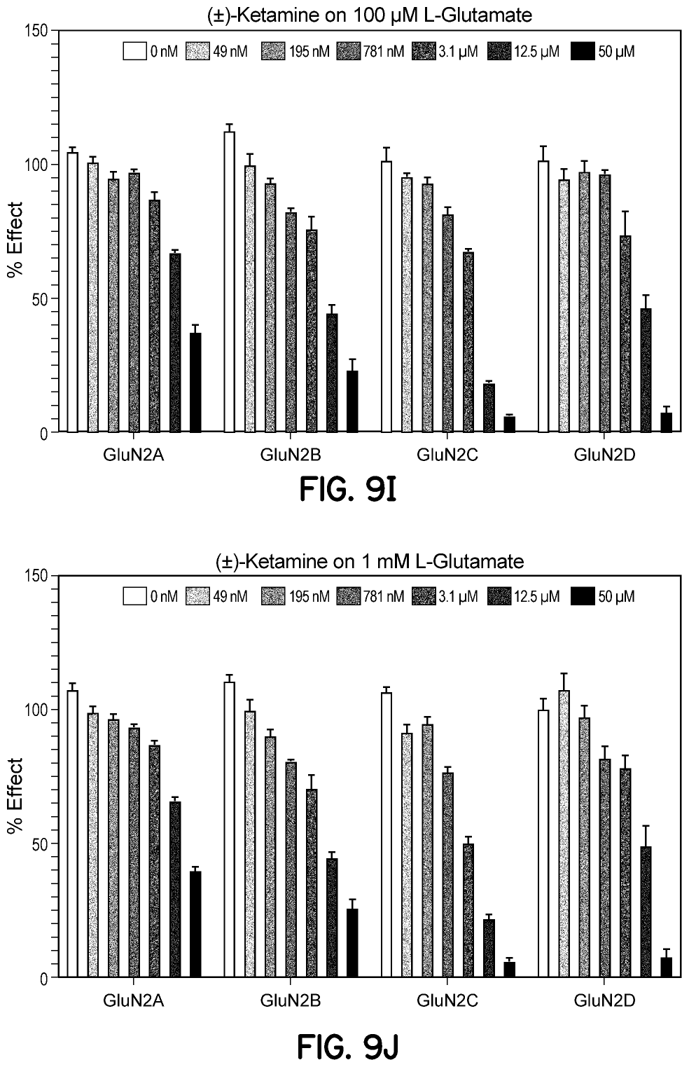

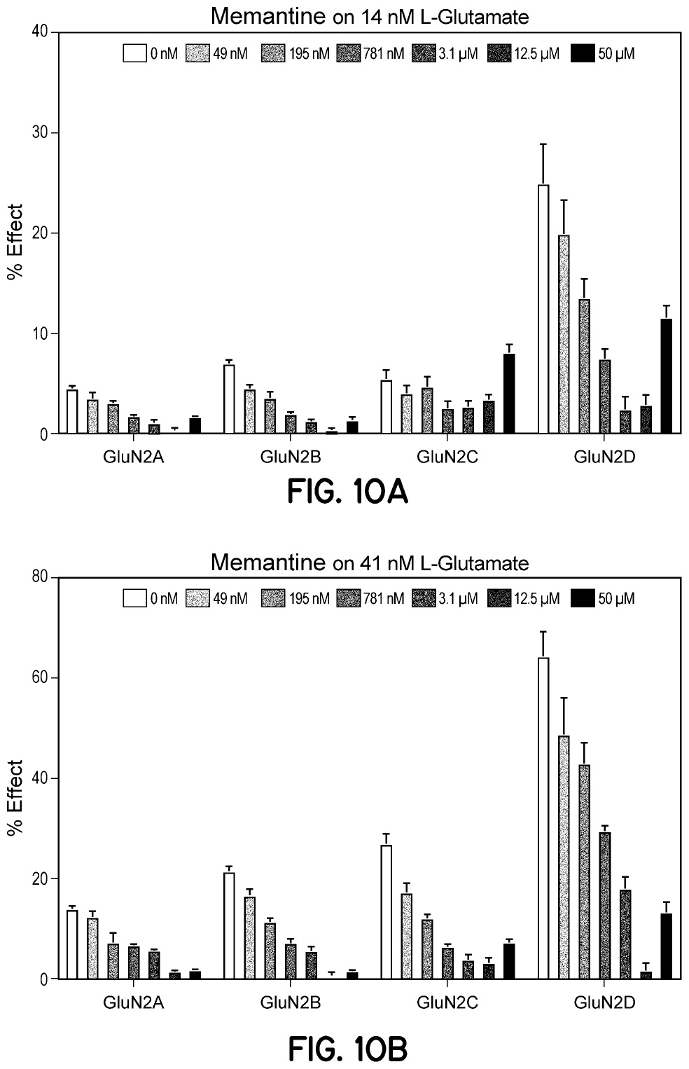

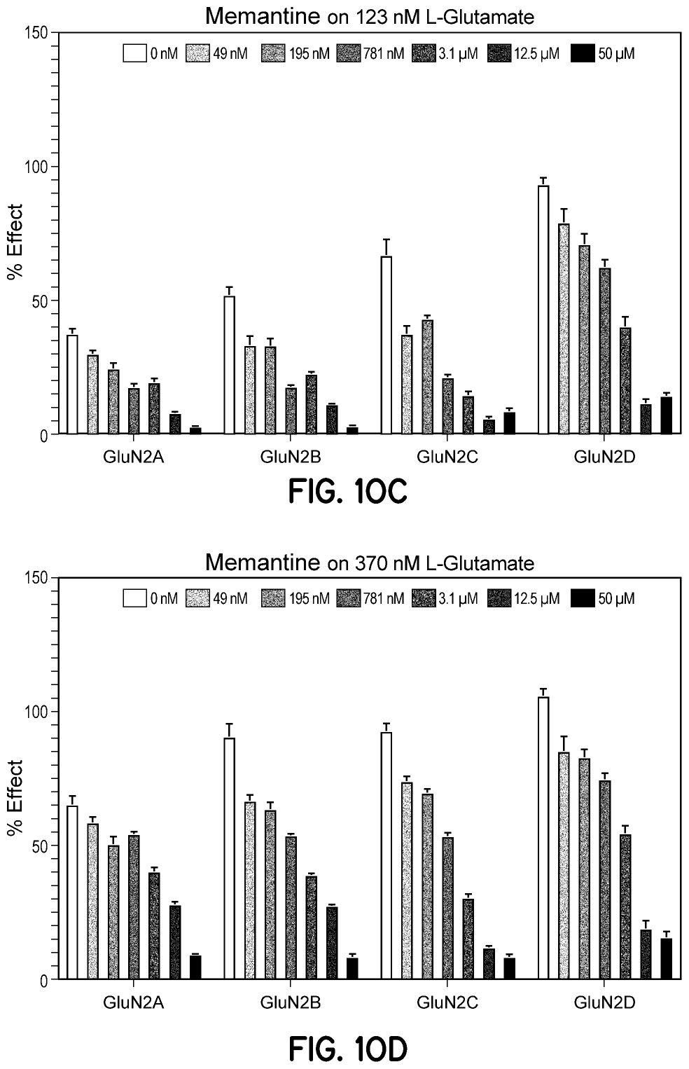

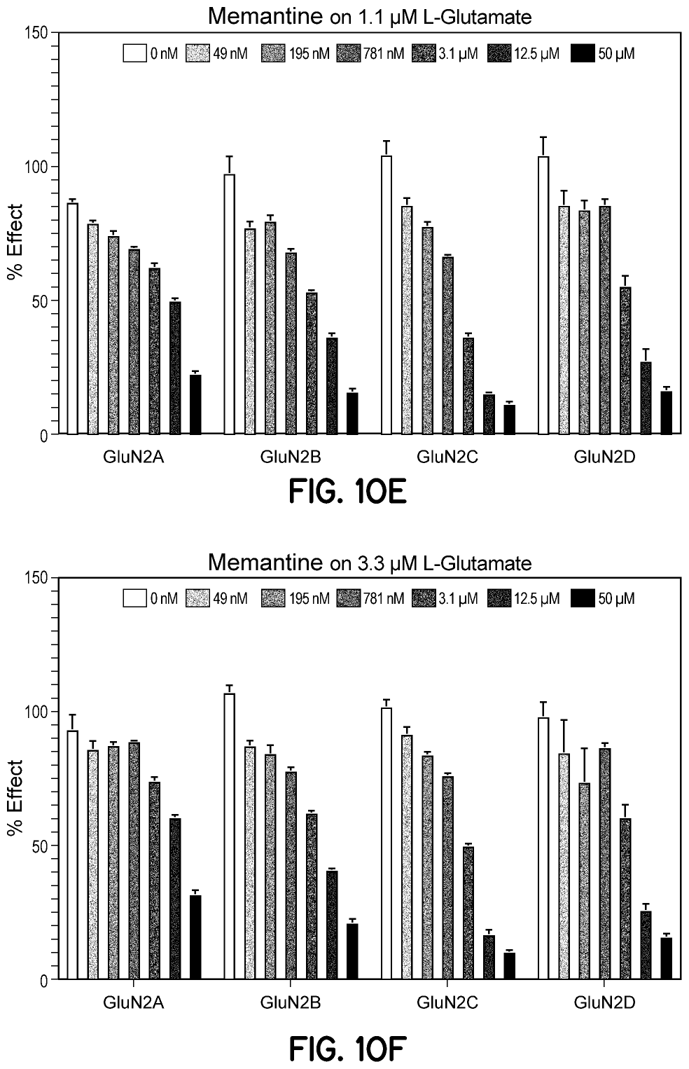

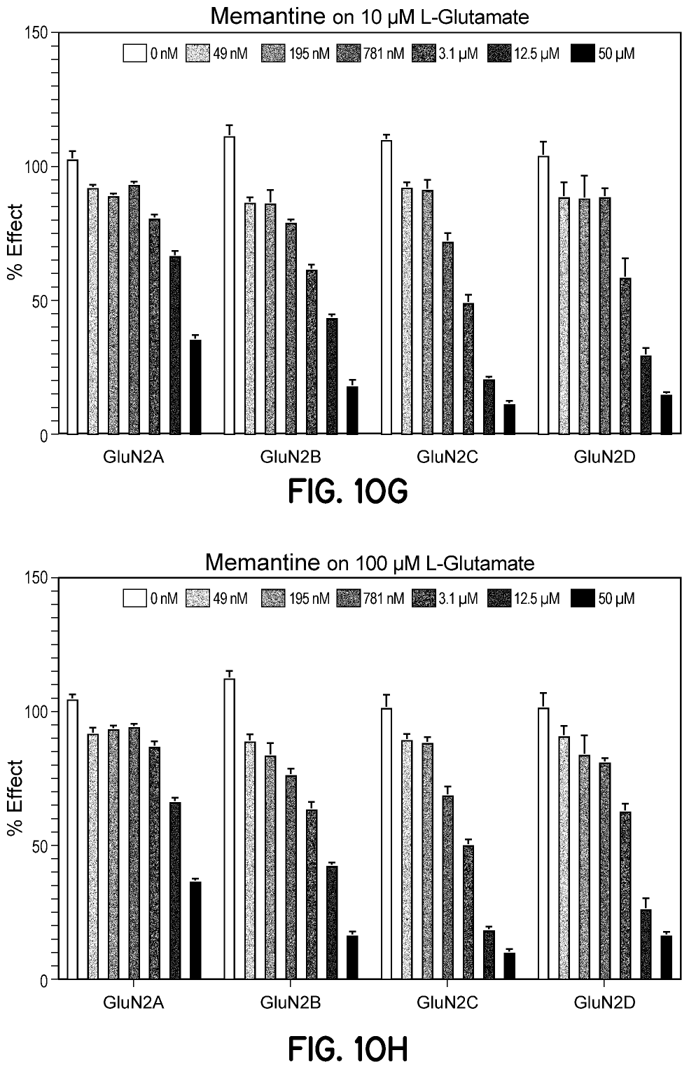

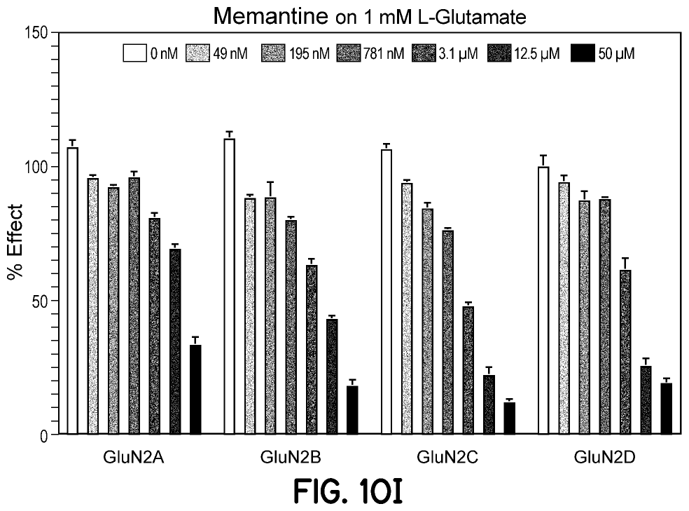

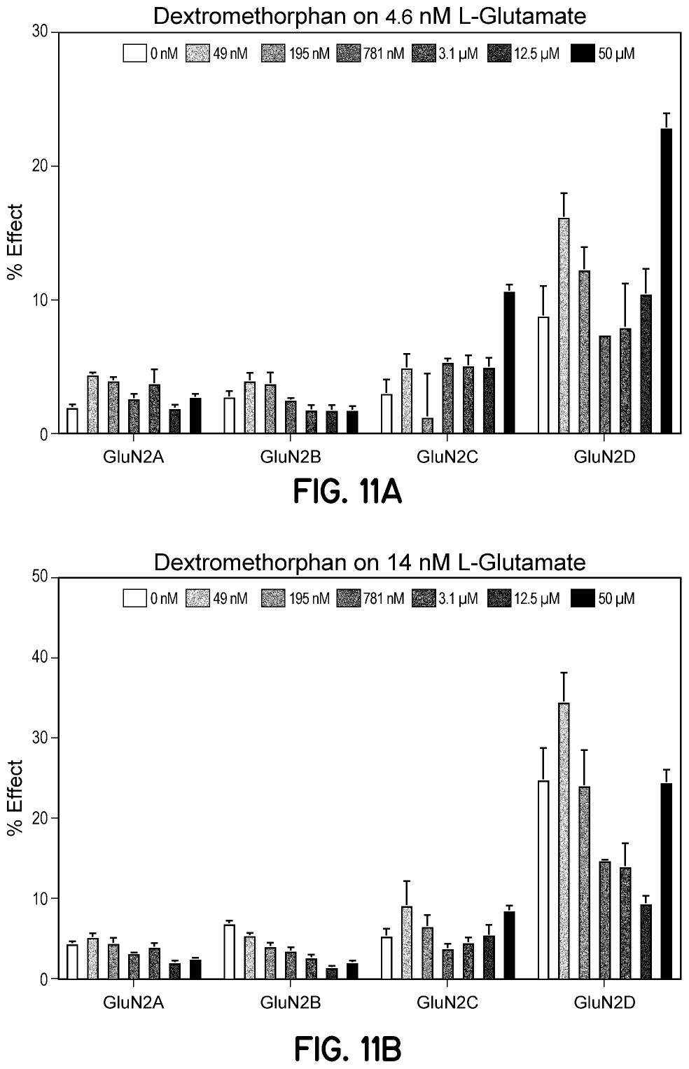

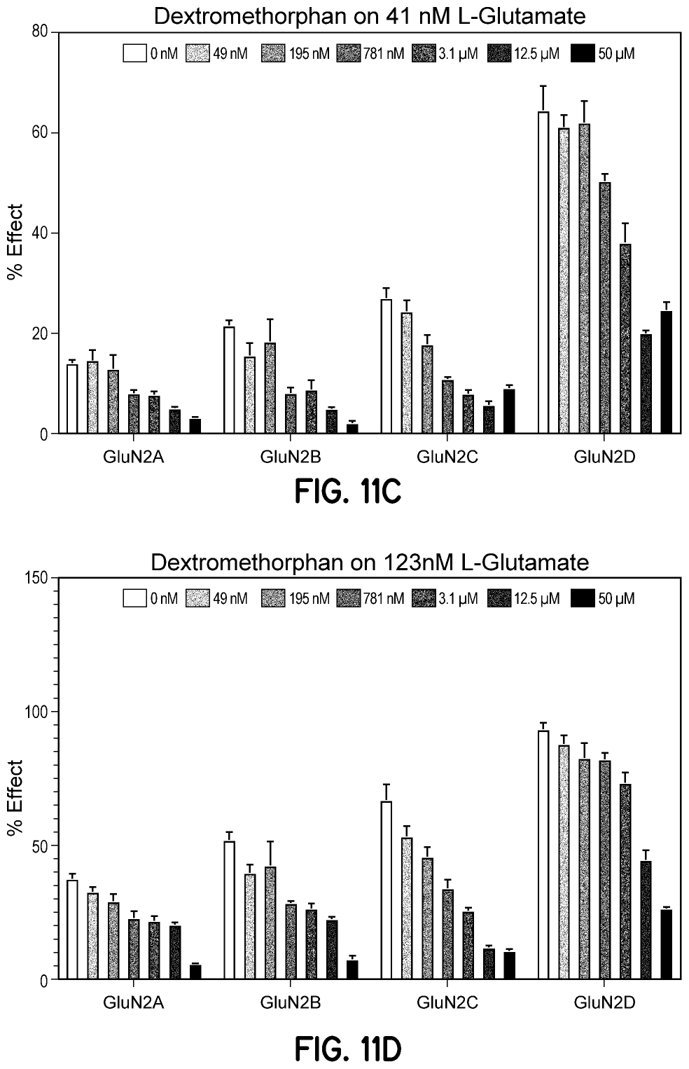

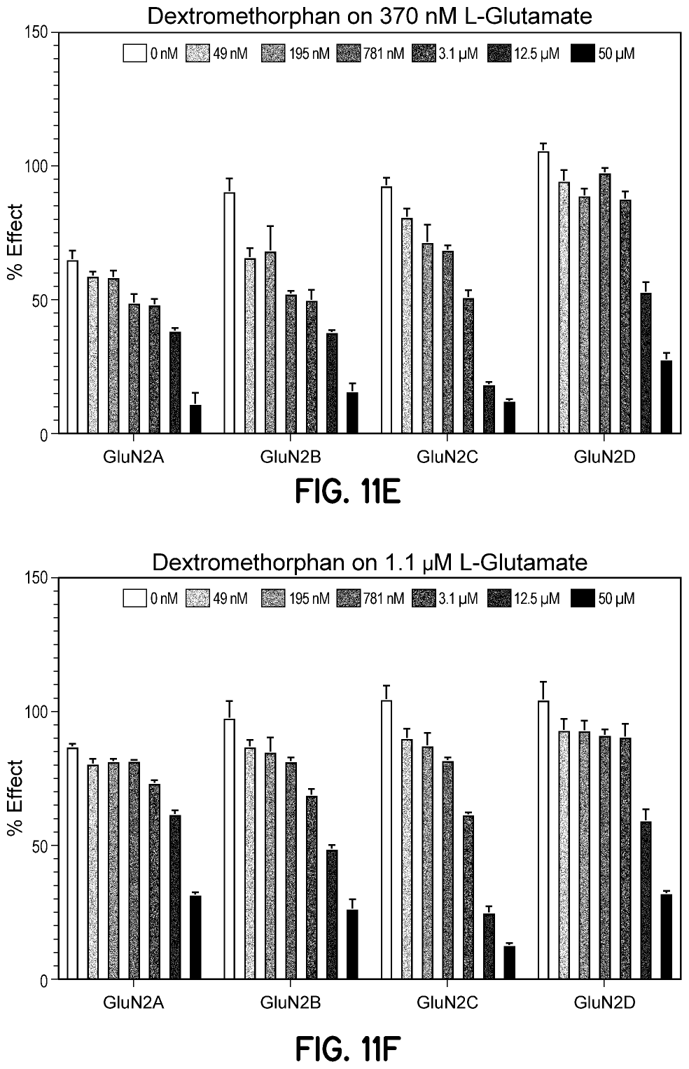

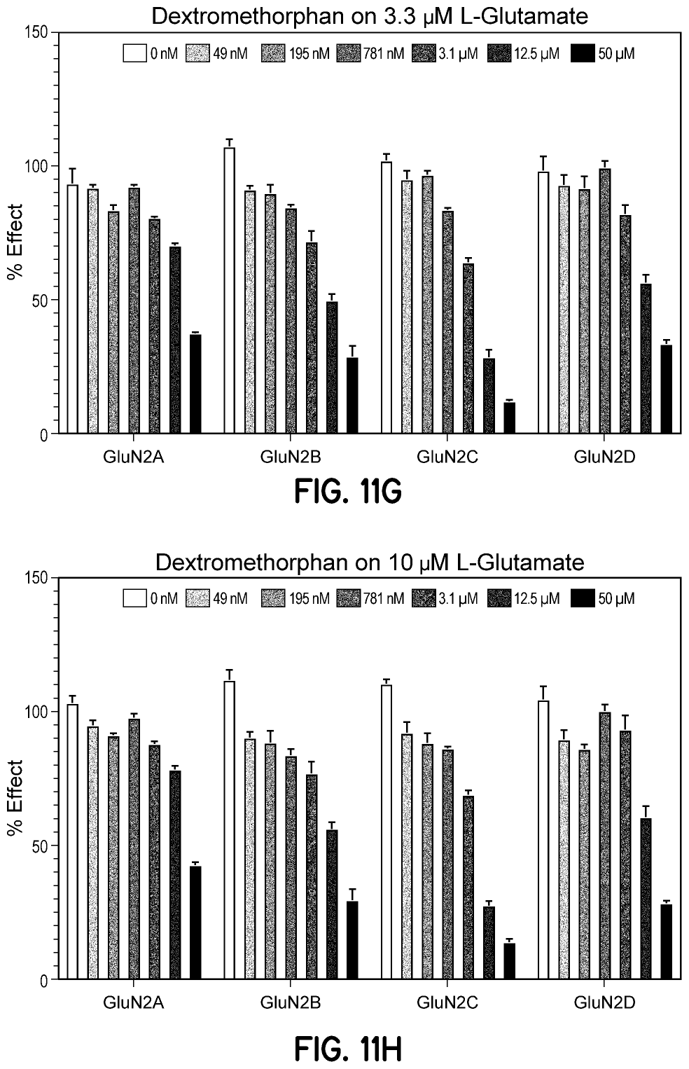

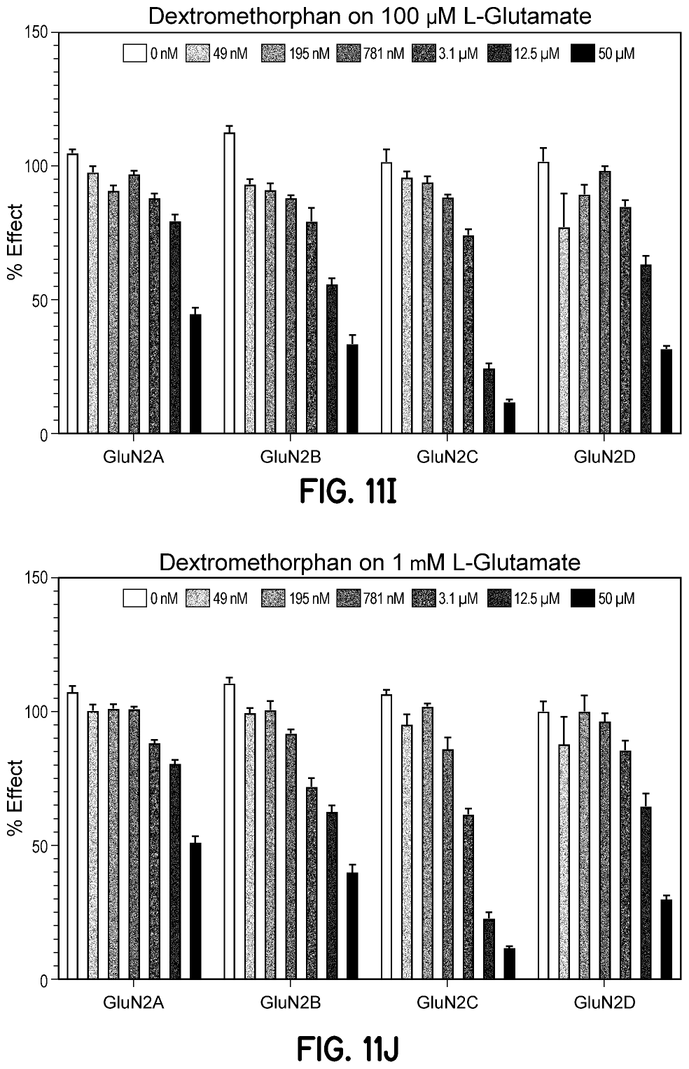

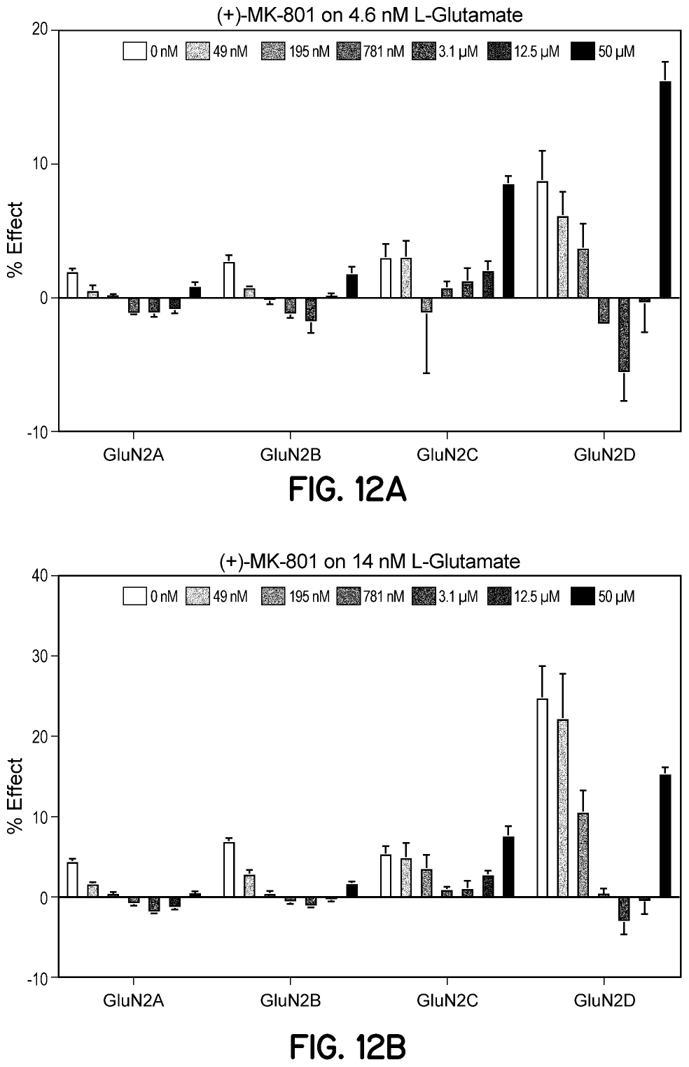

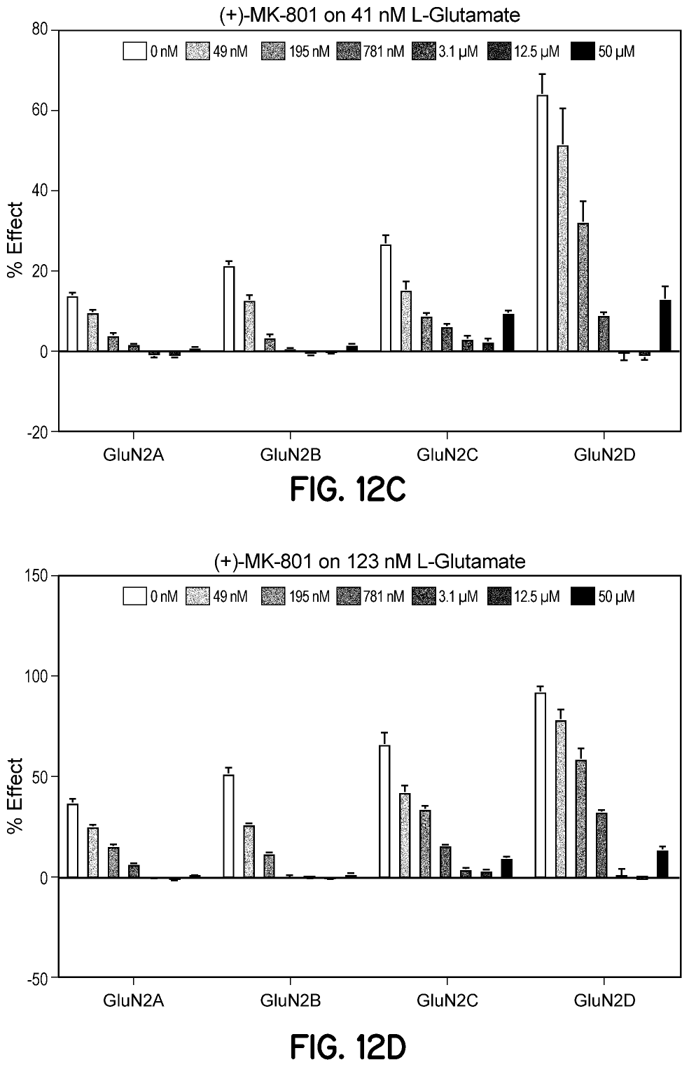

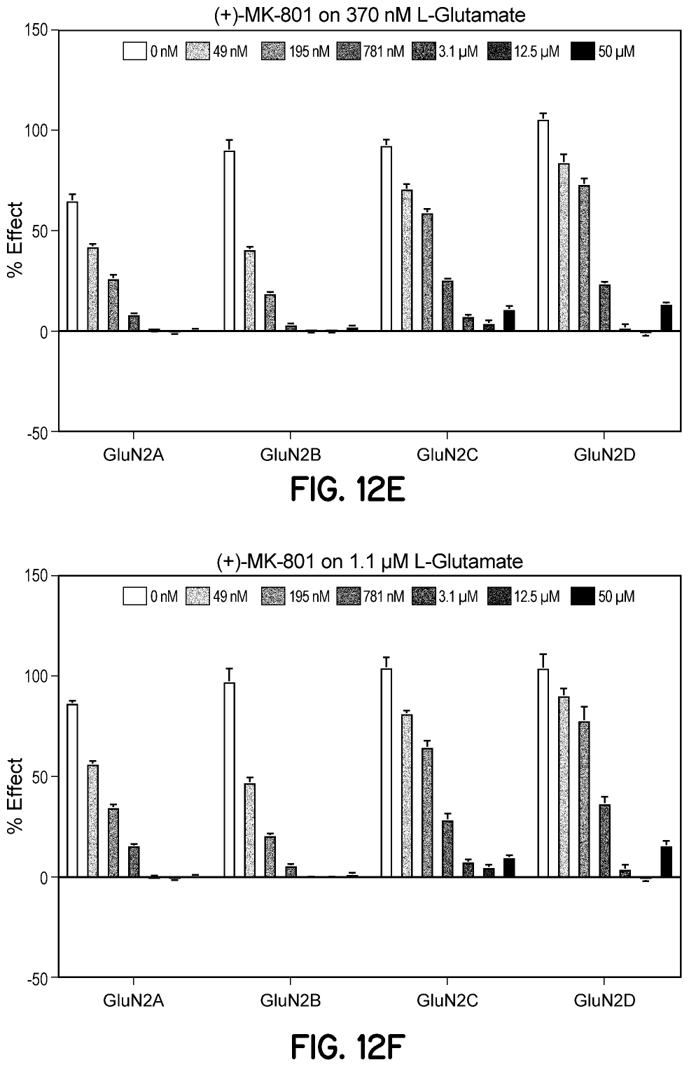

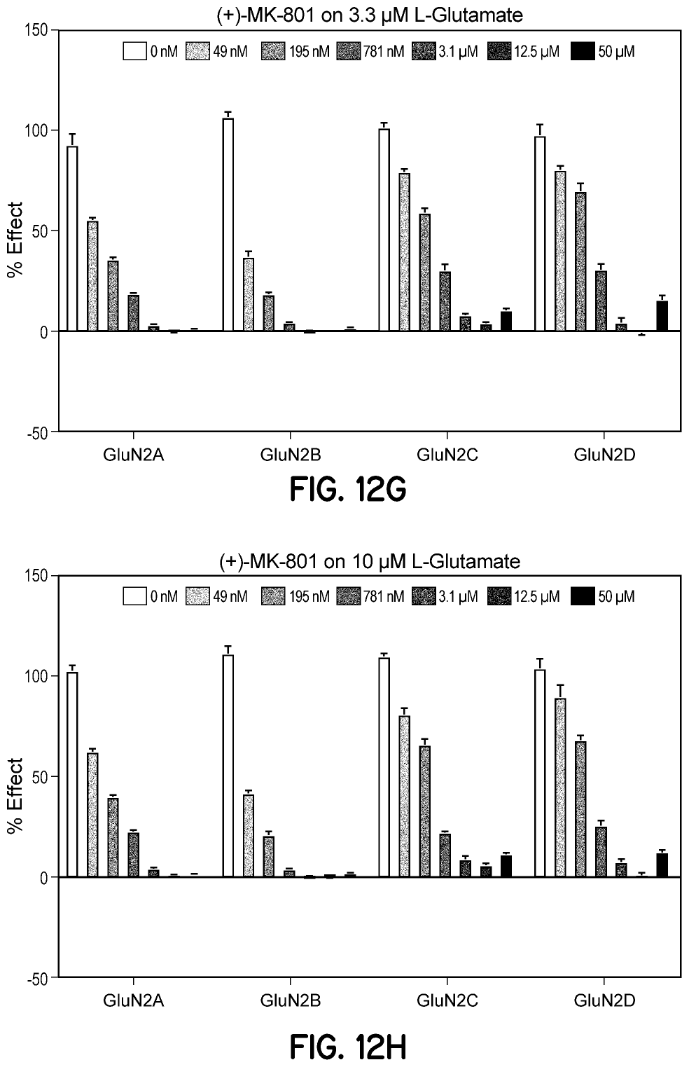

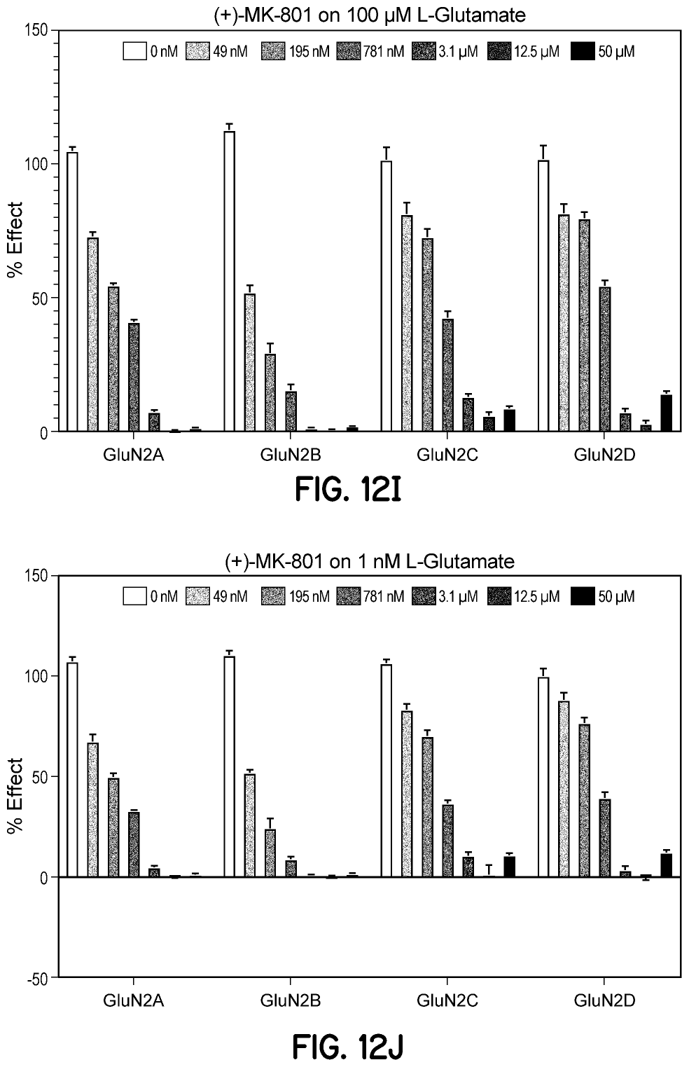

Fluorescence Imaging Plate Reader (FLIPR) Ca²⁺ assay: L-glutamate effect on calcium mobilization. The present inventors examined L-glutamate effect at ten concentrations: 1 mM, 100 μM, 10 μM, 3.3 μM, 1.1 μM, 370 nM, 123 nM, 41 nm, 14 nm, and 4.6 nM. The present inventors examined the effect on the above listed ten concentrations of glutamate (in additional to a concentration of 0) of 5 compounds (MK-801, memantine, ketamine, dextromethorphan, and dextromethadone) at 6 concentrations (50 μM, 12.5 μM, 3.1 μM, 781 nM, 195 nM, and 49 nM; a concentration of 0 is also shown). FIGS. 8A-12J showing the % effect on L-glutamate of the various compounds at the various concentrations.

L-glutamate effect on calcium mobilization showed differential activation of NMDA heterodimeric receptors subtypes, with EC₅₀ rank order GluN2A >GluN2B GluN2C>GluN2D. EC₅₀ was 2.5 μM, 1.3 μM, 870 nM, and 340 nM for GluN2A, GluN2B, GluN2C and GluN2D containing NMDAR, respectively. The potency rank order is in line with that described in literature with various methodologies (Paoletti et al., 2013).

Computing values for EC₅₀ e Hill Slope (H), the present inventors also calculated ECF (where 0<F<100, e.g., 5, 10, 20, 30, 40, 90, 95, 99) using the following formula:

$${EC}_{F} = {( \frac{F}{100 - F} )}^{\sqrt{H}} {EC}_{50}$$

The present inventors applied EC₅₀ e Hill slope values reported for NMDARs in Example 1 and obtained the following ECF values shown in Table 29:

TABLE 29
ECF table

| F | R | ECF | | H | Sub | ECF | | H | Sub | ECF | | H | Sub | ECF | | H |
|---|---|---|---|---|---|---|---|---|---|---|---|---|---|---|---|---|
| 5 | 2A | 160 | nM | 1 | 2B | 140 | nM | 1.3 | 2C | 120 | nM | 1.5 | 2D | 54 | nM | 1.6 |
| 10 | 2A | 280 | nM | 1 | 2B | 240 | nM | 1.3 | 2C | 200 | nM | 1.5 | 2D | 86 | nM | 1.6 |
| 20 | 2A | 630 | nM | 1 | 2B | 450 | nM | 1.3 | 2C | 350 | nM | 1.5 | 2D | 140 | nM | 1.6 |
| 30 | 2A | 1.07 | μM | 1 | 2B | 680 | nM | 1.3 | 2C | 500 | nM | 1.5 | 2D | 200 | nM | 1.6 |
| 40 | 2A | 1.67 | μM | 1 | 2B | 950 | nM | 1.3 | 2C | 660 | nM | 1.5 | 2D | 260 | nM | 1.6 |
| 50 | 2A | 2.5 | μM | 1 | 2B | 1.3 | μM | 1.3 | 2C | 870 | nM | 1.5 | 2D | 340 | nM | 1.6 |
| 90 | 2A | 23 | μM | 1 | 2B | 7.05 | μM | 1.3 | 2C | 3.76 | μM | 1.5 | 2D | 1.34 | μM | 1.6 |
| 95 | 2A | 48 | μM | 1 | 2B | 13 | μM | 1.3 | 2C | 6.19 | μM | 1.5 | 2D | 2.14 | μM | 1.6 |
| 99 | 2A | 250 | μM | 1 | 2B | 45 | μM | 1.3 | 2C | 19 | μM | 1.5 | 2D | 6.01 | μM | 1.6 |

Under physiological circumstances, the total Ca²⁺ influx into the cell following an excitatory stimulation is the sum of Ca²⁺ influx via the different NMDAR subtypes activated by glutamate. Also, the Ca²⁺ influx generally increases with the concentration of L-glutamate up to a maximal effect, as seen in this Example 1. In the present inventors' experiment, the maximal (99%) effect of glutamate concentration on Ca²⁺ influx was seen at 250 μM, 45 μM, 19 μM, and 6 μM for GluN2A, GluN2B, GluN2C, and GluN2D heterologous cells expressing NMDAR subtypes, respectively: At L-glutamate concentrations higher than the maximal effect concentration the Ca²⁺ influx did not increase, also in line with the literature (Paoletti et al., 2013).

From the ECF table (Table 29), it can be seen that the lower glutamate concentration preferentially activates GluN2C and GluN2D subtypes compared to GluN2A and GluN2B subtypes. The preferential activity of dextromethadone for GluN2C (K_{B} table—Table 28) and the developmental distribution of the GluN2C subtype in the brain (Hansen et al., 2019) potentially support the hypothesis of the block of tonically activated (at resting membrane potential, in the presence of low concentration glutamate and in the presence of Mg²⁺ block) pathologically hyperactive (as revealed by the lack of cognitive side effects, see Example 3) GluN2C channels (or GluN2D channels). Dysfunctional astrocytes (or a decrease in the number of functional astrocytes) with impairment in the glutamate/glutamine cycle and excessive residual extracellular synaptic glutamate (even at very low concentrations) may determine excessive Ca²⁺ influx (in particular, as disclosed above, in GluN2C and GluN2D subtypes) resulting in neuronal impairment with reduced neural plasticity that may trigger and or maintain MDD and related disorders (with or without PAMs and agonists). By preferentially targeting tonically and pathologically activated neurons part of the endorphin pathway (shepherding affinity, Example 10), dextromethadone downregulates excessive Ca²⁺ influx in select NMDARs and cellular functionality is restored in the endorphin pathway, resulting in improvement in MDD, as seen in Example 3.

It should be again pointed out that in the Fluorescence Imaging Plate Reader (FLIPR) Ca²⁺ assay, L-glutamate effect on calcium mobilization does not account for the effect of the physiologic Mg²⁺ block and that the in vivo preference for GluN2C and GluN2D of open channel blockers is enhanced several fold in the physiological presence of 1 mM of Mg²⁺ (Kotermanski S E, Johnson J W. Mg2+ imparts NMDA receptor subtype selectivity to the Alzheimer's drug memantine. J Neurosci. 2009; 29(9):2774-2779.). Also, NMDAR tri-eteromers (e.g., NR1-NR2A-NR2B) and tri and di-eteromers containing NR3A-B subunits, were not tested. Different splice variants of NR1 were also not tested. These additional NMDARs potential subtypes and isoforms add layers of complexity but also add potential for fine regulation of Ca²⁺ influx with increasingly precise downstream consequence [epigenetic code, defined above as environment-induced (stimulus-induced) differential patterns of Ca²⁺ cellular influx, with kinetics determined by the NMDAR framework].

Below are nine points that can be inferred from this FLIPR Ca²⁺ assay, and from Examples 2-7:

(1) L-glutamate concentration-dependent (M) effect on Ca²⁺ mobilization differs for each tested subtype of NMDAR, A-D, according to a subtype dependent ranking. Other NMDAR subtypes and isoforms such ad tri-eteromers (e.g., NR1-NR2A-NR2B) and di and tri-eteromers containing NR3A-B subunits, and different splice variants of NR1 are also likely to show differential rankings for Ca²⁺ mobilization effects. The following are examples of known and potential tetrameric NMDAR subtypes (possible NMDAR subtypes considering a tetrameric structure and at least 2 NR1 subunits as obligatory; each possible subtype has potentially distinct functional characteristics and developmental and regional distribution):

(NR1-NR1 tetrahomomer)
NR1-NR2A diheteromer
NR1-NR2A-NR2B triheteromer
NR1-NR2A-NR2C triheteromer
NR1-NR2A-NR2D triheteromer
NR1-NR2B dieteromer
NR1-NR2B-NR2C triheteromer
NR1-NR2B-NR2D triheteromer
NR1-NR2C diheeteromer
NR1-NR2C-NR2D triheteromer
NR1-NR2D diheteromer
NR1-NR3A diheteromer
NR1-NR2A-NR3A triheteromer
NR1-NR2B-NR3A triheteromer
NR1-NR2C-NR3A triheteromer
NR1-NR2D-NR3A triheteromer
NR1-NR3B diheteromer
NR1-NR2A-NR3B triheteromer
NR1-NR2B-NR3B triheteromer
NR1-NR2C-NR3B triheteromer
NR1-NR2D-NR3B triheteromer
NR1-NR3A-NR3B triheteromer

(2) The total post-synaptic Ca²⁺ influx at a given synapse is a function of the concentration/time of L-glutamate (M) in the synaptic cleft, i.e., the amount (stimulus dependent) of glutamate released by the presynaptic axon terminal (and its clearance by EAATs).

(3) Aside from the amount of presynaptic glutamate release, the Ca²⁺ influx in the post-synaptic cell is also a function of the NMDAR framework (density, subtype and location of postsynaptic glutamate receptors, including NMDAR density and subtypes within the synaptic “hotspot”, an approximately 100 nm area closest to the presynaptic glutamate release) of NMDARs (and AMPARs, under physiological circumstances) expressed by the postsynaptic cell membrane at the synaptic cleft (the NMDAR framework is closely related to the post-synaptic density). The expression of AMPARs will determine the voltage dependent activation of the NMDAR (release of the Mg²⁺ block): In this experiment, the absence of Mg²⁺ assumes that the voltage gating has been surpassed or that it is not needed (there are NMDAR subtypes not dependent or less dependent on Mg²⁺ block, such as GluN2C, GluN2D and GluN3 subunit containing subtypes: dextromethadone is likely to be active in these subtypes, because of incomplete Mg²⁺ block of the NMDAR channel pore at resting membrane potential). The NMDAR framework will determine (fine tuning of specific amounts of Ca²⁺ influx) the total Ca²⁺ influx (epigenetic code) for a given amount of glutamate released pre-synaptically and present in the synaptic cleft for a given amount of time (e.g., residual ambient glutamate and potential failure of astrocytes and EAATs).

(4) More generally, the total Ca²⁺ influx is related to the concentration of L-glutamate that reaches the NMDAR framework and the time constant of glutamate clearance from the synaptic cleft by EAATs.

(5) The postsynaptic pattern of Ca²⁺ influx determines the effect on neural plasticity, i.e., LTP and or LTD, including the effect of total Ca²⁺ influx on relative expression of synaptic proteins, including those necessary for assembly of glutamate receptors, including AMPARs, and more importantly NMDARs (see Example 2): total Ca²⁺ influx is therefore regulated by NMDARs and regulates NMDARs. This working hypothesis confers a backbone to neural plasticity (LTP/LTD, memory, connectome, individuality, self-awareness) and in wider terms confers a backbone to the NMDAR centered epigenetic regulation of the genetic code via finely tuned Ca²⁺ influx as an ongoing process from conception to death.

(6) If Ca²⁺ influx is excessive, (high concentration/prolonged glutamate exposure or glutamate+PAM or glutamate+agonist or defective glutamate clearance), cellular functions are impaired (including production of synaptic proteins and thus neural plasticity) and if this excessive Ca²⁺ influx reaches a certain level the cell may undergo apoptosis (excitotoxicity).

(7) Differential patterns of Ca²⁺ influx (the sum of Ca²⁺ entry via different NMDARs at a given synapse) regulate downstream effects. In some neurons, x mEq of Ca²⁺ influx [e.g., x=the mEq amount of Ca²⁺ influx determined by the EC100 for phasic glutamate (e.g., 1 mM, physiological amount released by the pre-synaptic cell, or even amounts as low as 6 μM, as shown in the present inventors' ECF table (Table 29) above for GluN2D subtypes may determine full activation)] into a post-synaptic (and pre-synaptic) neuron determines LTP, i.e. synaptic strengthening. In the same neurons, Ca²⁺ influx over x mEq of Ca²⁺ [e.g., x=the mEq amount of Ca²⁺ influx determined by the EC100 glutamate (e.g. physiological 1 mM or even as low as 6 μM as per ECF table—Table 29—above), maintained over time, may instead determine LTD and weakening of the synapse. The NMDAR framework, variable in different neurons and in different areas of the brain and according to different developmental phases (e.g., developmental switch) is crucial for determining either LTP or LTD (Sava A, Formaggio E, Carignani C, Andreetta F, Bettini E, Griffante C. NMDA-induced ERK signalling is mediated by NR2B subunit in rat cortical neurons and switches from positive to negative depending on stage of development. Neuropharmacology. 2012; 62(2):925-932).

(8) Each tested cell line in the FLIPR assay overexpresses one NMDAR subtype. A different cell line, e.g., ARPE-19, expressing all four subtypes (A-D) (and likely other subtypes and different isoforms) with differential densities (NMDAR framework), required differential concentrations (EC100) of L-glutamate for similar Ca²⁺ mobilization effects and downstream effect (see Example 2).

(9) Finally, pre-synaptic NMDAR receptors are also important for their regulatory effects on the amount of pre-synaptic glutamate release in response to stimuli.

Dextromethadone and four other test compounds were investigated for their effects on Ca²⁺ influx at 6 selected concentrations (50 μM, 12.5 μM, 3.1 μM 781 nM, 195 nM, and 49 nM; 0 is also shown) on L-glutamate Concentration Response Curve (CRC), 11 concentrations, in each heterologous cell line expressing one of the four different NMDAR subtypes, A-D. All tested compounds, including dextromethadone, showed an insurmountable profile, typical of NMDAR pore blockers in FLIPR calcium assays, with K_{B} (M) (a calculated estimate of receptor affinity) in the low micromolar range for dextromethadone for all of the tested NMDAR subtypes (A-D).

In the same FLIPR Ca²⁺ assay the present inventors tested the currently FDA-approved NMDAR pore blockers memantine, ketamine, and dextromethorphan and the high affinity experimental NMDAR pore blocker+)-MK-801. The K_{B} table (Table 28) reports a calculated estimate of NMDAR binding affinity in the absence of extracellular Mg²⁺.

None of the tested compounds was selective for NMDARs containing a specific GluN2 subunit, although all compounds, including dextromethadone, showed some NMDAR subtype preference.

The present inventors disclose that all tested FDA approved NMDAR blockers and dextromethadone show a relative preference for the subtype containing the 2C subunit. MK-801, a high affinity poorly tolerated NMDAR blocker, shows instead a preference for the subtype containing the 2B subunit. For the first time, the present inventors disclose that dextromethadone has a preference for the subtype containing the 2C subunit (K_{B} Table—Table 28). In the same table, the present inventors also show that dextromethadone has the least variability across tested subtypes: this may also be an important feature for safety, as signaled by Example 3 (side effect profile similar to placebo at MDD effective doses). As shown in the ECF table (Table 29) above, the concentrations of glutamate required for tonic activation of the subtype containing the 2C and 2D subunits are very low and therefore signal the potential importance of dextromethadone actions at these subtypes.

The clinically better tolerated NMDAR channel blockers, dextromethadone and dextromethorphan, at doses that are therapeutic for MDD, show K_{B} in the micromolar range for all subtypes, while ketamine, also therapeutic for MDD, shows a nanomolar K_{B} for GluN2C (and an approximately five fold higher affinity for GluN2D compared to dextromethadone and dextromethorphan), suggesting that excessive block of Glu2NC and or GluN2D may cause cognitive side effects, as suggested by dissociative effects seen in over 70% of patients treated with esketamine for MDD.

Considering 100% to be the estimated % affinity for GluN2C containing NMDAR, then estimated % affinity for GluN2A containing NMDAR resulted: 51%, 13%, 11%, and 8% for dextromethadone, dextromethorphan, (±)-ketamine, memantine, respectively.

Memantine, ineffective for MDD, shows a nanomolar K_{B} for GluN2B, GluN2C, and GluN2D.

Of note, all approved NMDAR blockers show micromolar K_{B} for GluN2A subtypes, but not the clinically poorly tolerated MK-801, suggesting that this subtype may be particularly important for cognitive function. A similar reasoning can be applied to the high affinity of MK-801 for GluN2B subtypes. Both of these subtypes, GluN2a and GluN2B, are highly sensitive to the physiologic Mg²⁺ block compared to GluN2C and GluN2D subtypes, making them less likely targets for channel pore blockers: if the channel is already completely blocked by Mg²⁺, the effect of other pore blockers may not be relevant.

The three NMDAR blockers effective for MDD show micromolar K_{B} for GluN2B, while memantine, ineffective for MDD, shows nanomolar K_{B} for GluNB, and MK-801, poorly tolerated clinically, also shows low nanomolar K_{B} for the same subtype.

Taken together these data suggest that clinically tolerated NMDAR blockers effective for MDD may act preferentially on GluN2C and/or GluN2D subtypes, while they relatively spare GluN2A and GluN2B. Of note, this sparing effect of clinically tolerated NMDAR blockers effective for MDD is likely even more relevant in vivo because of the physiologic Mg²⁺ block.

As expected, +)-MK-801, a high potency channel blocker, showed the highest estimated affinity for all NMDAR subtypes, reducing the % effect of L-glutamate to less than 50% with all NMDAR subtypes already at 781 nM. (+)-MK 801 estimated K_{B} resulted ≤50 nM with all of the tested NMDAR subtypes.

Compared to other tested NMDAR pore blockers, dextromethadone showed the least K_{B} NMDAR subtype preference. This relative lack of NMDAR subtype selectivity, while maintaining a slight preference for GluN2C over 2A (a characteristic shared by dextromethorphan, ketamine and memantine) could also contribute to explain the excellent tolerability and safety profile, indistinguishable from placebo at doses therapeutic for MDD (see Example 3). This excellent tolerability and safety profile, indistinguishable from placebo at doses therapeutic for MDD, signals that in the tested MDD patients [Example 3, patients screened with SAFER (Desseilles et al., Massachusetts General Hospital SAFER Criteria for Clinical Trials and Research. Harvard Review of Psychiatry. Psychopharmacology, September-October 2013; 21 (5) 1-6)], dextromethadone may have selectively blocked only hyperactive (pathologically hyperactive) NMDARs, without interfering with physiologically working NMDARs, thus the lack of side effects, including the lack of cognitive side effects typical for NMDAR blockers (over 70% of MDD patients treated with therapeutic doses of esketamine experience “dissociative” cognitive side effects, suggesting that this drug does instead act on physiologically operating NMDARs). GluN2C and 2D subtypes may be hyperactive tonically at low concentrations of glutamate (as seen in the present inventors' ECF table, Table 29, compared to GluN2A and GluN2B). These two subtypes 2A and 2B are instead more dependent on phasic stimulation (depolarization) triggered by stimulus dependent presynaptic release of high concentration glutamate and require release of the Mg²⁺ block before allowing any Ca²⁺ influx (Kuner T, Schoepfer R. Multiple structural elements determine subunit specificity of Mg2+ block in NMDA receptor channels. J Neurosci. 1996; 16(11):3549-3558). The GluN2C and GluN2D tonic Ca²⁺ permeability (low level) in the presence of Mg²⁺ block enhances several fold (Kotermanski et al., 2009) the relative preference for these subtypes (in particular type GluN2C disclosed by the present inventors' FLIPR assay (in the absence of Mg²⁺) for ketamine, dextromethorphan, memantine (all FDA approved drugs) and for dextromethadone, corroborating the present inventors' disclosed mechanism of action for disease-modifying effects.

The relative lesser block exerted by dextromethadone on the GluN2A subtype seen at higher glutamate concentrations compared to the block exerted on GluN2C (and GluN2D) subtype at lower concentration signals a preferential effect on pathologically tonically active NMDARs relatively to physiologically phasically active NMDAR (see tables above and Example 5).

Other potential explanations for the excellent safety and tolerability of dextromethadone may involve the “on” and “off” and “trapping” aspects of the interaction of dextromethadone with the NMDAR (see Example 6): Dextromethadone shows a tenfold lower GluN-GluN2C NMDAR subtype potency compared to ketamine, as disclosed by the inventors in these experiments (Example 1, Table 28, and Example 6, Part I: similar “onset” for 1/10 ketamine concentration compared to dextromethadone (Example 6, Part I). Dextromethadone matches ketamine in “trapping” (Example 6, Part II). When this finding is compared to the lower “trapping” of memantine, it suggests that relatively high “trapping” and relatively low micromolar affinity are both desirable features for a clinically effective drug in MDD and for a safe NMDAR channel blocker. Memantine with relatively low “trapping” (Mealing G A, Lanthorn T H, Small D L, et al. Structural modifications to an N-methyl-D-aspartate receptor antagonist result in large differences in trapping block. J Pharmacol Exp Ther. 2001; 297(3):906-914) does not work for MDD, however it appears to be relatively well tolerated compared to ketamine, a drug with similar affinity but higher trapping compared to memantine. Ketamine with both high potency and high “trapping” has dissociative effects. Dextromethadone with “trapping” similar to ketamine but lower potency is instead well tolerated, without cognitive side effects at therapeutic doses.

Furthermore, the lack of cognitive side effects at therapeutic doses (see Example 3), signals that physiological NMDAR functionality, e.g., phasic Glu2A-D activity, was not affected by dextromethadone. In Example 6, Part III, the present inventors show how in the presence of Mg²⁺ and low glutamate concentrations the effect of dextromethadone is related to membrane polarity, similarly to the block exerted by Mg²⁺. This novel disclosure also explains the lack of cognitive side effects for dextromethadone: like physiological Mg²⁺ dextromethadone works best around resting potential and is expelled from the pore, just like Mg²⁺ during the voltage gated phase of NMDAR activation.

Also, dextromethadone exerts Ca²⁺ influx reduction at very low concentrations of glutamate, with or without PAMs and or agonists (Example 5), indicating once more that its actions may not involve physiological phasic NMDAR function, when high concentrations of glutamate are present in the presence of Mg2+. In vivo this Ca²⁺ influx reduction may thus not pertain to GluN2A and GluN2B subtypes because very low concentrations of glutamate will not activate AMPARs and therefore will not relieve the Mg²⁺ block, and these subtypes are impermeable to Ca²⁺ while blocked by Mg²⁺ but may be relevant for GluN2C and Glun2D because of their relative independence (low level Ca²⁺ permeability) from the Mg²⁺ block (Kuner et al, 1996; Kotermanski et al., 2009). Taken together, these findings and observations suggest that dextromethadone's effects may be preferential for NMDARs tonically and pathologically activated by low concentrations of glutamate, including GluN2C and GluN2D (Example 6, Part III) and or other NMDAR subtypes that are less affected or not affected by Mg²⁺ block (e.g., subtypes containing Glun3 subunits).

As a further simplification, voltage gated NMDARs that open and close physiologically in response to various stimuli as directed by physiological phasic high glutamate concentrations may be relatively unaffected by dextromethadone's channel block. Additionally, the “on” kinetic of dextromethadone (several seconds) may not be fast enough for blocking stimulus evoked Ca²⁺ currents (this “on” timing hypothesis for dextromethadone is supported by Example 6, Part I and by the ranking of dextromethadone's block of Ca²⁺ influx for different NMDAR subtypes that follows the known kinetics of NMDARs GluN2D>GluN2C>GluN2B>GluN2A: while subtypes that stay open longer following stimulation may be blocked more effectively and thus Ca²⁺ influx via these channels is decreased more effectively by dextromethadone) (Example 1), the culprit of the blocking activity of dextromethadone is more likely to be at resting membrane potential. Therefore, dextromethadone is potentially selective for tonically and pathologically hyperactive NMDARs, i.e., NMDARs tonically activated by chronic low concentrations of glutamate, in the presence or absence of PAMs and other agonists, as seen in Example 5 at 0.04 and 0.2 microM L-glutamate, in the presence or absence of gentamicin and or quinolinic acid and in the absence of a MG²⁺ block.

Physiological concentrations of L-glutamate for brief time periods (e.g., phasic glutamate 1 mM) (the physiological decay time constant for glutamate is 1 ms) would instead be unaffected by dextromethadone, as signaled by the lack of cognitive side effects of dextromethadone at doses effective for the treatment of MDD (Example 3) and the long “onset” required for dextromethadone action (Example 6). The preference for GluN2C subtypes seen for ketamine is in the nanomolar range and this difference compared with dextromethadone and dextromethorphan, both micromolar, could explain ketamine's dissociative effects at therapeutic doses for MDD. The effects of dextromethadone were evident also when a PAM and or an agonist were added (see Example 5). The effects of dextromethadone on the downregulation of Ca²⁺ influx are likely to be evident not only when the cause is repeated presynaptic release of glutamate, both in the presence or in the absence of PAMs (e.g., gentamicin, Example 5), or in the presence or absence of an agonist substance such as quinolinic acid, but also when the chronic low glutamate extracellular concentration is due to defective clearance (e.g., by defective EAAT activity) due to a number of reasons, including astrocyte dysfunction or death, including apoptosis that could also be mediated by excitotoxicity and thus potentially preventable with dextromethadone. Effects of dextromethadone shown herein include the following:

(1) Dextromethadone exerts an insurmountable block of NMDARs (Example 1), similarly to the FDA approved NMDA channel blockers ketamine, dextromethorphan and memantine.

(2) Dextromethadone exerts rapid and robust therapeutic effects at doses with side effect comparable to placebo in patients with MDD (see Example 3), signaling selectivity for pathologically hyperactive NMDARs.

(3) The therapeutic effectiveness of dextromethadone for MDD persists after discontinuation of therapy, beyond receptor occupancy (see Example 3), signaling a neural plasticity effect that persists beyond receptor occupancy (including beyond any occupancy of receptors other than NMDAR).

From the points above, the present inventors conclude that at least for a subset of patients diagnosed with MDD, the disorder is potentially caused by excessive Ca²⁺ influx via hyperactive NMDARs. This excessive Ca²⁺ influx impairs neuronal functions, including synaptic plasticity (the homeostatic production and assembly of synaptic proteins and release of BDNF is impaired), in select neurons part of select circuits involved in memory of emotional states (this impairment in forming new memory of emotional states may be the determinant of the mood disorder). The block of excessive Ca²⁺ influx exerted by uncompetitive channel blockers (dextromethadone, ketamine, dextromethorphan), downregulates the excessive Ca²⁺ influx and restores neuronal plasticity, including synthesis of NMDAR proteins (Example 2). When environmental stimuli reach neurons within the endorphin pathways with restored synaptic ability (synaptic proteins ready for assembly and expression as functional receptors and BDNF ready for release) new emotional memories are produced and the MDD phenotype subsides. The excessive opening of NMDARs may be caused by excessive stimulus-induced presynaptic glutamate release (e.g., psychological stressors), and/or decreased glutamate clearance (EEAT deficit, astrocytic pathology) or NMDAR hyperactivity may be caused by a PAM, or an agonist, as shown with gentamicin in Example 5, or a combination of excessive glutamate and a PAM or an agonist such as quinolinic acid. The concept of “excessive” glutamate may thus be more related to the time of exposure (pathological and tonic activation) rather than to the concentration (e.g., 1 mM) reached for a brief time (e.g., 1 ms), during physiological and phasic operations. Dextromethadone effectively reduced Ca²⁺ influx caused by the PAM gentamicin (Example 5), a known ototoxic and nephrotoxic agent, and could thus potentially prevent these toxicities and similar toxicities exerted by PAMs on different cells, including CNS cells. Similarly, therefore, in a subset of patients with MDD (or other disorders and diseases), one or more known (e.g., morphine) or yet unknown PAMs (or agonists) of NMDARs, which may be selective for neurons implicated in the plasticity of emotional memory (e.g., opioids), may be implicated in triggering or maintaining the disorder or disease. Dextromethadone effectively counteracts the excessive Ca²⁺ entry determined by PAMs and agonists of NMDARs (Example 5).

Furthermore, dextromethorphan is FDA approved (in combination with quinidine) for the treatment of PBA, suggesting that at least for a subset of patients suffering from pseudobulbar syndrome, excessive influx of Ca²⁺ via hyperactive NMDARs impairs neural function (including neural plasticity) in select neurons part of circuits that regulate the expression of emotions (affect), which are integral part of emotional “memory” circuits.

Lastly, memantine, also tested in the present inventors' FLIPR Ca²⁺ assay, exerts uncompetitive (unsurmountable) NMDAR channel blocker actions similarly to dextromethadone (as shown in this Example 1). Memantine is FDA approved for the treatment of moderate to severe dementia and is thought to selectively regulate hyperactive glutamatergic pathways in these patients [Cacabelos R, Takeda M, Winblad B. The glutamatergic system and neurodegeneration in dementia: preventive strategies in Alzheimer's disease. Int J Geriatr Psychiatry. 1999 January; 14(1):3-47]. The present inventors can postulate that least for a subset of patients suffering from Alzheimer disease, an excessive influx of Ca²⁺ via hyperactive NMDAR impairs neural function (including neural plasticity) in select neurons part of select circuits involved in aspects of cognitive memory. A hyper-glutamatergic state in Alzheimer's disease is also compatible with the beta-amyloid increase seen in these patients (Zott B, Simon M M, Hong W, et al. A vicious cycle of β amyloid-dependent neuronal hyperactivation. Science. 2019; 365(6453):559-565).

All of the above evidence suggests that clinically tolerated NMDAR uncompetitive channel blockers may potentially be therapeutic for a multiplicity of diseases and disorders triggered or maintained by NMDAR dysfunction. Among all known agents, dextromethadone may be quite useful because of its favorable PK and PD profiles, as shown in Example 3 at therapeutic doses. The inventors for the first time disclose disease-modifying effects of dextromethadone and provide novel mechanisms to explain these novel effects (Examples 1-11). As disclosed by the inventors, the common therapeutic action exerted by all of the NMDAR channel blockers is the down-regulation of the excessive influx of Ca²⁺ via hyperactive NMDARs. Excessive Ca²⁺ influx impairs neural plasticity mechanisms in select neurons part of select circuits. While the opening of NMDAR channels and the subsequent Ca²⁺ influx are dependent on glutamate concentration (as shown in this Example 1), under physiological circumstances, high concentrations of glutamate for a brief time (e.g., 1 ms) do not cause excessive (pathological) Ca²⁺ influx. On the other hand, chronic (tonic) low concentration of glutamate may instead cause excessive (pathological) Ca²⁺ influx over time, especially via NMDARs not completely voltage gated, e.g., not 100% gated by the Mg²⁺ block (low level Ca²⁺ permeability in presence of Mg²⁺ within the channel pore). Dextromethadone is likely to act selectively (Example 3, lack of side effects at therapeutic doses) on tonically hyperactivated NMDARs, especially NR1-GluN2C and NR-1GluN2D or NR1-GluN3 subtypes, including in the presence or in the absence of one or more PAMs or agonists (Example 5).

Furthermore, in the case of dextromethadone, the present inventors show for the first time that one of the mechanisms of rescued neuronal plasticity is modulation of select NMDAR subunits (enhancement of transcription and synthesis of NR1 and NR2A subunits, Example 2). This finding not only contributes to explain dextromethadone's potential therapeutic effects for treating, preventing and diagnosing a multiplicity of diseases and disorders, but it also sheds light on the fundamental mechanism underlying neural plasticity: patterns of Ca²⁺ influx are not only regulated by NMDARs but in turn regulate NMDAR synthesis and expression, conferring a molecular basis for the concept of ongoing (from conception to death) evolving plasticity, including neural plasticity, directed by environmental (epigenetic) stimuli (G+E paradigm).

Based on the above evidence, the present inventors postulate that the common code for neural plasticity (LTP/LTD, memory, connectome, individuality, self-awareness) is represented by differential patterns of Ca²⁺ that are not only regulated by NMDARs but, in turn, regulate NMDARs. Each subsequent stimulus (glutamate release by the presynaptic neuron) will be received differently by the post-synaptic neuron (it will result in a different pattern of Ca²⁺ entry) and thus it will have a unique effect on neural plasticity. These ever differential (unique) effects of patterns of Ca²⁺ occur constantly (at any given moment an array of different stimuli reaches neurons) during the lifespan of individuals (each pattern of Ca²⁺ influx is different from the preceding one and from the subsequent one because of their influence on the NMDAR framework) and determine the individual's constantly reshaping connectome (memory), and thus determines individuality and consciousness.

J. Conclusions

(1) FLIPR calcium assay showed an insurmountable profile of dextromethadone, memantine, (±)-ketamine, (+)-MK 801, dextromethorphan on diheteromeric human recombinant NMDAR containing GluN1 plus one amongst GluN2A, GluN2B, GluN2C or GluN2D subunit. Differential preferences for specific GluN2 subunits were also shown.

(2) Dextromethadone acts as a low affinity (low micromolar as indicated by the calculated K_{B}) uncompetitive blocker (unsurmountable), as seen in Example 1. This finding, together with the results in Example 2-11, signals dextromethadone's selectivity for hyper-stimulated, pathologically hyperactive, NMDARs.

(3) Dextromethadone differential modulation of Ca²⁺ influx via NMDARs depending on the concentration of glutamate (Example 1) suggests a similar mechanism for other stimuli that potentially activate NMDARs, including PAMs, including toxins, including other agonists as confirmed by the findings outlined in Example 5: hyper-stimulated NMDARs (pathologically hyperactive, with excessive Ca²⁺ influx) are blocked more effectively than physiologically active NMDARs.

Dextromethadone (and potentially other NAMs disclosed by the inventors) may block the pore only in case of prolonged (tonic) opening, when the net effect on Ca²⁺ influx from the summation of different stimuli (glutamate and PAMs and toxins) is excessive.

(4) Compared to other tested NMDAR pore blockers, dextromethadone showed a lower potency and the least K_{B} NMDAR subtype variability (in this Example 1). This relative lack of NMDAR selectivity of the dextromethadone pore channel block could potentially contribute (together with points 1-2, above) to explain its excellent tolerability and safety profile (indistinguishable from placebo) at doses that effectively treat MDD (Example 3, MDD) by putatively blocking selectively only a subset of hyperactive (tonically and pathologically hyperactive) NMDARs.

(5) Notwithstanding point 3, there is a relative 2C preference. The subtype 2C preference could signal that the activity of dextromethadone is preferential for pathologically and tonically hyperactive 2C subtypes [the on/off kinetics for dextromethadone (Example 6) could restrict the molecule to tonically hyperactive channels because the opening/closing of physiologically functioning receptors, regulated by depolarization and Mg²⁺ block, is much faster, measured in milliseconds (e.g., NR1-NR2A subtype) compared to seconds (e.g., NR1-NR2D subtype) (Hansen et al, 2018)]. The preference for 2C and 2D subunit-containing subtypes is enhanced in vivo by the presence of a relatively lower Mg²⁺ block in these subtypes (Kotermanski and Johnson 2009; Example 6). Furthermore, the “on”/“off” kinetics for dextromethadone (Example 6), suggest that it may be unable to affect the much faster activation/deactivation of phasically operating NMDARs. The phasic opening of GluN1-GluN2A, GluN1-GluN2B, GluN1-GluN2C, GluN1-GluN2D subtypes is 50 msec, 400 msec, 290 msec and over 2 seconds respectively (Hansen et al., 2018). “Onset” for dextromethadone is measured in tens of seconds (Example 6) making it unlikely that this molecule could enter open channels during stimulus-triggered phasic opening. However, when GluN1-GluN2C and GluN1-GluN2D subtypes (or subtypes containing the N3 subunits), allow excessive inward Ca²⁺ flux at resting membrane potential in the presence of Mg²⁺ within the NMDAR channel, dextromethadone could potentially block this excessive Ca²⁺ influx (Example 6).

### Example 2

A. Overview

In the experimental study of this Example, the present inventors sought to determine whether (1) the membrane of human retinal pigment epithelial cells (the cell line ARPE-19) expresses NMDAR receptor subtypes (GluN1GluN2A, GluN2B, GluN2C, and GluN2D); (2) dextromethadone mitigates L-glutamate-induced cytotoxicity; (3) dextromethadone modulates transcription and synthesis of select NMDAR protein subunits; and (4) dextromethadone increases expression of NMDARs. The experiments detailed below demonstrate that dextromethadone upregulates NR1 subunits, which are essential for membrane expression of NMDARs, and thus neural plasticity.

B. Methods and Results

1. Expression of NMDAR Subtypes in ARPE-19 Cells

First, the present inventors assessed the expression of five NMDAR subunits (GluN1, GluN2A, GluN2B, GluN2C, GluN2D) by immunofluorescence coupled to confocal microscopy.

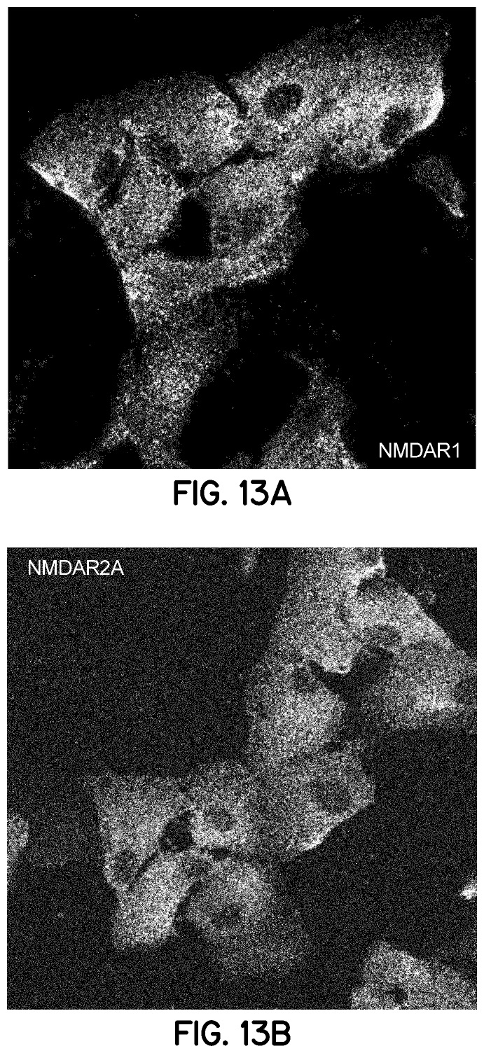

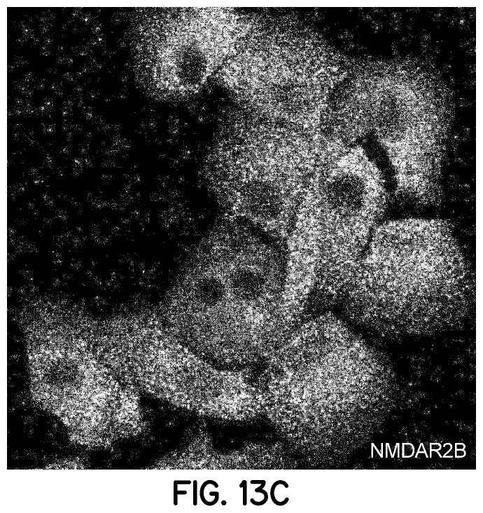

7,500 cells/well were plated in a 24-well plate on sterile glass coverslips. The next day, the immunofluorescence analysis was performed. The following primary antibodies were used: anti-NMDAR1A (Abcam, ab68144), anti-NMDAR2A (Bioss, bs-3507R-TR), anti-NMDAR2B (Bioss, bs-0222R-TR), anti-NMDAR2C (Invitrogen, PA5-77423) and anti-NMDAR2D (Invitrogen, PA5-77425) and the secondary antibody goat anti-rabbit IgG (GeneTex, GTX213110-04). The images of the immunostained cells (see FIGS. 13A-C) were acquired by means of a confocal microscope Zeiss LSM 800, using a 63× magnification. ImageJ software was used to quantify the intensity of the fluorescent signal.

2. Effect of Dextromethadone on Glutamate-Induced Cytotoxicity

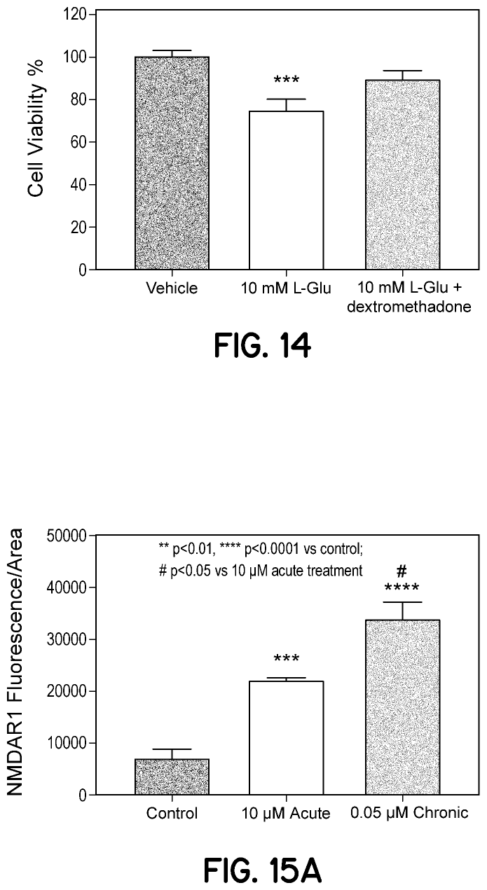

In order to ascertain the effect of dextromethadone on L-glutamate-induced cytotoxicity in ARPE-19 cells, the present inventors performed a cell viability assay. For this experiment, the ARPE-19 cells were seeded in a 96 wells plate (7000 cells/well). They were left overnight in a 37° C. incubator with 5% CO₂. The following day, the cells were pretreated with the dextromethadone solutions. After six hours all the wells (with the exception of control cells) were replaced with the L-glutamate at 10 mM concentration dissolved in a Tris-buffered Control Salt Solution (CSS). After 5 min, the exposure solution was washed out thoroughly and replaced with standard culture medium. After 24 hours of resting time, cell viability was assessed by a crystal violet assay. The present inventors observed that dextromethadone, tested at 30 microM, counteracted the observed reduction of cell viability induced by L-glutamate treatment, as shown in FIG. 14 [which shows cell viability of ARPE-19 cells after treatment with the NMDAR agonist L-glutamate, alone (10 mM L-Glu) or in combination with dextromethadone. *** P<0.001 vs control cells treated with vehicle (one-way ANOVA followed by Tukey's post hoc test)].

3. Effect of Dextromethadone on the Protein Expression of NMDAR Subunits

The present inventors performed additional immunocytochemical studies to ascertain whether dextromethadone induces synthesis of select proteins that form NMDARs.

In these additional studies, 7,500 cells/well were plated in a 24-well plate on sterile glass coverslips. The next day, cells were treated with either 10 μM dextromethadone for 24 hours followed by 5 days of rescue in standard culture medium or 0.05 μM of dextromethadone for 6 consecutive days. After 6 days an immunofluorescence analysis coupled to confocal microscopy was performed with the primary and secondary antibodies described above.

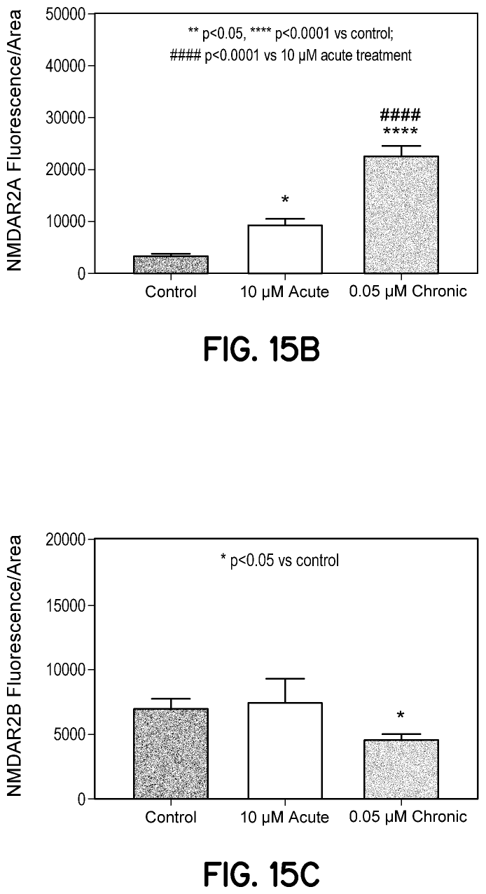

Results are shown in FIGS. 15A-C. ARPE-19 cells exposed to dextromethadone 0.05 μM for 6 days showed a dramatic increase in NMDAR1 and NMDAR2A subunits, whereas the present inventors observed a significant drop of NMDAR2B expression. ARPE-19 cells exposed to dextromethadone 10 μM for 24 hours also showed a significant increase of NMDAR1 and NMDAR2A, although this increase was less prominent compared to the increase observed with the chronic incubation. NMDAR2B subunits did not change with acute treatment.

C. Discussion and Conclusions

Based on the experimental work of this study, it is shown that ARPE-19 cells express of all tested NMDAR subunits (NMDAR1, NMDAR2A, NMDAR2B, NMDAR2C, and NMDAR2D); dextromethadone prevents glutamate excitotoxicity in ARPE-19 cells; and dextromethadone, at tested concentrations (10 μM and 0.05 μM), dramatically upregulates NR1 and NR2A subunits, but has no effect (10 μM) or down-regulates (0.05 μM) NR2B subunits.

The observed modulatory effects on NMDAR subunits are potentially determined by dextromethadone uncompetitive NMDAR block and down-regulation of excessive Ca²⁺ influx (see Example 1). In the absence of glutamate stimulation, the present inventors assume that the excessive Ca²⁺ influx counteracted by dextromethadone is mediated by the agonist effect of light on NMDARs expressed on the membrane of ARPE-19 cells.

Further, excessive Ca²⁺ entry via pathologically hyperactive NMDARs hyper-stimulated by high concentration glutamate (10 mM) causes excitotoxicity manifested by a reduction in ARPE-19 cell viability (as shown in FIG. 14).

For the first time now disclosed in this application, dextromethadone was found to exert rapid, sustained and robust antidepressant effects in patients diagnosed with MDD (see Example 3, below). The therapeutic effects in MDD appear to outlast the sharp decline in plasma levels after abrupt discontinuation of dextromethadone (as shown in Example 3), suggesting a neural plasticity-based mechanism of action.

And for the first time now disclosed in this application, dextromethadone has been shown to differentially modulate subunits in ARPE-19 cells, including GluN2C and GluN2D subunits.

The modulation of transcription and synthesis of NMDAR subunits, potentially resulting in modulation of NMDAR expression (NR1 subunits are necessary for NMDAR expression on the cell membrane), may not only contribute to explain the mechanism of action for the therapeutic effects in MDD of dextromethadone and other uncompetitive NMDAR channel blockers, but may offer important insight into the physiological and pathological role of NMDARs. The present inventors suggest that differential patterns of Ca²⁺ influx are regulated by NMDARs activated by glutamate (with or without PAMs or other glutamate agonists) or other stimuli (e.g., light) and these patterns of Ca²⁺ influx in turn regulate NMDAR expression on the cell membrane (NMDAR framework). Neural plasticity regulates and is regulated (coded) by differential patterns of Ca²⁺ influx via NMDARs (shared epigenetic code for neural plasticity).

Based on their experimental results in ARPE-19 the present inventors hypothesize that different glutamate concentrations may act as shown in FIG. 16.

NR1 was chosen as a measure of neural plasticity because this subunit is necessary for the expression of all NMDAR subtypes NR1-NR2A, NR1-NR2B, NR1-NR2C, and NR1-NR2D.

The Y axis of FIG. 16 shows hypothetic values, where 8000=NR1 at the lowest environmental stimulation (hypothetic), “e.g., dark room, no exposure to light” and 0 or very low nM glutamate concentration; 10000=NR1 at 0.37 μM glutamate concentration, 12000 at 1.1 etc. until 1-10 mM: around this glutamate concentration, especially when prolonged, NR1 (as a measure of neural plasticity) starts to decrease down to baseline levels (no glutamate) and lower.

The X axis of FIG. 16 shows glutamate at different concentrations (M) 0.001; 0.37 μM; 1.1 μM; 3.3 μM; 10 μM; 50 μM; 100 μM; 300 μM; 1 mM; 5 mM; 10 mM; 50 mM; 100 mM.

X values (glutamate μM) and Y values (hypothetic) NR1 subunits at different glutamate concentrations are shown in the legend of FIG. 16.

It should be considered that in vivo the amount of Ca²⁺ influx may be “excessive” (leading to excitotoxicity and halting of the neural plasticity machinery) even when the concentration of extracellular glutamate in the synaptic cleft is relatively low, e.g., low nM via GluN2C tonically and pathologically activated NMDARs relatively insensitive to the Mg²⁺ block.

Thus, in summary (1) dextromethadone differentially prevents glutamate induced excitotoxicity; (2) dextromethadone differentially modulates mRNA and synthesis of NMDAR receptor subunits; and (3) dextromethadone induction of mRNA and synthesis of NMDAR receptor subunits is differential for different subtypes and for the degree of stimulation.

### Example 3

A. Overview

This Example describes a Phase 2 study of two doses of dextromethadone in patients with MDD screened by SAFER. By this study, the present inventors demonstrate that dextromethadone is effective as a disease-modifying treatment for MDD. In particular, the inventors have determined: (1) Dextromethadone is safe and well tolerated in patients with MDD, with a side effect profile indistinguishable from placebo at disease-modifying doses, suggesting a selective action on hyper-stimulated NMDARs (pathologically hyperactive, with excessive Ca²⁺ influx) with sparing of physiologically active NMDARs; and (2) dextromethadone exhibits a persistent (sustained) therapeutic effect for at least seven days after discontinuation of treatment, signaling that its therapeutic effects are due to neural plasticity that persists beyond dextromethadone occupancy of the pore channel site of NMDARs or other receptors.

Thus, in light of (1) the known role of NMDARs in LTP, LTD, and formation of memories (Baez et al., 2018), including emotional memories (a subset of memory of interest in light of this Example 3); (2) the effects of dextromethadone (mediated by reduction of excessive Ca²⁺ influx via NMDARs), in particular NMDAR GluN1-GluN2C subtypes (Example 1), on activation of genes for production of synaptic proteins, including GluN2C subunits (Example 2); (3) dextromethadone's induced increase in neurotrophic factors, both in humans and experimentally, including BDNF; (4) experimental depressive phenotype improvement; and (5) the results of the Phase 2a study of this Example 3, the present inventors have determined, for the first time, that dextromethadone is disease-modifying, and thus potentially curative, for MDD.

And, as dextromethadone down-regulates Ca²⁺ influx via NMDARs (see Example 1) and in turn regulates NMDARs (see Example 2), the present inventors see profound implications on the role of differential patterns of Ca²⁺ influx as the epigenetic code for neural plasticity, in health and in disease.

Further, in view of these novel determinations, the present inventors also disclose that this can be applied to a multiplicity of diseases and disorders triggered, maintained, or worsened by NMDAR overstimulation/hyperactivity and excessive Ca²⁺ influx in select cells expressing NMDARs on the cell membrane (including extra CNS cells) by reversing the effects of excessive Ca²⁺ influx on the impairment of cellular physiological activity. In the case of neurons, cellular functions related to neural plasticity (LTP+LTD) were shown by the present inventors to resume at a molecular level in vitro. This was shown both in experimental models (see Example 2) and in patients (see this Example 3), without affecting normally (physiologically) functioning neurons, as signaled by the side effect profile comparable to placebo for therapeutically effective doses seen in patients with MDD (as in this Example 3).

B. Lessons from Dextromethadone in Health and in Disease

The molecular actions of dextromethadone outlined above can help explain brain activity, not only in pathological conditions, but also during health, and support the concept of continuum between health and diseases, with unbalanced states potentially triggered, maintained, or worsened by hyperactivated NMDARs.

The present disclosure reveals that dextromethadone may protect “normal” healthy subjects from potential CNS damage caused by intense psychological stress by preferential block of GluN1-GluN2C pathologically hyperactive NMDAR subtypes (Example 1). When a sufficient number of NMDARs are pathologically hyperactive in a sufficient number of neurons as part of a discrete CNS circuit, for a sufficient amount of time (e.g., during pathologic tonic activation of certain GluN2C subtypes, such as may result from a stressful condition), those neurons and that circuit will be impaired and clusters of symptoms (diseases or disorders) specific for the impaired circuit will manifest.

During “mental health” (an equilibrated mental state not altered by excessive or abnormal stimulations, including allosteric modulators), the differential patterns of Ca²⁺ influx triggered by the intensity and frequency of stimuli (presynaptic glutamate release) are regulated by a “normal” post-synaptic glutamate-framework. This framework depends on genetic determinants present from conception [7 genes: GRIN 1 (with 8 splice variants) Grin2A, 2B, 2C 2D, 3A, and 3B], and concurrently depends on epigenetic determinants, continuously shaping the framework, starting at conception. The different subunits coded by the seven genes are assembled in tetramers with obligatory NR1 subunits (necessary for membrane expression of NMDARs) and 2A-D and or 3A-B subunits. 3A and 3 B subunits, devoid of a glutamate agonist site, could also potentially substitute for NR1 subunits in the tetrameric structure.

Differential amounts of Ca²⁺ influx via Ca²⁺ channels, including NMDARs, are the epigenetic determinants that direct the cell's translational and synthetic activities, including the shaping of the synaptic framework itself, in a self-learning paradigm (see Example 2). Environmental stimuli, via excitatory stimuli mediated by glutamate, translate into differential amounts of Ca²⁺ influx. Environmental stimuli start at conception (NMDAR channels are present on gametocytes and zygote) and then continue for the lifespan of the individual and direct the NMDAR synaptic framework (among other epigenetic directions that direct development, they also direct the transcription of the seven NMDAR genes, as seen in Example 2). This continuous exposure to environmental stimuli (constantly translating into NMDAR-regulated precise amounts of Ca²⁺ influx in cells) starting at conception, including in utero embryo exposure, constantly regulates cellular functions and concomitantly auto-regulates the NMDAR framework. Even the same (identical) stimulus will have a differential effect because of the regulatory effects of differential Ca²⁺ patterns on the synaptic framework, including NMDAR expression. (The differential effect will generally fall within physiological parameters manifested by the vast variation in individuality within a species: the more possible variation in NMDARs subtypes and their combinations, the more individual variability is possible within a species sharing a given similar NMDAR framework.) This ion channel (NMDAR) regulated code (patterns of Ca²⁺ influx) commands the activation of genes from conception on, shaping the individual (by selecting which genes are activated) based on a constant interaction with the environment. This supports the long-held assumption that humans (and other species) are not only shaped by the environment, but we are a unit (albeit each individual represents a small contribution to that unit) with the environment.

The constant ongoing interaction between (1) environmental stimulation (translated into impulses onto presynaptic neurons resulting in presynaptic axonal glutamate release) and (2) the postsynaptic (and pre-synaptic, Baretta and Jones 1996) receiving synaptic glutamate framework (mediated by glutamate in the presence of glycine and modulated by a multiplicity of PAMs and NAMs and potentially other agonists), regulate differential patterns of Ca²⁺ influx. At the same time (in turn, that is) the same framework of NMDARs is regulated by these differential patterns of Ca²⁺ influx and thus patterns of Ca²⁺ precisely modulate cell activities based not only on present stimuli [glutamate+mediators (agonists)+modulators (PAMs and NAMs)] but also based on past environmental stimulation, including the immediately preceding stimulus. Learning/memory, including emotional memory and predictions (a form of learning/memory that fabricates the future based on past experience, as opposed to recollections, a fabrication of the past, also based on past experience) are forms of structural (synapses) neural plasticity precisely chiseled by environmental stimuli transduced into patterns of Ca²⁺ influx. These same patterns of Ca²⁺ influx regulate the effects of environmental input (the effects of each stimulus) by constantly shaping the NMDAR framework. Dextromethadone, by downregulating patterns of Ca²⁺ influx in pathologically hyperactivated NMDARs (Examples 1, 3), determines neural plasticity (Example 2), including long-term modifications of the NMDAR framework, e.g., neural plasticity effects (induction of synaptic proteins and neurotrophic factors) that manifest themselves as therapeutic for MDD (as shown in this Example 3).

Based on the present inventors' experimental findings in vitro (Examples 1, 2, 5, 6) and in patients with MDD treated with dextromethadone (Example 3), the present inventors can now postulate that differential patterns of Ca²⁺ influx are not only regulated by the NMDAR framework, but also, in turn, that these same patterns regulate and determine the NMDAR framework over time [neural plasticity (LTP and LTD) occurring over the life span of the individual, from conception to death]. This regulatory effect of dextromethadone on Ca²⁺ influx via select NMDARs (Example 1) and downstream neural plasticity (Example 2) is potentially curative for MDD (Example 3), by allowing cells to resume the neural plasticity machinery (synthesis and assembly of synaptic proteins, synthesis and release of neurotrophic factors) and by allowing formation of layers of new emotional memory, neutralizing or reversing the previous pathological emotional memory and its effects.

The physiologic LTD (pruning) that occurs during certain phases of sleep can also be explained by the same mechanism: Differential patterns of Ca²⁺ occurring during certain phases of sleep are regulated by NMDAR expression and regulate NMDAR expression. The actions of dextromethadone may also be therapeutic during sleep.

Memory formation, including cognitive, motor, emotional, social memory, including fabricated memory [memory (learning, LTP) constructed for predictions/expectations and during recollections], explained by NMDAR dependent LTP and LTD, starts with differential patterns of Ca²⁺ influx regulated by NMDARs. These differential patterns of Ca²⁺ influx, under physiological circumstances, are determined by stimulus-induced (environment) glutamate presynaptic release and result in synaptic protein and neurotrophic factor transcription-synthesis and assembly-expression (e.g., AMPAR and NMDAR) and release (neurotrophic factors). This physiological memory formation (LTP and LTD) shapes the connectome (wiring and unwiring of neurons through synapses) and is the basis of individuality and consciousness (see below).

Emotional memories may be conscious: The present mood, i.e. the mood at any given moment, is determined by existing memory (connectome)+present environmental stimuli (external and internal) reaching the brain, including body sensations, generally dominated by species preserving needs (awareness of dangers-stress; thoughts about food and sex). Emotional memories may also be subconscious (mood retrievable with prompting) or unconscious [synapses that are not structured (immature) and cannot reach consciousness at a given time but may emerge at a different time depending on ongoing (added-LTP or subtracted-LTD) neural plasticity and maturation of synapses]. The anticipation of these emotional memory constructs and their importance in determining mood and behavior are nicely described by Pontius, A. A., Overwhelming Remembrance of Things Past: Proust Portrays Limbic Kindling by External Stimulus—Literary Genius Can Presage Neurobiological Patterns of Puzzling Behavior. Psychological Reports, 73(2), 1993, pp. 615-621. This work can now be revisited in light of the disclosures presented by the inventors, including the selectivity of dextromethadone and certain open channel blockers for pathologically and tonically hyperactive channel subtypes (Examples 1, 3, 5) and or further selectivity of select pore blockers for NMDAR channels part of the endorphin system (Example 10). Dysfunctional emotional memories (conscious, subconscious and unconscious, which represent an interchanging continuum) that may manifest as select neuropsychiatric disorders, including MDD and related disorders, are of interest for this disclosure.

The known role of NMDARs in LTP, LTD, and thus in memory formation, is confirmed by the disclosed actions of dextromethadone at NMDARs in Examples 1-11: Dextromethadone actions are selective and differential relatively to intensity and frequency of stimulation and the receiving NMDAR framework (including the influence of agonists+modulators), including the selective block of tonically and pathologically hyperactive NMDAR pore channels and the downstream consequences of differential patterns of Ca²⁺ influx on neural plasticity. In particular, the disclosed therapeutic effects of dextromethadone without cognitive side effects in patients with MDD disclosed herein corroborate the inventors' hypothesis of a selective re-equilibrating action (down-regulation of excessive Ca²⁺ entry in cells) exerted by dextromethadone on hyperactivated NMDAR expressed by cells rendered dysfunctional (unable to function for production of new emotional memory: synaptic protein transcription-synthesis and assembly-membrane expression and neurotrophic factor transcription-synthesis and release) by excessive Ca²⁺ influx. These CNS cells rendered dysfunctional by excessive Ca²⁺ influx via hyperactivated NMDARs are part of neuronal circuits [circuits that physiologically continuously evolve (ongoing stimulus induced LTP-LTD) in the same patient overtime], and are the target for dextromethadone and explain its effectiveness for MDD and its potential effectiveness for a multiplicity of neuro-psychiatric disorders, including in particular its effectiveness for MDD related disorders.

Without being bound to any theory, the present inventors believe one of the reasons for the rapid therapeutic effect in patients with MDD may be the activation of neurons in the mPFC, e.g., by neurotrophic factors, such as BDNF. Another possible explanation for the rapid effect in MDD patients is the interruption (NMDAR block) of tonic stimulation of inhibitory interneuron projecting to the mPFC. While hyperactive NMDAR cause halting of the neural plasticity machinery at the dendrites of postsynaptic neurons, they may also allow for depolarization and electrochemical transmission along the axon of postsynaptic neuron reaching inhibitory interneurons projecting to mPFC neurons. Dextromethadone, by downregulating Ca²⁺ influx, not only allows resumption of the neural plasticity machinery in these tonically hyper-stimulated neurons, but also decreases electrochemical transmission, thereby potentially quieting inhibitory interneurons projecting to mPFC neurons. Furthermore, the hyperactivation of NMDARs may cause clustering of GABAaRs with excessive inhibitory activity reaching select neurons, e.g., neurons in the mPFC. It is generally believed that under conditions of chronic stress the activation of interneurons that inhibit the mPFC serves an evolutionary (species preserving) purpose, by decreasing active decision making during prolonged stress. In MDD, this chronic hyperactivation of inhibitory interneurons may instead be part of the pathologic process that is potentially corrected by dextromethadone.

C. Dextromethadone Regulates NMDARs and Neural Plasticity

In light of the above observations and experimental results, the present inventors hypothesize that, in health and disease, emotions (such as contentedness, happiness, sadness, anxiety, et cetera) originate from conscious or subconscious, or even unconscious, emotional memories (LTP and LTD in neurons part of emotional circuits). These emotional memories are “learned” via glutamate triggered Ca²⁺ influx patterns entering cells via NMDARs and determining structural LTP and LTD (these cells include neurons part of neural circuits). These circuits evolve during the lifespan by means of ongoing neural plasticity regulated by differential patterns of Ca²⁺ influx via NMDARs. Learned emotions (emotions are learned circuits, as other learned neuronal circuits, such as cognitive, motor, and social memory circuits) are encoded via stimulus-driven, NMDAR-regulated, differential patterns of Ca²⁺ influx (as indicated above, these differential patterns of Ca²⁺ influx also regulate the regulator, i.e., regulate the NMDAR framework by inducing production of NMDAR subunits and nerve growth factors, as shown in Example 2). Virtually all stimuli from the external environment, including stimuli that enter via sensory organs, such as light and sound and other stimuli, are translated into glutamate release that will activate NMDARs, triggering differential patterns of Ca²⁺ influx; other external environmental stimuli may enter the individual's blood stream, including pH, or may be molecules formed by metabolic pathways, and may function as NMDAR agonists or PAMs and/or as NAMs.) Learned (neural plasticity) circuits that control emotions and their manifestations (affective states) may be impaired by overly stimulated NMDARs causing excessive Ca²⁺ influx patters that alter the functionality and structure of cells and their circuits (e.g., excessive Ca²⁺ influx causing a decrease in neural plasticity—such as a decrease in transcription and production of synaptic proteins including NMDAR subunits and BDNF).

And now the present inventors have shown that, when the pathologically hyperactive (excessive Ca²⁺ influx) NMDAR channels of select neurons are blocked by dextromethadone and Ca²⁺ influx (inward Ca²⁺ current) is downregulated (as seen in Example 1), the neural plasticity machinery [synthesis of synaptic proteins, including NMDAR subunits (Example 2) and neurotrophic factors, such as BDNF], resumes, and the MDD phenotype is corrected (Example 3).

The interruption of overstimulation of NMDARs can happen without a pharmacologic NMDAR block. For example, in mild cases of depression or anxiety the removal of a triggering stressful psychological stimulus will, by itself, result in a sudden decrease in presynaptic glutamate release, and this decrease in “excessive” glutamate release will downregulate the previously excessive Ca²⁺ influx with an effect on neural plasticity similar to the decrease in Ca²⁺ influx exerted by dextromethadone's NMDAR channel block. As a result, the cell resumes neural plasticity activity, new channels are formed, BDNF is produced and released, and new “healthy” emotional memory is formed, neutralizing the prior “pathological” emotional memory. This explains the spontaneous recovery of patients with MDD and related neuropsychiatric disorders (e.g., GAD) and the high placebo response generally seen in the trial presented in this Example 3 (and in other clinical trials), where 15% and 5% of patients treated with placebo achieved remission at days 7 and 14, respectively. While the use of SAFER in the present inventors' Phase 2a trial (to exclude subsets of patients that may confound clinical trial results) was able to exclude some of the patients more likely to respond to placebo, it did not exclude all of them.

The constant epigenetic reshaping of neural plasticity (memory formation) is determined by experience [environmental stimuli reaching the individual (starting at conception) via a multiplicity of means (not limited to sensory organs)], mediated by presynaptic glutamate release, and the resulting differential patterns of Ca²⁺ influx (differential kinetics of Ca²⁺ influx into postsynaptic neurons) that regulate neuronal plasticity are regulated by neuronal plasticity through differential NMDAR frameworks that change constantly across the lifespan. This perennial change in membrane expression of NMDAR framework includes the developmental switch of NMDARs (Hansen et al., 2018), and is the basis of all forms of learning (cognitive, motor, emotional, and social memory/learning). Cognitive (e.g., language learning), motor (e.g., walking), emotional (e.g., contentedness), social (e.g., starting from non-verbal “imitation” as a communication tool) memories are structurally and functionally manifested as stronger or weaker synapses within more (stronger) or less (weaker) connected neuronal circuits. These circuits can be stronger or weaker and can be more or less interconnected (individuality of connectome). Thus, memories (the basis for individuality), including, but not limited to, emotional memories (though emotional circuits are considered more closely in the present inventors' experimental findings), are constantly changing from conscious to subconscious to unconscious (individuality and consciousness change across the lifespan, regulated by ongoing LTP and LTD).

Exposure to specific stimuli that, via glutamate and/or PAMs or NAMs, determine specific differential pattern of Ca²⁺ influx, regulated by NMDARs, and that in turn regulate the NMDAR framework, will continuously reshape synapses, structurally and functionally (e.g., via synthesis and membrane expression of synaptic proteins, and synthesis and release of NGF, including BDNF).

Differential patterns of Ca²⁺ influx via NMDARs are the shared code that ultimately determine differences and similarities among individuals of the same species (individuals in the same species have similar NMDAR frameworks, and individuals in the same society are exposed to similar environmental stimuli, including cultural stimuli, including imitation of similar behaviors). Differential patterns of Ca²⁺ influx represent the epigenetic code for determining and explaining individuality, consciousness, learned memories, emotions, etc., and preferential communication both within and across species, as discussed further below:

(1) Individuality: Even for identical twins, with identical NMDAR genes and subtypes and isoforms, differential experience (exposure to environmental influences, i.e., exposure to epigenetic influences) begins to differ when the zygote splits into two separate embryos. The differential exposure to environmental influences (anything outside the zygote and embryo) will determine differential pattern of Ca²⁺ influx via NMDARs, differentially regulating development, including neural plasticity, and determining CNS individuality in identical twins (while structural CNS differences may be difficult to prove in humans, it is a known fact that at birth identical twins have different fingerprints, signaling that differential environmental exposures (and their epigenetic influence) start very soon after the splitting of the zygote). Mutations can also differentially affect embryonic development and explain some differences among identical twins;

(2) Consciousness: Learning and recollection of the learned memory and the ability not only to recollect but also, based on learned memory, the ability to “reason”, fabricate, project and predict;

(3) Learned memories: These memories include cognitive, motor, emotional (individual), and social (collective) circuits;

(4) Individual and social emotions, and behaviors, beliefs, religions, political and cultural movements;

(5) Preferential communication within species: Similar NMDAR framework (genetic and epigenetic) expressed on the membrane of cells translates into similar patterns of Ca²⁺ influx generated by similar environmental stimuli (epigenetic) that produce learned memories that become recognizable and predictable across individuals of the same species living in contact with each other (e.g., tribes, local and regional communities, and nations);

(6) Preferential communication across different species: Similar environmental stimuli (epigenetic) fostered by closeness (e.g., man and dog) translate into patterns of Ca²⁺ influx across NMDARs (genetic and epigenetic) that produce learned memories that become recognizable and predictable across individuals of different species.

All of the above are examples of learning and memory formation at the molecular level determined by differential patterns of Ca²⁺ influx regulating gene expression and neural plasticity. Structural (synapses/connectome) and functional (NMDAR framework in action) neural plasticity (memory formation) is the ongoing real time effect of the external environment on the nervous system of the individual and is coded by differential patterns of Ca²⁺ influx across NMDARs. The same patterns of Ca²⁺ influx regulate themselves by modulating the NMDAR framework. Patterns of Ca²⁺ influx serve as the epigenetic code. And, in the CNS, the epigenetic code is represented by differential patterns of Ca²⁺ influx via the pore of NMDARs.

Lastly, the complex (but only apparently chaotic) constant brain activity during the lifespan of any individual can be best understood as the reverberation (via a multiplicity of neurotransmitters) of the downstream effects of differential patterns of Ca²⁺ influx elicited by environmental stimuli (epigenetic stimuli), via glutamate/glycine (agonist, mediators) and via PAMs-NAMs (allosteric modulators), which gate NMDARs. While voltage gating of NMDARs by Na⁺ influx via AMPA receptors is crucial for releasing the Mg²⁺ block from the pore of the NMDAR channel, cell activity, including gene regulation, is controlled by differential patterns of Ca²⁺ influx. NMDAR frameworks are regulators of (and are regulated by) these differential patterns of Ca²⁺ influx. These differential patterns of Ca²⁺ influx serve as the shared code for translating environmental stimuli into finely tuned neural plasticity (pre and post-synaptically) and are thus responsible for constantly reshaping the connectome (structural memory) in humans and other species.

Environmental stimuli, translated into glutamate release, are likely to first affect NMDAR channels tonically active not completely closed by Mg²⁺ (e.g., in C and D) and this physiological enhancement of tonic NMDAR activation (seen in the present inventors' Example 1 at very low glutamate concentrations) by low concentration glutamate (unable to release the Mg²⁺ block via AMPA activation but high enough to produce/enhance tonic Ca²⁺ influx, e.g., 40-200 nM) may modulate the neural plasticity machinery with production of LTP (maturation of synapses: with synaptic proteins and neurotrophic factor production, production/enhancement of spines) However, if the Ca²⁺ influx becomes excessive the physiological mechanisms of neural plasticity may be interrupted. The disclosures by the inventors of novel data from Examples 1-10 signal the disease-modifying effects of dextromethadone for a multiplicity of diseases and disorders triggered, maintained and worsened by excessive Ca²⁺ influx through hyperactivated NMDARs with potential therapeutic, preventive and diagnostic uses for dextromethadone and related compounds disclosed by the inventors.

Thus, dextromethadone, a very well-tolerated drug at doses that selectively target tonically and pathologically hyperactive NMDAR channels, is now being disclosed by the present inventors as a powerful research and clinical tool for understanding brain function in health and disease and for preventing, treating and diagnosing a multiplicity of diseases and disorders caused by pathologically hyperactive NMDAR and excessive Ca²⁺ influx in select cells integral to tissues, organs and circuits in humans and other species (as will be discussed in the “Lessons from Dextromethadone” section, below).

D. Lessons from Dextromethadone in “Disease”: MDD patients Phase 2a Study

The Phase 2a study looked at oral doses of dextromethadone at 25 mg and 50 mg administered daily to hospitalized patients with MDD (diagnosis confirmed with SAFER).

1. Methods

A Phase 2a, multicenter, RDBPC 3-arm study assessed the safety, tolerability, and PK of dextromethadone, and explored efficacy of two oral doses of dextromethadone (also referred to in this Example as REL-1017) as therapy in patients with MDD. Patients were adults age 18-65 with no response to 1 (87.1%), 2 (11.3%) or 3 (1.6%) adequate antidepressant treatments. Patients included in the trial included patients meeting criteria for TRD. After a screening period, 62 patients (x age=49.2 years, x HAMD score=25.3, x MADRS score=34.0) were randomized in a 1:1:1 ratio to either placebo, or dextromethadone) 25 mg QDay or dextromethadone 50 mg QDay in addition to their ongoing treatment with SSRIs, SNRIs, or bupropion (in particular, the sixty-two patients were taking one or more of fluoxetine, paroxetine, sertraline, escitalopram, citalopram, bupropion, vortioxetine, venlafaxine, and duloxetine). Patients in the dextromethadone groups received one loading dose of 75 mg (25 mg group) or 100 mg (50 mg group). All patients completed an inpatient 7-day treatment and were discharged after 2 days to return for follow up visits at Day 14 and 21. Potential efficacy was assessed with MADRS, SDQ and CGI scales at Day 2, 4, 7 and 14. Safety scales included 4-PSRS for psychotomimetic symptoms, CADSS for dissociative symptoms, COWS for withdrawal signs and symptoms and CSSRS for suicidality. All 62 randomized patients were part of the ITT population analysis.

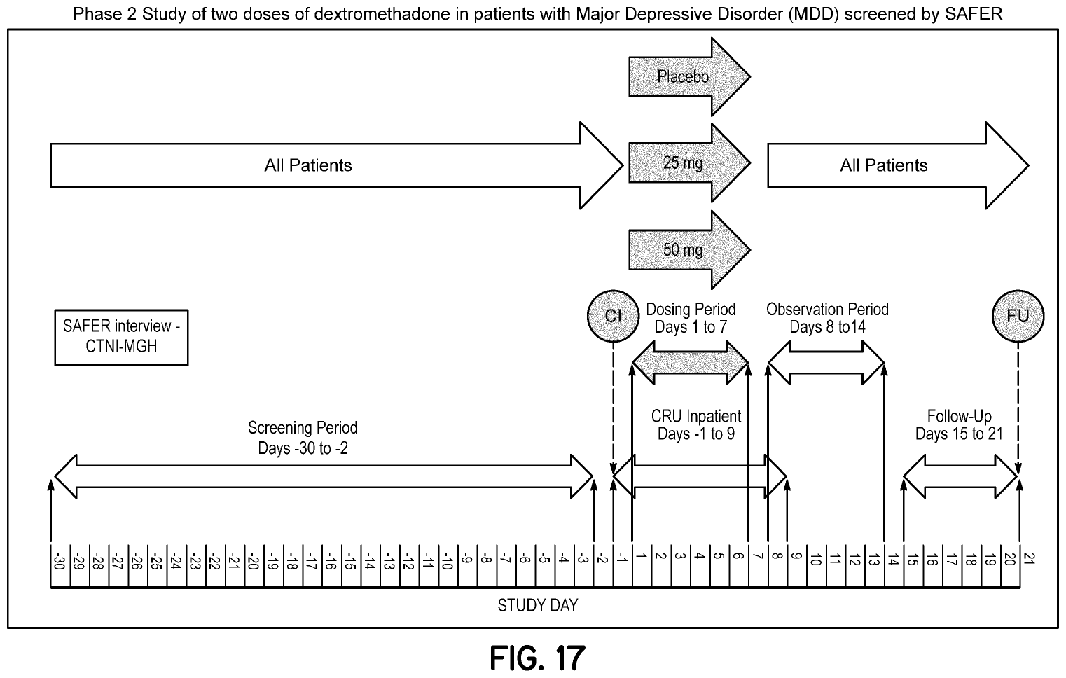

A schematic of the screening and dosing in patients in this study is shown in FIG. 17. The patients' disposition, demographic characteristics, and MDD severity were homogeneously distributed across arms, as shown in Table 30 below.

TABLE 30

| | | | REL-1017 | | REL-1017 | | All | |
|---|---|---|---|---|---|---|---|---|
| | Placebo | | 25 mg | | 50 mg | | Subjects | |
| Randomized Subjects | 22 | | 19 | | 21 | | 62 | |
| Completed all visits (Day 21) | 20 | | 18 | | 19 | | 57 | |
| Received all doses | 21 | | 19 | | 21 | | 61 | |
| Age: mean years (SD) | 49.7 | (11.1) | 49.4 | (12.4) | 48.6 | (10.9) | 49.2 | (11.3) |
| Females | 11 | (50%) | 8 | (42.1%) | 9 | (42.9%) | 28 | (45.2%) |
| Subjects ITT | 22 | | 19 | | 21 | | 62 | |
| Subjects PPP | 21 | | 19 | | 21 | | 61 | |
| Screening HAMD - Mean (SD) | 25.6 | (3.5) | 25.1 | (3.5) | 25.0 | (3.8) | 25.3 | (3.6) |
| Baseline MADRS -Mean (SD) | 33.8 | (4.0) | 32.9 | (6.0) | 35.2 | (3.9) | 34.0 | (4.7) |

Further, patients in the Phase 2 study experienced failure with previous antidepressant treatments. The number of failed previous antidepressant treatments per each group is shown in Table 31 below.

TABLE 31

| | | | REL- | | REL- | | | |
|---|---|---|---|---|---|---|---|---|
| | | | 1017 | | 1017 | | All | |
| | Placebo | | 25 mg | | 50 mg | | Subjects | |
| | (N = 22) | | (N = 19) | | (N = 21) | | (N = 62) | |
| % subjects | 22 | (100%) | 19 | (100%) | 21 | (100%) | 62 | (100%) |
| with ATRQ | | | | | | | | |
| Overall | | | | | | | | |
| Number of | | | | | | | | |
| Failed Prior | | | | | | | | |
| Treatments | | | | | | | | |
| 1 | 21 | (95.5%) | 17 | (89.5%) | 16 | (76.2%) | 54 | (87.1%) |
| 2 | 1 | (4.5%) | 2 | (10.5%) | 4 | (19.0%) | 7 | (11.3%) |

| | | | | | | |
|---|---|---|---|---|---|---|
| 3 | 0 | 0 | 1 | (4.8%) | 1 | (1.6%) |

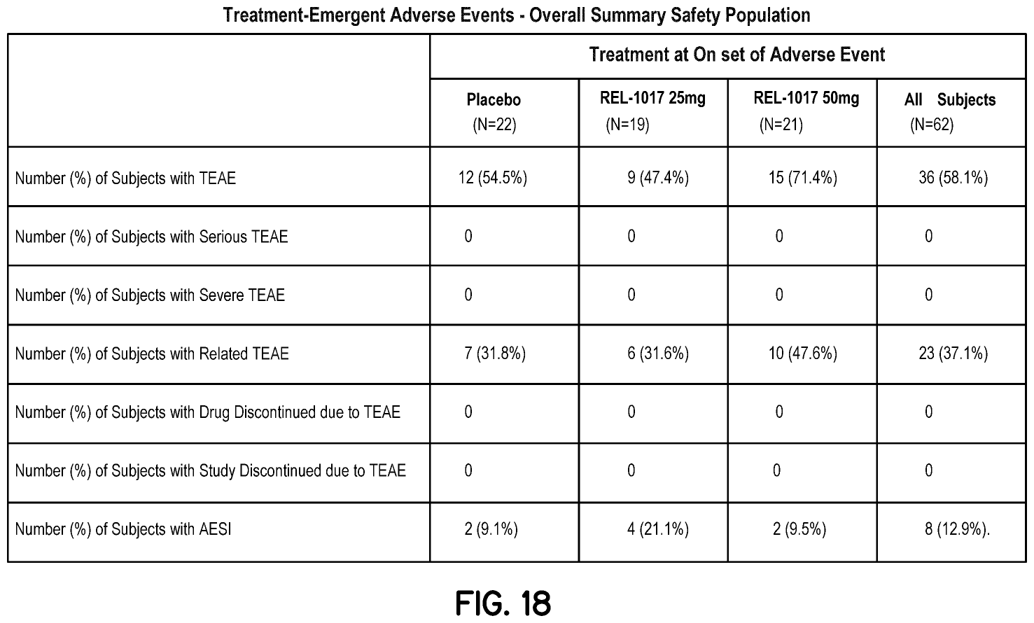

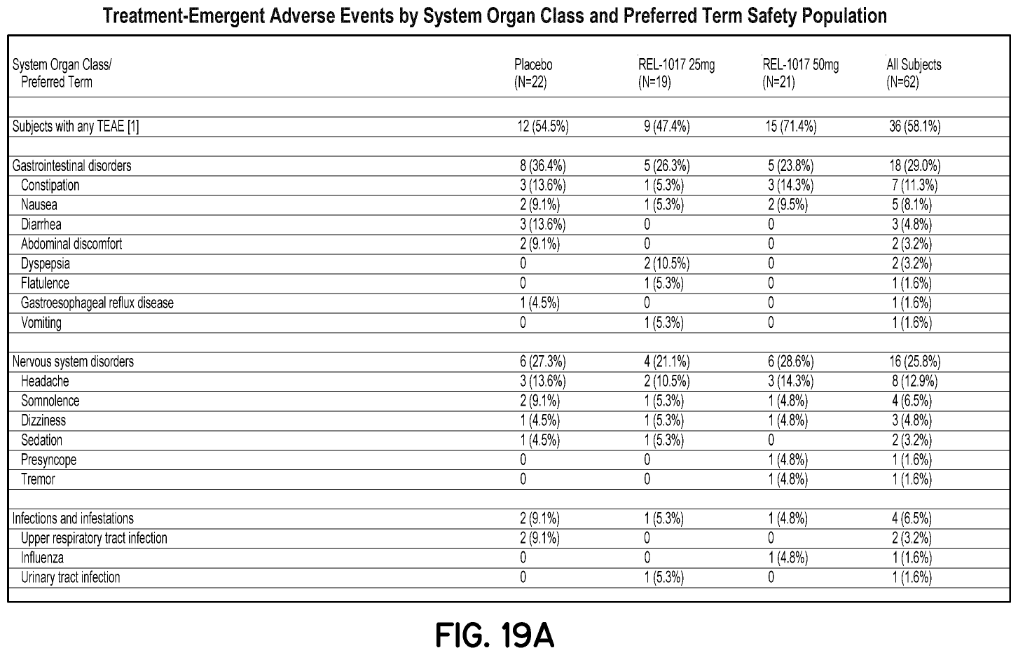

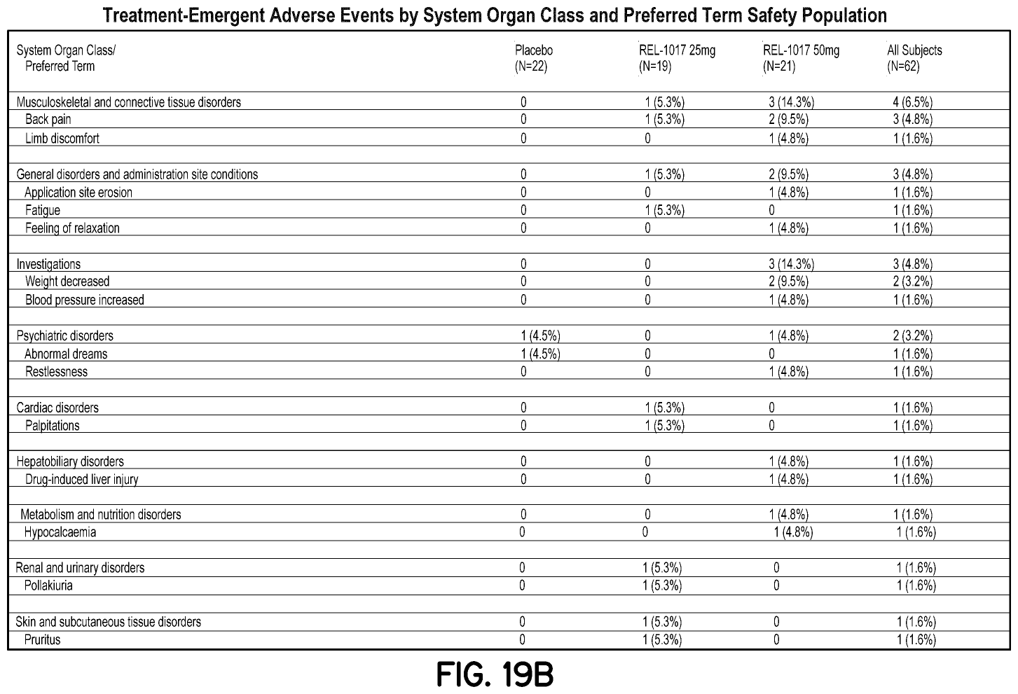

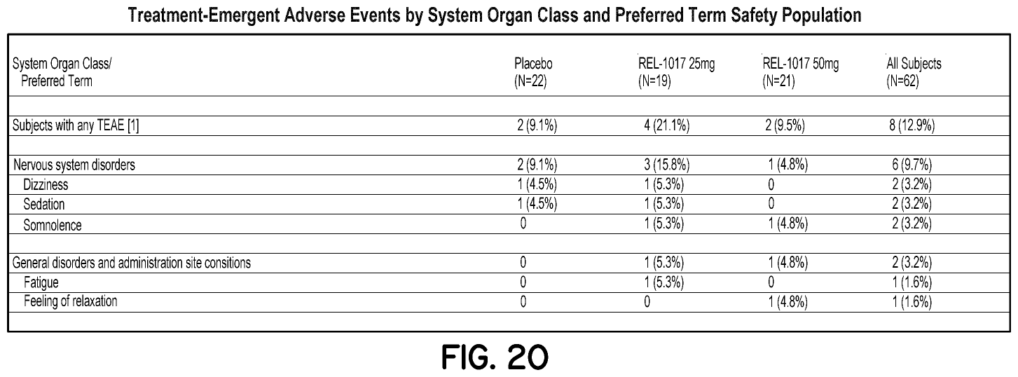

A table of treatment-emergent adverse events (overall summary safety population) is shown in FIG. 18. A table of treatment-emergent adverse events by system organ class and preferred term safety population is shown in FIGS. 19A and 19B. A table of adverse events of special interest (AESI) by system organ class and preferred term safety population is shown in FIG. 20.

2. Results

The data from this Phase 2a study showed strongly positive efficacy results, with highly statistically significant p values for all administered depression scales, with a large effect size, rapid efficacy (the first signals of efficacy unexpectedly started on day two for the 25 mg dose and were statistically significant for both doses, 25 mg and 50 mg, on day 4), and sustained efficacy (long lasting/persistent and statistically significant and clinically meaningful therapeutic effects and large effect size) persisting for at least one week after abrupt discontinuation of the 1-week treatment course.

The study also confirmed the favorable safety, tolerability and PK profiles of dextromethadone observed in Phase 1 studies. Patients experienced mild and moderate AEs, and no SAEs, with no higher prevalence of relevant organ group AEs in the REL-1017 (dextromethadone) groups vs placebo group. There was no evidence of treatment induced psychotomimetic and dissociative AEs or narcotic effects or withdrawal signs and symptoms. There was no evidence of clinically meaningful QTc prolongation, defined as 500 msec or an increase of 60 msec over baseline. Patients in the dextromethadone 25 mg and 50 mg groups experienced rapid (starting at day 2), sustained (up to day 14, last efficacy assessment), and statistically significant improvements with a large effect t size compared to patients in the placebo group on all efficacy measures, including MADRS, CGI-S scale, CGI-I scale, and SDQ. Improvement on MADRS appeared on Day 2 in the 25 mg group and were statistically significant in both dextromethadone dose groups on day 4 and continued through Day 7 and Day 14 (7 days after treatment discontinuation) with P values <0.03 and effect sizes from 0.7 to 1.0. Similar findings emerged from CGI and SDQ scales.

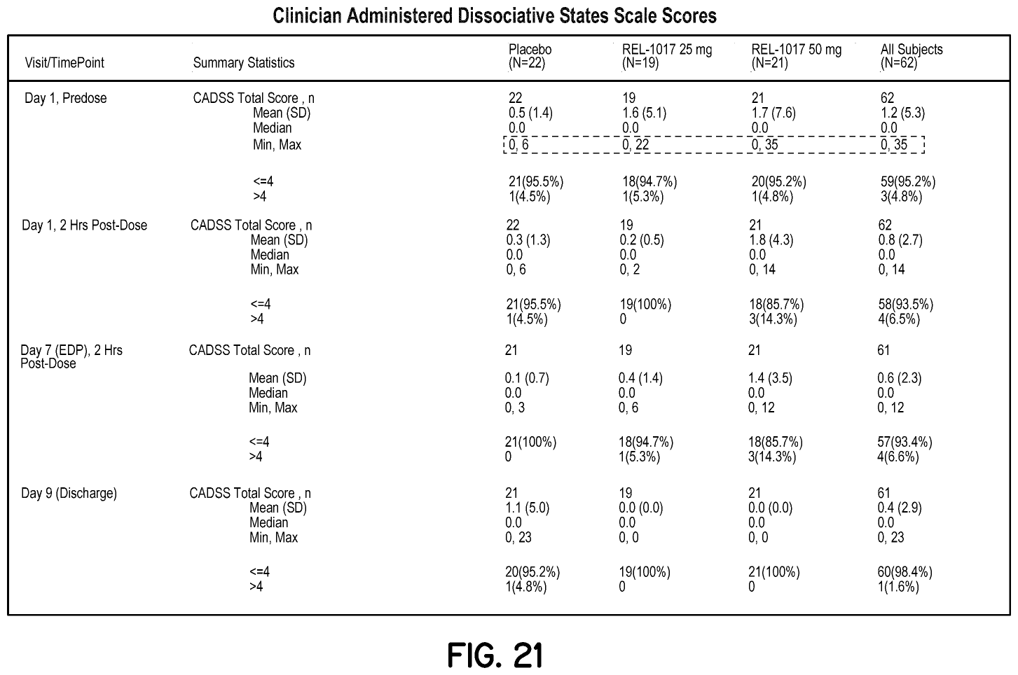

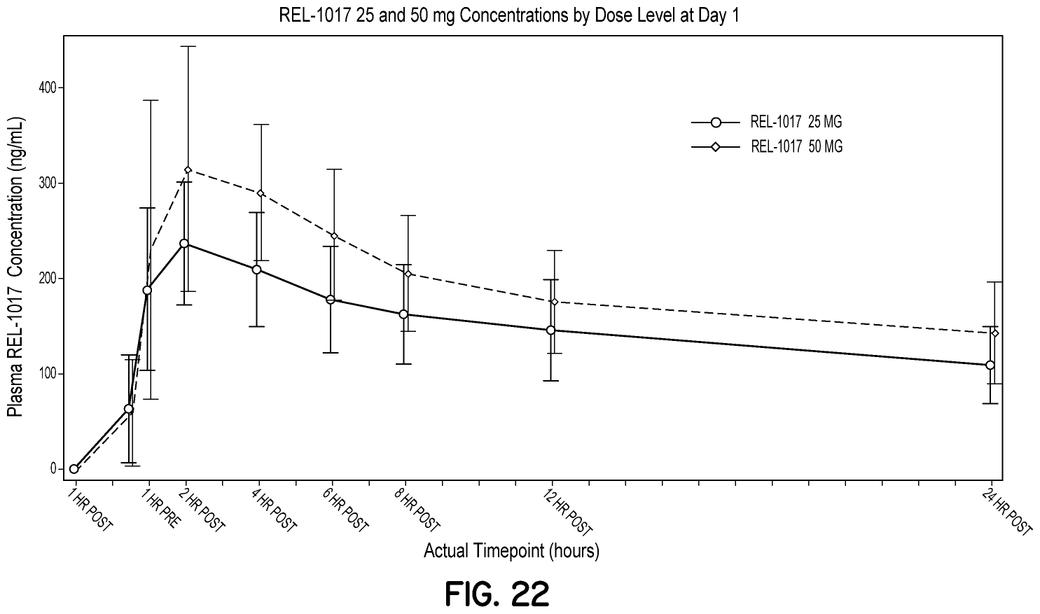

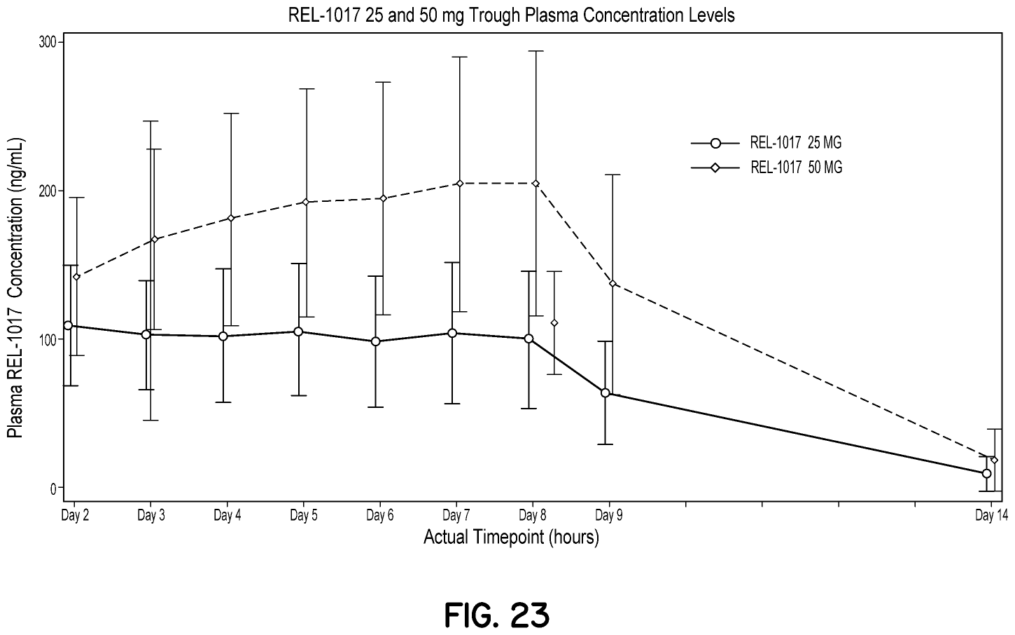

A table of clinician administered dissociative states scale scores during this study is shown in FIG. 21. And FIGS. 22 and 23 show plasma concentrations of dextromethadone by dose level (25 mg or 50 mg) at Day 1 (FIG. 22), and trough plasma concentration levels of dextromethadone for the two dose levels (FIG. 23). The findings shown in both of these figures are consistent with Phase 1 studies results.

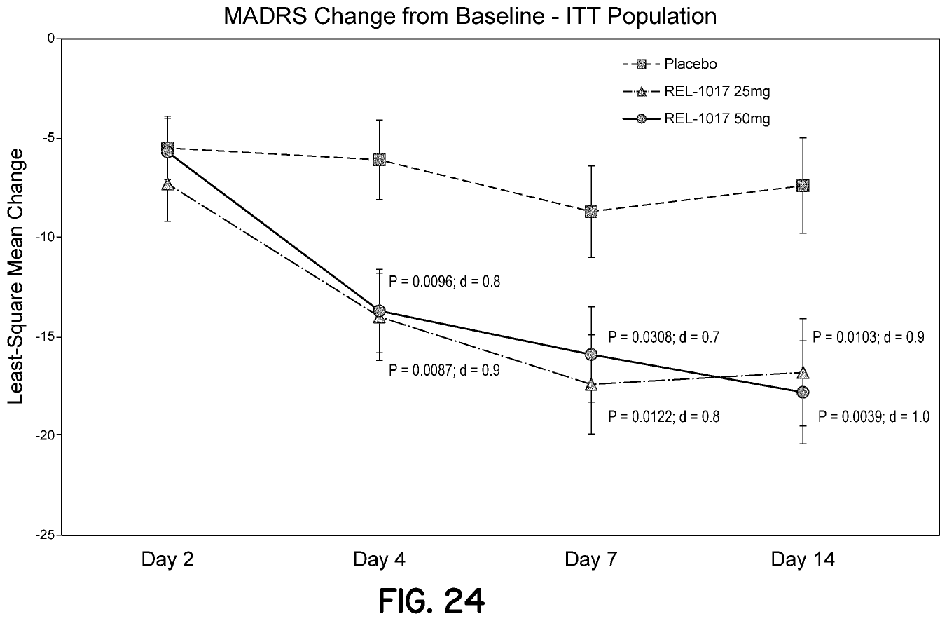

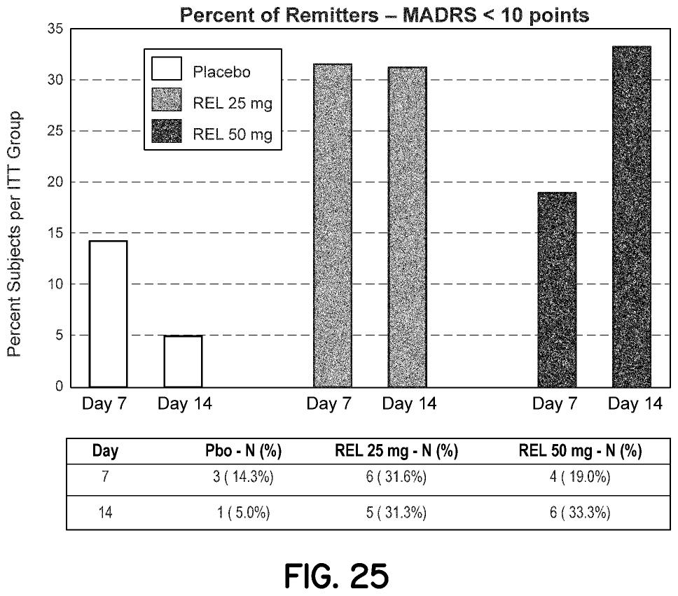

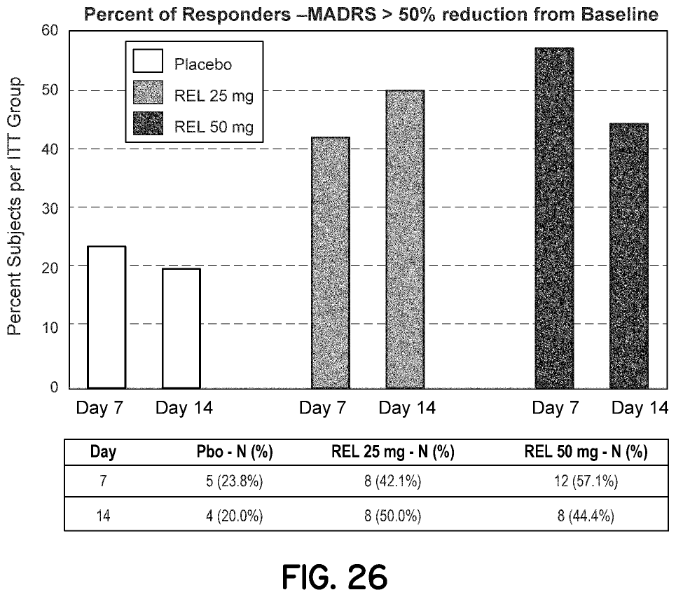

Furthermore, there was a signal for better efficacy from the 25 mg dose compared to the 50 mg dose. The drug was well tolerated at effective doses with side effects comparable to the placebo treated patients at the 25 mg dose and a signal for a higher incidence of side effects for the 50 mg dose, compared to placebo and compared to the 25 mg dose. The placebo response in the patients diagnosed with MDD and then screened by SAFER was lower (−7.4 points on MADRS) than the placebo response characteristically seen (typically −9-12 points on MADRS). Moreover, the magnitude of the response, independently from the relation to the placebo effect, was larger (−17.8) than that characteristically seen (typically −12-14). FIG. 24 shows that MADRS scores in the treatment groups achieved statistically significant difference versus placebo from Day 4 through Day 14. FIG. 25 shows the percent of remitters—MADRS<50% reduction from baseline.

E. Safety and Tolerability Findings

Study results confirm the favorable tolerability and safety profile observed in the Phase 1 SAD and MAD studies. These include: (1) only Mild and Moderate AEs—no SAEs; (2) no increased prevalence of specifically relevant organ group AEs in treatment groups vs placebo; (3) no evidence of treatment induced dissociative symptoms in the treatment groups vs placebo; (4) no evidence of treatment induced psychotomimetic symptoms in treatment groups vs placebo; and (5) no evidence of opiate withdrawal symptoms in treatment groups vs placebo.

F. Efficacy Findings

Dextromethadone 25 and 50 mg show rapid onset and sustained antidepressant efficacy in patients with MDD with statistically significant differences compared to placebo on all efficacy measures. These include: (1) solid efficacy results on MADRS with P values <0.03 and large effect sizes (0.7-1.0) from Day 4 to Day 14; (2) CGI-S and CGI-I solid findings consistent with MADRS results with P values and effect sizes of similar magnitude; (3) SDQ scores with moderate effect size differences (d=0.4 and 0.5) from Day 4 to Day 7 and with both statistically significant differences and large effect size for both 25 mg (P=0.0066; d=0.9) and 50 mg (P=0.0014; d=1.1) arms at Day 14; (4) rapid onset and long-lasting antidepressant efficacy; and (5) findings supporting continuing clinical development and strongly signaling efficacy for dextromethadone as mono-therapy for MDD.

G. Discussion and Conclusions

REL-1017 (dextromethadone) 25 and 50 mg confirmed very favorable safety, tolerability and PK profiles. Unexpectedly, the responses and remissions in patients with MDD induced by REL-1017 (dextromethadone) 25 and 50 mg were rapid, statistically significant with a large effect size, clinically meaningful and were sustained after discontinuation of therapy. The sustained improvements on multiple dimensions of MADRS, CGI-S scale, CGI-I scale, and SDQ seen on day 14 (1 week after the last treatment dose) at plasma levels of dextromethadone that would not result in effective NMDAR occupancy, signal a disease-modifying effect and mechanism of action that has never been shown before. Thus, the findings of this study signal for the first time that dextromethadone represents a disease-modifying treatment for MDD and related disorders (e.g., other disorders caused by excessive Ca²⁺ influx in select cells), and not simply a symptomatic treatment limited to receptor binding. In addition to the disease-modifying effects of dextromethadone as adjunctive treatment for MDD, the results strongly signal similar effects for dextromethadone as monotherapy in MDD and related disorders.

The unexpected efficacy results of this Phase 2a study, corroborated by findings on the mechanism of action and its downstream effects (as disclosed by the inventors in Examples 1-11 herein), taken together with the other evidence presented throughout this application suggest:

(1) In at least a subset of patients (with a diagnosis of MDD and further screened with the SAFER criteria), the disorder is caused and/or maintained by excessive Ca²⁺ influx in select neurons part of select circuits involved in emotional processing.

(2) The clinical effects of dextromethadone outlast receptor occupancy and are thus likely due to resumption of neuronal functions, including synthesis of synaptic proteins and neurotrophic factors, and resumption of neural plasticity and neuronal circuitry restoration.

(3) The 25 mg dose, resulting in dextromethadone plasma levels of approximately 50-150 ng/ml or a concentration of approximately 150-500 nM, results in therapeutic effects potentially stronger and with a more rapid onset compared to the therapeutic effects resulting from plasma levels obtained with the 50 mg dose, 150-450 ng/ml, or a concentration of approximately 500-1300 nM. This signal suggests that, for the average patient, the lower concentrations of dextromethadone are sufficient to block the pathologically hyperactive NMDAR channels that cause excessive Ca²⁺ influx and MDD and that daily oral doses higher than 25 mg may not be necessary for the achievement of therapeutic effects in the majority of patients with MDD.

(4) The inventors performed an additional sub analysis of the Phase 2a study data. The sub analysis correlated BMI, dose, response (Table 32, below), and plasma levels. Interestingly, patients defined by the CDC as normal or overweight according to their BMI responded very well to 25 mg of dextromethadone, while those defined as obese (BMI 30 or above) did not respond adequately. However, in both 25 mg and 50 mg dosage groups, unexpectedly, the plasma levels did not vary with BMI. Normal and overweight patients administered the higher dextromethadone dose, 50 mg, responded less adequately than the patients with the same BMI who were administered 25 mg. Furthermore, the obese patients administered 50 mg responded much better than the obese patients administered 25 mg. However, as stated above, even with the 50 mg dose, the plasma level did not vary with the BMI. Tables 32-34 below illustrate the effect of BMI on clinical outcome and plasma levels.

TABLE 32
CDC BMI definitions: normal (NL 18.5-24.9), overweight
(OW 25-29.9), obese (OB 30 and above); DM
ng/ml = dextromethadone plasma levels

| | | MADRS | DM ng/ml | MADRS |
|---|---|---|---|---|
| 25 mg | BMI | CFB day 7 | days 7/14 | CFB day 14 |
| N = 4 | NL | 21.75 | 77/5 | 15.6 |
| N = 12 | OW | 16.91 | 115/14 | 17.8 |
| N = 3 | OB | 8.6 | 113/14 | 7.6 |
| 50 mg | BMI | CFB day 7 | ng ml 7/14 | CFB day 14 |
| N = 6 | NL | 19.5 | 205/22 | 24.75 |
| N = 7 | OW | 15.3 | 120/7 | 12 |
| N = 8 | OB | 15.7 | 194/18 | 21.1 |

TABLE 33
Median BMI all patients 28.6

| | | MADRS | DM ng/ml | MADRS |
|---|---|---|---|---|
| 25 mg | BMI | CFB day 7 | days 7/14 | CFB day 14 |
| N = 12 | Below | 19.6 | 113/15 | 17.6 |
| | median | | | |
| N = 7 | Above | 13.3 | 101/9.4 | 13.4 |
| | median | | | |
| 50 mg | BMI | CFB day 7 | ng ml 7/14 | CFB day 14 |
| N = 10 | Below | 16.6 | 209/17 | 15.4 |
| | median | | | |
| N = 11 | Above | 16.7 | 200/22 | 20.8 |
| | median | | | |

TABLE 34

Day 7 (25 mg)
BMI below median: Placebo vs 25 mg * (p value = 0.0464)
BMI above median: Placebo vs 25 mg NS (p value = 0.5234)
Day 14 (25 mg)
BMI below median: Placebo vs 25 mg * (p value = 0.0460)
BMI above median: Placebo vs 25 mg NS (p value = 0.3786)
Day 7 (50 mg)
BMI below median: Placebo vs 50 mg NS (p value = 0.1171)
BMI above median: Placebo vs 50 mg NS (p value = 0.1357)
Day 14 (50 mg)
BMI below median: Placebo vs 50 mg NS (p value = 0.1675)
BMI above median: Placebo vs 50 mg * (p value = 0.0143)

The inventors have performed work on dextromethadone and its isomers for decades. In particular, one of the inventors, Charles Inturrisi, has previously defined the role of plasma proteins in the pharmacology of methadone and its isomers [Inturrisi C E, Colburn W A, Kaiko R F, Houde R W, Foley K M. Pharmacokinetics and pharmacodynamics of methadone in patients with chronic pain. Clin Pharmacol Ther. 1987; 41(4):392-401] and has studied the influence of diet on methadone metabolism, with more rapid methadone clearance in patients on western diet compared to macrobiotic diet [Wissel P S, Denke M, Inturrisi C E. A comparison of the effects of a macrobiotic diet and a Western diet on drug metabolism and plasma lipids in man. Eur J Clin Pharmacol. 1987; 33(4):403-407].

CNS penetration of certain drugs, including methadone, is determined by levels of alfa-1-glycoprotein (AAG) [Jolliet-Riant P, Boukef M F, Duché JC, Simon N, Tillement J P. The genetic variant A of human alpha 1-acid glycoprotein limits the blood to brain transfer of drugs it binds. Life Sci. 1998; 62(14):PL219-PL226]. Racemic methadone and its isomers are primarily bound to AAG, in particular the orosomucoid2 A variant [Eap C B, Cuendet C, Baumann P. Binding of d-methadone, l-methadone, and dl-methadone to proteins in plasma of healthy volunteers: role of the variants of alpha 1-acid glycoprotein. Clin Pharmacol Ther. 1990 March; 47(3):338-46; Hervé F, Duché JC, d'Athis P, Marche C, Barré J, Tillement J P, Binding of disopyramide, methadone, dipyridamole, chlorpromazine, lignocaine and progesterone to the two main genetic variants of human alpha 1-acid glycoprotein: evidence for drug-binding differences between the variants and for the presence of two separate drug-binding sites on alpha 1-acid glycoprotein. Pharmacogenetics. 1996; 6(5):403-415]. AAG levels influence the effects of methadone in pre-clinical experimental settings [Garrido M J, Jiminez R, Gomez E, Calvo R. Influence of plasma-protein binding on analgesic effect of methadone in rats with spontaneous withdrawal. J Pharm Pharmacol. 1996; 48(3):281-284]. AAG is increased and free methadone is decreased in patients with withdrawal [Garrido M J, Aguirre C, Trocóniz IF, Marot M, Valle M, Zamacona M K, Calvo R. Alpha 1-acid glycoprotein (AAG) and serum protein binding of methadone in heroin addicts with abstinence syndrome. Int J Clin Pharmacol Ther. 2000 January; 38(1):35-40]. Finally, levels of alfa-1-glycoprotein are increased in obesity, i.e., levels of alfa-1-glycoprotein are influenced by diet [Benedek I H, Blouin R A, McNamara P J. Serum protein binding and the role of increased alpha 1-acid glycoprotein in moderately obese male subjects. Br J Clin Pharmacol. 1984; 18(6):941-946] and diet impacts methadone PK (Wissel et al., 1987). Further, the free fraction of methadone is not significantly affected by elevated methadone concentrations or through displacement by other drugs that also bind to AAG [Abramson F P. Methadone plasma protein binding: alterations in cancer and displacement from alpha 1-acid glycoprotein. Clin Pharmacol Ther. 1982; 32(5):652-658].

Based on points (3) and (4), above, and other data disclosed throughout the application and the inventors' shared knowledge about methadone and its isomers and in particular dextromethadone, the inventors disclose that the therapeutic window for dextromethadone is narrower than its safety window, an unknown fact before the inventors' Phase 2a study and subsequent in-depth analysis of the Phase 2a data. Furthermore, this therapeutic window can be better defined by measurement of free dextromethadone levels and or measurement of AAG and or its variants, rather than by measuring total plasma levels (as had been done until this unexpended finding). Furthermore, the therapeutic free level (approximately 10% of the total plasma level) of dextromethadone for MDD and related disorders and possibly for other neuropsychiatric diseases is defined within a range 5-30 ng/ml or approximately 15-100 nM. Additionally, the inventors disclose that the potential therapeutic effects of dextromethadone in with MDD may be due to its metabolites and in particular to EDDP. The present inventors believe (based on data herein) that further study would find direct correlation between free dextromethadone levels and EDDP levels and therapeutic response, and would find an inverse correlation between AAG levels and therapeutic response.

Continuing from the list of points (1)-(4), above, of conclusions obtained from the inventors work—the results of the Phase 2 study, and the other Examples and evidence presented herein also suggest:

(5) There may be patients diagnosed with MDD that are less likely to respond to a drug that blocks excessive Ca²⁺ influx in select neurons part of select circuits. Based on low placebo response and robust efficacy results in the present inventors' Phase 2 trial, the SAFER screening tool may be helpful in selecting out MDD patients less likely to respond to a drug, such as dextromethadone, that selectively down-regulates excessive Ca²⁺ influx. This effect of SAFER screening may help researchers and clinicians better define the subset of MDD with a disorder triggered and/or maintained by excessive Ca²⁺ influx into neurons that are part of an emotional processing circuit (emotional memory circuit).

(6) The results of subjects and patients treated with dextromethadone may help researchers and physicians define not only subsets of neuropsychiatric disorders, but also subsets of metabolic (e.g., diabetes, NAFLD-NASH, osteoporosis), cardiovascular (e.g., angina, CHF, HTN), immunologic, inflammatory, infectious, oncologic, otologic and renal disorders triggered, maintained or worsened by excessive Ca²⁺ influx in select neurons or other cellular populations determined by hyperactivation of NMDARs by glutamate and/or PAMs and or agonists.

(7) Aside from the absence of side effects at effective doses, the selectivity of dextromethadone for pathologically hyperactive NMDARs is also signaled by the lack of withdrawal (signs and symptoms) seen in the Phase 2a study. Drugs that exert clinical effects by acting directly on receptors or receptor pathways, such as opioids, benzodiazepines, dopaminergic drugs or antidopaminergic drugs or even SSRIs [Henssler J, Heinz A, Brandt L, Bschor T. Antidepressant Withdrawal and Rebound Phenomena. Dtsch Arztebl Int. 2019; 116(20):355-361] generally result in clinically meaningful withdrawals signs and symptoms upon abrupt discontinuation.

The fact that NMDARs are shared across vertebrates [Teng H, Cai W, Zhou L, Zhang J, Liu Q, Wang Y, et al. (2010) Evolutionary Mode and Functional Divergence of Vertebrate NMDA Receptor Subunit 2 Genes. PLoS ONE 5(10)] also suggests potential therapeutic uses for dextromethadone for the treatment of a multiplicity of veterinary diseases and disorders triggered, worsened, or maintained by NMDAR hyperactivation.

Furthermore, the work of the present inventors also discloses in vitro results that show that dextromethadone can potentially modulate inflammatory biomarkers that are abnormal in neuropsychiatric diseases and disorders, including MDD and TRD, and in neurodegenerative diseases, such as dementias, including Alzheimer's disease, and in Parkinson disease and neurodevelopmental diseases, such as autism spectrum disorders, and other neuropsychiatric diseases and disorders such as schizophrenia and others. These potential anti-inflammatory effects of dextromethadone are potentially due to block of NMDARs by dextromethadone (signaling a potential NMDAR block of NMDARs expressed by immune cells, including glial immune cells), and may also help explain its efficacy for a multiplicity of neuropsychiatric, metabolic, cardiovascular disorders, inflammatory, immunological disorders and neoplastic disorders. In light of the known mechanism of action of dextromethadone as an uncompetitive NMDAR channel blocker, these anti-inflammatory effects of dextromethadone may be an effect of down-regulation of excessive Ca²⁺ influx in cells regulating immunity.

The present inventors have confirmed the anti-inflammatory in vitro actions detailed in Example 11 with a set of clinical measurement of markers in patients suffering from MDD and treated with dextromethadone (see also Example 7, below). The present inventors hypothesize that these effects on inflammatory markers are caused by modulation by dextromethadone of NMDARs expressed on the cell membrane of select neurons and immune cells, including glial cells. The modulation of inflammatory markers in patients with neuropsychiatric disorders treated with dextromethadone may result from a dextromethadone effect on immune cell effect (modulation of immunological memory) that mirrors the effects seen in neurons on different types of memory (cognitive, emotional, motor memory) and mediated by increases in BDNF and synaptic proteins. If dextromethadone is able to improve functionality (e.g., immunological memory and inflammatory responses) in immune cells, it may be therapeutic, at the appropriate dose, for diseases and disorders affected by a dysregulated immune system, including inflammatory disorders, autoimmune disorders and oncological disorders, among others.

In addition to the results presented in this Example 3 for dextromethadone as adjunctive treatments in patients with MDD, the present inventors also disclose dextromethadone monotherapy in patients with MDD. The effects of dextromethadone were very robust in patients with MDD and concurrent antidepressant treatment, signaling potentially curative actions of dextromethadone not only for CNS abnormalities associated with MDD but also for CNS abnormalities potentially associated with MDD treatments (as shown in this Example 3). In other words, the downregulation exerted by dextromethadone on excessive Ca²⁺ influx in select neurons with pathologically hyperactive NMDARs is likely to occur with or without concurrent neuropharmacological treatment.

The present inventors postulate that the selective regulatory actions of dextromethadone on excessive Ca²⁺ influx may be particularly useful for patients who have not yet received treatments that potentially may alter CNS neurotransmitter pathways. Furthermore, the inventors disclose that dextromethadone and behavioral psychotherapy may be successfully combined.

As previously disclosed, dextromethadone had not been considered as a potential safe and effective drug because of concerns about abuse liability and concerns about QTc prolongation and arrhythmias. In this Example 3, the present inventors now provide additional data that counters these concerns. In particular, Example 3 data show lack of opioid effects on cognitive and respiratory functions (narcotic effects) and lack of dissociative and/or psychedelic effects typical of some NMDAR channel blockers such as MK-801, PCP, and ketamine. Furthermore, there were no clinically meaningful signs and symptoms of opioid withdrawal (measured with COWS) upon abrupt discontinuation. The data from Example 3 also confirmed the overall cardiac safety and lack of clinically meaningful QTc prolongation from dextromethadone.

Example 6 below (electrophysiological testing to establish “on” and “off” rates and “trapping”) and Example 3 (lack of psychotomimetic and psychedelic side effects in addition to lack of narcotic side effects at therapeutic doses) suggest that the uncompetitive block afforded by dextromethadone at the intramembrane MK-801 site of select hyperactive NMDAR channels allows cells to resume the physiological LTP cellular activities (e.g., production and assembly of synaptic proteins and production and release of BDNF) necessary for physiological brain functions.

The present disclosure of the present inventors' clinical and experimental data strongly signals towards a novel pathophysiologic understanding for MDD, related disorders, and other disorders. This novel pathophysiologic understanding is likely to have profound and immediate implications on therapeutic, preventive, and diagnostic strategies—and even on development of new therapeutic agents. By selectively targeting hyperactivated ion channels (e.g., NMDARs), without interfering with physiologically active NMDARs, at therapeutic doses, as underscored by the lack of psychotomimetic side effects and very good tolerability profile, and the rapid, robust and sustained efficacy, and the mechanisms of action outlined in Examples 1-11, dextromethadone potentially restores functionality to neurons and circuits that cause, trigger, maintain, and/or worsen neuropsychiatric and other disorders.

A similar mechanism of action (NMDAR block) has been disclosed for esketamine, recently approved by the FDA for TRD. However, the block provided by esketamine (and ketamine), while effective for treating MDD/TRD, does not appear to be selective for hyperactivated NMDAR (or if selective, the block does not have substantially useful “on”/“off” and/or related “trapping” qualities as disclosed in Example 6) because esketamine and ketamine cause intense psychotomimetic symptoms (dissociative effects), typical of higher affinity uncompetitive channel blockers and also seen with competitive NMDAR channel blockers, signaling interference by ketamine and esketamine with physiological NMDAR activity.

Dextromethadone's unique actions at NMDARs [e.g., a more homogeneous effect on different NMDAR subtypes A-D with a preference for GluN1-GluN2C subtypes (Example 1)], its specific “on”-“off” kinetics at the channel pore and “trapping” qualities and preference for GluN1-GluN2C subtypes in the presence of physiological amounts of Mg²⁺ (Example 6), or its affinity for other receptors (Example 10), may be “just right” for selectively targeting and blocking pathologically hyperactive NMDAR and other receptors in select CNS circuits, and, importantly, it characteristics may be “just right” for unblocking the NMDAR channel during physiological activities (e.g., phasic glutamatergic transmission).

The coupling of behavioral psychotherapy with dextromethadone may be a very effective strategy for the treatment of neuropsychiatric diseases and disorders: dextromethadone, with its graded selective block, allows psychotherapy induced “healthy” neural plasticity to occur in cells, which before therapy with dextromethadone displayed pathologically hyperactive NMDAR channels and a circuit (in the case of MDD an emotional memory circuit) that was refractory to stimuli, including the positive stimuli of psychotherapy, that could otherwise potentially have resulted in therapeutic neural plasticity effects. In other words, an emotional memory circuit impaired by neurons with pathologically hyperactive channels is refractory to psychotherapy [and can also be refractory to de-stressing (i.e., favorable) life experiences, as is the case in MDD]; on the other hand, the same circuit, with cells that now display formerly hyperactive NMDARs now blocked by dextromethadone (with block of excessive Ca²⁺ influx) may offer fertile terrain (production of synaptic proteins and BDNF) for “healthy” neural plasticity (LTP) induced by psychotherapy.

The differential cellular expression of NMDAR subtypes 2A-D on the cell membrane (part of the NMDAR framework) explain how experience-driven release of glutamate from the presynaptic cell (with or without the action of a PAMs or other agonists) determines the influx of a specific pattern of Ca²⁺ that will then result in downstream effects (e.g., CaMKII mediated) on transcription (induction of mRNA) and protein synthesis and protein assembly that regulate the synaptic activity and strength (at the basis of LTP and LTD for learning and memory formation), and including reverberating effects via other neurotransmitters. All these effects ultimately determine the constant connectome evolution/involution (re-shaping) during the lifespan of individuals. Based on the present inventors' preclinical in vitro and in vivo data and clinical data the NMDARs regulate and are regulated by differential patterns of Ca²⁺ influx.

The communication between neurons, essential for the constant re-shaping of the connectome, is determined by presynaptic actions (experience-driven presynaptic glutamate release by the excited presynaptic neuron—including NMDAR modulation by endogenous or exogenous PAMs e.g., polyamines, gentamicin, or agonists, e.g., quinolinic acid) and post-synaptic actions: NMDAR channel opening of differentially expressed NMDAR subtypes resulting in differential patterns of Ca²⁺ influx with downstream effects, including neural plasticity effects, including effects of NMDAR framework, including CaMKII mediated effects.

Thus, glutamate release from presynaptic cells results in a tightly regulated Ca²⁺ influx for a set amount of time that depends on the differential postsynaptic NMDAR framework (e.g., NR1-2A-D, NR1-3A-B and their potential tri-heteromeric variations). There are subtype-dependent differences in (1) deactivation kinetics (GuN2D is the slowest—more time allowed for calcium influx when 2D receptors are activated—and GluN2A the fastest—less time allowed for calcium influx when these channels are activated by glutamate) and in (2) strength of voltage-dependent Mg²⁺ block across all four GluN2 subunits [2D and 2C have the least strong Mg²⁺ block and thus their opening may be triggered by very slight depolarization or may even happen spontaneously, in the absence of membrane depolarization and triggered by low ambient concentrations of agonist (e.g., glutamate or quinolinic acid) at the synaptic cleft]. Other subtypes vary in their resistance to PAMs, Mg²⁺ block and Ca²⁺ permeability, including subtypes that include splice variants (isoforms) of the NR1 subunit or subtypes that are tr-heteromeric (e.g., NR1-NR2A-NR2B) and/or include NR3A-B subunits.

Dextromethadone, by interacting and modulating selectively pathologically hyperactive NMDAR channels in a manner that allows resumption of physiologic cellular activities [the “on” rate of dextromethadone allows its channel block only when the channel is pathologically hyperactive, while the “off” rate (and receptor interaction “trapping” qualities) allows expulsion of dextromethadone (similarly to the expulsions of MG²⁺) and resumption of cellular ion currents and related cellular activities under physiological conditions, e.g., environmental stimulation].

Dextromethadone, a very well-tolerated NMDAR channel blocker, with unique differential receptor subtype blocking qualities (Example 1) and just-right “on”/“off” and “trapping” kinetics (Example 6), and actions with or without PAMs and agonists (Example 5), and effects on synaptic protein induction, assembly and release (Example 2) and with selectivity for hyperactivated pathologically hyperactive NMDARs (Example 3), and thus selective downregulation of excessive Ca²⁺ influx, is now (due to the work of the inventors disclosed herein) revealing itself as “best in its class” (new emerging class of uncompetitive NMDAR blockers) for treatment of patients, for use as a research tool in healthy subjects (physiology of memory), and for prevention, treatment, and diagnosis of patients suffering from a multiplicity of disorders related to NMDAR hyperactivity.

Dextromethadone is likely to stimulate progress in the understanding of the role of tightly regulated patterns of Ca²⁺ influx (regulated by differential stimulation of the presynaptic cell and differential cellular expression of NMDARs 2A-D on the post-synaptic cell). These patterns of Ca²⁺ influx may represent the shared (across species) code that allows the connectome to constantly reshape itself (evolution and involution of synapses, LTP and LTD). The strengthening and formation of synapses is the basis of memory and learning, including learning of emotions and learning of social interactions, including emotional involvement in events and interpersonal relations, or even involvement in religions and political movements, resulting in behaviors and activities and moods ranging from ego-syntonic/society syntonic (“mentally healthy”) to ego-dystonic/society dystonic (“mentally unhealthy”) disruptive and pathologic behaviors and activities and moods, source of personal and social distress. The patterns of Ca²⁺ entry triggered by glutamate are thus regulated not only by the amount of glutamate released pre-synaptically [which among individuals of the same species (with similar NMDAR framework) is potentially similar for similar environmental stimulation], but is also precisely regulated by the NMDAR framework on the postsynaptic cell.

This expression of synaptic proteins (NMDAR framework) is similar among individuals in the same species but is differentiated according to the individual's genes for NMDARs, and environmental factors (G+E). Epigenetic (environmental influences) translate, via patterns of Ca²⁺ influx through NMDARs, into neural plasticity. Even among cells of the same type and topographically close to one another, the differential expression of NMDARs (part of the NMDAR framework) results in unique patterns of Ca²⁺ influx following a stimulation and presynaptic glutamate release. While the selectivity of dextromethadone seems to be directed to pathologically hyperactive NMDARs, its affinity for the different subtypes differs and thus it is likely to differentially block the pathologically hyperactive different receptor subtypes.

Furthermore, different doses of dextromethadone (see also plasma levels, Example 3, and FIGS. 22 and 23) may have differential effects on different subtypes. These differential effects, when fully elucidated, may uncover the full potential of dextromethadone and related compounds for the treatment of select disorders and diseases.

In experimental models, NMDAR channel blockers have been associated with neuronal vacuolation and other cytotoxic changes (“Onley lesions”). The potency of the drugs in producing these neurotoxic changes is related to their potency as NMDA antagonists: i.e. MK-801>PCP>tiletamine>ketamine [Olney J W, Labruyere J, Price M T (1989) “Pathological Changes Induced in Cerebrocortical Neurons by Phencyclidine and Related Drugs”. Science. 244: 1360-1362]. Dextromethorphan has been shown to cause vacuolization in rats' brains when administered at doses of 75 mg/kg [Hashimoto, K; Tomitaka, S; Narita, N; Minabe, Y; lyo, M; Fukui, S (1996) “Induction of heat shock protein Hsp70 in rat retrosplenial cortex following administration of dextromethorphan”. Environmental Toxicology and Pharmacology. 1 (4): 235-239]. The potential for NMDAR antagonists to cause permanent brain lesions has tempered development of NMDAR antagonist agents as therapeutic agents. The inventors for the first time have performed a test in rats to investigate the chronic CNS toxicity potential for dextromethadone. Dextromethadone doses were 0, 31.25, 62.5, and 110 mg/kg/day for males and 0, 20, 40, and 80 mg/kg/day for females. Methadone racemate was included as a comparator at 31.25 mg/kg/day in males and 20 mg/kg/day in females. MK-801 was tested as the positive control agent at 5 mg/kg (males) and 2 mg/kg (females). Of note, the smallest tested dose for dextromethadone (32.25 mg/kg/day) was over ten times the equivalent therapeutic human dose. Necropsies were conducted at 8, 48, and 96 hours after initial doses with daily dosing. Brains were evaluated by a neuropathologist with expertise in identifying Olney lesions (hematoxylin & eosin plus Fluoro Jade B stains). Dextromethadone at any tested dose did not cause Olney lesions, while the active control MK-801 caused Olney lesions in all tested animals (Relmada data on file). These data signal that dextromethadone can be safely used in humans, without concerns for CNS damage potentially seen with other NMDAR channel blockers in development for MDD, including dextromethorphan.

Furthermore, the NMDAR framework on the cell membrane of select neurons of an individual, which is determined both genetically [7 genes coding for the different subunits and numerous splice variants (isoforms) and vast mutation possibilities] and epigenetically (environmental influences from embryonic formation on) will determine the “mental traits” for that individual (individual reaction to environmental stimuli). The ongoing experience-driven neural plasticity (regulated by differential patterns of Ca²⁺ influx in the postsynaptic cell through postsynaptic NMDARs, triggered by presynaptic glutamate release) and other environmental effects on NMDAR (e.g., PAMs and NAMs at modulating sites, e.g., the polyamine site or agonists at agonist sites, e.g., quinolinic acid at the NMDA/glutamate site) contribute to determine the “mental state” for the individual (“trait” and “state” include the definitions by Desseilles et al., 2013), and, in light of the present inventors' present and previous disclosures, reflects the G+E paradigm at the basis of learning (memory formation, LTP, LTD) and of the unique connectome for each individual.

The availability of a new class of well-tolerated, safe and effective NMDAR blockers (e.g., dextromethadone and the compounds and methods previously and presently disclosed by the inventors) with actions at NMDARs that are differential for the different NMDAR subtypes, and that preferentially target certain circuits, can potentially treat and prevent and diagnose mental disorders and may also improve social function and work abilities which may be part of unfavorable “mental traits” due to dysfunctional NMDARs resulting in pathologically hyperactive NMDAR channels in select cells part of select circuits (e.g., reduced ability to perform tasks requiring a certain level of mental concentration).

NMDARs have a central role in learning (memory formation, LTP, LTD). Certain learning disabilities are potentially secondary to G+E determined dysfunction of NMDARs. In conjunction with addressing and correcting the environmental factors that trigger and/or maintain certain learning disabilities (e.g., ADHD), a well-tolerated and safe drug like dextromethadone may effectively regulate pathologically hyperactive NMDARs expressed by neurons that are part of neuronal circuits deputed to learning cognitive, social and motor skills. For example, aside for regulating hyperfunctioning NMDARs disrupting a particular neuronal circuitry involved with learning and memory formation of cognitive and motor skills, the preferential induction of synthesis of NR1 And NR2A subunits by dextromethadone (as seen in Example 2 for ARPE-19 cells—and likely to be differential when a different cell line is tested) may favorably impact on CNS maturation (e.g., NMDAR developmental switch) and provide further disease-modifying effects for ADHD.

The spectrum encompassing normal and pathological mental development and cognitive, social, emotional, sensory and motor functions and skills, depends on the NMDAR framework and its working condition, i.e. on the physiological activity versus deregulated pathological activity, e.g., pathologically hyperactive NMDARs of said NMDAR framework. When a certain threshold of hyperactivated NMDAR channels expressed by a neuron (or even an astrocyte or an extra CNS cell), part of a circuit (or a tissue or organ) is surpassed for that cell (or those cells, because it is likely that more than one cell needs to be dysfunctional before a tissue, organ or circuit is affected), the circuit (organ or tissue) is likely to fail and a disease or disorder may manifest itself. In the case of neurons involved in certain cognitive circuits involved in academic performance, ADHD may manifest itself. In the case of hair cells in the inner ear, hearing loss may manifest itself (Example 5), et cetera.

The abnormal background electrical CNS activity and abnormal connectivity described in certain neurodevelopmental and neurodegenerative diseases and in aging brains may be secondary to abnormally functioning NMDARs and at least initially (before neuronal loss occurs) may be correctable by a drug like dextromethadone.

The results of the present inventors' Phase 2a study (rapid onset, robust and sustained disease-modifying effects) not only for the first time confirms that NMDAR hyperactivation is the culprit for MDD in a substantial subset of patients but is also potentially revealing for the pathophysiology of disorders related to MDD. For example, the present inventors may now disclose that in bipolar disorder, the manic phase is caused by pathologically hyperactive channels that allow inflow of excessive amount of calcium that initially result in some degree of function (in some milder cases—very mild hypomania—the circuit functionality in relation to individual and societal well-being may be “improved” by hypomania, possibly caused by a very slight increase Ca²⁺ influx beyond physiological levels).

However, either because of increasing presynaptic release of glutamate (experience driven release), or impaired re-uptake by astrocytes, or actions of PAMs or agonists, or even because of a post-synaptic change in cellular expression of NMDAR absolute number or relative subtypes, the “excessive” Ca²⁺ influx can increase beyond a certain limit, leading now to cellular dysfunction (altered LTP signaling) and circuitry disruption manifesting as a dysfunctional maniac episode. As the excessive Ca²⁺ influx progresses further, and cell functions, including the LTP machinery (transcription, synthesis, assembly, transportation of synaptic proteins) become progressively impaired, the manic episode, in the case of bipolar disorder, is then followed by the depressive phase of the bipolar disorder (MDE). The cellular dysfunction caused by excessive Ca²⁺ influx may further progress to apoptosis and cell death, explaining the neuroimaging and post-mortem findings of brain atrophy in patients with MDD and in patients with bipolar disorder. A drug like dextromethadone may prevent excessive Ca²⁺ influx, dysfunctional maniac and depressive phases, and neuronal death, modifying the course of the disorder.

Another example of a related disorder potentially improved by dextromethadone is PTSD. In this disorder, which shares several phenotypic features with MDD, the culprit may be an event-driven activation of NMDARs resulting in excessive Ca²⁺ influx in select neurons part of an emotional circuit. Another example of related disorders is represented by Generalized Anxiety Disorder (GAD) and Social Anxiety Disorder (SAD): in these related disorders, as in all MDD related disorders listed, the therapeutic target in patients (subjects with a predisposed NMDAR framework) is likely to be an event-driven (with or without a PAM or agonist) excess Ca²⁺ influx in select neurons part of an emotional circuit.

The same mechanism that has been indicated by the present inventors' clinical results for MDD and other studies, excessive Ca²⁺ influx across a pathologically hyperactive NMDAR, is likely to be therapeutic for MDD related neuropsychiatric disorders, including Persistent Depressive Disorder, Disruptive Mood Dysregulation Disorder, Premenstrual Dysphoric Disorder, Postpartum Depression Disorder, Bipolar Disorder, Hypomania and Mania disorder, Generalized Anxiety Disorder, Social Anxiety Disorder, Somatic Symptom Disorder, Bereavement Depressive Disorder, Adjustment Depressive Disorder, Post-traumatic Stress Disorder, Obsessive Compulsive Disorder, Chronic Pain Disorder, and Substance Use Disorder.

Yet another potential pathologic mechanism is represented by a primary dysfunction of astrocytes. Astrocytes exert a very important role in maintaining extracellular glutamate concentrations very low (low nM range), thus preventing excessive opening of NMDAR and excitotoxicity.

Astrocytes take in any extracellular glutamate released by presynaptic neuron, convert glutamate to glutamine via the glutamine synthetase pathway and release glutamine into the extracellular space where glutamine is taken into neurons converted into glutamate and stored for future uses including future release, at the time of transduction and transmission of stimuli from one cell to another. If astrocytes are dysfunctional for any reason (including because of excessive activation of astrocytic NMDARs and excessive Ca²⁺ entry into astrocytes, e.g., caused by quinolinic acid), this important function (part of the glutamate-glutamine cycle) could be impaired and excessive glutamate can accumulate in the extracellular space causing excitotoxicity and neuronal dysfunction and further astrocytic dysfunction in a self-maintaining vicious cycle. When NMDARs expressed by the membrane of astrocytes are hyperactivated (pathologically hyperactive, for example from a PAM or an agonist) excessive Ca²⁺ enters into the astrocytes and the glutamate-glutamine cycle may be impaired by astrocytic NMDAR dysfunction.

Dextromethadone, acting as an NMDAR channel blocker, may not only preserve neurons from excitotoxicity but may also restore astrocytic function by blocking their hyperactive NMDARs. Astrocytes are thus returned to their physiological function and are once again able to lower extracellular glutamate at physiologic low nanomolar levels within m-seconds from glutamate presynaptic release (the concentration of glutamate in the synaptic cleft after presynaptic release reaches 1 mM). Excitotoxicity is therefore prevented by excitatory amino acid transporter (EAAT) and functional astrocytes under physiological circumstances. Of note astrocytes are integral part of the blood brain barrier and their extensions make contact with the CNS capillaries. Astrocytic disfunction from NMDAR hyperactivity may thus disrupt the BBB with pathological consequences on CNS cells and circuits. This astrocytic hypothesis offers additional potential mechanisms for the effectiveness of dextromethadone for MDD in the absence of side effects.

When a certain percentage (e.g., >30%) of NMDARs of one or more given subtypes expressed on the membrane of a given neuron become hyperactivated (allowing excessive Ca²⁺ influx), the neuron will stop working efficiently, e.g., the neuron will slow down the production of BDNF and will slow down its constant production of new channels (e.g., transcription, synthesis and assembly of NMDAR, AMPA, Kainate subunits) and/or the neuron will stop communicating efficiently with other neurons. Neurons need to constantly maintain physiological synthesis, assembly transport, membrane expression of synaptic proteins and synthesis transport and release of growth factors that are necessary to modulate synaptic strength. These neuronal functions are regulated by NMDAR patterns of calcium influx and if the pattern is altered (NMDAR hyperactivity) these neuronal functions are compromised.

To further clarify, aside from the regulation of synaptic protein synthesis and assembly, the tightly regulated synthesis and transport of neurotransmitters is also controlled by the same patterns of calcium currents across the cell membrane. When a certain percentage (e.g., over 30%) of ion channels expressed by select neurons are hyperactivated, the neuron becomes inefficient (excessive Ca²⁺ influx). When a sufficient number of neurons that are part of the same circuit are inefficient the flow of information and the circuit itself become inefficient, disrupting essential inter-neuronal communication pathways (circuits). When a certain brain circuit is impaired to a sufficient degree a cluster of symptoms will emerge (neuropsychiatric condition, disorder, disease). If the pathophysiologic mechanisms described above (pathologically hyperactive NMDAR channels) happen in certain hypothalamic neurons (altered blood pressure and metabolic disorders), hepatocytes (NAFLD, NASH), in Langerhans cells (impaired glucose tolerance and diabetes) urogenital tract (infertility, premature ovarian failure, bladder disorders, including overactive bladder disorder, renal insufficiency) or lymphocytes and macrophages (inflammatory conditions, immune system disorders, cancer) or in vascular and cardiac cells (CAD, heart failure, arrhythmias) or in platelets (DIC), then corresponding disorders or diseases will emerge, including but not limited to CNS diseases and disorders and including but not limited to diseases and disorders listed above.

The cluster of symptoms and signs caused by the impairment of a neuronal circuit may represent a neuropsychiatric disorder, as defined by DSM 5, e.g., MDD, MDD related disorders and other neuropsychiatric disorders disclosed in this application. Dextromethadone is therefore not merely a symptomatic treatment but a drug that modulates replacement of defective ion channels in neurons and restores functionality in neurons (and other cells) and restores functionality of neuronal circuits (and other circuits, tissues, and organs).

The therapeutic actions of dextromethadone in the absence of clinically meaningful side effects are the result of selective targeting of hyperactivated NMDARs and modulation of their function, i.e., blocking the pathologically open channels of hyperactivated NMDARs, and return to physiological induction of synthesis, assembly, transport and expression of new functional NMDARs, and thus restoring neuronal function and restoring neuronal circuits and correcting and preventing disorders and diseases. These actions by dextromethadone are all the more remarkable because they occur in the absence of clinically meaningful side effects, underscoring the selective targeting of hyperactivated, pathologically open NMDARs. The inventors disclose that dextromethadone induces the synthesis of proteins that form NMDARs (Example 2) and thus potentially restores neuronal function and connectivity essential for functional neuronal circuits. While NMDAR dysfunction is the culprit of a multiplicity of diseases and disorders primarily in the nervous system but also extra nervous system, there is a scarcity of drugs that can safely and effectively modulate the NMDAR receptor.

Dextromethadone and the other drugs with a similar postulated mechanism of action can now also be considered potential disease-modifying treatments for a multiplicity of diseases and disorders. The safety and efficacy of dextromethadone and its derivatives and other enantiomers of opioid drugs that do not produce clinically meaningful opioid effects but may have shepherding effects (see Example 10) is linked to their ability to selectively target hyperactivated, pathologically hyperactive ion channels, while sparing physiologically working channels. Dextromethadone's receptor binding kinetics, with favorable “on” and “off” intra-channel binding and favorable “trapping” characteristics (Example 6), compares favorably for example to ketamine a drug that may have too rapid “onset” for safe use in routine outpatient setting, where it can be administered only under health provider supervision.

Additionally, when the drugs disclosed by the applicants are administered early in the course of the disease caused by NMDAR dysfunction, before there is severe or even irreversible neuronal damage, they will potentially prevent disease manifestations and disease progression. Due to the constant and complex interaction between G+E (e.g., genetic predisposition to ion channelopathies, including NMDAR channelopathies and environmental insults to channels, including chemical and physical toxins and psychological trauma), cells are constantly working towards the maintenance of homeostasis characterized by a certain percentage of tonically open ion channels, including NMDARs, that direct the cell's physiologic functions, including synthesis and assembly of proteins. In particular neurons are constantly changing their connections based on environmental stimuli (e.g., stimuli that reach neurons from body organs or external environment). In order to be able to rapidly express the membrane receptors that allow plasticity, the building blocks, e.g., synaptic proteins, must be ready to be assembled and expressed at all times. A precise amount of tonic Ca²⁺ influx (modulated by the NMDAR with incomplete block at resting membrane potential (NMDAR with GluN2C, GluN2D and possibly GluN3 subunits) is likely to instruct on synthesis and assembly of synaptic proteins that are ready in the post-synaptic density so when a stimulus is transmitted via glutamate release by the presynaptic neuron the postsynaptic neuron can react timely and build memory (rapid assembly and expression of membrane receptors and other synaptic strengthening actions, e.g., release of BDNF, release of adhesion proteins et cetera). When tonic Ca²⁺ influx is excessive the preparatory work is not productive (there is impairment in synaptic protein production) and real time constantly incoming stimuli are not effectively translated into memory. Dextromethadone may downregulate excessive tonic Ca²⁺ influx and restore neural plasticity and potentially cure MDD.

Dextromethadone and potentially other drugs, such as other isomers of opioids and derivatives of dextromethadone, maintain and restore ion channels, including NMDAR channel homeostasis, and therefore, aside from representing a potential disease-modifying treatment for all of these diseases and disorders, when administered very early in the course of NMDAR dysfunction, before the NMDAR dysfunction reaches the threshold that would result in functional impairment of the neuron, may be effective preventive treatments. These primary and secondary preventive actions for a multiplicity of diseases and disorders may be exerted at lower than expected doses, or even with the use of intermittent dosages as disclosed in this application.

And so, the inventors now disclose that dextromethadone has robust, rapid and sustained and statistically significant efficacy with a large effect size for MDD and potentially for TRD. The experimental clinical trial is detailed in this Example 3. This unexpected result signals a potential efficacy ceiling effect at 25-50 mg, similarly to the ceiling for ketamine at 0.5-1 mg/Kg [Fava M, Freeman M P, Flynn M, et al. Double-blind, placebo-controlled, dose-ranging trial of intravenous ketamine as adjunctive therapy in treatment-resistant depression (TRD) Mol Psychiatry. 2018]. In addition, there is a signal towards a “pulse” weekly treatment as opposed to a continuous treatment: at the end of the second week for the 25 mg group there is a signal towards a need to resume treatment. This PD signal (25 mg group: MADRAS—17.4 day 7 versus MADRAS—16.8 day 14), taken together with the PK results (Example 3 MDD, PK, 25 mg group: by day 14 the plasma levels of dextromethadone are in the very low ng/ml range) and complemented with the literature data for the NMDAR channel blocker ketamine, with evidence for efficacy with pulse treatment rather than continuous treatment, indicate that a similar posology (weekly pulse therapy as opposed to continuous therapy), may also be indicated for dextromethadone.

Further, the inventors disclose for the first time that dextromethadone does not only block hyperactive NMDARs but also potentially induces the expression of new NMDARs and particularly 2A subtypes in ARPE-19 cells, potentially explaining the unexpected long-lasting clinical effects seen in the MDD human study.

The inventors also disclose that dextromethadone decreases NAFLD and modulates inflammatory markers in rats on “western diet” (as shown in Example 11).

The inventors also disclose that dextromethadone is also effective when certain inflammatory biomarkers are altered and thus dextromethadone potentially modulates inflammatory states and inflammatory states associated with neuro-psychiatric disorders.

The inventors show for the first time that oral dextromethadone administration daily for one week has rapid, robust, sustained and statistically significant efficacy with a large effect size for patients with a diagnosis of MDD and/or TRD. In order to ensure a proper diagnosis of MDD the inventors utilized SAFER, a validated tool to screen patients and improve the probability of a proper diagnosis of MDD. SAFER improves the probabilities that patients enrolled in clinical studies will have been diagnosed correctly and thus can be adequately assessed for trial outcomes, thus minimizing the risk that factors unrelated to treatment will determine the patients' course of illness and thereby confound study results (Desseilles et al., 2013). This double-blind, placebo controlled, prospective, randomized clinical trial reinforced by SAFER shows that dextromethadone, within the first week of treatment, can induce remission of disease (MADRS<10) in over 30% of patients with MDD diagnosed with the aid of SAFER, compared to a remission rate of 5% in patients randomized to placebo (see FIG. 25). Additionally, the remission persisted for at least one week after discontinuation of treatment, despite a drastic reduction in plasma levels of dextromethadone to levels not expected to exert clinically meaningful pharmacologic actions (single digit ng/ml range). The improvements induced by dextromethadone are likely to have lasted well beyond the 14th day for some of these patients. The MADRS rating scale measures not only depressed mood but also an array of other symptoms, which taken together and integrated with other diagnostic parameters, including SAFER, can diagnose the severity of MDD. The array of symptoms measured in the different scales used in this trial can also contribute to the diagnosis of other neuropsychiatric disorders defined by the DMS5 and listed in the claims below. This persistence of disease remission after discontinuation of treatment signals a disease-modifying mechanism of action for dextromethadone (e.g., modulation of neuroplasticity), rather than the improvement of isolated psychiatric symptoms.

### Example 4

A. Overview

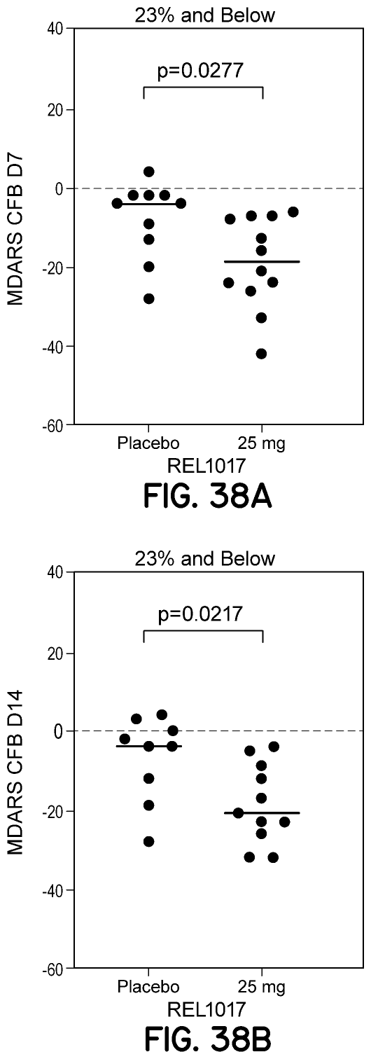

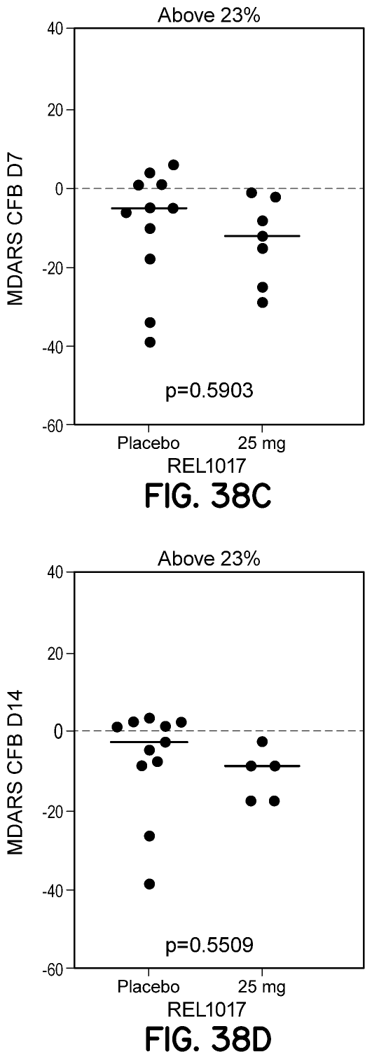

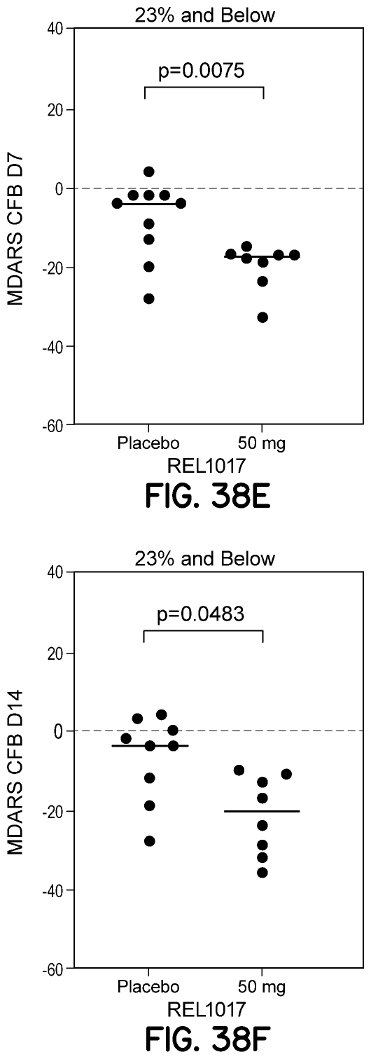

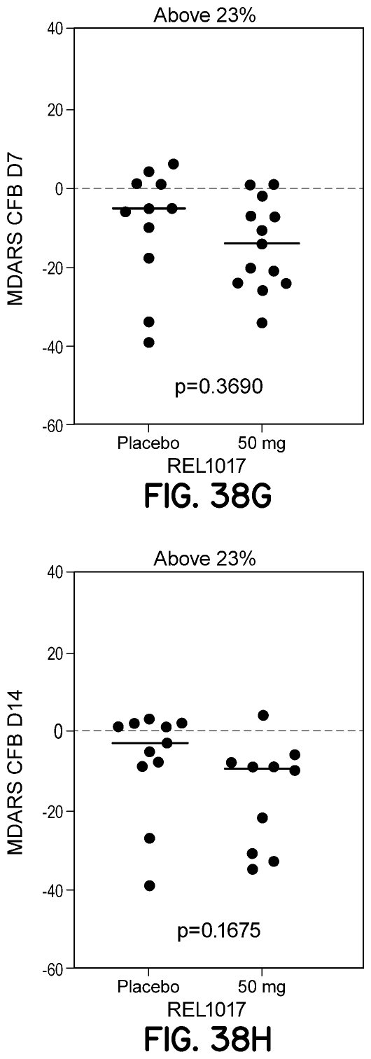

The inventors performed a sub-analysis (detailed below, and in Table 35 below and FIGS. 38A-D, and 38E-H) of the data from the Phase 2 study described in Example 3. This sub-analysis demonstrated that dextromethadone (REL-1017) is more effective in patients treated earlier in the course of MDD compared to patients treated later in the course of MDD. This unexpected finding (never demonstrated before for any other antidepressant drug) signals that dextromethadone is a potentially disease-modifying treatment for MDD and related disorders and potentially other neuropsychiatric disorders. While symptomatic treatments are equally effective early and late in the MDD, a specific disease-modifying treatment will have better results when administered early in the course of the disorder, before permanent damage occurs. Given the prevalence of MDD in the general population and its heavy toll on patients and society, the introduction of the first well-tolerated potentially disease-modifying treatment within the current landscape of symptomatic treatments may revolutionize the neuropharmacology field.

And so, in this study, the present inventors examined the effect of dextromethadone on the percentage of life years from the start of MDD. In that regard, chronicity of depression has not proven to be a reliable predictor of response to standard antidepressant treatments (SATs) or response to placebo (Papakostas G I, Fava M. Predictors, moderators, and mediators (correlates) of treatment outcome in major depressive disorder. Dialogues Clin Neurosci. 2008; 10(4):439-451).

In contrast with SATs and atypical antipsychotics, dextromethadone may be more effective in MDD patients with a lower percentage of life-years from the start of MDD.

B. Methods

The present inventors reviewed historical data on the start date of MDD for the randomized population of the Phase 2a study of dextromethadone as adjunctive treatment in patients with MDD who failed 1-3 adequate SATs (described above in Example 3). The percentage of life-years spent from the start of depression was calculated by computing the number of years from the start date of MDD divided by age and multiplied by 100. Patients were then divided below and above the median value. The MADRS CFB of patients in the treatment group were compared to the MADRS CFB in the placebo group by Student's t test for unpaired data with comparisons indicated on each of FIGS. 38A-D and 38E-H. The analysis was performed by means of the software GraphPad Prism ver. 8.0.

C. Results

The median percentage of life years from the start date of MDD for the 62 randomized patients was 23%. In the dextromethadone Phase 2 study, at both tested doses, 25 mg and 50 mg, patients below the median percentage of life-years from the start of MDD were significantly more responsive to dextromethadone active treatment compared to the placebo group. In the same dextromethadone Phase 2 study, at both tested doses (25 mg and 50 mg) the response to active treatment compared to the placebo group was not statistically significant for patients above the median percentage of life-years from the start of MDD. (see Table 35; FIGS. 38A-H).

Referring to FIGS. 38A-D: Patients treated with 25 mg of dextromethadone who were below the median percentage of life years from the start date of MDD (below 23%) showed a significant improvement of MADRS mean scores at day 7 (p=0.0277) (FIG. 38A) and at day 14 (p=0.0217) (FIG. 38B) when compared with placebo patients who were also below the median percentage of life years from the start date of MDD (below 23%). The treatment effects were not statistically significant when the same analyses were performed in patients above the median percentage of life-years from the start of MDD (p>0.5 at all recorded time points) (FIGS. 38C and 38D).

Referring to FIGS. 38E-H: Patients treated with 50 mg of dextromethadone who were below the median percentage of life years from the start date of MDD (below 23%) showed a significant improvement of MADRS mean scores at day 7 (p=0.0075) (FIG. 38E) and at day 14 (p=0.0483) (FIG. 38F) when compared with placebo patients who were also below the median percentage of life years from the start date of MDD (below 23%). The treatment effects were not statistically significant when the same analyses were performed in patients above the median percentage of life-years from the start of MDD (p>0.1 at all recorded time points) (FIGS. 38G and 38F).

D. Conclusion

In this sub-analysis of data from a Phase 2 trial, dextromethadone at a daily dose of 25 and 50 mg was significantly effective in reducing MADRS scores compared to placebo in patients below the median (23%) for percentage of life-years from the start of MDD. When the same data were analyzed for patients above the median (23%) for percentage of life-years from the start of MDD results did not reach statistical significance at either of the tested doses. This differential therapeutic effect related to chronicity of MDD has not been previously reported for monoaminergic drugs nor for atypical antidepressants and has not been described for ketamine or esketamine. Disease-modifying treatments typically achieve the best results when administered early on in the course of the disease, e.g., antibiotics for bacterial infections, thyroid hormone for hypothyroidism. Symptomatic treatments, e.g., SSRI for depression and benzodiazepines for anxiety, will produce a symptomatic effect at any time during the course of the disease. The statistically significant therapeutic effect of dextromethadone when administered earlier compared to later in the course of MDD confirms its disease-modifying effects anticipated by Example 3. Furthermore, this finding may help selecting patients with a higher likelihood of response to dextromethadone therapy and other therapies, including psychotherapy.

Finally, when a clinical variable has a large effect on treatment response, stratification may prevent type I error and improve power for small trials (<400 patients), especially when an interim analysis is planned [Kerman et al., 1999; Broglio K. Randomization in Clinical Trials: Permuted Blocks and Stratification. JAMA. 2018; 319(21):2223-2224; Saint-Mont U. Randomization Does Not Help Much, Comparability Does. PLoS One. 2015; 10(7):e0132102. Published 2015 Jul. 20]. In the context of the planned clinical trials, stratification of patients above or below the median for years of life from the start of MDD may not only improve comparability between groups but may also signal treatment with potentially disease-modifying effects. Furthermore, in the context of MDD clinical trials, stratification of patients above or below the median for years of life from the start of MDD may signal treatments with potentially disease-modifying effects. As a result of these findings by the present inventors, dextromethadone and potentially other safe and well tolerated oral NMDAR channel blockers could rapidly become a first line treatment for MDD and related disorders.

TABLE 35
CFB = change from baseline

| | % life-years from | MADRS | MADRS |
|---|---|---|---|
| | start of MDD: | mean CFB | mean CFB |
| Treatment | 23% = median | Day 7 | Day 14 |
| 25 mg | | | |
| N = 12 | 23% and below | −18.91 | −18.54 |
| | Mean: 12.93% | | |
| N = 7 | Above 23% | −13.14 | −11.4 |
| | Mean: 42% | | |
| 50 mg | | | |
| N = 8 | 23% and below | −20 | −21.5 |
| | Mean: 12.89% | | |
| N = 13 | Above 23% | −14.46 | −15.9 |
| | Mean: 47% | | |
| 25 + 50 mg | | | |
| N = 20 | 23% and below | −19.35 | −19.78 |
| | Mean:12.91 | | |
| N = 20 | Above 23% | −14 | −14.4 |
| | Mean: 46% | | |
| Placebo | | | |
| N = 11 | 23% and below | −8 | −6.8 |
| | Mean: 8.25% | | |
| N = 11 | Above 23% | −9.5 | −7.5 |
| | Mean: 49% | | |

### Example 5

Overview: This Example 5 demonstrates that gentamicin quinolinic acid is effective for modulating NMDAR channels pathologically activated by endogenous substances (e.g., inflammatory intermediates) and exogenous substances (e.g., drugs and other toxins).

Part I: Positive Allosteric Modulators (PAMs) at the NMDAR

A. Background

The ototoxic and nephrotoxic drug gentamicin acts as a Positive Allosteric Modulator (PAM) of the NMDAR in stable cell lines expressing diheteromeric recombinant human NMDARs, containing GluN1 plus one amongst GluN2A, GluN2B, GluN2C or GluN2D subunit.

Dextromethadone counteracts the toxic effect of gentamicin (and other PAMs of NMDARs) by reducing Ca²⁺ influx via hyperactivated NMDARs. In particular dextromethadone counteracts excessive Ca²⁺ influx via NMDARs hyperactivated by the PAM nephrotoxic and ototoxic drug gentamicin.

Select disorders and diseases may be caused by PAMs and or agonists of NMDARs, e.g., disorders and diseases may be caused by toxin-induced hyper-activation of select NMDARs in select cells part of select tissues or circuits via allosteric modulation and or via agonist actions ant the NMDA site of NMDARs.

Sensory-neural hearing impairment may be caused by impairment of spiral ganglion neurons (SGNs). SGNs are bipolar neurons that transmit auditory information from the ear to the brain. Physiologically functioning SGNs are indispensable for the preservation of normal hearing and their function and survival depend on genetic and environmental interactions.

NMDA antagonism with MK-801 ameliorated renal damage after exposure to short-term gentamicin in experimental conditions (Leung J C, Marphis T, Craver R D, Silverstein D M. Altered NMDA receptor expression in renal toxicity: Protection with a receptor antagonist. Kidney Int. 2004; 66(1):167-176).

And, NMDARs are expressed not only in the CNS but also peripherally (Du et al., 2016).

Nephrotoxic and or ototoxic medications, such as gentamicin, may result in sensorineural hearing impairment and nephrotoxicity by acting as PAMs of NMDARs expressed by SGNs and renal cells. PAMs may cause excessive Ca²⁺ influx in cells and excitotoxicity (epigenetic dysregulation of Cam-CaMKII, RAS, and PI3K signaling). Dextromethadone, a novel potentially effective drug, shown to have NMDAR uncompetitive channel blocker actions (Example 1), shown to result in rapid, robust and sustained clinical effects in patients with MDD (Example 3), and shown to exert neural plasticity effects (Example 2), could potentially prevent ototoxic and nephrotoxic effects when co-administered with gentamicin or other PAMs affecting the same cells or other cells.

In addition, by the same mechanism, downregulation of excessive Ca²⁺ influx in select cells part of select tissues or circuits, hyperactivated by excessive stimulation with NMDAR agonists (e.g., glutamate or glycine or the glutamate agonist quinolinic acid) and or by a multiplicity of PAMs, dextromethadone may prevent, treat or diagnose disorders triggered, maintained or worsened by excessive Ca²⁺ influx, including select cases of MDD caused by PAMs and or NMDA agonists. The roles of quinolinic acid as a glutamate agonist in triggering, worsening or maintaining MDD and as a neurotoxic agent by other mechanisms are well known [Guillemin et al., 2012; Schwarcz R, Bruno J P, Muchowski P J, Wu H Q. Kynurenines in the mammalian brain: when physiology meets pathology. Nat Rev Neurosci. 2012; 13(7):465-477; Lovelace M D, Varney B, Sundaram G, et al. Recent evidence for an expanded role of the kynurenine pathway of tryptophan metabolism in neurological diseases. Neuropharmacology. 2017; 112(Pt B): 373-388].

B. Framework of Study

A FLIPR calcium assay was used to profile gentamicin using stable cell lines expressing diheteromeric recombinant human NMDARs, containing GluN1 plus one amongst GluN2A, GluN2B, GluN2C or GluN2D subunit. 10 μM gentamicin effect was evaluated on three different L-glutamate concentrations: 0.04, 0.2 and 10 μM, using the 4 NMDAR cell lines. And 10 μM dextromethadone addition was evaluated on the three L-glutamate concentrations, with and without 10 μM gentamicin.

C. Results

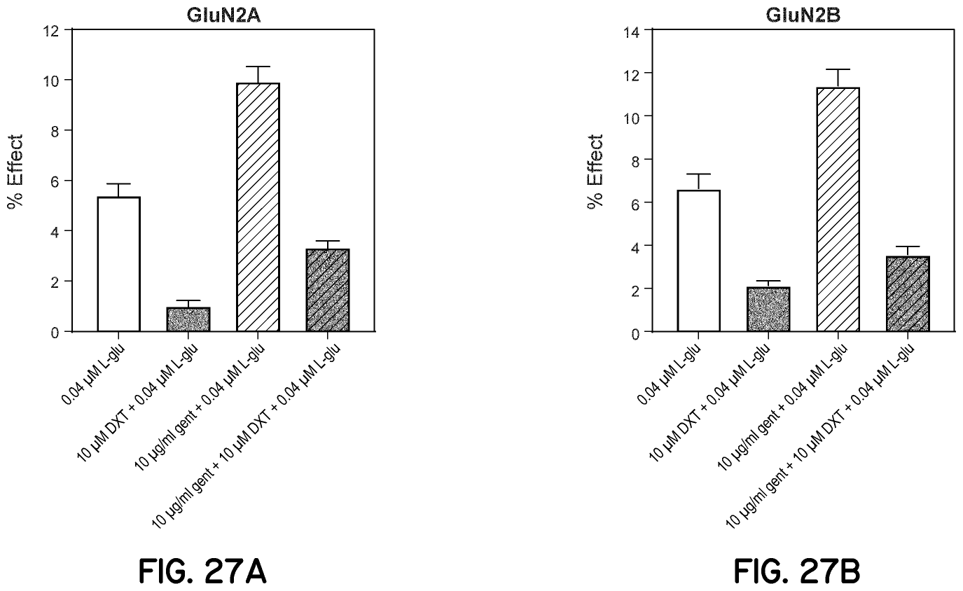

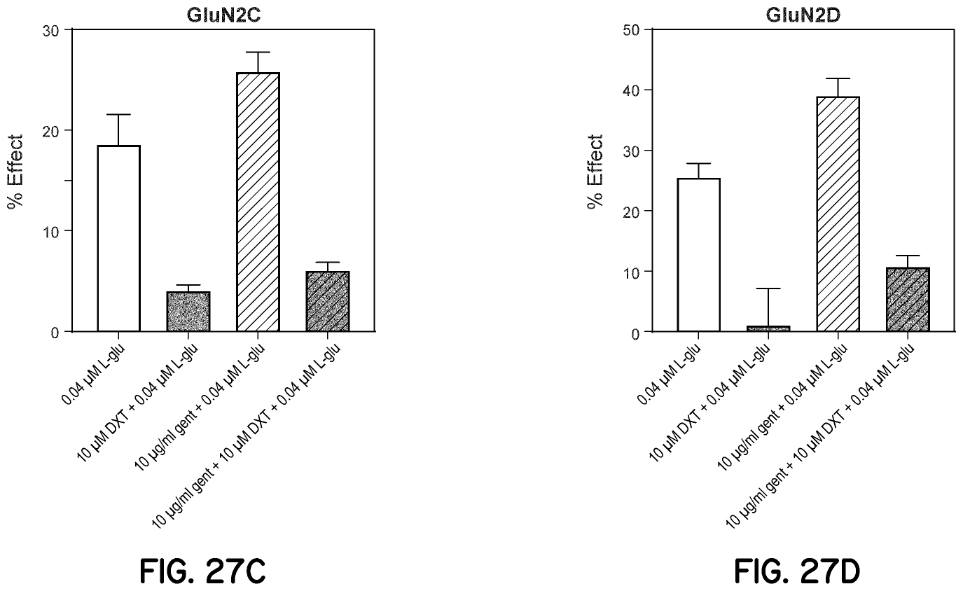

The effect of 10 μM gentamicin on 0.04 μM L-glutamate (data are mean±SEM, n=30 for each group) is shown in FIGS. 27A-D for the different cell lines. As can be seen in the figures, very low concentration glutamate (0.04 μM) induced calcium entry in all cell lines [GluN2D>GluN2C>GluN2B>GluN2A]. Further, 10 μM gentamicin significantly increased calcium entry induced by 0.04 μM L-glutamate with P<0.0001 for GluN2A and GluN2B cell lines, but with only P<0.05 for GluN2C and GluN2D cell lines. And, 10 μM dextromethadone significantly reduced calcium entry elicited by 0.04 μM L-glutamate in presence and in absence of 10 μM gentamicin, with P<0.0001 for all cell lines.

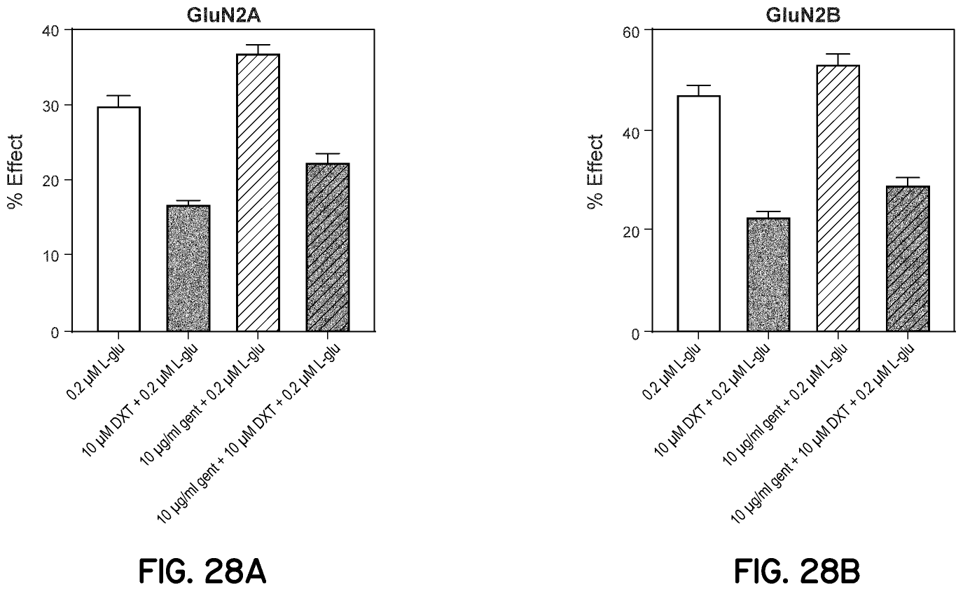

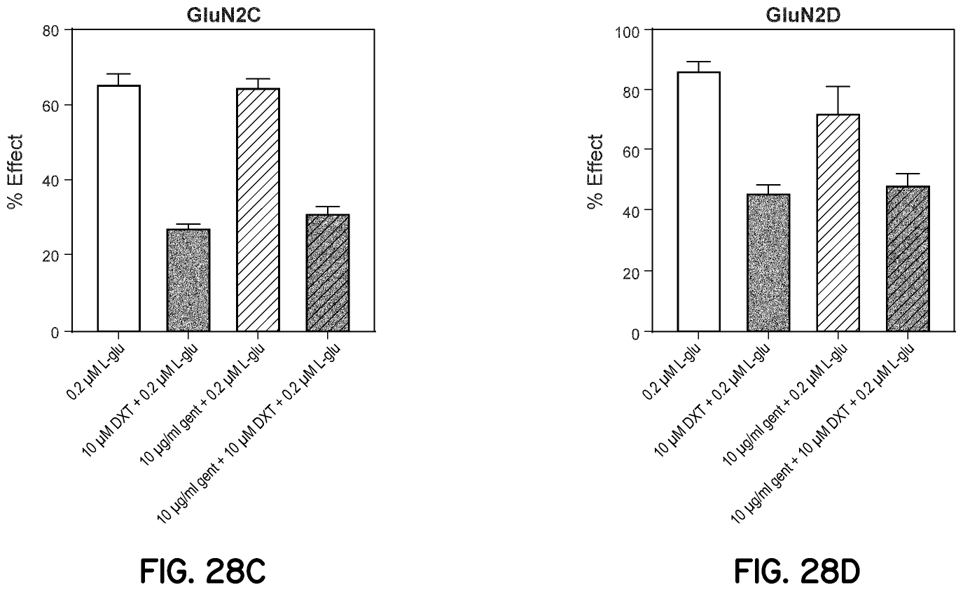

Next, the effect of 10 μM gentamicin on 0.2 μM L-glutamate (data are mean±SEM, n=30 for each group) is shown in FIGS. 28A-D for the different cell lines: As can be seen in the figures, low concentrations of glutamate 0.2 μM induced calcium entry in cell all lines [GluN2D>GluN2C>GluN2B>GluN2A]. Further, 10 μM gentamicin significantly increased calcium entry induced by 0.2 μM L-glutamate only for GluN2A (P<0.0001) and GluN2B (P<0.05) cell lines but decreased calcium entry in GluN2D cell line (P<X,X), thus acting as a Negative Allosteric Modulator (NAM) for this line. And, 10 μM dextromethadone significantly reduced calcium entry elicited by 0.2 μM L-glutamate in presence and absence of 10 μM gentamicin, with P<0.0001 for GluN2A, GluN2B, GluN2C cell lines, but with P<0.005 in presence of gentamicin for GluN2D cell line.

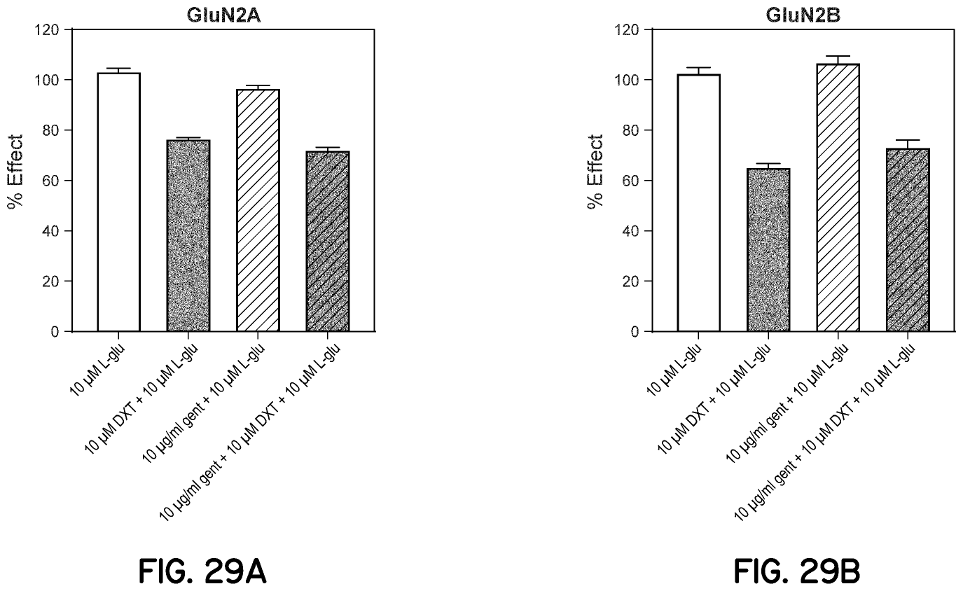

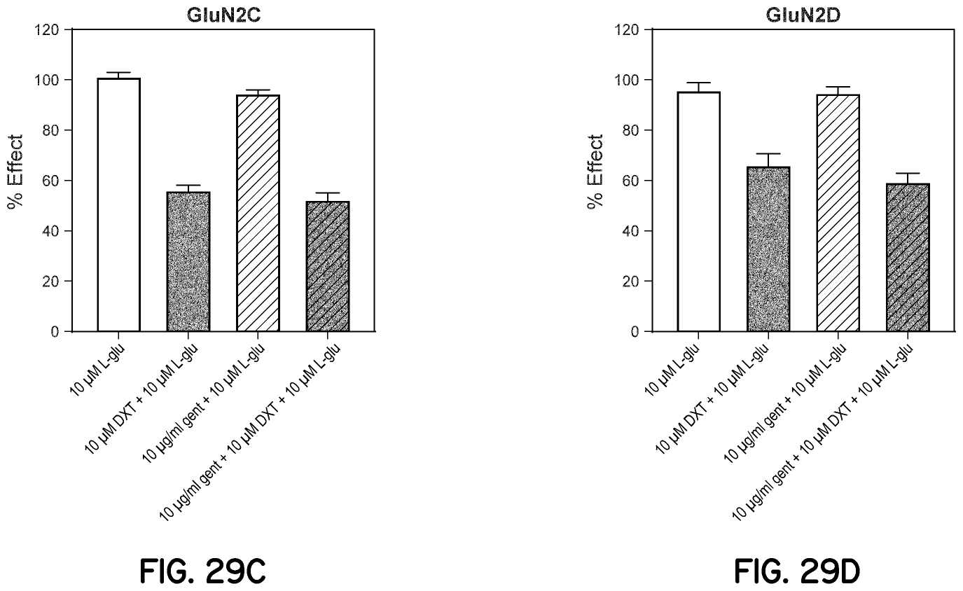

Finally, the effect of 10 μM gentamicin on 10 μM L-glutamate (data are mean±SEM, n=30 for group without dextromethadone, n=20 for remaining groups) is shown in FIGS. 29A-D for the different cell lines: As can be seen in the Figures, glutamate 10 μM maximally induced Ca²⁺ influx in all cell lines except for Glu2D. Further, 10 μM gentamicin did not modify calcium entry induced by 10 μM L-glutamate for GluN2B and GluN2D cell line, while it significantly decreased calcium entry in GluN2A (P<0.0001) and GluN2C (P<0.05) cell lines. Thus, in contrast with its effects in the presence of very low glutamate concentration, when glutamate exerts its maximal Ca²⁺ influx inducing effects, gentamicin acted as a NAM, although only in two of the 4 tested lines (Glu2A and Glu2C). And, 10 μM dextromethadone once again significantly reduced calcium entry elicited by 10 μM L-glutamate in presence and absence of 10 μM gentamicin, with P<0.0001 for all cell lines.

D. Discussion

As noted above, low concentrations of glutamate (0.04 μM and 0.02 μM) induced calcium entry in all cell lines GluN2D>GluN2C>GluN2B>GluN2A. Glutamate 10 μM maximally induced Ca²⁺ entry in all cell lines. 10 μM gentamicin significantly increased calcium entry induced by 0.04 μM L-glutamate with P<0.0001 for GluN2A and GluN2B cell lines, and with P<0.05 for GluN2C and GluN2D cell lines. And, 10 μM dextromethadone significantly reduced calcium entry elicited by 0.04 μM L-glutamate in presence and in absence of 10 μM gentamicin, with P<0.0001 for all cell lines.

10 μg/ml gentamicin effect on NMDARs appeared to be dependent on L-glutamate concentration: Positive modulation was detected in all tested cell lines at 0.04 μM L-glutamate, with P<0.0001 for GluN2A and GluN2B cell lines, and with P<0.05 for GluN2C and GluN2D cell lines; positive modulation was detected only in GluN2A (P<0.0001) and GluN2B (P<0.05) cell lines at 0.2 μM L-glutamate and negative modulation was detected for the Glu2D line. Positive modulation was absent in all tested cell line at 10 μM L-glutamate but negative modulation was detected for Glu2A and Glu2C.

10 μM dextromethadone was able to lower intracellular calcium level induced by 0.04, 02 or 10 μM L-glutamate, with or without 10 μM gentamicin, in all tested cell lines.

The effectiveness of dextromethadone for the treatment of diseases and disorders caused by excessive Ca²⁺ influx may be determined by its ability to selectively block NMDARs that remain excessively open, independently from the concentration of glutamate or the presence of PAMs or NAMs, as shown by the results above and by Example 1. These results for a drug like gentamicin, with NMDAR mediated ototoxic and nephrotoxic effects, signal that the primary cause for diseases and disorders triggered or maintained by excessive Ca²⁺ influx may be caused by prolonged (tonic and pathologic) activation of the NMDAR. The tonic activation that potentially induces excitotoxicity may be caused by presynaptic glutamate release even at very low concentrations with or the presence of PAMs at post-synaptic NMDARs or by defective glutamate clearance by EAAT in the synaptic cleft.

E. Conclusion

Gentamicin positive modulation of NMDAR activity (Ca²⁺ influx) appeared to be dependent on both L-glutamate concentrations and the NMDAR subtype (differential modulation with different concentrations of glutamate and differential modulation with differential NMDAR subtype).

The effect of gentamicin as a modulator of the NMDAR appears to be dependent on the differential activation of NMDARs exerted by different concentrations of glutamate.

Interestingly, very low and low concentrations of glutamate (0.04 and 0.2 microM) induction of Ca²⁺ entry followed known NMDAR channel subtype kinetics [GluN2D>GluN2C>GluN2B>GluN2A]. Glutamate 10 μM maximally induced Ca²⁺ influx in all cell lines except for Glu2D.

Gentamicin 10 μg/ml showed positive modulation effect of intracellular calcium levels at very low L-glutamate concentrations, such as 0.04. This very low glutamate concentration may be present tonically at the synapse of hair cells with nerve cells forming the auditory pathways and pathological increases in glutamate or allosteric NMDAR enhancement may lead to hair cell loss (Moser T, Starr A. Auditory neuropathy—neural and synaptic mechanisms. Nat Rev Neurol. 2016; 12(3):135-149; Sheets L. Excessive activation of ionotropic glutamate receptors induces apoptotic hair-cell death independent of afferent and efferent innervation. Sci Rep. 2017; 7:41102. Published 2017 Jan. 23).

10 μM dextromethadone was able to lower intracellular calcium level induced 0.04, 02, 10 μM L-glutamate in all tested cell lines with or without gentamicin.

The demonstration that gentamicin increases Ca²⁺ via NMDARs at very low and low L-glutamate concentration supports PAM of NMDARs in SGN (renal cells) as the mechanism for gentamicin ototoxicity (nephrotoxicity). Hyper-activation of NMDARs by toxins (PAMs) selective for certain cells is thus a possible cause for excessive Ca²⁺ influx in triggering and or maintaining a multiplicity of disorders and diseases. For example, in some of the patients presented in Example 3, MDD may have been caused by PAMs and or agonists at the NMDA site or glycine site of the NMDAR. In the patients with MDD presented in Example 3, the downregulation of Ca²⁺ influx in select neurons caused a resolution of the disorder. While the precise individual cause for excessive Ca²⁺ for these patients is unknown, potential causes are: excessive presynaptic glutamate release, PAMs at the postsynaptic domain, agonists at the NMDAR, defective glutamate clearance by EAATs from the synaptic cleft or any combination of the above causes.

Subsets of disorders and diseases, especially neuropsychiatric diseases and disorders, but also ophthalmological, otological, metabolic, cardiovascular, respiratory, renal, liver, pancreas, lung, bone, disorders of coagulation, can be caused by abnormal patterns of Ca²⁺ influx via NMDARs activated by PAMs (e.g., gentamicin or other toxins) and or agonists (e.g., quinolinic acid or other toxins) leading to excessive Ca²⁺ influx with various levels of excitotoxicity, cell impairment and even cell death. In particular, the present inventors' findings in Example 3 strongly suggests that, at least for a subset of patients with MDD, the cause for the disorder was excessive Ca²⁺ influx in select cells, part of select circuits. In turn, this strong signal for excessive Ca²⁺ influx as the cause of MDD, and the findings in Examples 1-11 suggest that a multiplicity of CNS and extra-CNS disorders are potentially caused by excessive Ca²⁺ influx in select cells part of select tissues and or circuits and that this excessive Ca²⁺ influx via hyperactivated (by glutamate, other endogenous or exogenous agonists and or endogenous or exogenous PAMs) ion channels can be selectively downregulated by NMDAR blockers such as dextromethadone. A selective action of the NMDAR channel blocker on pathologically hyperactivated channels, such that exerted by dextromethadone, is crucial in order to minimize side effects.

The finding that the positive modulation of Ca²⁺ influx by the ototoxic drug gentamicin was evident at very low glutamate concentrations is noteworthy. It suggests that for certain cells there is a state of physiologically tonic low levels of Ca²⁺ influx that may be vulnerable to the effects of toxic PAMs and or agonists.

The down-regulation effect of dextromethadone on intracellular calcium level induced by glutamate 0.04, 0.2 and 10 μM L-glutamate in all tested cell lines suggests a potentially preventive or curative effect for a multiplicity of diseases and disorders caused by excessive influx of Ca²⁺ in select cells in the presence or absence of PAMs and or agonists.

The results presented in Examples 1-11 (including in this Example 5) signal disease-modifying effects of dextromethadone for diseases and disorders caused by excessive NMDAR activation by glutamate (even at very low concentrations) and or PAMs and or agonists, in select cells specific for select diseases triggered or maintained by excessive Ca²⁺ influx. The availability of a well-tolerated drug like dextromethadone with select activity for hyperactivated NMDARs will help identify, categorize, diagnose, prevent and treat diseases caused by excessive Ca²⁺ entry.

Furthermore, dextromethadone was always able to surmount the potentially toxic effects of gentamicin, signaling potentially very effective preventive and disease-modifying effects not only for hearing impairment and renal impairment caused by gentamicin and other PAMs, but for a multiplicity of diseases and disorders caused by toxic PAMs, and may help identify PAMs specific for select disorders.

Part II: Agonists and PAMS at the NMDAR

This part of Example 5 looks at dextromethadone, quinolinic acid, and gentamicin via mode of action FLIPR calcium assay using GluN1-GluN2A, -2B, -2C, and -2D cell lines.

The following is a list of abbreviations used in this Part II of Example 5.

| Abbreviation | Definition or Expanded Term |
|---|---|
| AUC | Area under the curve |
| CHO | Chinese hamster ovary |
| CRC | Concentration response curve |
| DMSO | Dimethyl sulfoxide |
| EC₅₀ | Drug concentration that gives half-maximal response |
| FLIPR | Fluorescence imaging plate reader |
| Gly | Glycine |
| GLP | Good laboratory practice |
| IC₅₀ | Half maximal inhibitory concentration for a drug |
| Log | Base 10 logarithm |
| L-glu | L-glutamate |
| MW | Molecular weight |
| NA | Not available |
| NMDA | N-methyl-D-aspartate |
| NMDAR | N-methyl-D-aspartate receptor |
| MOR | μ-opioid receptors |
| pEC₅₀ | negative log of the molar EC₅₀ value |
| LTP LTD | Long Term Potentiation Long Term Depression |
| SEM | Standard error of the mean |

A. Introduction

A FLIPR-calcium assay was used to evaluate the effect of dextromethadone, or quinolinic acid, in presence of 10 μM glycine, with or without 40 or 200 nM glutamate or 10 μM gentamicin, in four human recombinant NMDA receptor types: GluN1-GluN2A, GluN1-GluN2, GluN1-GluN2C, GluN1-GluN2D. Quinolinic acid or gentamicin CRCs were also produced, in presence of 10 μM glycine.

B. Test Items

2.1 Test Items are shown in Table 36 (below).

TABLE 36

| Name | MW | Supplier | Code | CAS |
|---|---|---|---|---|
| Dextromethadone | 345.91 | Padova | | 5653-80-5 |
| hydrochloride | | University | | (base) |
| Quinolinic acid | 167.12 | Merck Sigma- | P63204- | 89-00-9 |
| | | Aldrick | 100G | |
| Gentamicin sulfate | ~681.58 | Merck Sigma- | G1264- | 1405-41-0 |
| | | Aldrick | 250MG | |
| Glutamic acid | 187.1 | Merck Sigma- | G1626 | 142-47-2 |
| | | Aldrick | | (anhydrous) |
| Glycine | 75.07 | Merck Sigma- | G7403 | 56-40-6 |
| | | Aldrick | | |

Test items were dissolved in H₂O (gentamicin, L-glutamate, glycine), or compound buffer (quinolinic acid) at suitable concentration, and then immediately used or stored at −20° C. till use.

Stock concentrations were: 50×=50 mM for quinolinic acid; 400×=40 or 4 mg/ml for gentamicin; 400×=4 mM for L-glutamic acid and glycine; 2.000×=20 mM for dextromethadone.

C. Test System

Test items were evaluated in FLIPR for their ability to modulate, alone or in combination, calcium entry in presence of 10 M glycine, using four CHO cell lines expressing diheteromeric human NMDA receptor (NMDAR): GluN-/GluN2A-CHO, GluN1-GluN2B-CHO, GluN1-GluN2C-CHO, GluN1-GluN2D-CHO.

D. Experimental Design

The first aim of the study was to evaluate quinolinic acid or gentamicin CRC effect in the presence of 10 μM glycine. 11 concentrations of quinolinic acid were assessed: 1,000 μM, 333 μM, 111 μM, 37 μM, 12 μM, 4.1 μM, 1.4 μM, 457 nM, 152 nM, 51 nM, and 17 nm. And 11 concentrations of gentamicin were assessed: 100 μM, 33 μM, 11 μM, 3.7 μM, 1.2 μM, 412 nM, 137 nM, 46 nM, 15 nM, 5.1 nM, and 1.7 nM.

An ad hoc test was also designed to evaluate quinolinic acid (0.1, 1-, 10, 100, 1000 μM) effect in presence of 10 μM glycine, with or without 10 μM dextromethadone.

The combined effect of 40 or 200 nM glutamate or 10 μM gentamicin was also evaluated in addition to quinolinic acid (0.1-1-10-100-1000 μM) and 10 μM glycine, with or without 10 μM dextromethadone.

FLIPR determination of intracellular calcium level was used as a read-out for NMDAR activation.

E. Methods and Procedures

400× compound plates were prepared by Echo Labcyte system, containing in every well: 300 nl/well of 400×L-glutamate/glycine solution in H₂O and 300 nl/well of 400×test item solution in DMSO. 400× compound plate was stored at −20° C. till FLIPR experimental day.

A 4× compound plate was generated from 400× compound plate by addition of up to 30 μl/well of compound buffer on FLIPR experimental day.

A FLIPR system was used to monitor intracellular calcium level in NMDAR cell lines, pre-loaded for 1 hour with Fluo-4, and then washed with assay buffer. Intracellular calcium level was monitored for 10 seconds before and 5 minutes after test item addition, in presence of L-glutamate and glycine.

F. Data Handling and Analysis

AUC values of fluorescence were measured by ScreenWorks 4.1 (Molecular Devices) FLIPR software, to monitor calcium level during the 5 minutes after test item addition (AUC 10-310 s). Then, data were normalized by Excel 2013 (Microsoft Office) software, using wells added with 10 μM L-glutamate plus 10 μM glycine (column 23) as high control, and wells added with assay buffer only (column 24) as low control.

To assess plate quality, Z′ calculations were performed in Excel. Z′ was calculated according to following equation:

Z′=1−3(σₕ+σₗ)/|μₕ−μₗ|

where μ and σ are the means and the standard deviations of the means of high (h) and low (l) controls, respectively.

Test item IC₅₀ values were calculated using four parameter logistic equation by XLfit, for every NMDA receptor type, when minimal response resulted less than 50%, so that maximal inhibition resulted more than 50%:

Y=Bottom+(Top−Bottom)/(1+10\hat{ }((LogEC50−X)*HillSlope))

where Y is % effect, respect to 10 μM L-glutamate plus 10 μM glycine, and X is test item molar concentration.

Test item CRC data were plotted by Prism 8 GraphPad software, in the different experimental conditions. And, column analysis, performed by Prism 8 GraphPad software, was one way ANOVA followed by Tukey's multiple comparisons test, with a single pooled variance.

G. Protocol Deviations

The preparation of 2000× concentrated solution (20 mM) of dextromethadone occurred in H₂O, rather than in DMSO. This protocol deviation neither affected the overall interpretation nor compromised the integrity of the study.

H. Results

1. Plate Z′ Values

6 cell plates for every cell line (GluN1-GluN2A, GluN1-GluN2B, GluN1-GluN2C, GluN1-GluN2D) were tested with the same compound plate, containing all test items. All cell plates resulted with Z′ values >0.5, and were accepted.

Z′ values for GluN1-GluN2A plates resulted as follows: 0.78-0.81-0.78-0.82-0.87-0.80.

Z′ values for GluN1-GluN2B plates resulted as follows: 0.72-0.63-0.68-0.71-0.75-0.69.

Z′ values for GluN1-GluN2C plates resulted as follows: 0.57-0.62-0.57-0.61-0.70-0.63.

Z′ values for GluN1-GluN2D plates resulted as follows: 0.74-0.81-0.83-0.80-0.80-0.81.

2. Quinolinic Acid

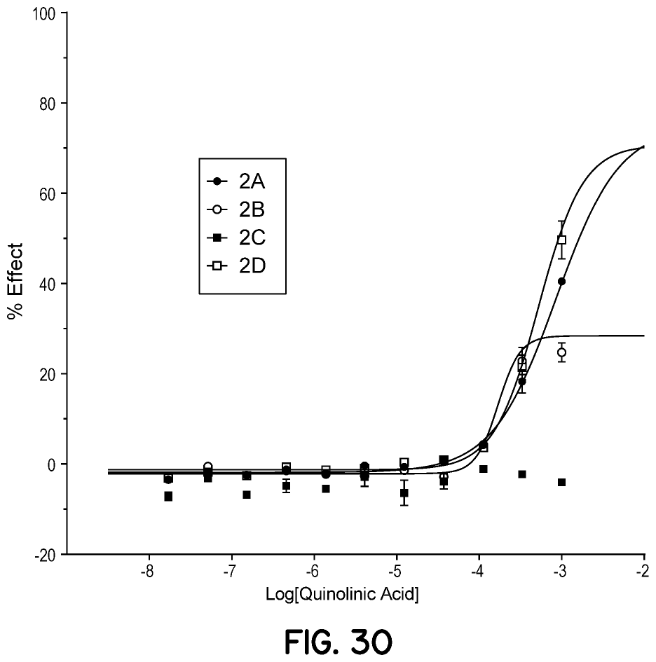

A quinolinic acid CRC plot in 4 NMDA receptor types by GraphPad Prism is presented in FIG. 30. Quinolinic acid CRC was obtained in presence of 10 μM glycine. And data are reported as mean±SEM, n=6.

Quinolinic acid best-fit values in 4 NMDA receptor types were calculated by GraphPad Prism and resulted as follows in Table 37:

TABLE 37

| | 2A | 2B | 2C | 2D |
|---|---|---|---|---|
| pEC₅₀ | 3.1 | 3.8 | <3 | 3.3 |
| EC₅₀ (μM) | 850 * | 170 | >1000 | 520 |
| Minimal response (%) | −1.9 | −2.1 | −4.0 | −1.3 |
| Response at max conc (%) | 40 | 25 | −4.0 | 50 |

* GluN1-GluN2A fit was obtained by constraining maximal response at 75%.

3. Gentamicin

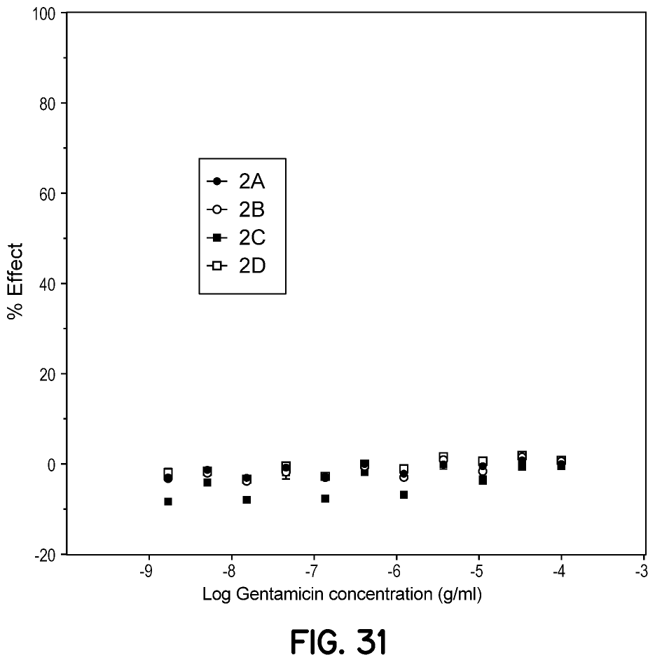

A gentamicin CRC plot in 4 NMDA receptor types by GraphPad Prism is presented in FIG. 31. Gentamicin CRC was obtained in presence of 10 μM glycine. Data are reported as mean±SEM, n=6.

Gentamicin best-fit values in 4 NMDA receptor types were calculated by GraphPad Prism, and resulted as follows in Table 38:

TABLE 38

| | 2A | 2B | 2C | 2D |
|---|---|---|---|---|
| pEC₅₀ | <4 | <4 | <4 | <4 |
| EC₅₀ (μg/ml) | >100 | >100 | >100 | >100 |
| Minimal response (%) | −3.3 | −3.7 | −8.2 | −3.2 |
| Response at max cone (%) | 0.1 | 0.7 | 0.03 | 1.0 |

4. Quinolinic Acid Effect in Presence of 10 μM Glycine and Interaction with Dextromethadone

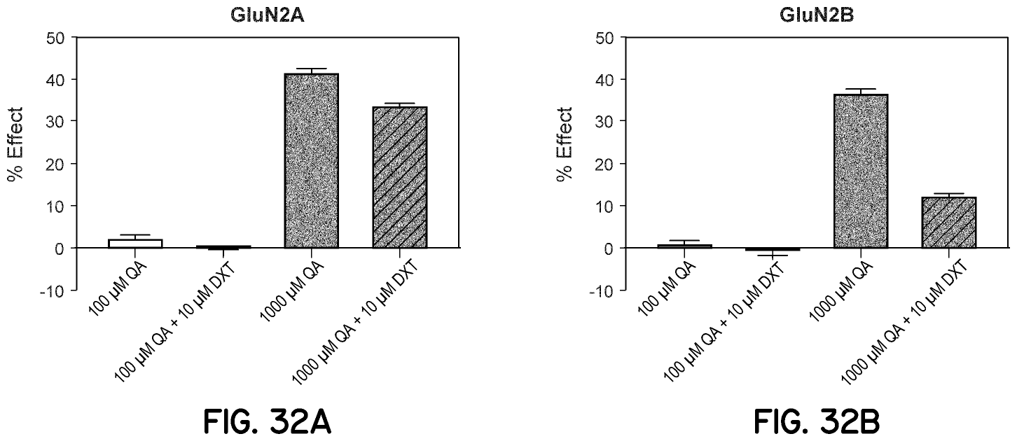

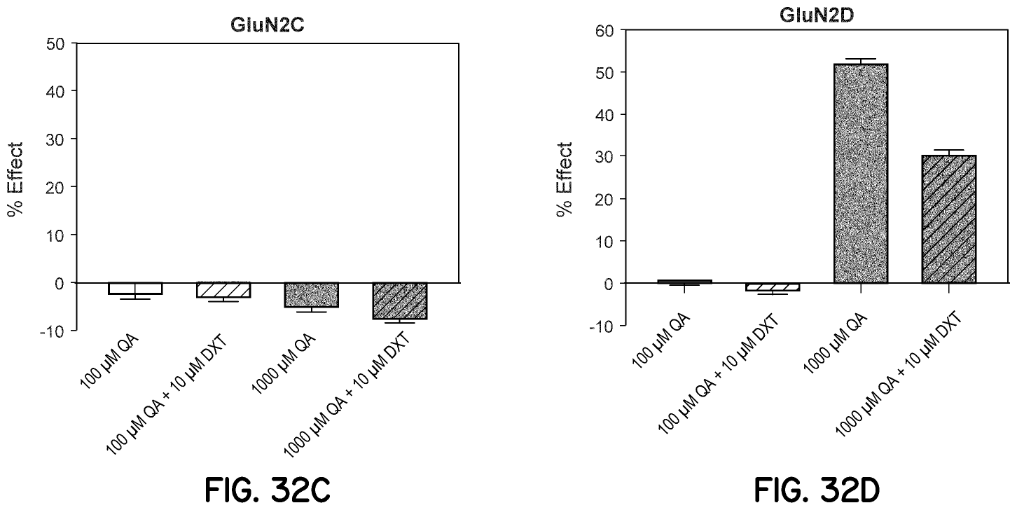

100-1000 μM quinolinic acid (QA) effect was evaluated in presence of 10 μM: glycine, using the 4 NMDAR cell lines, and results are shown in FIGS. 32A-32D. 10 μM dextromethadone (DXT) addition was also evaluated. Data shown are mean±SEM, n=42 for each group.

The same data shown in FIGS. 32A-32D is tabulated in Table 39 below, including dextromethadone statistical results.

TABLE 39

| | | 100 QA + | | 1000 QA + |
|---|---|---|---|---|
| | 100 QA | 10 DXT | 1000 QA | 10 DXT |
| GluN2A | 2.5 ± 0.3 | −0.2 ± 0.2 (*) | 41 ± 1.2 | 34 ± 0.6 (****) |
| GluN2B | 0.9 ± 0.7 | −1.0 ± 0.8 (ns) | 37 ± 1.3 | 12 ± 0.7 (****) |
| GluN2C | −2.8 ± 0.6 | −3.3 ± 0.5 (ns) | −5.5 ± 0.4 | −8.0 ± 0.4 (*) |
| GluN2D | −0.1 ± 0.4 | −2.4 ± 0.2 (ns) | 55 ± 1.1 | 32 ± 0.9 (****) |

Tabulated data are mean±SEM (P value), n=42 for each group. Title concentrations are in micromolar. Legend: ns, not significant; * is P<0.05;**** is P<0.0001. QA is quinolinic acid. DXT is dextromethadone hydrochloride.

5. 40 nM L-Glutamate and 10 μM Glycine: Effect of 100 μM Quinolinic Acid and/or 10 μM Dextromethadone

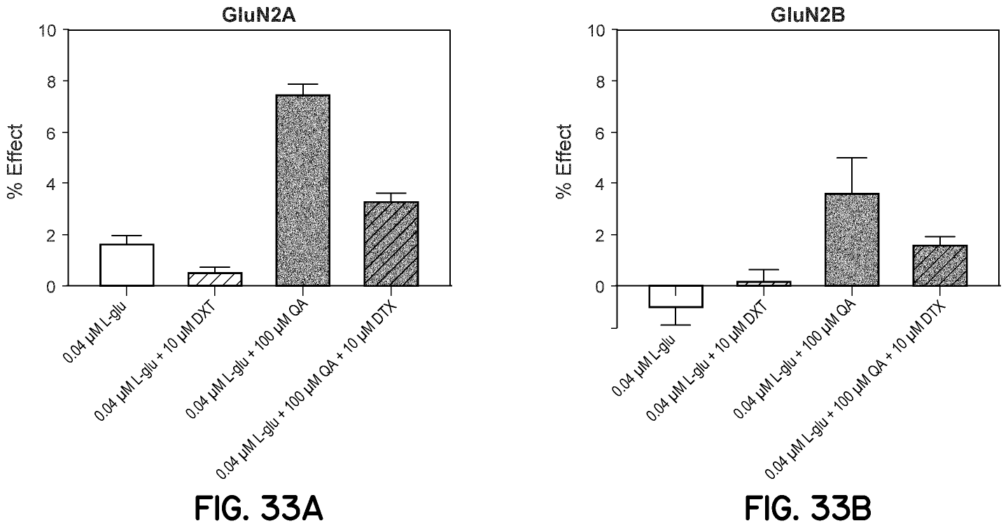

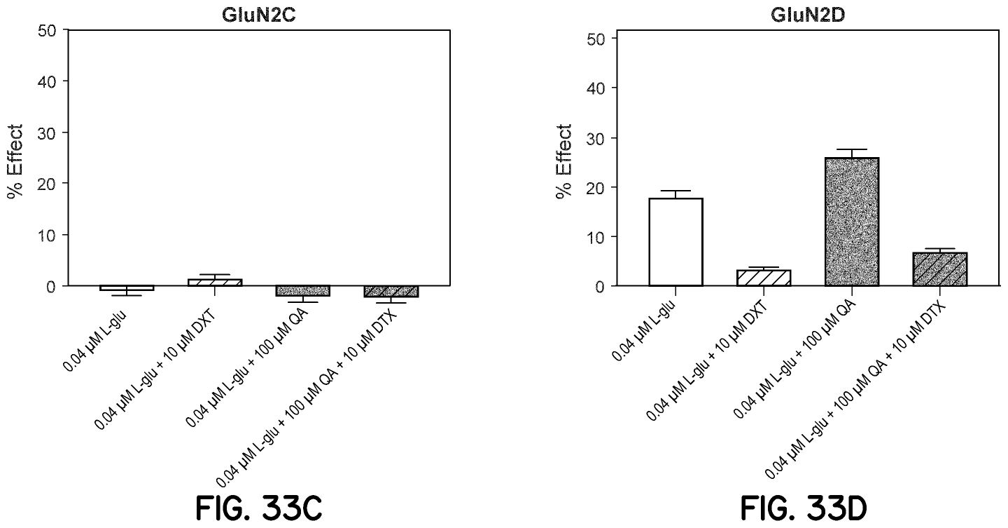

40 nM L-glutamate effect in presence of 10 μM: glycine was evaluated. Addition of 100 μM quinolinic acid (QA) and/or 10 μM dextromethadone (DXT) was also evaluated, using the 4 NMDAR cell lines, and results are shown in FIGS. 33A-33D.

The same data shown in FIGS. 33A-33D is tabulated in Table 40 below, including dextromethadone statistical results.

TABLE 40

| | | | | 0.04 L-Glu + |
|---|---|---|---|---|
| | | 0.04 L-Glu + | 0.04 L-Glu + | 100 QA + |
| | 0.04 L-Glu | 10 DXT | 100 QA | 10 DXT |
| GluN2A | 1.7 ± 0.3 | 0.5 ± 0.2 (ns) | 7.5 ± 0.4 | 3.4 ± 0.2 (****) |
| GluN2B | −0.9 ± 0.6 | 0.2 ± 0.4 (ns) | 3.7 ± 1.3 | 1,6 ± 0.3 (ns) |
| GluN2C | −1.2 ± 0.6 | 1.6 ± 0.4 (ns) | −2.0 ± 1.1 | −2.5 ± 0.7 (ns) |
| GluN2D | 18 ± 1.2 | 3.5 ± 0.2 (****) | 26 ± 1.2 | 7.1 ± 0.4 (****) |

Data are mean±SEM (P value), n=42 for each group. Title concentrations are in micromolar. Legend: ns, not significant;**** is P<0.0001. L-Glu is L-glutamate. QA is quinolinic acid. DXT is dextromethadone hydrochloride.

6. 40 nM L-Glutamate and 10 μM Glycine: Effect of 1000 μM Quinolinic Acid and/or 10 μM Dextromethadone

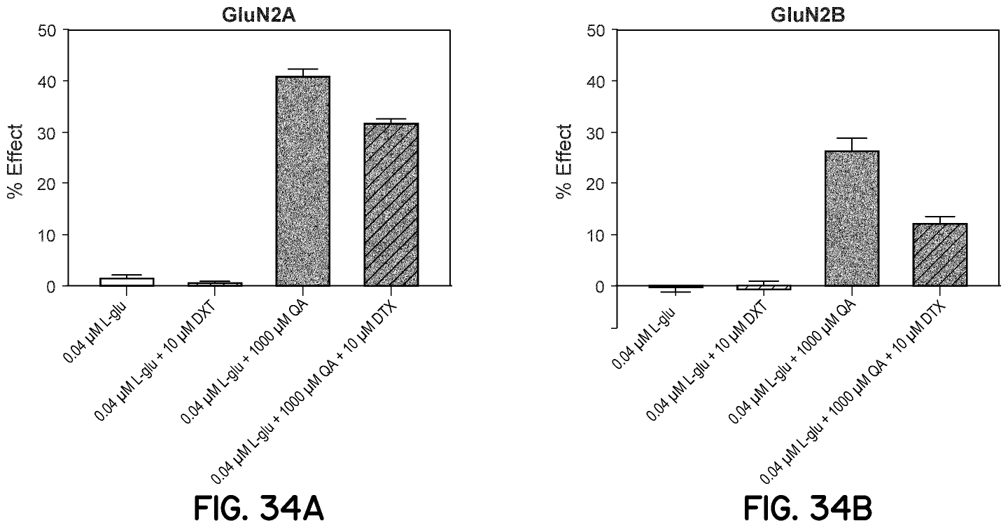

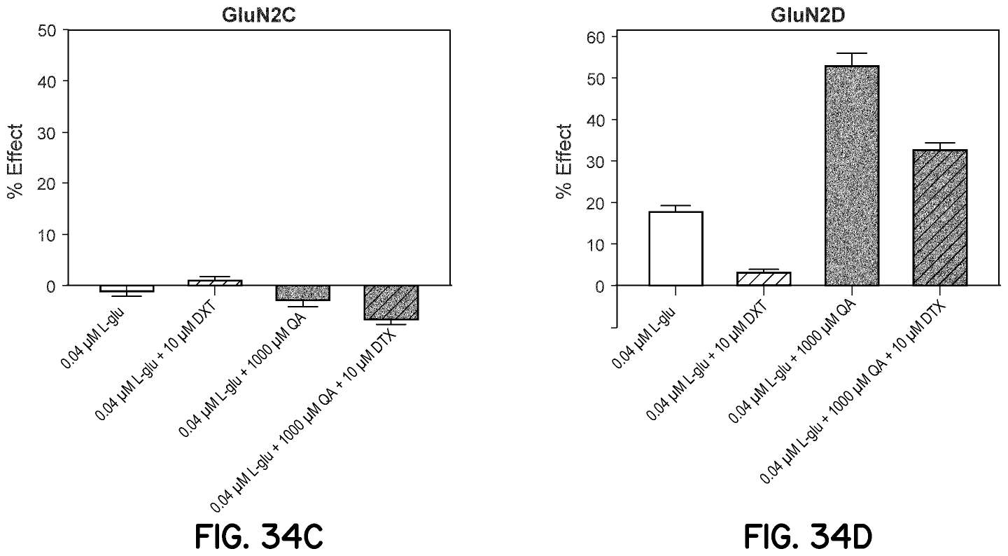

40 nM L-glutamate effect in presence of 10 μM: glycine was evaluated. Addition of 1000 μM quinolinic acid (QA) and/or 10 μM dextromethadone (DXT) was also evaluated, using the 4 NMDAR cell lines, and results are shown in FIGS. 34A-34D.

The same data shown in FIGS. 34A-34D is tabulated in Table 41 below, including dextromethadone statistical results.

TABLE 41

| | | | | 0.04 L-Glu + |
|---|---|---|---|---|
| | | 0.04 L-Glu + | 0.04 L-Glu + | 1000 QA + |
| | 0.04 L-Glu | 10 DXT | 1000 QA | 10 DXT |
| GluN2A | 1.7 ± 0.3 | 0.5 ± 0.2 (ns) | 41 ± 1.0 | 32 ± 0.7 (****) |
| GluN2B | 0.9 ± 0.6 | 0.2 ± 0.4 (ns) | 27 ± 2.2 | 13 ± 1.0 (****) |
| GluN2C | −1.2 ± 0.6 | 1.6 ± 0.4 (ns) | −4.1 ± 0.9 | −8.6 ± 1.5 (**) |
| GluN2D | 18 ± 1.2 | 3.5 ± 0.2 (****) | 53 ± 2.3 | 33 ± 1.6 (****) |

Data are mean±SEM (P value), n=42 for each group. Title concentrations are in micromolar. Legend: ns, not significant; ** is P<0.01;**** is P<0.0001. QA is quinolinic acid. DXT is dextromethadone hydrochloride.

7. 200 nM L-Glutamate and 10 μM Glycine: Effect of 100 μM Quinolinic Acid and/or 10 μM Dextromethadone

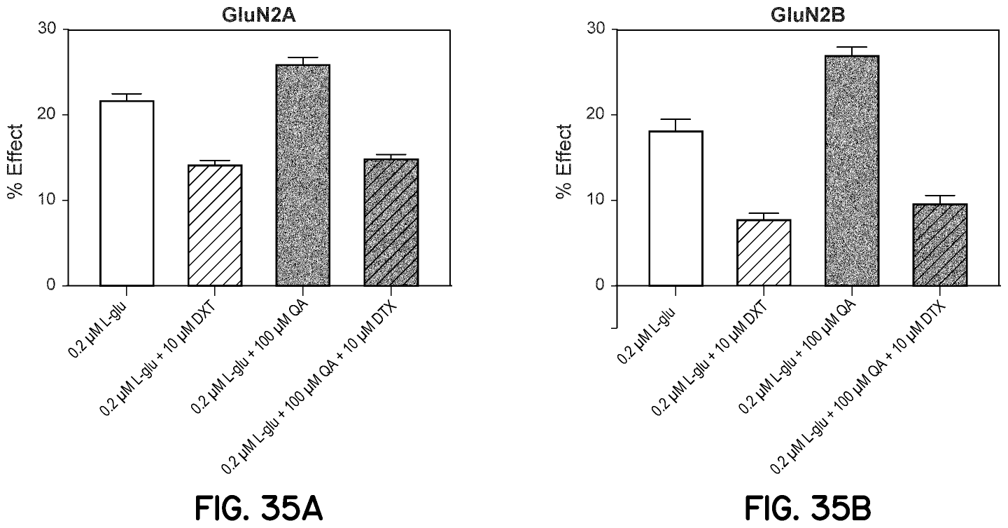

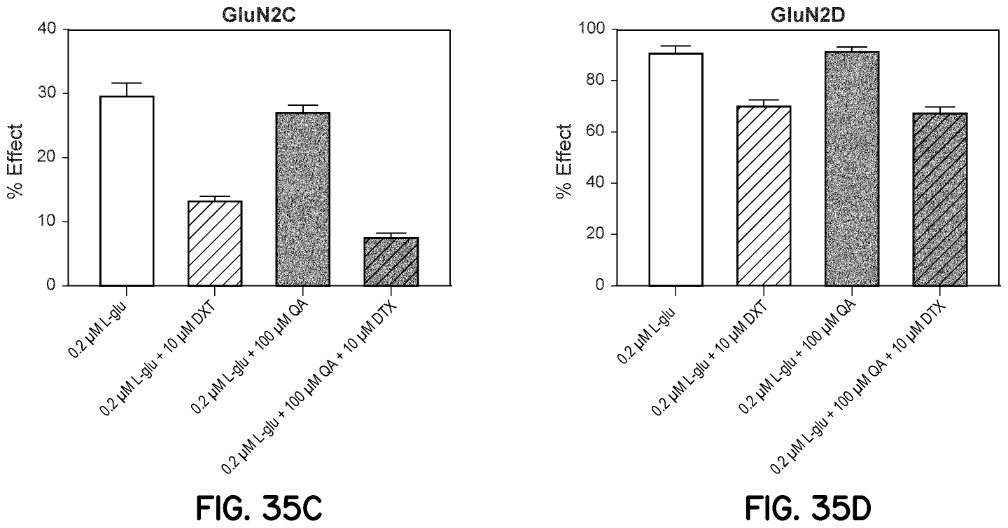

200 nM L-glutamate effect in presence of 10 μM: glycine was evaluated. Addition of 100 μM quinolinic acid (QA) and/or 10 μM dextromethadone (DXT) was also evaluated, using the 4 NMDAR cell lines, and results are shown in FIGS. 35A-35D.

The same data shown in FIGS. 35A-35D is tabulated in Table 42 below, including dextromethadone statistical results.

TABLE 42

| | | 0.2 L-Glu + | 0.2 L-Glu + | 0.2 L-Glu + 100 |
|---|---|---|---|---|
| | 0.2 L-Glu | 10 DXT | 100 QA | QA + 10 DXT |
| GluN2A | 22 ± 0.7 | 14 ± 0.4 (****) | 26 ± 0.6 | 15 ± 0.5 (****) |
| GluN2B | 18 ± 1.2 | 8.0 ± 0.5 (****) | 27 ± 0.8 | 9.9 ± 0.8 (****) |
| GluN2C | 30 ± 1.7 | 13 ± 0.7 (****) | 27 ± 1.1 | 7.7 ± 0.6 (****) |
| GluN2D | 92 ± 2.0 | 71 ± 2.3 (****) | 93 ± 1.0 | 69 ± 1.4 (****) |

Data are mean±SEM (P value), n=42 for each group. Title concentrations are in micromolar. Legend:**** is P<0.0001. QA is quinolinic acid. DXT is dextromethadone hydrochloride.

8. 200 nM L-Glutamate and 10 μM Glycine: Effect of 1000 μM Quinolinic Acid and/or 10 μM Dextromethadone

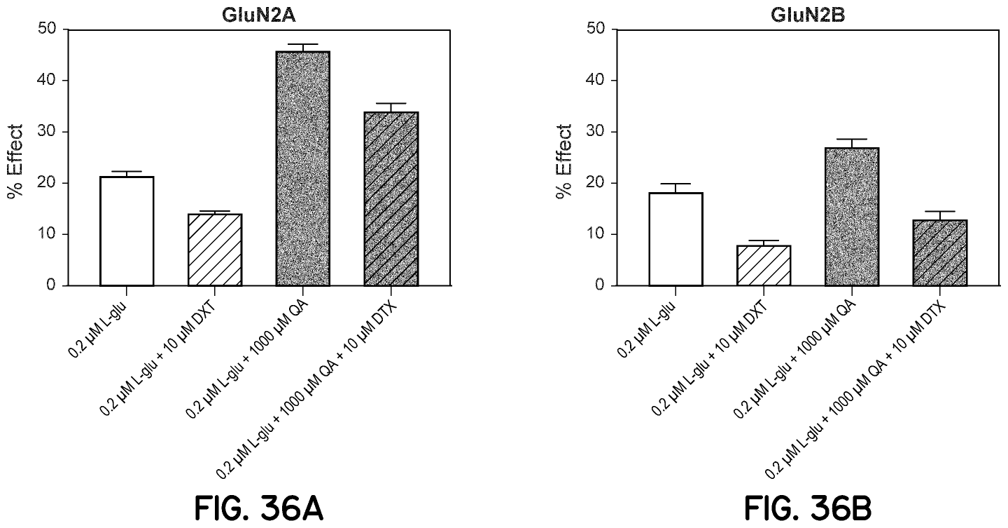

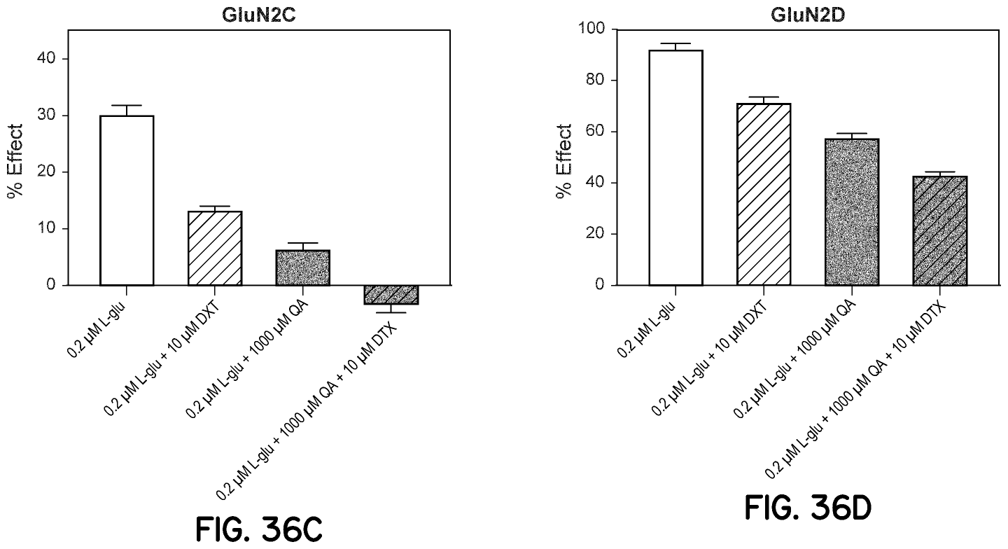

200 nM L-glutamate effect in presence of 10 μM: glycine was evaluated. Addition of 1000 μM quinolinic acid (QA) and/or 10 μM dextromethadone (DXT) was also evaluated, using the 4 NMDAR cell lines, and results are shown in FIGS. 36A-36D.

The same data shown in FIGS. 36A-36D is tabulated in Table 43 below, including dextromethadone statistical results.

TABLE 43

| | | 0.2 L-Glu + | 0.2 L-Glu + | 0.2 L-Glu + 1000 |
|---|---|---|---|---|
| | 0.2 L-Glu | 10 DXT | 1000 QA | QA + 10 DXT |
| GluN2A | 22 ± 0.7 | 14 ± 0.4 (****) | 46 ± 0.9 | 35 ± 1.0 (****) |
| GluN2B | 18 ± 1.2 | 8.0 ± 0.5 (****) | 27 ± 1.5 | 13 ± 1.0 (****) |
| GluN2C | 30 ± 1.7 | 13 ± 0.7 (****) | 6.6 ± 0.8 | −3.8 ± 0.9 (****) |
| GluN2D | 92 ± 2.0 | 71 ± 2.3 (****) | 58 ± 1.6 | 43 ± 1.1 (****) |

Data are mean±SEM (P value), n=42 for each group. Title concentrations are in micromolar. Legend:**** is P<0.0001. QA is quinolinic acid. DXT is dextromethadone hydrochloride.

9. 1000 μM Quinolinic Acid and 10 μM Glycine: Effect of 10 μg/ml Gentamicin and/or 10 μM Dextromethadone

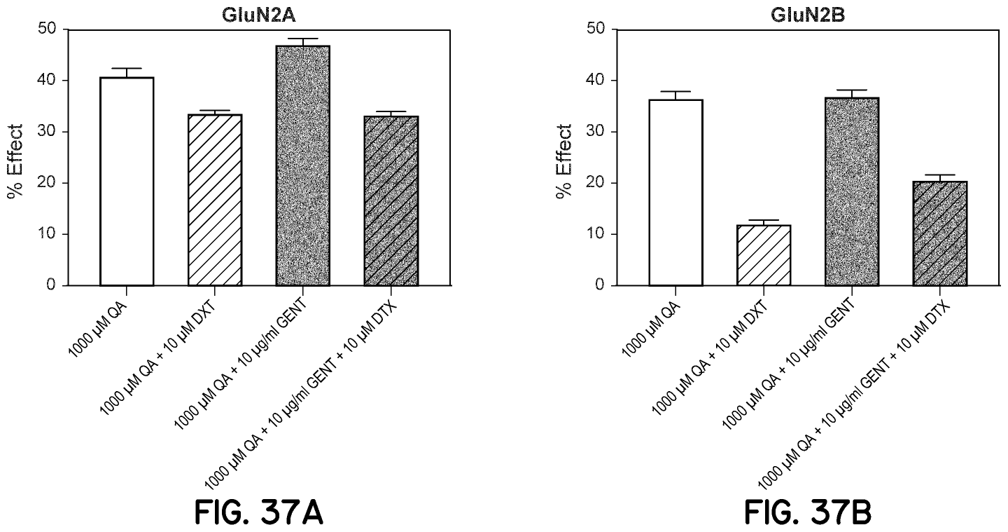

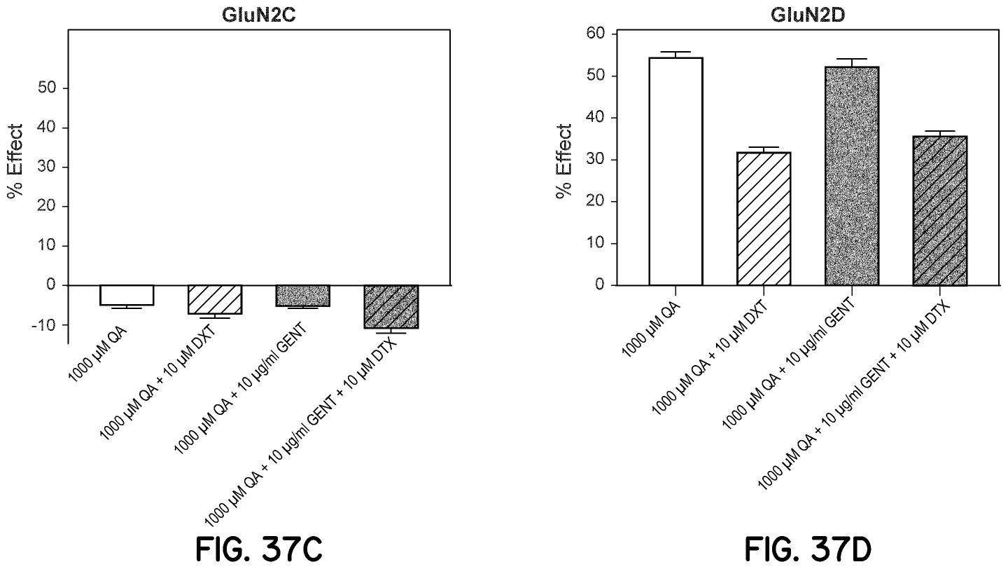

1000 μM quinolinic acid (QA) effect in presence of 10 μM glycine was evaluated. Addition of 10 g/ml gentamicin and/or 10 μM dextromethadone (DXT) was also evaluated, using the 4 NMDAR cell lines, and results are shown in FIGS. 37A-37D.

The same data shown in FIGS. 37A-37D is tabulated in Table 44 below, including DXT statistics.

TABLE 44

| | | 1000 QA + | 1000 QA + | 1000 QA + 10 |
|---|---|---|---|---|
| | 1000 QA | 10 DXT | 10 GENT | GENT + 10 DXT |
| GluN2A | 41 ± 1.2 | 34 ± 0.6 (****) | 47 ± 1.1 | 34 ± 0.6 (****) |
| GluN2B | 37 ± 1.3 | 12 ± 0.7 (****) | 37 ± 1.4 | 21 ± 0.9 (****) |
| GluN2C | −5.5 ± 0.4 | −8.0 ± 0.4 (*) | 5.6 ± 0.4 | −11 ± 0.9 (****) |
| GluN2D | 55 ± 1.1 | 32 ± 0.9 (****) | 53 ± 1.7 | 36 ± 0.8 (****) |

Data are mean±SEM (P value), n=42 for each group. Title concentrations are in μM (QA and DXT) or in μg/ml (GENT). Legend:* is P<0.05;**** is P<0.0001. QA is quinolinic acid. DXT is dextromethadone hydrochloride, GENT is gentamicin sulphate.

I. Discussion

A FLIPR calcium assay was used to profile test items using stable cell lines expressing diheteromeric recombinant human NMDAR, containing GluN1 plus one amongst GluN2A, GluN2B, GluN2C or GluN2D subunits.

10 μM dextromethadone inhibited NMDAR mediated calcium entry induced by glutamate, quinolinic acid or their combination and quinolinic acid+gentamicin.

Quinolinic acid showed partial agonist mode action on GluN2A, GluN2B, GluN2D containing diheteromeric NMDAR in FLIPR calcium assay. Quinolinic acid EC₅₀ resulted 850, 170 and 520 μM in GluN2A, GluN2B and GluN2D cell lines, respectively. Quinolinic acid 1000 μM instead decreased intracellular calcium increase elicited by 0.2 μM L-glutamate in GluN2C cell line.

Quinolinic acid, in presence of 10 μM glycine induced an increase in calcium entry in GluN2A, GluN2B and GluN2D cell lines at starting at approximately 100 μM and up to 1000 μM. Lower quinolinic acid concentrations resulted ineffective, in GluN2A, GluN2B and GluN2D cell lines. Quinolinic acid did not increase intracellular calcium in GluN2C cell line at tested concentration but appeared to act as a NAM on this cell line.

Quinolinic acid maximal % effect (mean±SEM) on calcium entry resulted at 1000 μM in presence of 10 μM glycine: 41±1.1%, 37±1.3% and 55±1.1% on GluN2A, GluN2B and GluN2D cell lines, respectively, compared to 100% effect elicited by 10 μM L-glutamate plus 10 μM glycine.

Quinolinic acid CRC in the GluN2B cell line suggests a partial agonist behavior, since 333 μM and 1000 μM quinolinic acid elicited similar submaximal calcium entry (23±3.0 and 25±2.1%, respectively).

Partial agonism behavior is also supported by quinolinic acid complex interactions with L-glutamate, depending on agonists concentrations and NMDAR subunit. 100 μM quinolinic acid showed positive interaction with 0.04 μM L-glutamate at GluN2A, GluN2B and GluN2D subunits, but 1000 μM quinolinic acid showed negative interaction with 0.2 μM L-glutamate at GluN2D subunit, where 0.2 μM L-glutamate alone reached nearly maximal efficacy (92±2.0%).

In addition, 1000 μM quinolinic acid decreased intracellular calcium increase elicited by 0.2 μM L-glutamate in GluN2C cell line (from 30±1.7% down to 6.6±0.8%, P<0.0001), surprisingly, acting as an antagonist, see below.

Quinolinic acid, at lower concentrations, such as 0.1, 1, 10 μM, did not elicit any response in any cell line, nor did modify cell line response to 0.04 μM or 0.2 μM L-glutamate, nor to 10 μM gentamicin.

Quinolinic acid observed effects in FLIPR are compatible with a partial agonist action on GluN2A, GluN2B, GluN2D diheteromeric NMDA receptors, in agreement with previous literature papers about GluN2A or GluN2B containing diheteromeric NMDA receptors (Banke T G, Traynelis S F. Activation of NR1/NR2B NMDA receptors. Nat Neurosci. 2003; 6(2):144-152; Blanke M L, VanDongen A M. Constitutive activation of the N-methyl-D-aspartate receptor via cleft-spanning disulfide bonds. J Biol Chem. 2008; 283(31):21519-21529; Kussius and Popescu, 2009) using electrophysiological techniques. Banke and Traynelis, 2003 reported a quinolinic acid potency of 518±35 μM by outside out electrophysiological measures on rat GluN1-GluN2B receptor, in good agreement with the present inventors' reported values. The inability of quinolinic acid to activate GluN1-GluN2C receptor in FLIPR is in agreement with data from De Carvalho et al. (De Carvalho L P, Bochet P, Rossier J. The endogenous agonist quinolinic acid and the non endogenous homoquinolinic acid discriminate between NMDAR2 receptor subunit. Neurochem. Int. 1996; 28:445-452), showing no electrophysiological response to 100 or 1000 μM quinolinic acid in oocytes injected with rat GluN1 and GluN2C subunits. The ability of 1000 μM quinolinic acid to decrease intracellular calcium increase elicited by 0.2 μM L-glutamate in GluN2C cell line by FLIPR suggests that it retains some ability to bind glutamate binding site of GluN2C subunit, but with zero efficacy, thus behaving as an antagonist and not simply a lower potency agonist at GluN2C subunit.

Gentamicin, tested in presence of 10 μM glycine but in absence of glutamate, did not elicit calcium entry at any tested concentration (from 1.7 nM to 100 μM) in all tested cell lines. Therefore, gentamicin, a PAM (Example 5, Part I), appears devoid of agonist activity at the NMDAR glutamate binding site.

10 μg/ml gentamicin slightly potentiated 1000 μM quinolinic acid only in GluN2A cell line (from 41±1.2% up to 47±1.1%, P<0.0001). This confirms that there may be potentiation from concomitant application of agonist+PAM combinations and that dextromethadone can effectively block the enhanced Ca²⁺ currents elicited by a combination of agonist and PAM, at least at GluN2A subtypes.

It is not surprising that gentamicin positive modulation effect on NMDARs is agonist dependent, since for allosteric modulators, affinity can be conditional in that the magnitude of the effective K_{B} is dependent on the type of cobinding agonist and its concentration (as reported by Kenakin T, Strachan R T. PAM-Antagonists: A Better Way to Block Pathological Receptor Signaling? Trends Pharmacol Sci. 2018; 39(8):748-765).

10 μM dextromethadone confirmed its ability to significantly decrease intracellular Ca²⁺ influx induced by 200 nM L-glutamate in all four cell lines, and by 40 nM L-glutamate in GluN2D cell line (see also Part I of this Example 5).

10 μM dextromethadone did also reduce intracellular Ca²⁺ influx increased by 333 and 1000 μM quinolinic acid in GluN2A, GluN2B and GluN2D cell lines, as well as by combinations of quinolinic acid and glutamate or gentamicin that elicited sufficiently high intracellular calcium levels. This pattern of activity of dextromethadone confirms its activity as a uncompetitive channel blocker effective for decreasing Ca²⁺ influx elicited by l-glutamate, other agonists at the glutamate site and PAMs and their combinations, when sufficient amounts of Ca²⁺ influx are elicited.

Braidy et al. (Braidy N, Grant R, Adams S, Brew B J, Guillemin G J. Mechanism for quinolinic acid cytotoxicity in human astrocytes and neurons. Neurotox Res. 2009; 16(1):77-86), describes submicromolar effects of quinolinic acid [inhibited by MK-801, an open channel blocker with uncompetitive activity similar but more potent compared to dextromethadone (see Example 1)] on various parameters of astrocytes and neurons: intracellular nicotinamide adenine dinucleotide (NAD⁺) and poly(ADP-ribose) polymerase (PARP) levels; extracellular lactate dehydrogenase (LDH) levels; iNOS and nNOS expression levels in astrocytes and neurons, respectively.

The present inventors' results, testing GluN2A, GluN2B, GluN2D and GluN2C cell lines, do not show effects of quinolinic acid at concentrations lower than 100 μM. The present inventors hypothesize that the cultured human astrocytes and neurons sensitive to submicromolar concentrations of quinolinic acid studied by Braidy et al., 2009 express NMDAR subtypes with subunit combinations that may be more sensitive to quinolinic acid, such as subtypes containing GluN3A and GluN3B subunits (e.g., tri-heteromers NR1-NR2A or B or C or D-NR2A or B). NMDAR containing GluN3A and GluN3B subunits have been shown to be present in astrocytes (Skowrońska K, Obara-Michlewska M, Zielińska M, Albrecht J. NMDA Receptors in Astrocytes: In Search for Roles in Neurotransmission and Astrocytic Homeostasis. Int J Mol Sci. 2019; 20(2):309).

Interestingly, the GluN3A subunit is considered key to Huntington's disease (HD) pathophysiology, which is also mimicked by quinolinic acid brain injection. Quinolinic acid neurotoxicity is well known to replicate neurochemical characteristics of HD (Beal M F, Kowall N W, Ellison D W, Mazurek M F, Swartz K J, Martin J B. Replication of the neurochemical characteristics of Huntington's disease by quinolinic acid. Nature. 1986; 321(6066):168-171). GluN3A-receptor expression was enhanced in both Huntington's disease (HD) animal models (due to PACSIN adaptor protein sequestration by mutated huntingtin), as well as in human HD patient striatal tissue (Mackay J P, Nassrallah W B, Raymond L A. Cause or compensation?-Altered neuronal Ca2+ handling in Huntington's disease. CNS Neurosci Ther. 2018; 24(4):301-310), and suppressing aberrant GluN3A expression rescued synaptic and behavioral impairments in HD models (Marco S, Giralt A, Petrovic M M, et al. Suppressing aberrant GluN3A expression rescues synaptic and behavioral impairments in Huntington's disease models. Nat Med. 2013; 19(8):1030-1038). Therefore, based on the present inventors' results, quinolinic acid could preferentially target GluN3A containing NMDARs.

Koch et al., 2019 reported that 7.2 mM quinolinic acid was able to activate GluN1-GluN3B subtype in oocytes (Koch A, Bonus M, Gohlke H, Klocker N. Isoform-specific Inhibition of N-methyl-D-aspartate Receptors by Bile Salts. Sci Rep. 2019 Jul. 11; 9(1):10068). Quinolinic acid is considered a NMDAR partial agonist at the glutamate binding site, as confirmed by our FLIPR study and therefore the results by Koch et al. appear to contrast with the assumption that both subunits present in the GluN1-GluN3B subtype only contain the glycine binding site.

The pharmacology of GluN3 containing NMDAR is in its infancy, as exemplified by a recent paper (Grand T, Abi Gerges S, David M, Diana M A, Paoletti P. Unmasking GluN1/GluN3A excitatory glycine NMDA receptors. Nat Commun. 2018; 9(1):4769) showing that classical glycine site antagonists (such as 7-CKA or CGP-78608) can instead unmask a glycine excitatory role at GluN1-GluN3A receptor.

In relation to Example 10 (below), it is interesting to note that select astrocytic populations (e.g., those in CA1 hippocampal area) highly express MOR (Nam et al., 2018). These MORs are thought to play a central role in astrocyte glutamate release and memory formation (Nam et al., 2019). Astrocyte role in extracellular glutamate homeostasis is well recognized, and astrocyte derived glutamate is key to NMDAR mediated potentiation of inhibitory synaptic transmission (Kang J, Jiang L, Goldman S A, Nedergaard M. Astrocyte-mediated potentiation of inhibitory synaptic transmission. Nat Neurosci. 1998; 1(8):683-692), as well as key to NMDAR mediated neuronal slow inward current (SIC) and LTD (Fellin T, Pascual O, Gobbo S, Pozzan T, Haydon P G, Carmignoto G. Neuronal synchrony mediated by astrocytic glutamate through activation of extrasynaptic NMDA receptors [published correction appears in Neuron. 2005 Jan. 6; 45(1):177]. Neuron. 2004; 43(5):729-743; Navarrete et al., 2019).

Corroborating the Example 10 disclosure, the preferential targeting (Shepherd Affinity) by dextromethadone for structurally associated, physically coupled, NMDAR-MOR expressed on the membrane of select astrocytic populations, might contribute to the antidepressant mechanisms of dextromethadone by mediating a balanced control of extracellular glutamate levels.

J. Conclusions Based on Parts I and II of Example 5

Conclusions based on the study of the present inventors in this Example 5 are as follows: First, large amounts (mM concentration) of presynaptic glutamate release (physiological stimulus induced release), when rapidly cleared by a functional EAAT system, are not excitotoxic (physiologic neural transmission), while small amounts (low nM range) that result in tonic hyper-activation (tonic and pathologic) of NMDARs, may cause excessive Ca²⁺ influx and chronic low grade excitotoxicity in select cells with halting of LTP and cell impairment and cell loss.

Second, dextromethadone was able to downregulate Ca²⁺ influx at all levels of glutamate concentration, even at concentrations as low as 40 nM, both in presence and absence of a toxic PAM (in this case gentamicin).

Third, the very low concentrations of glutamate tested may be representative of tonic and pathologic concentrations in select cells and may cause Ca²⁺ influx that is excessive for select cells when prolonged over time.

Fourth, the very low concentrations of glutamate tested may be representative of tonic concentrations that may determine tonic stimulation of interneurons, e.g., inhibitory interneurons projecting to the mPFC, involved in the pathogenesis of MDD, or other interneurons, involved in the pathogenesis of other neuropsychiatric disorders.

Fifth, the effects of tonic low concentration of glutamate on Ca²⁺ influx may be enhanced by PAMs, as seen with gentamicin.

Sixth, excessive (pathologic) exposure to presynaptic glutamate at low concentrations (low nM range) may be caused by serial presynaptic depolarizing events (e.g., eEPSCs) or may even be spontaneous (e.g., mEPSCs) and/or by a defect in clearance from the synaptic cleft, e.g., a defect in EAAT.

Seventh, the disease-modifying effects of dextromethadone may be exerted independently of the cause of excessive Ca²⁺ influx: 1) excessive presynaptic release (persistent excessive “low concentration” glutamate), 2) postsynaptic enhancement (toxic PAMs or agonists at the NMDAR enhancing the effects of very low concentration ambient synaptic glutamate), 3) synaptic cleft defective clearance of glutamate (EAAT defect).

Eighth, Examples 1, 2, 3, 6, 7, 9, and 10, along with the first through seventh conclusions (above), signal that dextromethadone may selectively target select NMDAR channels when their kinetics are abnormal: dextromethadone blocks (see Example 6, “on” kinetics for dextromethadone action) the channel only when NMDARs on select cells remain open too long or too widely (hyperactive) and result in excessive Ca²⁺ influx.

Ninth, substantially useful “on,” “off” kinetics of methadone's NMDAR channel block: the side effect profile for dextromethadone, comparable to placebo at effective doses for MDD (Example 3, MDD), suggests that not only the “on” kinetic of dextromethadone (point 8 above), selective for blocking only pathologically hyperactivated (hyperactivated for too much time) NMDARs is useful, but also its “off” kinetics is such that it allows resumption of NMDAR activity without causing a prolonged complete block that would impede physiologic NMDAR activity and cause side effects (e.g., depersonalization/dissociation effects, as seen with ketamine, a more potent NMDAR channel blocker).

Characterization of “on,” “off,” and “trapping” for dextromethadone are described in detail in Example 6, Part I and Part II.

The electrophysiology on-/off-rate assay was designed to establish test item onset and offset kinetic, relative to the block of 10/10 μM L-glutamate/glycine induced whole-cell current in GluN1/GluN2C NMDAR cell line. 10 μM Dextromethadone onset and offset kinetic parameters tau-on and tau-off resulted 46.4 and 174 s, respectively. 1 μM (±)-Ketamine (one tenth of the concentration of dextromethadone) tau-on and tau-off resulted 47.1 and 151 s, respectively, signalling a potency×10 compared to dextromethadone, corroborated by the Example 1 results.

Electrophysiology assay was designed to establish test item “trapping”, relative to the block of 10/10 μM L-glutamate/glycine induced whole-cell current in GluN1-GluN2C NMDAR cell line. Dextromethadone and (±)-ketamine were selected as test items. Dextromethadone “trapping” resulted 85.9%. (±)-Ketamine “trapping” resulted 86.7%.

Based on the above novel and unexpected findings and their correlation with Example 1 results, in particular the results illustrated in the K_{B} table (Table 28), more specifically the results illustrated in the GluN1-GluN2C column of Table 28, and the correlation with MDD efficacy and safety and PK parameters available in the literature for the different drugs tested in the assay, the present inventors disclose that clinically tolerated and MDD effective NMDAR channel blockers that are able to decrease Ca²⁺ permeability even in the presence of physiological Mg²⁺ concentrations in the resting membrane potential state should have the following characteristics: 1) Low potency (low micromolar) at GluN1-GluN2C subtypes [the potency of dextromethadone is 1/10 compared to ketamine (nanomolar): Example 1 (K_{B} table, Table 28) and Example 6A (“on” and “off”)]. 2) Relatively high “trapping”: lower than MK-801 and lower than PCP but comparable to ketamine and higher than memantine (memantine is ineffective for MDD).

In summary, the characteristics for the substantially useful NMDAR channel blocker for the MDD indication are: a small molecule with low micromolar preferential affinity for GluN1-GluN2C and GluN2D subtypes (1-12 micromolar); and 80-90% “trapping”; and the following “onset” and “offset” kinetic parameters: tau-on and tau-off: 40-50 s and 145-180 s, respectively; and low affinity (Example 10) for mu opioid receptors (e.g., 1/10 or less compared to morphine)

### Example 6

Overview: This Example 6 demonstrates characteristics of MDD-effective NMDAR channel blockers: (1) slow onset (low potency): so not to interfere with phasic physiological NMDAR activation which is very fast and therefore unaffected by a slow onset; and (2) relatively high trapping: so the drug will stick in the channel and exert a steady block of tonically and pathologically open channels.

Part I: Electrophysiology On-/Off-Rate Assay On GluN1/Glu2C NMDAR

A. Overview

In this Part I of Example 6, dextromethadone and (±)-ketamine were evaluated in manual patch clamp, to assess their onset and offset kinetic on recombinant diheteromeric human NMDAR containing GluN2C subunit.

1. Methods

Manual patch clamp recordings occurred at −70 mV. Cells were exposed for 5 s to 10/10 μM L-glutamate/glycine in absence of Mg²⁺, followed by a 30-s co-application of L-glutamate/glycine plus test item and a 50 s re-exposure to L-glutamate/glycine. Tau-on and tau-off were estimated by curve fitting to first order exponential equations.

2. Results

10 μM dextromethadone and 1 μM (±)-ketamine produced a similar 74.6%±1.9% (n=12) and 74.6%±2.2% (n=3) NMDAR current block, while 10 μM (±)-ketamine resulted in a block of 97.2%±0.3% (n=3).

Tau-on resulted 46.4 (n=11) and 47.1 s (n=10) for 10 μM dextromethadone and 1 μM (±)-ketamine, respectively. 10 μM (±)-ketamine tau-on was down to 9.9 s (n=4).

Offset kinetic of 10 μM dextromethadone (173.5 s, n=11) resulted similar to both 1 and 10 μM (±)-ketamine (151.0, n=10, and 163.2 s, n=4, respectively).

3. Conclusions

Lower potency of dextromethadone with respect to (±)-ketamine is due to dextromethadone slower onset kinetic, which suggests that dextromethadone action at NMDAR is activated by ambient L-glutamate and potential sparing of phasically activated NMDAR.

B. Summary

An electrophysiology on-/off-rate assay was designed to establish test item onset and offset kinetic, relative to the block of 10/10 μM L-glutamate/glycine induced whole-cell current in GluN1/GluN2C NMDAR cell line.

Dextromethadone and (±)-ketamine were selected as test items. 10 μM dextromethadone produced a 75% inhibition of GluN1/GluN2C mediated current, while 10, 3, 1, and 0.3 μM (±)-ketamine produced a 97%, 90%, 75% and 44% inhibition, respectively. And so, kinetic parameters of the two items were evaluated using test items at concentration eliciting similar effect, that is 10 and 1 μM for dextromethadone and (±)-ketamine, respectively.

10 μM Dextromethadone onset and offset kinetic parameters tau-on and tau-off resulted 46.4 and 174 s, respectively. 1 μM (±)-Ketamine tau-on and tau-off resulted 47.1 and 151 s, respectively.

Finally, 10 μM dextromethadone added to intracellular, rather than to extracellular solution, was unable to inhibit 10/10 μM L-glutamate/glycine induced current.

The following lists of abbreviations on the data are used in this Example.

| Abbreviation | Definition or Expanded Term |
|---|---|
| CHO | Chinese hamster ovary |
| Gly | Glycine |
| GLP | Good laboratory practice |
| L-glu | L-glutamate |
| MW | Molecular weight |
| NA | Not available |
| NMDA | N-methyl-D-aspartate |
| NMDAR | N-methyl-D-aspartate receptor |
| CHO | Chinese hamster ovary |
| Gly | Glycine |
| OECD | Organisation for economic co-operation and development |
| QC | Quality control |
| s | seconds |
| SEM | Standard error of the mean |
| SOP | Standard operating procedure |

An electrophysiology manual patch clamp methodology was used to set up on-/off-rate assay for dextromethadone and (±)-ketamine. Test item onset and offset kinetic were investigated relative to the block of 10/10 μM L-glutamate/glycine induced whole-cell current in GluN1/GluN2C NMDAR cell line.

Dextromethadone intracellular application effect was also evaluated.

Test Items are shown in Table 45, below.

TABLE 45

| Name | MW | Supplier | Code | CAS |
|---|---|---|---|---|
| Dextromethadone | 345.91 | Padova | NA | 5653-80-5 |
| hydrochloride | | University | | (base) |
| (±)-Ketamine | 274.19 | Merck Sigma- | K2753 | 1867-66-9 |
| hydrochloride | | Aldrich | | |
| L-Glutamate | 187.1 | Merck Sigma- | G1626 | 142-47-2 |
| | | Aldrich | | (anhydrous) |
| Glycine | 75.07 | Merck Sigma- | G7403 | 56-40-6 |
| | | Aldrich | | |

Test items were dissolved in H₂O at suitable concentration, and then stored at −20° C. until use.

Stock concentrations were: 100 mM=10 mg/289 μl for dextromethadone; 100 mM=10 mg/365 μl for (±)-ketamine; 1 M=100 mg/534 μl for L-glutamic acid; 1 M=100 mg/1332 μl for glycine.

C. Test System

Test items were evaluated using a manual patch clamp whole-cell recording methodology, using HEKA Elektronik Patchmaster system, coupled to BioLogic RSC-160 perfusion device (BioLogic, Seyssinet-Pariset, France). CHO cell line expressing diheteromeric human GluN1/GluN2C NMDA receptor was used in this study.

D. Experimental Design

On-/off-rates of dextromethadone and (±)-ketamine were measured by electrophysiology manual patch procedure as described in Mealing G A et al, 2001, using NMDAR cell line expressing hGluN1/hGluN2C diheteromeric receptor.

The ability of dextromethadone to block hGluN1/hGluN2C receptor was also evaluated when added intracellularly.

E. Methods and Procedures

hGluN1/hGluN2C-CHO cells grown on poly-D-lysine coated glass coverslips were studied by manual patch clamp whole cell recording. Extracellular and intracellular solutions for patch clamp recording had following composition:

(1) Intracellular solution (in mM): 80 CsF, 50 CsCl, 0.5 CaCl₂), 10 HEPES, 11 EGTA, adjusted to pH 7.25 with CsOH; and
(2) Extracellular solution (in mM): 155 NaCl, 3 KCl, 1.5 CaCl₂), 10 HEPES, 10 D-glucose adjusted to pH 7.4 with NaOH.

Recordings occurred at −70 mV fixed voltage equal to holding potential.

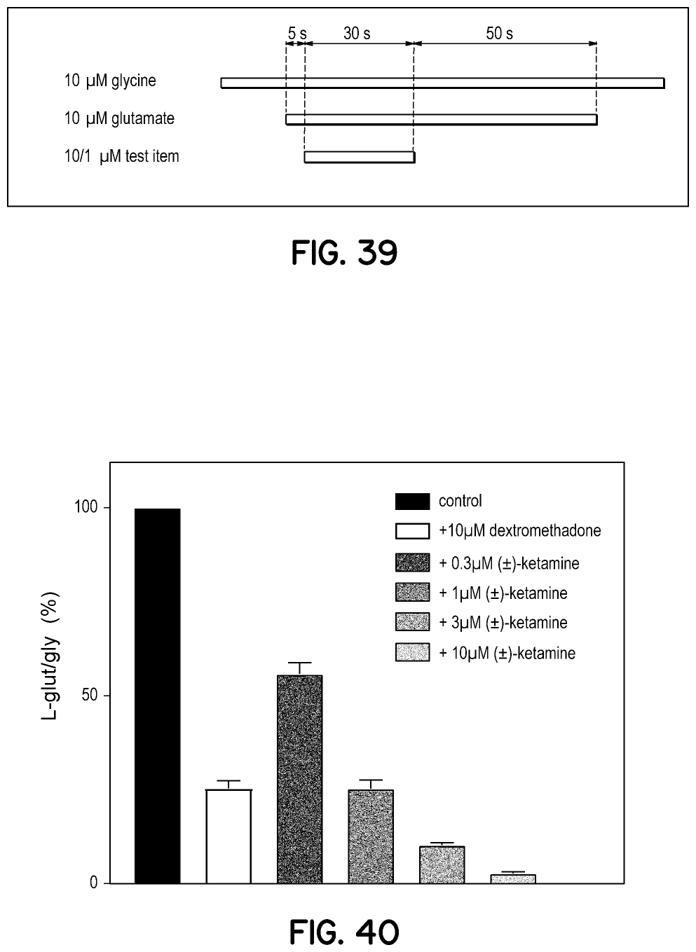

hGluN1/hGluN2C-CHO cells were exposed for 5 s to 10/10 μM L-glutamate/glycine, followed by a 30-s co-application of L-glutamate/glycine plus test item and a 50 s re-exposure to L-glutamate/glycine, as sketched in FIG. 39.

Test item on-/off-rates were measured by curve fitting the development of their induced current block, or relief from it.

F. Data Handling and Analysis

At least n=10 independent cells were analysed. For each cell, the current in the presence of 10 μM glycine only was set as 0%, while the steady state current induced, after 5 seconds application, by 10 μM L-glutamate and 10 μM glycine was set as 100%. Time constant of onset (tau-on, seconds) and offset (tau-off, seconds) of test item inhibition of glutamate induced current were calculated using first order exponential equations as shown below:

First order equation for test item onset:

I(t)=I₁+(I₀−I₁)×e^{-t/τon}

First order equation for test item offset:

I(t)=I₁+(I₂−I₁)×(1−e^{-t/τoff})

where I(t) is current at time t; t is time (seconds) after test item application or removal, in onset or offset equation, respectively; l₀ is current after 5 seconds application of 10 μM L-glutamate and 10 μM glycine and before test item application; Ii is current after 30 seconds application of test item, in presence of 10 μM L-glutamate and 10 μM glycine; l₂ is current after 50 seconds removal of test item, in continuous presence of 10 μM L-glutamate and 10 μM glycine; τon (aka tau-on) is time constant (seconds) of onset; and, τoff (aka tau-off) is time constant (seconds) of offset.

G. Results

1. Test Item % Current Block

The block produced by 10 μM dextromethadone was initially determined. 10 μM dextromethadone produced a block of 10/10 μM L-glutamate/glycine induced current in hGluN1/hGluN2C-CHO cells of 74.6%±1.9% (n=12). Then, (±)-ketamine effect was studied and resulted in a block of 97.2%±0.3% (n=3), 89.7%±0.6% (n=3), 74.6%±2.2% (n=3) and 44.2%±3.0% (n=7) at 10, 3, 1, and 0.3 μM, respectively. A graph of the residual % current in presence of 10 μM dextromethadone or various concentrations of (±)-ketamine, and relative data table, are reported in FIG. 40. The same data reported in the graph of FIG. 40 are tabulated in Table 46, below:

TABLE 46

| | % Current | |
|---|---|---|
| Test item | (mean ± SEM) | N |
| Control | 100 | 28 |
| 10 μM dextromethadone | 74.6 ± 1.9 | 12 |
| 10 μM (±)-ketamine | 97.2 ± 0.3 | 3 |
| 3 μM (±)-ketamine | 89.7 ± 0.6 | 3 |
| 1 μM (±)-ketamine | 74.6 ± 2.2 | 3 |
| 0.3 μM (±)-ketamine | 44.2 ± 3.0 | 7 |

Control current (100%) was induced by 10/10 μM L-glutamate/glycine and resulted of −594.2±103.7 pA (mean±SEM, n=28).

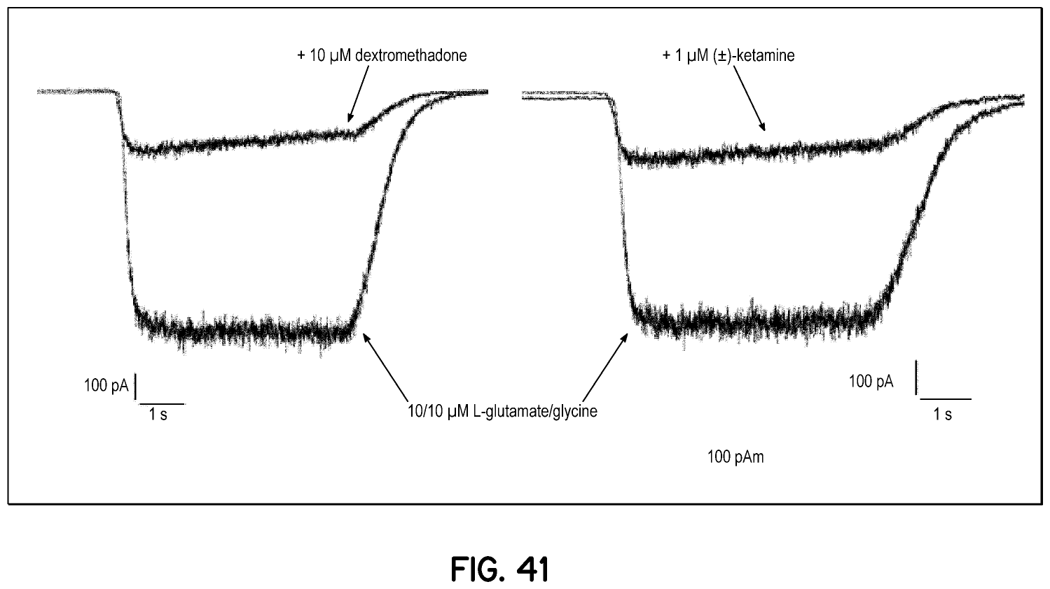

Sample traces of hGluN1/hGluN2C-CHO cells added with 10/10 μM L-glutamate/glycine alone or in combination with 10 μM dextromethadone or 1 μM (±)-ketamine are reported in FIG. 41.

2. Test Item Onset and Offset Kinetic

Since test item concentrations eliciting similar % block are to be used to produce comparable kinetic data (Mealing et al, 2001), then 10 μM dextromethadone and 1 μM (±)-ketamine were tested in tau-on and tau-off experiments.

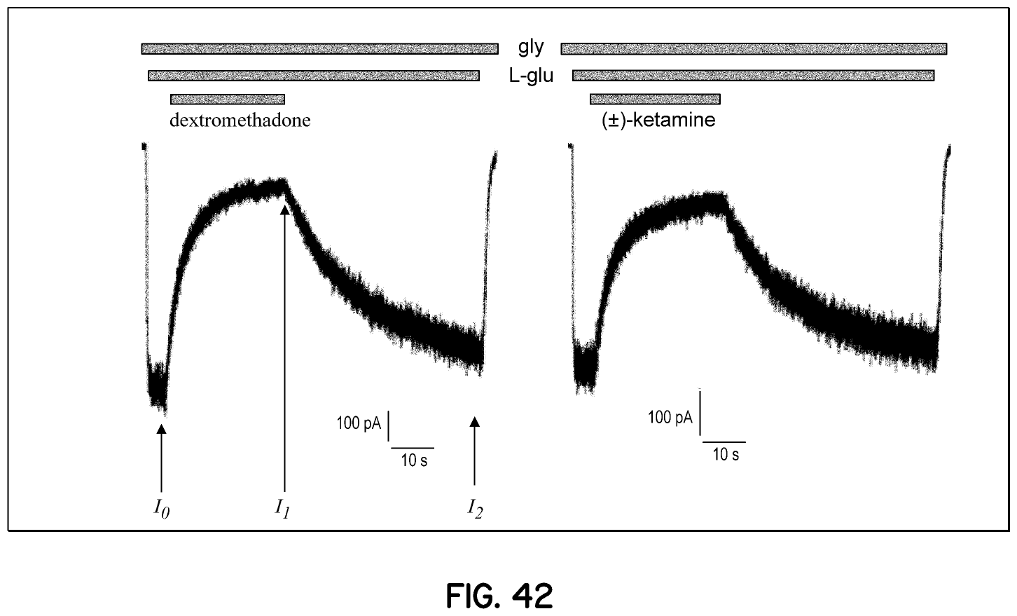

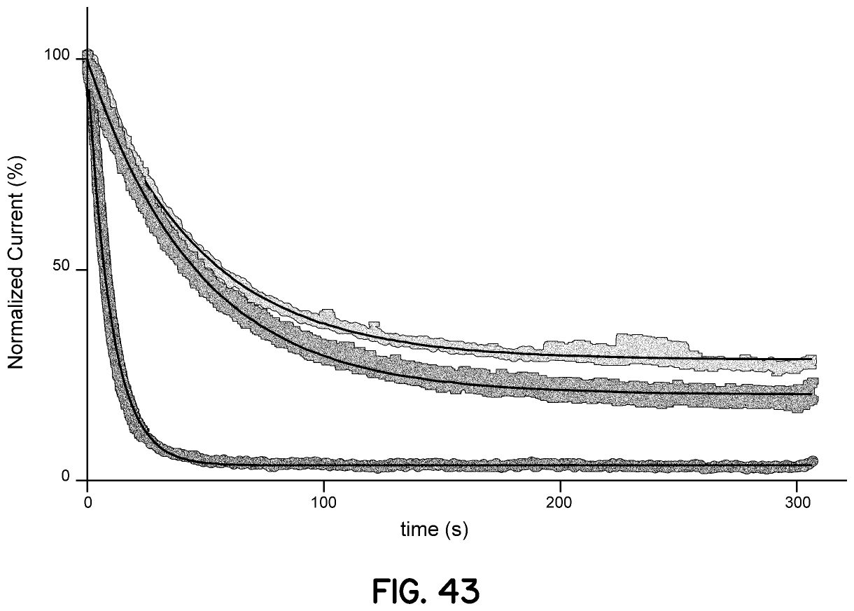

Typical traces obtained with test items in kinetic experiments are reported in FIG. 42. 10 μM dextromethadone resulted with 46.4 and 173.5 s, tau-on and tau-off, respectively. 1 μM (±)-ketamine resulted with 47.1 and 151.0 s, tau-on and tau-off, respectively. Time course of averaged % current following test item addition, in continuous presence of 10/10 μM L-glutamate/glycine, used for onset parameter estimation, is reported in FIG. 43 together with the comparison and statistical analysis of 10 μM dextromethadone and 1 μM (±)-ketamine effect, performed on the average tau values derived from single trace fittings (46.7±2.1 s and 47.3±1.4 s for 10 μM dextromethadone and 1 μM (±)-ketamine, respectively).

In FIG. 43, traces represent % current recorded for 10 μM dextromethadone (middle line; grey shading), 10 μM (±)-ketamine (bottom line; black shading), and 1 μM (±)-ketamine (top line; light grey shading), while internal black lines are relative fittings.

The following equation was used for fitting:

I(t)=I₁+(I₀−I₁)×e^{-t/τon}

Fittings data results are reported in Table 47, below:

TABLE 47

| | Tau-on | I₀ (% | I₁ (% | |
|---|---|---|---|---|
| Test item | (s) | current) | current) | N |
| 10 μM dextromethadone | 6.4 | 100 | 20.4 | 11 |
| | | (constrained) | | |
| 1 μM (±)-ketamine | 47.1 | 100 | 28.7 | 10 |
| | | (constrained) | | |
| 10 μM (±)-ketamine | 9.9 | 100 | 3.6 | 4 |
| | | (constrained) | | |

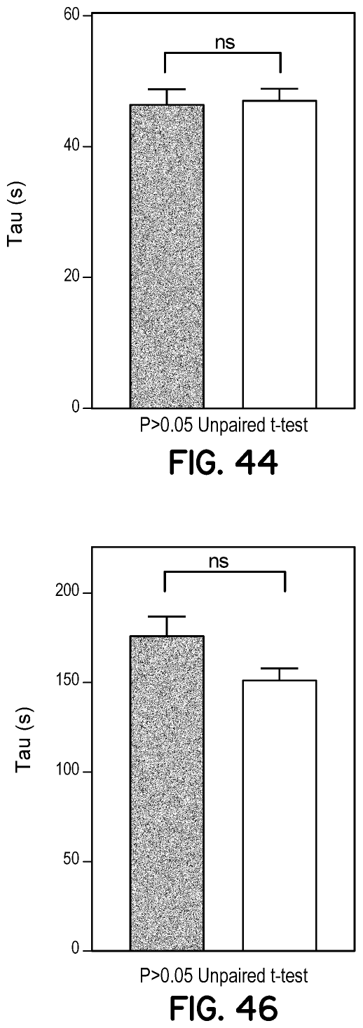

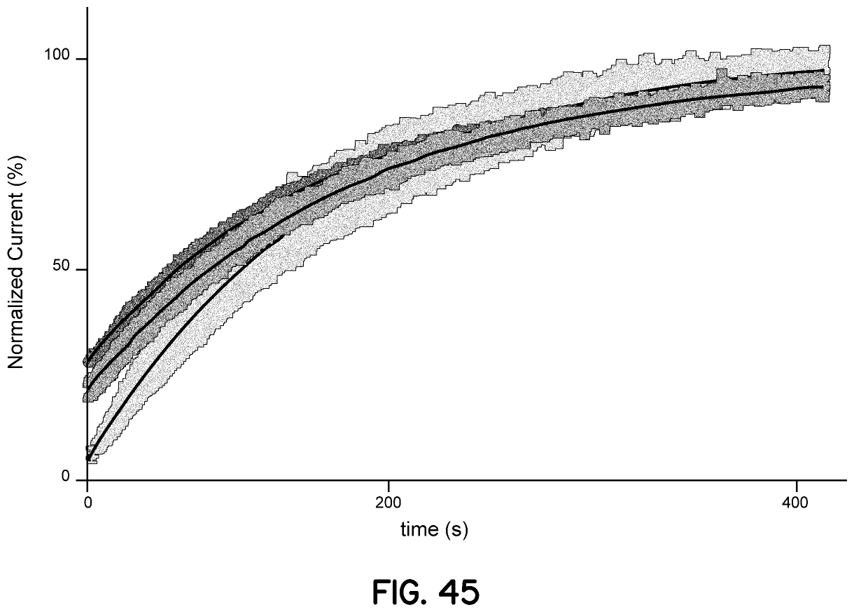

FIG. 44 then shows a comparison of the tau-on of 10 μM dextromethadone (left column) and 1 μM (±)-ketamine (right column) experiments: Time course of averaged % current following test item removal, in continuous presence of 10/10 μM L-glutamate/glycine, used for offset parameter estimation, is reported in FIG. 45 together with the comparison and statistical analysis of 10 μM dextromethadone and 1 μM (±)-ketamine effect, performed on the mean tau values derived from single trace fittings (176.5±10.5 s and 151.7±6.3 s for 10 μM dextromethadone and 1 μM (±)-ketamine, respectively). In FIG. 45, traces represent % current recorded for 10 μM dextromethadone (grey shading), 1 μM (±)-ketamine (black shading) and 10 μM (±)-ketamine (light grey shading), while internal black lines are relative fittings.

The following equation was used for fitting:

I(t)=I₁+(I₂−I₁)×(1−e^{-t/τoff})

and fittings data results are reported in Table 48, below.

TABLE 48

| | Tau-off | I₁ (% | I₂ (% | |
|---|---|---|---|---|
| Test item | (s) | current) | current) | N |
| 10 μM | 173.5 | 21.7 | 98.5 | 11 |
| dextromethadone | | | | |
| 1 μM (±)-ketamine | 151.0 | 28.5 | 95.5 | 10 |
| 10 μM (±)-ketamine | 163.2 | 4.9 | 102.9 | 4 |

Comparison of the Tau-off of 10 μM dextromethadone (left column of FIG. 46) and 1 μM (±)-ketamine (right column of FIG. 46) experiments is shown in FIG. 46.

To verify that recorded slow test item kinetic effect was not due to experimental constraints, then 10 μM (±)-ketamine effect on onset kinetic was also tested. 10 μM (±)-ketamine resulted with tau-on of 9.9 s, showing that a fast kinetic could be recorded by the experimental set-up whereas tau-off resulted 163.2 s.

3. Dextromethadone Intracellular Application

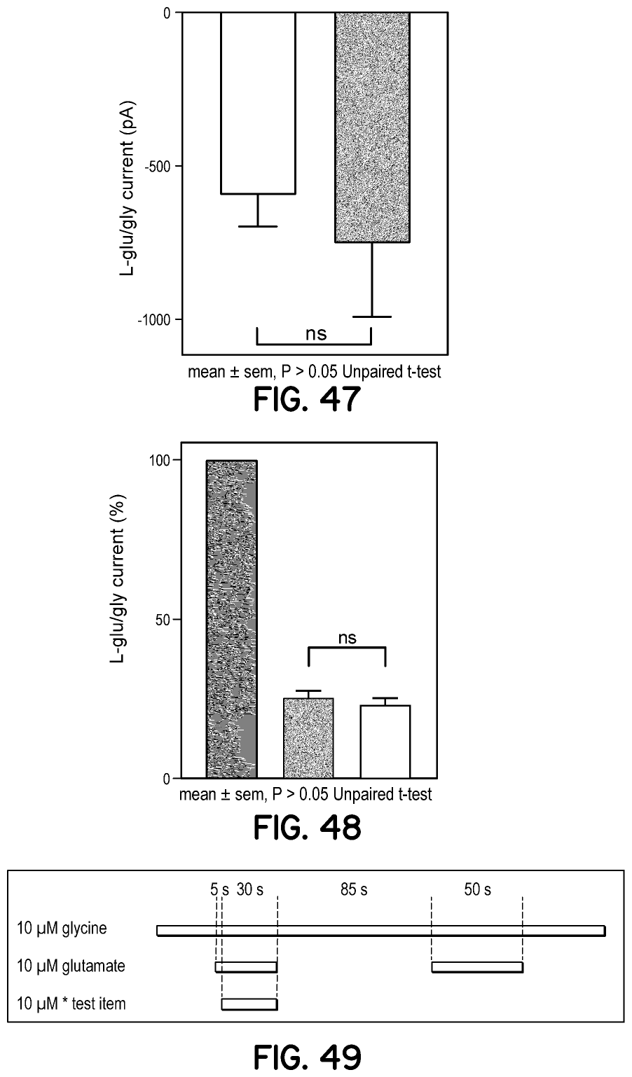

With the aim of evaluating a possible intracellular effect of dextromethadone, 10 μM test item was added to the intracellular solution and the 10/10 μM L-glutamate/glycine induced current in such condition compared to control. The amplitude of current in the presence of intracellular 10 μM dextromethadone was −752.1±240.5 (n=7) pA compared to −647.5±215.5 (n=12) pA in control condition. The difference of these two values is not significant (P>0.05, unpaired t-test). As further evidence, the amount of inhibition of 10/10 μM L-glutamate/glycine induced current by 10 μM dextromethadone is not increased in the presence of intracellular 10 μM test item. Both experiments are reported in FIGS. 47 and 48, with FIG. 47 showing that intracellular dextromethadone did not modify 10/10 μM L-glutamate/glycine induced current, and FIG. 48 showing that intracellular dextromethadone did not increase current block by extracellular dextromethadone. More specifically, FIG. 47 is a graph of the 10/10 μM L-glutamate/glycine induced current in control condition (left column, n=12) and in the presence of 10 μM intracellular dextromethadone (right column, n=7). And FIG. 48 is a graph of the effect of 10 μM dextromethadone normalized with respect to 10/10 μM L-glutamate/glycine induced current in the presence (center column, n=12) and absence (right column, n=7) of 10 μM intracellular dextromethadone.

H. Discussion

10 μM dextromethadone and 1 μM (±)-ketamine elicited similar % inhibition of 10/10 μM L-glutamate/glycine elicited current in hGluN1/hGluN2C-CHO cells. This result is in line with previous FLIPR study (Example 1) showing a nearly 10-fold higher potency of (±)-ketamine with respect to dextromethadone on hGluN1/hGluN2C NMDAR.

Onset kinetic of the two test items produced very similar results when comparing test item concentrations inducing similar % block, that was 10 and 1 μM for dextromethadone and (±)-ketamine, respectively. Indeed, tau-on were 46.4 and 47.1 s for 10 μM dextromethadone and 1 μM (±)-ketamine, respectively. 10 μM (±)-ketamine tau-on was 9.9 s, as expected since tau-on is concentration dependent, unlike tau-off.

Also offset kinetic of 10 μM dextromethadone (173.5 s) produced similar results to 1 and 10 μM (±)-ketamine (151.0 and 163.2 s, respectively).

Recorded data suggest that 10-fold higher potency of (±)-ketamine with respect to dextromethadone is due to (±)-ketamine faster onset kinetic when tested at same dextromethadone concentration, with no significantly different offset kinetic.

I. Conclusions

10 μM dextromethadone and 10 μM (±)-ketamine induced a block of 74.6% and 97.2%, respectively of hGluN1/hGluN2C receptor. 3, 1 and 0.3 μM (±)-ketamine blocked 89.7, 74.6 and 44.2%, respectively.

10 μM dextromethadone blocking and unblocking tau-on and tau-off parameters resulted 46.4 s and 173.5 s, respectively. Similarly, 1 μM (±)-ketamine blocking and unblocking tau-on and tau-off parameters resulted 47.1 s and 151.0 s, respectively.

10 μM (±)-ketamine tau-on and tau-off parameters resulted 9.9 and 163.2 s, respectively.

Intracellular 10 μM dextromethadone did not show blockade of 10/10 μM L-glutamate/glycine induced current.

Part II: Electrophysiology Trapping Assay on GluN1-Glu-2C NMDAR

A. Overview

In this Part II of Example 6, dextromethadone and (±)ketamine were evaluated in manual patch clamp, to assess their trapping level on recombinant diheteromeric human NMDAR containing GluN2C subunit.

1. Methods

Manual patch clamp recordings occurred at −70 mV. Test item trapping was determined by exposing hGluN1/hGluN2C-CHO cells to 10/10 μM L-glutamate/glycine for 5 s, followed by a 30-s co-application of L-glutamate/glycine plus test item, then by 85 s application of glycine only, and finally 50 s re-exposure to L-glutamate/glycine.

2. Results

Dextromethadone and (±)-ketamine showed 85.9%±1.9% (n=13) and 86.7%±1.8% (n=11) trapping, respectively, on GluN1/GluN2C receptor.

3. Conclusions

Dextromethadone and ketamine showed similar trapping in the present inventors' experimental conditions, which may be relevant to their reported efficacy as antidepressant drugs (to the isolated symptom of depression). Interestingly, memantine, another NMDAR antagonist more potent than ketamine and dextromethadone but with reported low trapping, is FDA approved for the treatment of late stage dementia but was reported to be devoid of antidepressant effect. The present inventors' results suggest that high trapping may be desirable for therapeutic efficacy of NMDAR channel blockers in MDD.

B. Summary

An electrophysiology assay was designed to establish test item trapping, relative to the block of 10/10 μM L-glutamate/glycine induced whole-cell current in GluN1-GluN2C NMDAR cell line.

Dextromethadone and (±)-ketamine were selected as test items.

The dextromethadone trapping result was 85.9%.

The (±)-ketamine trapping result was 86.7%.

Electrophysiology manual patch clamp methodology was used to set up trapping assay for dextromethadone and (±)-ketamine. Test item trapping was investigated relative to the block of 10/10 μM L-glutamate/glycine induced whole-cell current in GluN1-GluN2C NMDAR cell line.

Test Items are shown in Table 49, below.

TABLE 49

| Name | MW | Supplier | Code | CAS |
|---|---|---|---|---|
| Dextromethadone | 345.91 | Padova | | 5653-80-5 |
| hydrochloride | | University | | (base) |
| (±)-Ketamine | 274.19 | Merck Sigma- | K2753 | 1867-66-9 |
| hydrochloride | | Aldrich | | |
| L-Glutamate | 187.1 | Merck Sigma- | G1626 | 142-47-2 |
| | | Aldrich | | (anhydrous) |
| Glycine | 75.07 | Merck Sigma- | G7403 | 56-40-6 |
| | | Aldrich | | |

Test items were dissolved in H₂O at suitable concentration, and then stored at −20° C. till use.

Stock concentrations were: 100 mM=10 mg/289 μl for dextromethadone; 100 mM=10 mg/365 μl for (±)-ketamine; 1 M=100 mg/534 μl for L-glutamic acid; 1 M=100 mg/1332 μl for glycine.

C. Test System

Test items were evaluated using manual patch clamp whole-cell recording methodology, using HEKA Elektronik Patchmaster system coupled to BioLogic RSC-160 perfusion device (BioLogic, Seyssinet-Pariset, France), as detailed in protocol of Part I of this Example 1. CHO cell line expressing diheteromeric human GluN1-GluN2C NMDA receptor was used in this study.

D. Experimental Design

The aim of Part II of this Example 6 was to evaluate the trapping of dextromethadone and (±)-ketamine, at concentrations eliciting similar % current blockade on GluN1-GluN2C receptor.

10 μM dextromethadone and 1 μM (±)-ketamine were selected as test item concentrations, based on results reported in Part I of this Example 6.

E. Methods and Procedures

hGluN1/hGluN2C-CHO cells grown on poly-D-lysine coated glass coverslips were studied by manual patch clamp whole cell recording. Extracellular and intracellular solutions for patch clamp recording had following compositions: (1) Intracellular solution (in mM): 80 CsF, 50 CsCl, 0.5 CaCl₂), 10 HEPES, 11 EGTA, adjusted to pH 7.25 with CsOH; and (2) Extracellular solution (in mM): 155 NaCl, 3 KCl, 1.5 CaCl₂), 10 HEPES, 10 D-glucose adjusted to pH 7.4 with NaOH.

Recordings occurred at −70 mV fixed voltage equal to holding potential.

Trapping of the initial block was measured using the appropriate concentration of test item, as described by Mealing et al 2001. Test item trapping was determined by exposing hGluN1/hGluN2C-CHO cells to 10/10 μM L-glutamate/glycine for 5 s, followed by a 30-s co-application of L-glutamate/glycine plus test item, then by 85 s application of glycine only, and finally 50 s re-exposure to L-glutamate/glycine. A diagram of test item application protocol is sketched in FIG. 49.

F. Data Handling and Analysis

The block of 10/10 μM L-glutamate/glycine-evoked currents was calculated according to the formula:

B=[(I−I_{B})/I]×100 (1)

where I was be determined as the current value derived from a linear extrapolation to the end of the L-glutamate antagonist co-application, and I_{B} was the current measured at the end of L-glutamate/blocker co-application.

The residual block of L-glutamate-evoked currents was calculated according to the formula:

B_{R}=[(I₁ₛₜ−I_{2nd})/I₁ₛₜ]×100 (2)

where I₁ₛₜ was the maximal current measured during 1 s after onset of the first L-glutamate exposure and I_{2nd} was the maximal current measured during 1 s after onset of the delayed second L-glutamate exposure after washout of blocker from the bath.

The block trapped (B_{T}), or the amount of block remaining at the beginning of the second L-glutamate application as a percent of the initial block produced at the end of the previous L-glutamate/antagonist co-application, was calculated according to the formula:

B_{T}=B_{R}/B×100 (3)

where B and B_{R} were defined as above.

Data were expressed as means±S.E.M. (n≥10 number of cells).

G. Protocol Deviations

I value in equation (1) was determined from a linear extrapolation rather than a first order exponential curve to the end of the L-glutamate antagonist co-application.

I₁ₛₜ and I_{2nd} in equation (2) were measured 1000±100 ms, rather than 200±25 ms after onset of the first or second L-glutamate exposure as reported in Protocol for Example 6, since the present inventors' hGluN1-hGluN2C response onset to L-glutamate was sensibly slower than what reported by Mealing et. al. 2001 in cultured rat cortical neurons.

H. Results

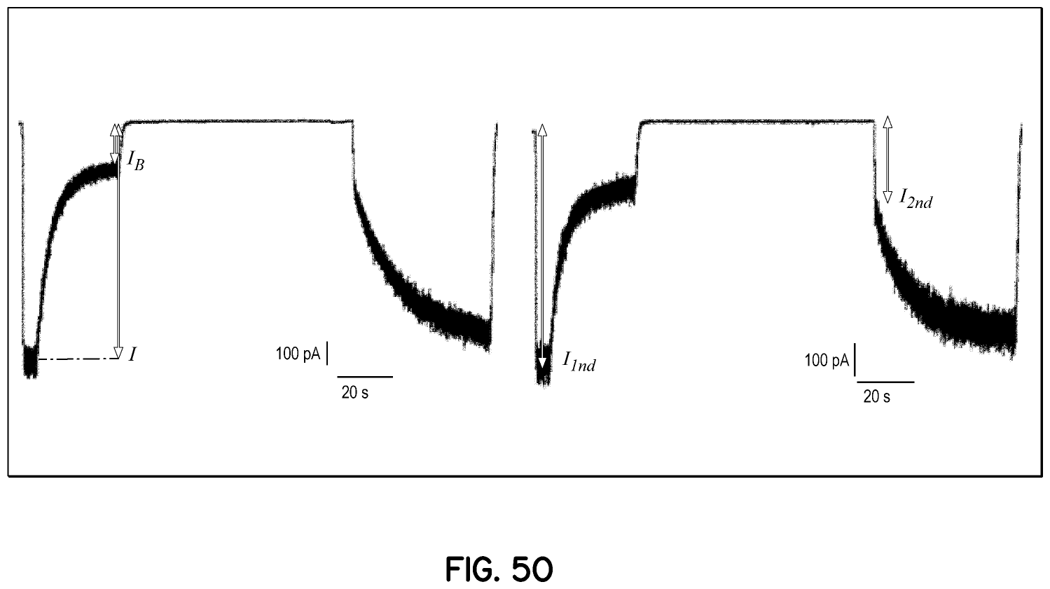

FIG. 50 shows the representative traces obtained in trapping assay experiments in response to the indicated applications of test items.

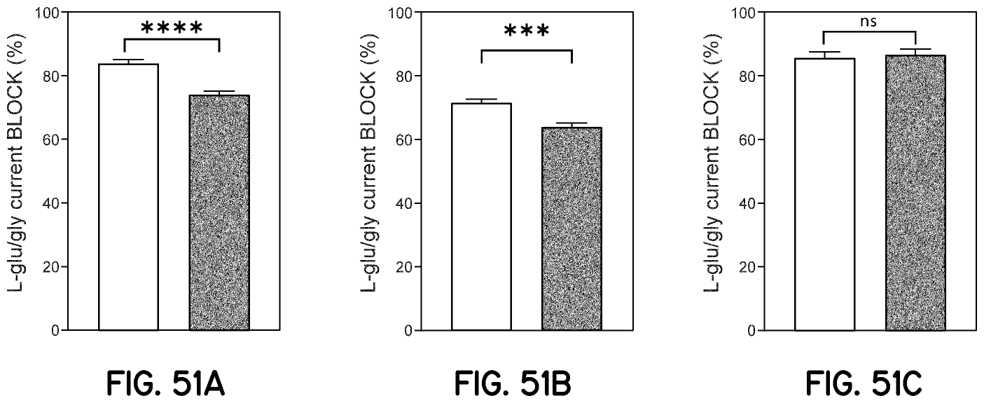

As shown in FIGS. 51A-51C (left), the block of 10/10 μM L-glutamate/glycine-evoked currents produced by 10 μM dextromethadone was 83.8%±1.2% with respect to control current [equation (1)], extrapolated to the end of L-glutamate co-application with antagonist. The block observed in the presence of 1 μM (±)-ketamine was 74.0%±1.2%. The two figures were statistically different.

A statistically significant difference was also obtained for the residual block, calculated using equation (2), resulting to be 71.8%±1.1% and 64.1%±1.3% for 10 μM dextromethadone and 1 μM (±)-ketamine, respectively, as shown in FIG. 51B.

The block trapped, obtained from equation (3), was 85.9%±1.9% and 86.7%±1.8% for 10 μM dextromethadone and 1 μM (±)-ketamine, respectively (right). The amount of this effect has to be considered equivalent for the two test items.

I. DISCUSSION

(±)-Ketamine showed 86.7% trapping, in the present inventors' experimental conditions on GluN1/GluN2C receptor, in optimal agreement with 86.0% value reported by Mealing G A, Lanthorn T H, Murray C L, Small D L, Morley P. Differences in degree of trapping of low-affinity uncompetitive N-methyl-D-aspartic acid receptor antagonists with similar kinetics of block. J Pharmacol Exp Ther. 1999; 288(1):204-210, using cultured rat cortical neurons.

The present inventors also obtained a similar 85.9% trapped block value for dextromethadone on GluN1/GluN2C receptor.

Trapped antagonist have been suggested to produce NMDAR tonic block (Mealing et al, 2001). NMDAR tonic block might be functional to ambient glutamate inhibition, which in turn might be relevant for NMDAR blocker antidepressant effect.

Safer dextromethadone profile respect to ketamine cannot be explained in terms of differential trapping on GluN1/GluN2C receptor. Instead, it is likely that lower dextromethadone potency at different subtypes, including GluN2C and GluN2D, might determine lower level of NMDAR tonic block than ketamine, at similar free brain concentrations, considering both blockers are trapped in NMDARs at similar level.

J. Conclusions

10 μM dextromethadone and 1 μM (±)-ketamine induced a block of 83.8 and 74.0%, respectively of hGluN1/hGluN2C receptor.

The residual block was 71.8 and 64.1% for 10 μM dextromethadone and 1 μM (±)-ketamine, respectively.

Consequently, the block trapped was 85.9 and 86.7% for 10 μM dextromethadone and 1 μM (±)-ketamine, respectively.

Part III: Dextromethadone Automated Electrophysiology Study in Presence of Magnesium

A. Background

In physiological conditions, NMDAR pore is blocked by extracellular magnesium. The present inventors therefore attempted to characterize dextromethadone blockade of diheteromeric human NMDAR in presence of extracellular magnesium and at different membrane potentials.

B. Methods

Automated patch clamp experiments were performed in QPatch HTX (Sophion Bioscience A/S, Ballerup, Denmark) using CHO cells stably expressing recombinant diheteromeric human NMDAR. Cells were clamped at −80 mV holding potential in presence of 1 mM extracellular magnesium. Voltage protocol included a depolarizing 2 s step pulse to +60 mV, to check quality of the seal and cell NMDAR expression level, followed by a 2 s ramp back to holding potential. L-glutamate induced currents were measured at different voltages during the protocol, in absence or in presence of 10 μM dextromethadone.

C. Results

10 μM dextromethadone effect was studied on 10 μM or 1 μM L-glutamate induced currents. GluN1/GluN2D receptor resulted as the human diheteromeric NMDAR more sensitive to dextromethadone blockade: 10 μM or 1 μM L-glutamate elicited current was significantly reduced by dextromethadone at all measured negative voltages, ranging from −30 mV to −80 mV. In particular, residual current in presence of 1 μM L-glutamate at −80 mV after dextromethadone application resulted 62.5±4.1% (n=4) of pre-application level, while in control cells the value was 102.5±3.9% (n=4). The block exerted by dextromethadone was voltage dependent, similarly to the block exerted by magnesium.

D. Conclusions

Dextromethadone preferentially reduced L-glutamate currents at GluN1/GluN2D receptor in presence of 1 mM extracellular magnesium, which suggests dextromethadone action at NMDAR activated by ambient L-glutamate and potential sparing of phasically activated NMDAR.

### Example 7—Biomarkers

A. Background

As has been discussed above, dextromethadone increases BDNF in healthy subjects. In this Example 7, the present inventors postulate that an analysis of BDNF and additional biomarkers may add to the results outlined throughout this application disclosing dextromethadone as a disease-modifying treatment. Notably, BDNF was not enhanced by dextromethadone in the MDD patients discussed herein, therefore BDNF plasma levels are unlikely to be a reliable marker of dextromethadone effects in MDD. However, dextromethadone, by showing higher efficacy in patients with higher levels of inflammatory biomarkers, may exert disease-modifying effects on these patients and not only symptomatic effects (symptomatic effects are not generally specific for patients with certain disease biomarkers but are seen across different patient populations sharing the same symptoms but not necessarily the same disease, and the same pathophysiology for said disease).

B. BMI, Biomarkers and Therapeutic Effects of Dextromethadone

1. Methods

In this experiment, patients were divided into the following populations according to their BMI (under 30=non obese; equal or above 30=obese): Population 1: non obese (39 patients) and Population 2: obese (21 patients).

2. Summary of Results from Day 1 Pre-Treatment Baseline Levels:

A general decreasing tendency of the measured biomarkers could be observed in obese patients with respect to non-obese patients (i.e., in patients with a diagnosis of MDD, higher levels of inflammatory markers were observed in non-obese patients compared to obese patients). Statistically significant differences could be evidenced between non obese and obese patients: (1) GM-CSF *p-value 0.024 (57.129±75.891 vs 4.673±12.943 in non-obese and obese patients, respectively); (2) IL-2 **p-value 0.004 (6.882±9.602 vs 2.086±1.932 in non-obese and obese patients, respectively); and (3) IL-7 **p-value 0.004 (1.359±1.382 vs 0.628±0.481 in non-obese and obese patients, respectively).

Other inflammatory cytokines (IL-13, IL-4, IL-6, MIP-1a, TNF-a) were close to statistical significance in the two groups, again with higher levels in non-obese patients.

The above results, when correlated with the lack of response shown by obese patients (see Tables 32-34), indicate that dextromethadone, by showing higher efficacy in patients with higher levels of inflammatory biomarkers, may exert disease-modifying effects on these patients and not only symptomatic effects (symptomatic effects are not generally specific for patients with certain disease biomarkers but are seen across different patient populations sharing the same symptoms but not necessarily the same disease).

It is generally accepted by those skilled in the art that the effects of purely symptomatic drugs for the treatment of chronic conditions tend to rapidly decrease in magnitude or abruptly cease after discontinuation of the drug (especially after abrupt discontinuation, as was the case in the dextromethadone Phase 2 clinical trial disclosed in this application by the inventors, Example 3). The abrupt discontinuation of symptomatic drugs may even determine a phenomenon of augmentation or rebound of symptoms (worsening of symptoms compared to pre-treatment baseline). An example of symptomatic treatment is morphine for the treatment of pain, e.g., morphine for the treatment of post-operative pain. If morphine is stopped while the post-surgical inflammatory state is still active, the pain will resume within a couple of hours.

On the other hand, improvements caused by disease-modifying treatments, including improvement in symptoms, tend to persist upon completion of the treatment cycle, e.g., immunotherapy for cancer, for multiple sclerosis, or for rheumatoid arthritis, even after discontinuation of treatment. If the immunotherapy cycle is adequate, the patient's symptoms, e.g., pain and inflammation at disease sites, generalized malaise, etc., will generally not recur upon abrupt discontinuation of treatment, as was the case in the patients described in Example 3.

The fact that the remission induced by dextromethadone in patients with MDD unexpectedly persisted after discontinuation of treatment signals that the action of dextromethadone is not purely symptomatic, i.e., dextromethadone does not simply symptomatically lift the mood of patients by binding to certain receptors, an effect that would cease upon discontinuation of the drug and unbinding of receptors, as may happen for example with the use of opioids or even alcohol in subjects with depressed mood. The sustained remission induced by dextromethadone in patients with MDD (as determined by improvements on multiple dimensions of MADRS and other scales, and thus not limited to an improvement in depression as an isolated symptom) signals that the effects of dextromethadone are likely secondary to disease-modifying effects, including neural plasticity mechanisms first proven clinically in the Phase 2a trial discussed above in Example 3 (e.g., neuroplasticity mechanisms that may be related to the synthesis of new NMDAR channels) see also, e.g., Example 2.

The new experiments in vivo (rats) and in vitro (below in Example 11) also suggest dextromethadone effects may modulate inflammatory biomarkers that may be increased in MDD. The plasma analyses from MDD patients treated with dextromethadone further confirm its disease-modifying effects in neuropsychiatric diseases, including MDD. Finally, with symptomatic treatments that alleviate symptoms via binding to receptors, a higher dose is expected by those skilled in the art to exert more powerful effects, because more receptor binding will occur with higher plasma levels of the drug.

Unexpectedly, this was not the case in the present inventors' Phase 2a trial where the lower dose (25 mg) appeared to work just as well or better compared to the higher (double) dose of 50 mg. The higher dose resulted in approximately double plasma levels and a trend towards more side effects, but did not improve efficacy over that seen with 25 mg. The unexpected observation of a 25 mg “therapeutic ceiling effect” for dextromethadone in MDD again signals a disease-modifying effect, as seen for example with immunotherapy for cancer, for multiple sclerosis, or for rheumatoid arthritis—disease states where doubling the dose of a disease-modifying treatment does not necessarily result in improved efficacy in individual patients or an increase in the percentage of patients cured. The higher dose, above the “ceiling effect,” may however increase side effects, depending on the safety and tolerability profile of the drug. In the case of dextromethadone, this increase in side effects was present, but its clinical meaningfulness was low, if any, because the safety window for dextromethadone is large. On the other hand, in the case of symptomatic treatments, e.g., opioid treatment for acute pain, doubling the morphine dose will generally result in better pain control, although usually at the cost of more severe side effects. The present inventors herein have disclosed that an even lower dose of dextromethadone (e.g., less than 25 mg per day, e.g., 0.1-24 mg) administered daily, or even intermittently, could effectively treat MDD in a subset of patients not responding to higher doses. Additionally, it is possible that a higher dose of dextromethadone, e.g., doses titrated up to 1000 mg per day, could benefit a subset of patients in the 25 or 50 mg group that did not improve (e.g., obese patients).

Furthermore, drugs acting directly on neurotransmitter receptors, such as benzodiazepines, opioids and dopamine antagonists, or on their pathways, including transporter pathways, e.g., SSRIs, appear to exert their effects by influencing specific neurotransmitter pathways, and their effects abruptly cease or even rebound when these drugs are discontinued. A persistence of therapeutic effects for a full week after discontinuation of treatment, especially in the absence of withdrawal effects, as seen in the Phase 2a study patients treated with dextromethadone, strongly signals disease-modifying effects via neural plasticity mechanisms. Furthermore, the persistence of effects also signals potential efficacy of intermittent chronic therapy (e.g., weekly) as opposed to continuous (e.g., daily) chronic therapy.

The unexpected disease-modifying effects seen in the present inventors' Phase 2a study are postulated by the present inventors to be due to a multiplicity of mechanisms of action, including an interaction and synergy of said effects and mechanisms of action (including allosteric interactions), and these effects may be determined by the multiplicity of actions of dextromethadone at multiple receptors and pathways including NMDARs and their subtypes, nicotinic receptors (Talka et al., 2015), sigma-1 (Maneckjee R, Minna J D. Characterization of methadone receptor subtypes present in human brain and lung tissues. Life Sci. 1997; 61(22)), SET, NET, MOP, DOP, KOP (Codd et al., 1995) serotonin receptors and their subtypes, including especially 5-HT2A and 5-HT2C receptors (Rickli A, Liakoni E, Hoener M C, Liechti M E. Opioid-induced inhibition of the human 5-HT and noradrenaline transporters in vitro: link to clinical reports of serotonin syndrome. Br J Pharmacol. 2018; 175(3):532-543), and histamine receptors (Codd et al., 1995; Kristensen K, Christensen C B, Christrup L L. The mu1, mu2, delta, kappa opioid receptor binding profiles of methadone stereoisomers and morphine. Life Sci. 1995; 56(2):PL45-PL50). Finally, the effects of dextromethadone could be direct or through its metabolites EDDP and EMDP, and their isomers. Forcelli et al., 2016, (Forcelli P A, Turner J R, Lee B G, et al. Anxiolytic- and antidepressant-like effects of the methadone metabolite 2-ethyl-5-methyl-3,3-diphenyl-1-pyrroline (EMDP). Neuropharmacology. 2016; 2015.09.012), disclose methadone metabolites and particularly EMDP, for the treatment of the symptoms of anxiety and depression based on preclinical models and receptor binding data at nAChR channels, and based on the symptomatic actions of nicotine as found in tobacco products on relieving symptoms of anxiety and depression.

Based on the present inventors' data disclosed above, and their data on NMDAR docking results presented below in Example 8, the present inventors disclose that metabolites of methadone, including those presented in Example 8 may be effective not only for the treatment of symptoms but may be effective as disease-modifying treatments for neuropsychiatric diseases and disorders and other diseases and disorders disclosed in this application and triggered, maintained or worsened by excessive Ca²⁺ influx. These disease-modifying effects are a reflection of neural plasticity induced by dextromethadone.

The current understanding is that dextromethadone acts predominantly as an NMDAR open channel uncompetitive blocker with favorable PD profile (as shown in the Examples herein) and that the channel blocking action at NMDARs causes modulation of hyperactive channels (NMDARs are potentially pathologically hyperactive in a multiplicity of diseases and disorders). By blocking hyperactive NMDA receptors and thereby modulating calcium influx, dextromethadone treatment determines downstream neuroplasticity as demonstrated by the novel in vitro experimental findings on induction of synthesis of NMDAR protein subunits by dextromethadone (Example 2). These downstream effects of NMDAR modulation result in potential disease-modifying therapeutic benefits, both rapid and sustained, as shown by in the present inventors' Phase 2a study results in MDD.

The 5-HT2A serotonin receptor subtype 5-HT2A (and to a lesser extent, 5-HT2C) is associated with the psychedelic/psychotomimetic effects and potential therapeutic effects of serotonin receptor agonists [Halberstadt A L, Geyer M A. Multiple receptors contribute to the behavioral effects of indoleamine hallucinogens. Neuropharmacology. 2011; 61(3): 364-381]. Psychedelic drugs have now been associated with neural plasticity effects (Ly et al., 2018). Rickli et al., 2018, report that dextromethadone is a 5-HT2A agonist (Ki 520 nM) and 5-HT2C agonist (Ki 1900 nM). There is thus a new mechanism by which dextromethadone could induce neural plasticity, or alternatively there could be synergy or even overlap (allosteric interactions) between the two mechanisms (NMDAR antagonism and 5-HT2A agonism). Aside or in addition to dextromethadone positioning within the pore of the NMDAR, at the PCP site as shown by binding studies disclosed by the inventors, the present inventors postulate allosteric interactions between activated 5-HT receptors 2A and 2C and the Ca²⁺ permeable NMDAR: when 5-HT2A-C agonists (e.g., dextromethadone) bind to these receptors they result in the closure of the structurally associated NMDAR pathologically hyperactive channel.

The concentrations of racemic l,d-methadone and l-methadone required for NMDAR channel block are higher than those required to activate opioid receptors [Matsui A, Williams J T. Activation of μ-opioid receptors and block of Kir3 potassium channels and NMDA receptor conductance by L- and D-methadone in rat locus coeruleus. Br J Pharmacol. 2010; 161(6)1403-1413]: both racemic methadone and levomethadone are in clinical use for the treatment of pain and their clinical effects are dominated by powerful mu opioid effects. Dextromethadone has over 20-fold less affinity for opioid receptors compared to levomethadone (Codd et al., 1995). The concentrations of dextromethadone that are therapeutic in patients with MDD are sufficient to exert NMDAR block (low micro molar range, Gorman et al., 1997) and may also mediate neural plasticity effects induced by 5-HT2A and 5-HT2C agonist actions (high nano-molar and low micro molar range for 5-HT2A and 5-HT2C receptors, respectively, Rickli et al., 2019), without clinically meaningful side-effects from opioid agonist actions or serotonin receptor agonist effects, i.e., without the sedation and respiratory depression effects typical of opioids and without psychotomimetic/psychedelic effects typical of certain NMDAR channel blockers (e.g., PCP and ketamine) and certain psychedelic 5-HT2A agonist drugs (e.g., psilocybin, DOI and LSD) (Example 3 demonstrates the lack of cognitive side effects from doses of dextromethadone therapeutic for MDD).

The lack of clinically meaningful opioid related side effects and psychedelic/psychotomimetic effects at doses that result in sustained therapeutic benefits for MDD is now shown by the Phase 2a results presented herein (see Example 3). The above results and observations from the Phase 2a study signal that the rapid and sustained antidepressant effects of dextromethadone may be determined by its concomitant actions as an NMDAR channel blocker (Gorman et al., 1997) and potentially also by its actions as a 5-HT2A and 5-HT2C agonist (Rickli et al., 2018). Both of these actions potentially induce neural plasticity and modulate the activity of hyperactive NMDAR channels in patients suffering from MDD, while promoting neural plasticity and neural connectivity via both, NMDAR channel block and possibly serotonin agonism (5-HT2A and 5-HT2C receptor agonist action) and possibly other serotonin receptors and pathways (experiments to better define the role of 5-HT2A and 5-HT2C receptors in neural plasticity modulation in ARPE-19 cells are in progress, including the verification of structural association between serotonin and NMDA receptors).

The present inventors have thus provided not only a strong signal for rapid and sustained therapeutic actions of dextromethadone in patients with MDD but also a novel mechanism of action that explains dextromethadone's highly effective neural plasticity effects that are potentially at the basis of its therapeutic efficacy. In particular, the present inventors' clinical and experimental results signal sustained, disease-modifying effects of dextromethadone in MDD and related disorders, such as the disorders listed herein, and confirms the potentially therapeutic disease-modifying effects in other MDD-related disorders discussed in this application.

The present inventors now also disclose the use of dextromethadone for the treatment of somatic symptom disorder (SSD) for the treatment of adjustment disorder (AD) and for the treatment of substance use disorder (SUD). When the inventors explored the effect of dextromethadone in patients with cancer pain (Morley et al., 2016) originating from stimulation of CNS and/or PNS neurons (neuropathic pain), somatic nociceptors (somatic pain) and visceral nociceptors (visceral pain), there was no measurable effect on pain intensity.

The present inventors' novel clinical and experimental results, disclosed herein, signal that dextromethadone, while perhaps ineffective for reducing pain intensity, is potentially disease-modifying for SSD and AD, including when the most prominent symptoms of these disorders is pain. For further clarification, dextromethadone's efficacy for SSD and AD with a pain component is not a direct effect on pain caused by ongoing stimulation of CNS or PNS neurons (neuropathic pain), somatic nociceptors (somatic pain) and visceral nociceptors (visceral pain), for which classic analgesics work best (e.g., racemic methadone). However, when ongoing stimulation of CNS or PNS neurons (neuropathic pain), somatic nociceptors (somatic pain) and visceral (pain) nociceptors is not the primary culprit, as is the case in both SSD and AD with a pain component (in contrast for example with post-operative pain or even chronic cancer pain) dextromethadone, with its potential disease/disorder modifying effects and mechanism of action, defined throughout this application and disclosed in Examples 1-11, could be potentially curative, as seen in patients with MDD (as seen in Example 3 herein).

Along the same line of reasoning, the present inventors now disclose that dextromethadone is potentially a disease-modifying treatment for SUD, especially in the absence of “tolerance to and a physical dependence on, and/or a physical craving for a narcotic analgesic”. Based on new clinical and experimental evidence, “when a subject has a tolerance to and a physical dependence on, and/or a physical craving for a narcotic analgesic and/or addictive substance” opioid substitution therapy may work best, e.g., racemic methadone or levomethadone, as confirmed by Isbell H, Eisenman A J: The addiction liability of some drugs of the methadone series. J Pharmacol Exp Ther. 1948; 93: 305-313; Fraser and Isbell, 1962. Based on the above, the present inventors now disclose that dextromethadone is not indicated “when a subject has a tolerance to and a physical dependence on, and/or a physical craving for a narcotic analgesic and/or addictive substance”. The present inventors now disclose that when a subject no longer has tolerance to an addictive substance and no longer has a physical dependence on an addictive substance, and no longer has a physical craving for an addictive substance, but nevertheless suffers from SUD, dextromethadone, with its potential disease/disorder modifying effects, could be potentially curative for SUD, as seen in patients with MDD.

The unexpected similar effects between 25 and 50 mg dosages with a signal towards a better efficacy of the lower dosage (ceiling effect) prompted the new in vitro study detailed in Example 2 and a review of the previous PD e PK findings for dextromethadone, including a new review of the Phase 1 PD and PK results in Bernstein et al., 2019. The results of the new in vitro study in Example 2 and the review of PK/PD modeling also point toward potential efficacy for lower doses. Furthermore, when the present inventors measured BDNF plasma levels in normal volunteers treated with dextromethadone, they found a strongly statistically increase in BDNF in subjects treated with 25 mg but not in subjects treated with 50 and 75 mg. Finally, only a very low 5 mg single dose of dextromethadone was associated with a signal for nootropic effects. Taken together, these findings signal a possible therapeutic effect of even very low doses of dextromethadone, e.g., dosages that would results in plasma levels even lower than those shown in Example 3 for the 25 mg dose on day 7 and closer to the plasma levels seen in the same patients on day 14 (when therapeutic effects were still present), and around plasma levels for 5 mg doses. Based on studies performed by the inventors and the results disclosed in the present application (see Examples 1-7), the therapeutic concentrations of dextromethadone for MDD may spare physiologically functional NMDARs (the rapid physiological opening and closing of NR1-GluN2A and NR1-GluN2B channels does not allow dextromethadone to enter and block the phasically open channel, but the same therapeutic concentrations are sufficient and effective to act on select pathologically and tonically hyperactive channels e.g., NR1-GluN2C and possibly NR1-GluN2D.

The NMDAR channel block effects of racemic methadone, d-methadone, !-methadone, racemic ketamine, and [S]-ketamine have been demonstrated in vitro measuring the electrophysiological response of human cloned NMDA NR1/NR2A and NR1/NR2B receptors expressed in HEK293 cells. The approximately equivalent half maximal inhibitory concentrations (IC50) for each of these compounds were in the low micromolar range (see Table 1 of Bernstein et al., 2019). The nanomolar affinity for mu opioid receptors of dextromethadone is 1/10th to 1/30 compared to levomethadone (Gorman et al., 1997; Kristensen et al., 1994) and the mu opioid related analgesic effects of racemic methadone at commonly prescribed doses are ascribed to levomethadone (its potency at the opioid receptor is listed as double the potency of racemic methadone, thus the contribution of dextromethadone to the opioid effects is considered negligible). Due to the micromolar (NMDAR) and nanomolar (mu opioid receptor) affinities, the doses of dextromethadone used in the present inventors' clinical study (25 and 50 mg) (which did not have clinically meaningful opioid effects) are unlikely to block normally functioning phasically activated NMDAR channels. High receptor occupancy may be desirable for certain drugs for the treatment of certain diseases and disorders. In the case of dextromethadone and other NMDAR modulators, the therapeutic target is limited to pathologically and tonically hyperactive NMDAR (e.g., GluN2C or 2D) and not the phasically hyperactive NMDARs (e.g., GluN2A, 2B). Therefore, the receptor occupancy of normally functioning phasic NMDARs should be very low, or even better, none, at doses free of opioid side effects or other clinically meaningful side effects and effective for the treatment of MDD (as shown in Example 3) and for modulating pathologically and tonically hyperactive NMDARs (the pathologically and tonically hyperactive NMDAR containing 2c and 2d subunits allows the binding dextromethadone, “on” kinetics, as seen in Example 6). This promising mode of action, selective targeting of hyperactivated receptors while sparing normally functioning receptors, is also supported by a signal for better outcomes from lower doses compared to higher doses, as seen in the present inventors' clinical results in patients (Example 3).

Furthermore, the ion channel region of the NMDAR is highly conserved across the different receptor subunits, which is likely the reason for the low subtype selectivity of the clinically effective (MDD) tested NMDAR blockers (less than 10-fold)—as seen in Example 1. However, it has been shown that physiological levels of Mg²⁺ decrease memantine inhibition of GluN2A or GluN2B-containing receptors nearly 20-fold, so the selectivity for NMDA receptors containing GluN2C and GluN2D subunits increases up to 10-fold (Kotermanski and Johnson, 2009). The combination of Mg²⁺ with dextromethadone may increase the selectivity of dextromethadone for the same receptors and thus improve its efficacy.

The present inventors have also determined that the NMDAR block by dextromethadone is extracellular and that intracellular block, after the dextromethadone penetrates the cell membrane, is unlikely to be a substantial contributor (Example 6).

In conclusion, dextromethadone, by working on pathologically open and tonically hyperactive receptors [at excitatory and inhibitory neurons and possibly at astrocytes, and other cells] and downregulating excessive Ca²⁺ influx, results in a resumption of neural plasticity, allowing new memory to form on top of dysfunctional memory (emotional depressive memory in the case of MDD) and other dysfunctional memory microcircuits in the case of other diseases and disorders. Chronic excessive Ca²⁺ influx, as seen with hyperactivated tonically and pathologically open NMDARs determine excessive Ca²⁺ influx which has an inhibitory effect on physiological neural plasticity (similarly to a complete lack of stimulation with no presynaptic glutamate release and no post-synaptic Ca²⁺ influx, resulting in reduced neural plasticity). Too much and too little Ca²⁺ influx interfere with neural plasticity, both phasically (too much or too little stimulation, stimulus evoked LTP-eEPSCs) and tonically (too much or too little Ca²⁺ influx, stimulus independent “maintainance” LTP-mEPSCs). Furthermore, the actions of dextromethadone on downregulation of excessive Ca²⁺ may prevent more severe cellular dysfunction, including apoptosis, with prevention of diseases and disorders associated with cell loss, including neurodevelopmental and neurodegenerative disorders and apoptosis associated with aging. Of note there is evidence that MDD is also associated with neuronal and astrocytic cell loss, as detailed above.

### Example 8—Molecular Modeling

In this study, based on the disease-modifying actions of dextromethadone derived from Example 3 (above) and other Examples disclosed in the present application, the present inventors disclose that methadone metabolites, e.g., EDDP, may also be disease-modifying. To confirm the mechanism of action for this disclosure, the inventors tested the hypothesis that dextromethadone metabolites potentially interact with the NMDAR channel pore in silico by using molecular modeling to investigate binding to the trans-membrane site of the NMDA receptor GluN1-GluN2B tetramer subtype in its closed state. The computational NMDAR subtype built for this in silico testing is the GluN1-GluN2B tetramer composed by 2 GluN1 subunits and 2 GluN2B subunits. Of note, N2B subunits are essential for formation of super-complexes that include NMDARs. To improve the computational efficiency of calculations, only the trans-membrane region of the receptor was modeled. This was done because the trans-membrane region of the receptor is (1) where the presumed PCP binding site is located, (2) where the tested FDA-approved and clinically tolerated NMDA channel blockers (dextromethorphan, ketamine, memantine) also are likely to act, and (3) where the present inventors hypothesize methadone and its isomers and metabolites may also act.

The inventors used the structure identified by the Protein Data Bank (PDB) code 4TLM as the starting point for the computational studies to investigate the drugs shown in Table 50, below, all of which are known NMDAR open channel blockers presumed to act at the PCP site at the trans-membrane domain with known affinities and known clinical effects. PCP is a schedule I drug and MK-801 is a high affinity NMDAR channel blocker with severe side effects that impede its clinical use. The other four drugs are in clinical use for various indications, as indicated throughout the application. As seen in this Example 8, as shown in Table 50, the docking scores for the tested dextromethadone metabolites are in a similar range as those of established NMDAR channel blockers.

TABLE 50

| | Predicted Affinity |
|---|---|
| Molecule | (Docking) (Delta G, kcal/mol) |
| MK-801 | −6.8 |
| PCP | −6 |
| Ketamine | −5.8 |
| Memantine | −5.8 |
| Amantadine | −5.23 |
| Dextromethorphan | −6.3 |
| Dextromethadone | −6.5 |

Further, all tested metabolites showed predicted affinity results (shown in Table 51, below) in a range similar to compounds with known NMDAR blocking actions (approximately −5 to −7 predicted affinity, as shown in Table 50, above). These in silico results signal potential NMDAR blocking effects at the pore channel for dextromethadone metabolites.

TABLE 51

| | |
|---|---|
| 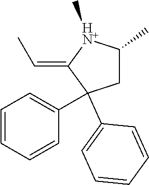 title: R-EDDP-trans glide gscore: −7.085 | 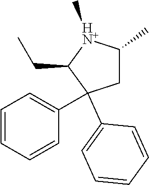 title: R,S-EMPD glide gscore: −6.969 |
| 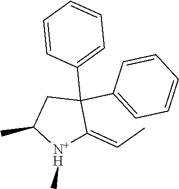 title: S-EDDP-cis glide gscore: −6.967 | 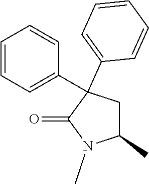 [SMILES: C[C@@H]1CC(c2ccccc2)(c2ccccc2)C(=O)N1C] title: R-DDPP glide gscore: −6.853 |
| 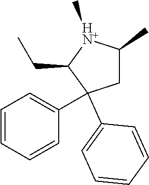 title: S,R-EMPD glide gscore: −6.783 | 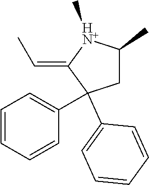 title: S-EDDP-trans glide gscore: −6.74 |
| 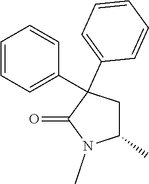 [SMILES: C[C@H]1CC(c2ccccc2)(c2ccccc2)C(=O)N1C] title: S-DDPP glide gscore: −6.56 | 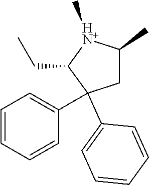 title: S,S-EMPD glide gscore: −6.445 |

Given the results shown in Table 51 (above), as compared to the scores shown for other NMDAR channel blockers in Table 50, the present inventors suggest that similar metabolites would exhibit similar affinity results. Such metabolites may include, but are not limited to:

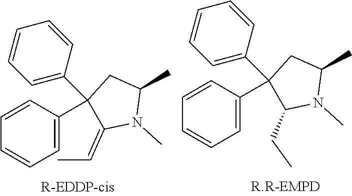

### Example 9: Additional Disease-Modifying Signals from the Example 3 Phase 2 Study

This Example 9 provides sub-analyses of indicators that suggest that the effects of dextromethadone are not limited to mood improvement, thus corroborating the present inventors' demonstrated disease-modifying effects, which are more likely to cause improvement in different symptoms, not only one symptom such as mood.

The sub-analyses of data from the Phase 2 study presented in Example 3 (see patient data for MADRS and SDQ individual and composite indicators below), informs on the potential for dextromethadone for treating diseases and disorders as MDD and related disorders, and other disorders listed herein. This data signals: (1) cognitive improvement in patients with MDD (signaling potential for nootropic effects); (2) therapeutic effects for sleep disorders; (3) potential therapeutic efficacy for social functioning; (4) therapeutic efficacy for ability to perform at work, including for improvement in energy and motivation; and (5) potential therapeutic efficacy for sexual dysfunction. Effects (1)-(5) are unlikely to be merely symptomatic and are likely to be part of MDD or related disorders (the Mini International Neuropsychiatric Interview specifically rules out medical, organic, drug causes for psychiatric symptoms and the SAFER interview confirms the diagnosis of MDD not secondary to known medical causes). Symptomatic treatments are more likely to act on one symptom rather than on a constellation of symptoms. Standard antidepressants generally improve mood but not motivation or sexual function. Aspirin for infection may improve fever but not cough or other infection specific symptoms. Antibiotics for infection are a disease modifying treatment that will improve fever and eventually even cough from a bacterial pneumonia.

A. Overview

1. Background

REL-1017 (dextromethadone HCl) is an N-methyl-D-aspartate receptor (NMDAR) channel blocker recently tested in patients with Major Depressive Disorder (MDD) at oral daily doses of 25 mg and 50 mg in a double blind randomized multicenter placebo controlled three arm phase 2 study. Both tested doses of REL-1017 were administered orally once a day with a loading dose on day 1 of 75 mg or 100 mg followed from day 2 to day 7 by 25 mg or 50 mg, respectively (Example 3). Both tested doses were found to have rapid, robust and sustained efficacy according to all tested scales. Noteworthy, both doses showed a favorable tolerability and safety profile with no evidence of cognitive side effects or withdrawal upon abrupt discontinuation. The importance of improving functional outcomes is increasingly recognized, especially in the field of neuropsychiatric disorders.

2. Objective

To analyze the effects of REL-1017 on select functional indicators part of the MADRS and SDQ scales.

3. Methods

The present inventors selected items from the MADRS and SDQ scales and created Composite Indexes of cognitive and motivational functions: Cognitive composite index: [MADRS 6 (concentration difficulties), SDQ 16 (wakefulness), SDQ 22 (slowed down feeling, SDQ 35 (ability to focus), SDQ 36 (ability to remember), SDQ 37 (ability to find words), SDQ 38 (sharpness), SDQ 39 (ability to make decisions), SDQ 42 (ability to work)]; Motivation-energy composite index: [MADRS 7 (lassitude), SDQ 7 (motivation), SDQ 20 (energy)]; Mood composite index: [MADRS 1 (reported sadness), SDQ 1, 2, 3 (mood)]; Sleep composite index: [MADRS 4 (reduced sleep), SDQ 13 (ability to fall asleep), SDQ 14 (ability to stay asleep in the middle of the night), SDQ 15 (ability to stay asleep around the time before waking up)]; The present inventors also separately analyzed two additional single functional items part of the SDQ. 1) Social function, single question (SDQ 41, social functioning); 2) sexual function, single question (SDQ 40, sexual functioning).

4. Statistical Analysis

Analysis of Change from Baseline at different times after the onset of treatment: The likelihood-based method applied is the Mixed-Effect Model Repeated Measures (MMRM) model with fixed-effect terms for treatment, visit (Day 2, Day 4, Day 7, Day 14) and the interaction between treatment and visit. The LS means and the LS means difference (the difference between REL-1017 and Placebo in the LS means) are provided along with the p-value for testing the hypothesis of no difference and the Cohen's effect size (calculated based on the LS mean differences and the pooled standard deviations). The 25 mg and 50 mg dosages are considered separately and combined: 25 mg+50 mg, Combined Treatment Group (CTG).

5. Results

Cognitive Composite Index: Day 7: the least-squared mean difference compared to the placebo group was: 25 mg treatment group −10.23 (p value 0.1; effect size 0.49) 50 mg treatment group: −11.41 (p value 0.07; effect size 0.53) CTG, 25 mg+50 mg: −10.85 (p value 0.05; effect size 0.51) Day 14: the least-squared mean difference compared to the placebo group was: 25 mg treatment group: −14.71 (p value 0.01; effect size 0.86) 50 mg treatment group: −20.61 (p value 0.0008; effect size 1.15) CTG, 25 mg+50 mg: −17.83 (p value 0.0009; effect size 1.02). Motivational Composite Index: Day 7: the least-squared mean difference compared to the placebo group was: 25 mg treatment group −17.37 (p value 0.02; effect size 0.73); 50 mg treatment group: −17.41 (p value 0.01; effect size 0.74; CTG, 25 mg+50 mg: −17.39 (p value 0.006; effect size 0.74); Day 14: the least-squared mean difference compared to the placebo group was: 25 mg treatment group: −26.5 (p value 0.0003; effect size 1.33); 50 mg treatment group: −26.27 (p value 0.0002; effect size 1.34); CTG, 25 mg+50 mg: −26.38 (p value 0,000029; effect size 1.35); Mood Composite Index, Day 7: the least-squared mean difference compared to the placebo group was: 25 mg treatment group −12.3 (p value 0.08; effect size 0.51); 50 mg treatment group: −16.1 (p value 0.02; effect size 0.72); CTG, 25 mg+50 mg: −14.3 (p value 0.02; effect size 0.62); Day 14: the least-squared mean difference compared to the placebo group was: 25 mg treatment group: −16.5 (p value 0.02; effect size 0.71); 50 mg treatment group: −18.0 (p value 0.01; effect size 0.85). CTG, 25 mg+50 mg: −17.3 (p value 0.006; effect size 0.79): Sleep Composite Index, Day 7: the least-squared mean difference compared to the placebo group was: 25 mg treatment group −6.6 (p value 0.44; effect size 0.22); 50 mg treatment group: −9.18 (p value 0.27; effect size 0.38); CTG, 25 mg+50 mg: −7.96 (p value 0.27; effect size 0.3); Day 14: the least-squared mean difference compared to the placebo group was: 25 mg treatment group: −21.7 (p value 0.001; effect size 1.09; 50 mg treatment group: −21.7 (p value 0.0009; effect size 1.2). CTG, 25 mg+50 mg: −21.74 (p value 0.0001; effect size 1.17). Social function, single question (SDQ 41, social functioning), Day 7: the least-squared mean difference compared to the placebo group was: 25 mg treatment group: −1.07 (p value 0.04; effect size 0.65; 50 mg treatment group: −1 (p value 0.05; effect size 0.57); CTG, 25 mg+50 mg: −1,034 (p value 0.021; effect size 0.61); Day 14: the least-squared mean difference compared to the placebo group was: 25 mg treatment group: −1,246 (p value 0.003; effect size 0.99); 50 mg treatment group: −1,137 (p value 0.006; effect size 0.98); CTG, 25 mg+50 mg: −1.19 (p value 0.0009; effect size 0.99). Sexual function, single question (SDQ 40, sexual functioning) 25 mg treatment group: −0.66 (p value 0.15; effect size 0.48); 50 mg treatment group: −0.28 (p value 0.52; effect size 0.19)

CTG, 25 mg+50 mg: −0.46 (p value 0.23; effect size 0.32); Day 14: the least-squared mean difference compared to the placebo group was: 25 mg treatment group: −1.32 (p value 0.006; effect size 0.93); 50 mg treatment group: −0.4 (p value 0.35; effect size 0.29)

CTG, 25 mg+50 mg: −0.86 (p value 0.037; effect size 0.59)

6. Conclusion

In patients with MDD, aside from improving the overall CFB compared to placebo in all tested scales, REL-1017 (dextromethadone) resulted in rapid, clinically meaningful, sustained, and statistically significant improvements in cognitive, motivational, social and sexual functions. The rapid, robust and sustained efficacy of REL-1017 for MDD is not limited to improving mood but potentially extends to cognitive, motivational, social and sexual functions with meaningful socioeconomic implications, aside for corroborating a mechanism of action based on disease modifying mechanisms. These encouraging results signal potential disease modifying effects of dextromethadone and signal potential advantages over treatment with standard antidepressant treatments.

B. MDD: Customizing Posology of NMDAR Channel Blockers with the Use of a Digital Application

Data from the Phase 2 trial (Example 3), including data from the PK/PD relationship, and sub-analyses of single patient responses, suggest therapeutic efficacy potentially starting on day 2 or earlier, and wide inter-subject variability in magnitude of effect and/or sustainability/duration of response.

In order to customize treatment to best meet individual needs, the present inventors disclose the coupling of a dextromethadone treatment with a digital application that monitors the patient's symptoms and signs and informs caregivers in real time, and even patients or their relatives, on the appropriate posology and duration of treatment for individual patients. Among other questions and instructions, the digital application may utilize one or more questions, and modifications thereof, derived from questionnaires administered to MDD patients in the Phase 2 study (Example 3) and during other dextromethadone trials (Bernstein et al. 2019; Moryl et al., 2016), and in particular those questions found to be influenced by treatment with dextromethadone (Example 3 and this Example 9): ATRQ, Antidepressant Treatment Response Questionnaire; CADSS, Clinician-Administered Dissociative States Scale; CGI-I, Clinical Global Impressions of Improvement; CGI-S, Clinical Global Impressions of Severity; COWS, Clinical Opiate Withdrawal Scale; C-SSRS, Columbia-Suicide Severity Rating Scale; HAM-D-17, Hamilton Depression Rating Scale-17; IWRS, Interactive Web Response System; MADRS, Montgomery-Asberg Depression Rating Scale; MGH, Massachusetts General Hospital MINI, Mini International Neuropsychiatric Interview; SDQ, Symptoms of Depression Questionnaire; BPI, Brief Psychiatric Interview; ESAS, Edmonton Symptom Assessment Scale; VAS, visual analogue scale; MGH-CPFQ=Massachusetts General Hospital—Cognitive and Physical Functioning Questionnaire; Digit Symbol Substitution Test (DSST); Sheehan Disability Scale (SDS); and Bond-Lader scale.

C. Radiolabeled NMDAR Channel Blockers as a Diagnostic Tool and as a Drug Selection Tool

Pathologic NMDAR receptor activation (NMDAR hyperactivity) may be selective for certain neuronal or extra-neuronal populations, and may trigger, worsen, or maintain a multiplicity of diseases and disorders. NMDAR hyperactivity may be caused by higher-than-normal levels of glutamate and/or PAMs and/or agonist substances and may be corrected by NMDAR channel blockers, e.g., dextromethadone (see Examples 1 and 5).

The pattern of distribution of radiolabeled dextromethadone and or other NMDAR channel blockers with low affinity for opioid receptors may be diagnostic for MDD or other neuropsychiatric disorders or even extra CNS diseases. The pattern of distribution of radiolabeled dextromethadone and or other NMDAR channel blockers with low affinity for opioid receptors administered alone or even with an opioid agonist or antagonist may be diagnostic for select diseases caused by hyper-activation of select neurons (or other cells), including non-neuronal cells) part of the endorphin system. In the case of dextromethadone, the administration of naloxone may allow to detect a particular distribution of radiolabeled dextromethadone outside of the endorphin pathway and part of a different system or pathway or circuitry involved in a specific disease for which NMDAR and receptors other than the opioid receptor, are central. The pattern of distribution of radiolabeled dextromethadone and or other NMDAR channel blockers may thus be employed as a diagnostic tool for diagnosing diseases and disorders in patients. The pattern of distribution of radiolabeled dextromethadone and or other radiolabeled NMDAR channel blockers with low affinity for opioid receptors and or radiolabeled investigational drugs may also be employed as a drug selection tool for selecting effective disease-modifying drugs.

D. Coupling Magnetic Resonance Spectroscopy and Other Radiological Techniques with NMDAR Channel Blockers as a Diagnostic Tool and as a Drug Selection Tool

Magnetic Resonance Spectroscopy (MRS) has been used to understand the mechanisms of diseases potentially associated with increased glutamate and pathologic NMDAR receptor activation. NMDAR hyperactivity may be selective for certain neuronal (or even extra-neuronal) populations, and may trigger, worsen, or maintain a multiplicity of diseases and disorders. NMDAR hyperactivity may be caused by higher-than-normal levels of glutamate and/or PAMs and/or agonist substances and may be corrected by NMDAR channel blockers, e.g., dextromethadone (e.g., Examples 1 and 5).

The modification of the MRS results by dextromethadone and or other NMDAR channel blockers may be employed as a diagnostic tool for diagnosing diseases and disorders in patients and for following treatment efficacy. The modification of the MRS results by dextromethadone and or other NMDAR channel blockers and in particular by investigational drugs may be employed as a drug selection tool for selecting effective disease-modifying drugs.

E. NMDARs and Extra CNS Diseases and Disorders

Aside from CNS, PNS, and certain specialized receptors, peripheral NMDAR have also been demonstrated on the membrane of most cells, including cells that are part of the respiratory, cardiovascular, and urogenital systems, and on hepatocytes, Langerhans cells, and immune system cells [Du et al., 2016; Dickens et al., 2004; McGee M A, Abdel-Rahman A A. N-Methyl-D-Aspartate Receptor Signaling and Function in Cardiovascular Tissues. J Cardiovasc Pharmacol. 2016; 68(2):97-105; Miglio G, Varsaldi F, Lombardi G. Human T lymphocytes express N-methyl-D-aspartate receptors functionally active in controlling T cell activation. Biochem Biophys Res Commun. 2005; 338(4)1875-1883], and on platelets [Kalev-Zylinska M L, Green T N, Morel-Kopp M C, et al. N-methyl-D-aspartate receptors amplify activation and aggregation of human platelets. Thromb Res. 2014; 133(5):837-847]. Diseases and disorders may be caused by hyperactivation of peripheral NMDARs [Du et al., 2016; Ma et al., Excessive activation of NMDA receptors in the pathogenesis of multiple peripheral organs via mitochondrial dysfunction, oxidative stress, and inflammation. SN Comprehensive Clinical Medicine (2020) 2:551-569].

Based on the present inventors' disclosure (including Examples 1-11), dextromethadone, a very well tolerated and safe drug with clinically meaningful therapeutic effects on diseases such as MDD via NMDAR blocking actions, in the absence cognitive side effects and abuse liability, may be potentially useful for preventing, treating and diagnosing diseases and disorders caused by hyperactivation of NMDARs, including peripheral, extra CNS, NMDARs, including diseases and disorders listed by Du et al., 2016 and Ma et al., 2020 (those diseases and disorders being incorporated by reference herein). In particular, body aches, including headaches, and GI symptoms caused by infections, including viral infections, caused by hyperactivation of peripheral NMDARs, could be relieved by dextromethadone.

In an experimental murine model, dextromethadone, while not analgesic (hot plate latencies), inhibits splenocyte proliferation (significantly more than levomethadone) not affected by naloxone administration, signaling a non-opioid mediated mechanism for immuno-modulatory effect [Hutchinson M R, Somogyi A A. (S)-(+)-methadone is more immunosuppressive than the potent analgesic (R)-(−−)-methadone. Int Immunopharmacol. 2004; 4(12)1 525-1530]. Furthermore, the activity of levomethadone decreases this effect of dextromethadone. Based on Example 1 and other observations outlined in this application, the present inventors postulate that this immuno-modulating action is due to the NMDAR block of dextromethadone, without meaningful PAM actions at NMDARs.

In another study [Toskulkao T, Pornchai R, Akkarapatumwong V, Vatanatunyakum S, Govitrapong P. Alteration of lymphocyte opioid receptors in methadone maintenance subjects. Neurochem Int. 2010; 56(2):285-290], chronic opiate exposure was associated with down-regulation of G-protein-coupled opioid receptor gene expression in human lymphocyte. Per the Taskulkao et al. 2010 study, the mechanism by which opiates induce changes in the number of opioid receptors present on lymphocytes may be similar to the one of the mechanisms by which opiates induce tolerance and dependence in target neurons. Based on the current disclosure the present inventors suggest that the mechanism for immune cell receptor modulation is also potentially related to NMDAR block.

Finally, based on He et al. [He L, Kim J, Ou C, McFadden W, van Rijn R M, Whistler J L. Methadone antinociception is dependent on peripheral opioid receptors. J Pain. 2009; 10(4):369-379], the anti-nociceptive effects of methadone are predominantly peripheral (not blocked by centrally administered naloxone methiodide), as opposed to morphine (levomorphine) which acts predominantly in the CNS. These peripheral actions of methadone are potentially related to the NMDAR block of peripheral receptors coupled to opioid receptors [Narita M, Hashimoto K, Amano T, et al. Post-synaptic action of morphine on glutamatergic neuronal transmission related to the descending antinociceptive pathway in the rat thalamus. J Neurochem. 2008; 104(2):469-478; Rodríguez-Muñoz M, Sánchez-Blázquez P, Vicente-Sánchez A, Berrocoso E, Garzón J. The mu-opioid receptor and the NMDA receptor associate in PAG neurons: implications in pain control. Neuropsychopharmacology. 2012; 37(2):338-349], including NMDARs expressed by inflammatory cells, an action not shared by levomorphine, which is not active at NMDARs (Gorman et al., 1997). Thus, the shepherd affinity, introduced above and described in detail in Example 10, can also direct dextromethadone to target peripheral cells with opioid receptors, including immune cells.

Furthermore, glutamate is stored in platelet dense granules and large amounts (>400 μM) are released during thrombus formation. NMDAR agonists facilitate and NMDAR channel blockers inhibit platelet activation and aggregation. The presence of NMDAR transcripts in platelets (Kalev-Zylinska et al., 2014) implies platelet ability to regulate NMDAR expression. Flow cytometry and electron microscopy demonstrated that in non-activated platelets, NMDAR subunits are contained inside platelets but relocate onto platelet blebs, filopodia and microparticles after platelet activation (Kalev-Zylinska et al., 2014).

Disseminated intravascular coagulation (DIC) is a condition in which blood clots form throughout the body, blocking small blood vessels affecting organs and systems, such as heart, lungs, liver, kidney, brain et cetera. Symptoms may include chest pain, shortness of breath, leg pain, problems speaking, or problems moving parts of the body. As clotting factors and platelets are used up, bleeding may occur. This may include hemorrhage in the urine, blood in the stool, or bleeding into the skin. Complications include multi-organ failure. Relatively common causes include infection, surgery, major trauma, burns, cancer, and complications of pregnancy. There are two main types: acute (rapid onset) and chronic (slow onset). Diagnosis is typically based on blood tests. Findings may include low platelets, low fibrinogen, high INR, or high D-dimer. Treatment is mainly directed towards the underlying condition. Other measures may include giving platelets, cryoprecipitate, or fresh frozen plasma. Evidence to support these treatments, however, is poor. Heparin may be useful in the slowly developing form. About 1% of people admitted to hospitals are affected by the condition. In those with sepsis, rates are between 20% and 50%, with high mortality rates. Based on Kalev-Zylinska et al., 2014, DIC could be triggered, maintained, or worsened by hyperactivation of NMDARs expressed by platelets. Dextromethadone and other NMDAR channel blockers and their metabolites, by blocking hyperactivated platelet NMDARs, may be potentially useful for preventing and treating DIC (Examples 1-11).

F. COVID 19

DIC is implicated in the majority of COVID-19 fatalities (Wang J, Hajizadeh N, Moore E E, et al. Tissue Plasminogen Activator (tPA) Treatment for COVID-19 Associated Acute Respiratory Distress Syndrome (ARDS): A Case Series [published online ahead of print, 2020 Apr. 8]. J Thromb Haemost. 2020; 10.1111/jth.14828. doi:10.1111/jth.14828).

A subset of patients with COVID-19 will develop life-threatening complications. Older patients, male patients and patients with respiratory, cardiovascular and metabolic co-morbidities are at higher risk. While co-morbidities and advanced age are associated with increased risk for COVID-19 complications and death, the pathophysiological mechanisms that determine highly variable inter-individual outcomes are unclear.

NMDARs are expressed on the membrane of cells from all systems, including immune, respiratory, cardiovascular, renal, neurons and also platelets. NMDAR hyperactivity is associated with pulmonary, cardiovascular, renal, metabolic, CNS and coagulation pathology. NMDAR channel blockers significantly attenuate acute lung injury caused by various factors (Du et al., 2016; Dickman K G, Youssef J G, Mathew S M, Said SI. Ionotropic glutamate receptors in lungs and airways: molecular basis for glutamate toxicity. Am J Respir Cell Mol Biol. 2004; 30(2):139-144). DIC may be implicated in the majority of COVID-19 fatalities (Wang et al., 2020). Glutamate is stored in platelet and released during thrombus formation. NMDAR agonists facilitate and NMDAR channel blockers inhibit platelet activation and aggregation (Kalev-Zylinska et al., 2014).

An abnormal immunological response has been implicated in the risk for complications in patients with infections, including COVID-19. Dextromethadone has immune system modulating effects (He et al., 2004; Hutchinson et al., 2009; Toskulkao et al., 2009) potentially related to NMDAR block of receptors expressed by cells part of the immune system.

Hyperactivity of NMDAR may be enhanced by positive allosteric modulators and by agonists, exogenous (e.g., drugs and or toxins) and or intermediates of metabolic pathways increased in inflammation (e.g., quinolinic acid), including inflammation caused by infections. A multiplicity of inflammatory substances, including substances produced and or released during viral infections (including COVID-19), or drugs, including antiviral drugs, potentially act as positive allosteric modulators and agonists of the NMDAR and trigger, maintain or worsen complications.

In a subset of patients, complications from COVID-19 may be triggered, maintained, or worsened by hyperactivation of NMDARs in a multiplicity of cell populations and in platelets. Dextromethadone and other NMDAR uncompetitive channel blockers may mitigate inflammatory, respiratory, cardiovascular, gastrointestinal, CNS, metabolic and coagulation (e.g., DIC) complications in patients with COVID-19 by down regulating Ca²⁺ influx through hyperactive N-methyl-D-aspartate receptors (NMDARs) expressed on the membrane of cells part of the immune system, respiratory system, cardiovascular system, renal system, and gastrointestinal and metabolic systems, including liver, pancreas, and CNS (Du et al., 2016; Dickens et al., 2004; Mcgee et al., 2016; Welters A, Lammert E, Mayatepek E, Meissner T. Need for Better Diabetes Treatment: The Therapeutic Potential of NMDA Receptor Antagonists. Bessere Diabetesmedikamente sind erforderlich: therapeutisches Potenzial von NMDAR Antagonisten. Klin Padiatr. 2017; 229(1):14-20; Miglio et al., 2005) and platelets (Kalev-Zylinska et al., 2014).

A recent online publication signals paucity of COVID-19 complications in an opioid addicted population followed at an opioid maintenance facility, Villa Maraini, Rome, Italy (“Coronavirus, i tossicodipendenti sembrano immuni: l'ipotesi degli esperti di Villa Maraini-Cri” II Messaggero, May 4, 2020, Caltagirone Editore). While the authors attribute this finding to the abnormal immune system in these patients, in light of the present inventors' findings and disclosures, the present inventors disclose protection against COVID-19 complications conferred by racemic methadone may be due to its NMDAR channel blocking activity. As disclosed in Example 7, dextromethadone may offer enhanced immunomodulatory actions over methadone and, more importantly, it has the advantage of not having the opioid effects of racemic methadone.

Patients with pre-existing co-morbidities may be more vulnerable because of NMDAR hyperactivity in cells part of affected systems, organs and tissues (Du et al., 2016).

There may be a favorable temporal therapeutic window between the onset of symptoms and the development of complications that can be accessed with medications that could prevent the development of complications, e.g., NMDAR channel blockers.

One potential explanation for the relative protection against COVID-19 complication seen very in young patients potentially rests in the developmental age differential NMDAR framework seen in younger subjects compared to adults (Hansen et al., 2017; Swanger S A and Traynelis S F. Synaptic Receptor Diversity Revealed Across Space and Time. Trends in Neurosciences, August 2018, Vol. 41, No. 8: 763-765). Younger patients may thus be less susceptible to NMDAR hyperactivation by inflammatory mediators, PAMs and/or agonists and/or excessive glutamate extracellular concentrations induced by COVID-19. Of note, glutamate and glutamate agonists (substances acting as agonists at the glutamate site of the NMDAR) are not agonist at juvenile GluN3A subunits (these subunits do lack the glutamate agonist site) and thus NMDAR subtypes with these subunits are insensitive to glutamate (e.g., di-heteromers GluN1-GluN3) or are relative insensitive to glutamate (e.g., tri-heteromers GluN1-GluN2-GluN3) and to other agonists at the NMDA site. NMDAR subtype that are less calcium permeable and or insensitive or less sensitive to glutamate may render cells less vulnerable to excitotoxicity, including excitotoxicity due to PAMs and agonists at the glutamate site. GluN3A subunit containing NMDAR subtypes are less permeable (tri-heteromeric, e.g., GluN1-GluN2-GluN3) or impermeable (e.g., GluN1-GluN3) to Ca²⁺ (Roberts, A. C. et al. Downregulation of NR3A-containing NMDARs is required for synapse maturation and memory consolidation. Neuron 63, 342-356 (2009)). Thus, patients with higher expression of NMDAR containing the GluN3 subunit, e.g., pediatric patients, may be relatively protected against complications induced by increased Ca²⁺ influx via NMDARs (e.g., DIC, respiratory, cardiac, renal, metabolic complications) because their NMDAR framework is less affected by Ca²⁺ currents compared to the NMDAR framework of adults. Gender related differential NMDAR framework may also explain the lesser burden of COVID-19 complications seen in female patients compared to males.

Open channel NMDAR channel blockers (dextromethadone and other select isomers of opioids, their metabolites and their derivatives, ketamine and memantine and amantadine) and especially dextromethadone with its favorable safety, tolerability, PK profiles at effective doses [influx via hyperactive NMDARs (Examples 1-11)] by selectively blocking Ca²⁺, may mitigate, treat and/or prevent DIC from COVID-19 and from other causes of DIC listed above, and other COVID-19 complications, including immunological (inflammatory response), respiratory (cough, lung inflammation, ARDS, respiratory failure), cardiovascular (HTN, ischemic heart disease, and heart failure), metabolic (impaired glucose tolerance and diabetes), renal (renal insufficiency) and nervous system complications (taste and smell deficits, headache, neuropsychiatric deficits, CVAs).

Furthermore, dextromethadone and other NMDAR uncompetitive channel blockers could prevent NMDAR mediated complications form antivirals or other therapies with molecules with positive allosteric modulating or agonist effects at NMDARs (Hama R, Bennett C L. The mechanisms of sudden-onset type adverse reactions to oseltamivir. Acta Neurol Scand. 2017; 135(2):148-160).

In analogy with NMDAR mediated toxicity on hair cells in the inner ear potentially caused by gentamicin, a PAM, (Example 5), the loss of sense of smell and taste associated with COVID-19 could signal NMDAR mediated toxicity in special sensory olfactory cells caused by the virus or its treatment in the presence or absence of a PAM and/or an agonist at NMDAR.

Dextromethadone and its sulphone derivative may symptomatically treat cough (Winter C A, Flataker L. Antitussive action of d-isomethadone and d-methadone in dogs. Proc Soc Exp Biol Med. 1952; 81(2):463-465; Noel, Peter Ret General Practitioner Research Panel. «The sulphone analogue of d-methadone: Assessment of antitussive activity in general practice», British Journal of Diseases of the Chest. 1963, vol. 57 no 1. p. 48-52). Based on the present inventors' disclosures the effectiveness for cough may not only be symptomatic but may signal disease-modifying treatment effects at NMDARs on cells next to the port of entry for the pathogen. Of note, in a subset of patients with COVID-19 the primary symptoms are not respiratory but gastro-intestinal, and, for those patients, dextromethadone may offer symptomatic treatment of gastrointentinal symptoms (GI). The treatment of GI however, as in the case of cough, would not be merely symptomatic but could potentially be disease-modifying, by blocking overstimulated NMDAR receptors coupled with opioid receptors in the GI tract that are causing the complications of the disease.

The mechanism of action of dextromethadone remains downregulation of excessive Ca²⁺ influx via overstimulated NMDARs expressed by cells that are part of any organ, tissue, and system, and in particular, overstimulated NMDARs expressed on the membrane of immunological cells (inflammatory response), respiratory system cells (airway inflammation), cardiac and vascular cells (HTN and heart failure), Langerhans and liver cells (impaired glucose tolerance and diabetes and liver insufficiency), GI cells, renal (renal impairment) and NS cells (neuropsychiatric symptoms, including impairment of special senses), cells part of the hypothalamic-pituitary adrenal axis (hyperadrenergic state) and platelets (DIC). Ketamine IV could be used at sedative dissociative doses in mechanically ventilated patients for both sedative purposes and for NMDAR channel blocker actions for treatment and prevention of COVID-19 complications. Dextromethadone can be used to prevent and treat COVID-19 complications and in addition will exert antitussive effects. As outlined above, and confirmed in the findings of Example 7, the immunomodulating actions described for racemic methadone (Toskulkao T, Pornchai R, Akkarapatumwong V, Vatanatunyakum S, Govitrapong P. Alteration of lymphocyte opioid receptors in methadone maintenance subjects. Neurochem Int. 2010; 56(2):285-290) may be even more marked for dextromethadone (Hutchinson et al., 2004), and may be clinically useful because of lack of opioid and psychotomimetic effects, as confirmed by Example 3. These immunomodulating effects, aside from providing therapeutic actions for MDD and neuropsychiatric disorders, for autoimmune disorders, for infectious disorders, including for COVID-19 complications, can also be therapeutic for cancer and its complications.

Dextromethadone could also have antiviral effects, similarly to the effects of other NMDAR uncompetitive channel blockers such as amantadine and memantine, e.g., by blocking viral pore channels.

Furthermore, the actions of dextromethadone at peripheral NMDARs may profit from its shepherd affinity for peripheral opioid receptors (see Example 10, below) and reach target peripheral receptors (He et al., 2009). All of the tissues and systems listed by Du et al., 2016 are composed by cells expressing opioid receptors, including, respiratory, renal, cardiac, pancreatic, liver, GI and immune cells.

G. Patients of Asian Descent

In order to gain approval for new drug applications, the Japan Pharmaceuticals and Medical Devices Agency requires supplemental pharmacokinetic (PK) safety and/or pharmacodynamic (PD) efficacy studies because FDA (USA) and EMA (Europe) new drug applications are generally based on studies with limited data from Asian/Japanese subjects. Differences in PK and PD, determined mainly by differences in drug metabolism between different populations due to genetic variance, are the basis for the Japanese Agency requirements for supplemental clinical studies in Japanese subjects. Because of the requirement for additional studies, applications for marketing of new drugs to the Japanese population may depend on the addition of novel data supportive of a drug for a development program in Japan. The novel data presented in this application substantiate the hypothesis of efficacy specifically in patients of Asian descent and define the type, design and extent of additional studies required.

Known genetic differences between Japanese and Caucasian subjects (Hiratsuka M I, Takekuma Y, Endo N, Narahara K, Hamdy S I, Kishikawa Y, Matsuura M, Agatsuma Y, Inoue T, Mizugaki M. Allele and genotype frequencies of CYP2B6 and CYP3A5 in the Japanese population. Eur J Clin Pharmacol. 2002 September; 58(6):417-21) are likely to determine differential PK and PD responses to racemic methadone and dextromethadone among different populations.

In September 2012, over 60 years after its discovery and widespread use in the United States and Europe, racemic methadone was approved in Japan for the treatment of pain.

Takagi and Aruga (Takagi Y, Aruga E. New Opioid Options in Japan—Methadone, Tapentadol and Hydromorphone]. Gan To Kagaku Ryoho. 2018 February; 45(2):205-211) point out how diversity of pharmacokinetics among individuals requires close monitoring of adverse events. The PK and PD racemic methadone diversity described by Takagi et al., 2018 potentially pertain also to dextromethadone. Racemic methadone undergoes hepatic N-demethylation to produce the stable and opioid-inactive metabolite, 2-ethylidene-1,5-di-methyl-3,3-diphenylpyrrolidine, by cytochrome P450 (CYP) iso-forms CYP3A4, CYP2B6, CYP2C19, and to a lesser extent by CYP2C9 and CYP2D6.

Stereoselective metabolism of racemic methadone by CYP2B6, CYP2C19, and CYP3A4 was studied using an enantiospecific methadone assay, where CYP2B6 preferentially metabolized dextromethadone, CYP2C19 preferentially metabolized levomethadone, and CYP3A4 showed no preference (Gerber J G, Rhodes R J, Gal J. Stereoselective metabolism of methadone N-demethylation by cytochrome P4502B6 and 2C19. Chirality. 2004; 16: 36-44).

Bridging studies are typically used to interpret the results of PK and PD results from studies performed in predominantly Caucasian populations and apply these results to patients of Asian descent.

The present inventors present new data and new data analyses for dextromethadone suggesting that differential PK and PD responses may not result in clinically significant negative outcomes that would impede development of dextromethadone for therapeutic uses in Asian and/or Japanese patients. The present inventors also present new data and new data analyses suggestive of potential efficacy, including efficacy in Asian patients. The data presented in this application, aside from teaching that further development of dextromethadone in the Asian and/or Japanese population may have potentially beneficially therapeutic uses, informs on pathways for further development of dextromethadone as a new chemical entity for therapeutic uses in Asian and/or Japanese patients.

1. Single Dose and Multiple Dose Ascending Studies

The present inventors performed supplemental analyses of data from the single dose and multiple dose ascending studies (SAD and MAD studies) shown in Bernstein et al., 2019 and disclose that in racially diversified subjects [SAD (42 subjects): Caucasian 57.1%, Black-African American 28.6%, Asian 11.9%, mixed 2.4%; MAD (24 subjects): Caucasian 62.5%, Black-African American 20.8%, Asian 12.5%, mixed 4.1%] dextromethadone exhibits linear pharmacokinetics with dose proportionality for most single-dose and multiple-dose parameters. Single doses up to 150 mg and daily doses up to 75 mg for 10 days were well tolerated with mostly mild treatment-emergent adverse events and no severe or serious adverse events. Dose-related somnolence and nausea occurred and were mostly present at the higher dose level. There was no evidence of respiratory depression, dissociative and psychotomimetic effects, or withdrawal signs and symptoms upon abrupt discontinuation. An overall dose-response effect was observed, with higher doses resulting in larger QTcF (QT interval corrected using Fridericia formula) changes from base-line, but none of the changes were considered clinically significant by the investigators. No detectable conversion of dextromethadone to levomethadone occurred in vivo in these subjects, including in patients of Asian descent. Specifically, to this application, among the 6 Asian subjects included in these studies receiving at least one dose of dextromethadone, single doses up to 150 mg and daily doses up to 75 mg for 10 days were well tolerated with mostly mild treatment-emergent adverse events and no severe or serious adverse events.

The present inventors also performed a pharmacogenomic analysis (detailed below) in subjects (SAD and MAD studies) treated with dextromethadone and the present inventors were able to conclude that despite the high PK variability, the accumulation ratios for all parameters and dose levels were less than 20%, thus demonstrating that inter-individual variabilities affect the PK parameters but do not influence overall drug accumulation. These pharmacogenomic analysis results thus suggest that dextromethadone PK and PD results in patients are likely to be reproducible in patients of Asian descent and/or Japanese patients.

2. Pharmacogenetic Analysis

A blood sample for DNA extraction was obtained from each subject. Samples were stored at −70° C. or colder pending shipment to the genomics laboratory (LabCorp Clinical Trials—Genomics Lab [Seattle, Wash]). Based on blinded analysis, certain subjects were identified as either slow or fast metabolizers. DNA from blood samples of these subjects was extracted and subjected to microarray analysis to determine the specific expression of certain metabolic enzymes (see below).

Pharmacogenomics testing was done using a DMET microarray (Affymetrix, Santa Clara, Calif.). An exploratory analysis was performed by assigning activity scores to the different metabolizers: poor metabolizer=0, intermediate metabolizer=1, extensive metabolizer (EM)=2, and ultrarapid metabolizer=3, with inter-mediate scores for uncertainties, such as intermediate metabolizer or EM=1.5, EM or ultrarapid metabolizer=2.5. Pharmacokinetic parameters common for both SAD and MAD studies were pooled. DMET profiling included polymorphism for multiple metabolism-related genes and their interpretation for the phenotype and activity for related genes. However, information on gene activity based on the presence of gene polymorphism was not available for all genes. Pharmacogenomics reporting was limited to a subset of metabolizing enzymes relevant for Dextromethadone metabolism as reported in the literature (Fernandez C A, Smith C, Yang W, et al. Concordance of DMET plus genotyping results with those of orthogonal genotyping methods. Clin Pharmacol Ther. 2012; 92:360-365), specifically the CYP enzymes CYP1A2, CYP2B6, CYP2C18, CYP2C19, CYP2D6, CYP3A4, CYP3A5, and CYP3A7.

A total of 9 samples from the SAD study and 10 samples from the MAD study were selected for pharmacogenomic analysis, and PK parameters common for both studies were pooled for comparison (one of the selected samples was from an Asian subject). The CYP3A4 phenotype exhibited normal metabolism for all subjects tested and thus did not affect dextromethadone metabolism. The analysis suggested a tentative correlation between elimination half-life and CYP2B6 metabolic activity and possibly CYP1A2 activity. CYP2B6 extensive and ultrafast metabolizers (activity score 1.5-2.5) had a noticeably shorter elimination half-life compared with poor and intermediate metabolizers. A similar trend was observed for CYP1A2. The CYP2C19 relationship with elimination was opposite to what would be expected: increased activity coincided with prolonged dextromethadone elimination. A tentative trend was observed for CYP1A2 and exposure over the 24 hours following the first dose of dextromethadone in that increased activity correlated with less exposure. No other CYP enzymes had an effect on exposure.

The dose proportionality of dextromethadone had not previously been well characterized in the literature. Although the high variability in the PK parameters prevented determination of statistical significance, the linearity of PK was tentatively demonstrated for single-dose parameters and was conclusively demonstrated for multiple-dose parameters. Dose proportionality for the MAD study was demonstrated for single-dose Cmax and AUCtau on day 1 and for steady-state Cmax, AUCtau, and Css on day 10. Despite the confirmed dose proportionality for the MAD study, comparison of concentration and exposure between the 50 and 75 mg treatment groups demonstrated very slight differences. The higher variability within the 50 mg subjects, based on demographic/pharmacogenomic characteristics, or fast absorption of the drug from the bloodstream into peripheral compartments based on dose level, with slow release back into the systemic circulation could possibly explain this observation. Separate elimination in the peripheral compartments could also have contributed.

The attainment of steady-state occurred following 6 or 7 daily doses of dextromethadone. In the SAD study, the ratio of AUC0-inf to AUC0-24 was approximately 2.5-fold, with a percent coefficient of variation of 25%. This was considered an expected accumulation ratio for steady-state exposure, assuming linear PK. Accumulation ratios calculated using Cmax, Cmin, and AUCtau demonstrated an accumulation of dextromethadone over the 10 days of dosing. Accumulation ratios were the highest for AUCtau at the 50-mg dose level but were generally in the range of 2.3- to 3.4-fold. Thus, the observed accumulation of dextromethadone was close to or slightly exceeded the expected accumulation at the 50-mg dose level. Despite the high PK variability, the accumulation ratios for all parameters and dose levels were less than 20%, thus demonstrating that inter-individual variabilities affect the PK parameters but do not influence overall drug accumulation.

Cytochrome P450 enzymes have preferences for one of the racemate stereoisomers, as is the case of racemic methadone. CYP2B6 plays a greater role in metabolizing dextromethadone than L-methadone, and CYP2B6 polymorphism was shown to affect the exposure of dextromethadone. In the MAD study, CYP2B6 extensive and ultrafast metabolizers had a noticeably shorter elimination half-life. Although previous data showed no effect of CYP1A2 on racemic methadone disposition in methadone maintenance patients, the present inventors observed that higher activity correlated with shorter elimination half-life and less exposure in healthy normal volunteers. However, differences in study populations may have influenced these results as the present inventors excluded smokers from the studies, and tobacco smoke is a known inducer of CYP1A2.

Potentially complex mechanisms are involved in the distribution and elimination of dextromethadone, with interactions between metabolizing enzymes and transporters such as the efflux drug transporter P-glycoprotein, encoded by the ABCB1 gene. It has been suggested that polymorphism in this gene drastically affects the PK of methadone; however, the effects are inconclusive, in part due to the high number of single-nucleotide polymorphisms in the coding region that have varying population frequencies. The high PK variability the present inventors observed is consistent with the complex metabolism of dextromethadone by multiple CYP enzymes and the diversity of the CYP2B6 polymorphism.

In summary, taken together with genetic variance specific to the Japanese population and known to influence dextromethadone exposure (Hiratsuka et al., 2002), the present inventors' novel data analyses detailed above, indicate the safety of dextromethadone treatment in the Asian descent and/or Japanese population (SAD and MAD data from 6 Asian patients treated with dextromethadone doses up to 150 mg detailed above) and are encouraging of further development of dextromethadone in the Asian and/or Japanese patient population.

3. PK and Safety Experimental Data in the Rat

The present inventors performed novel PK studies in the rat and novel safety studies in the rat. These studies (Studies A, B, and C, discussed briefly below) provide novel information essential for the proper design of studies in human subjects, including human subjects of Asian and/or Japanese descent.

Study A was a pharmacokinetic study of a single text article following oral and/or subcutaneous administration to rats. In Study A, a total of 255 study samples were analyzed for methadone (dextro and levo enantiomers). The results from calibration standards and quality control samples demonstrated acceptable performance of the method for all reported concentrations.

Study B was a study for effects of d-methadone on embryo fetal development in rats with a toxicokinetic evaluation. In this embryo fetal development study in Sprague-Dawley rats administered d-methadone orally from GD 6-17, no test article-related effects were observed on maternal survival, clinical findings, ovarian and uterine parameters, or maternal macroscopic findings at any dose level evaluated. Test article-related, but non-adverse, decreases in maternal body weight and/or bodyweight change were observed at 10, 20, and 40 mg/kg/day and decreases in maternal food consumption at 40 mg/kg/day. No evidence of developmental toxicity based on fetal survival, sex ratios, body weights, and external, visceral, and skeletal examinations was observed at any dose level evaluated. Based upon these findings, the no-observed-adverse-effect level (NOAEL) for both maternal and developmental toxicity was considered to be 40 mg/kg/day (GD 17 Cmax=738 ng/mL; GD 17 AUC0-24 hr=9920 hr*ng/mL), the highest dose level evaluated.

Study C was a 91 day safety study in the rat describing the long term safety of different doses of dextromethadone in the rat. This study provided new long term safety data, in particular lack of CNS effects and respiratory depressant effects compared to racemic methadone,

In particular, Study A showed marked PK differences in the rat, including differences based on sex, which will be taken into consideration for the analysis of human data, including studies and data from Asian and/or Japanese subjects, including female subjects. In particular, Studies B and C demonstrated novel safety data indicative for the design of human studies and the analysis of human data, including studies and data from Asian and/or Japanese subjects, including studies and data in women of childbearing age.

These novel PK and safety experimental data in the rat, taken together with the human PK, PD and pharmacogenomic data presented above lend new support and new teaching useful for the development of dextromethadone in the Asian and/or Japanese population, including in female subjects, including in female subjects of childbearing age. Finally, studies A, B and C encourage and teach the development of dextromethadone in patient populations potentially more pharmacologically sensitive, including in patients of Asian descent and in particular of Japanese descent.

4. Efficacy Experimental and Clinical Data

The new experimental data presented in this application (Example 3) further support and teach the next steps for the clinical development program of dextromethadone in Asian countries, including Japan. Examples 1-9 all support development of dextromethadone for a multiplicity of diseases and disorders, including development in subjects of Asian descent, including Japanese patients.

In particular, the data presented show that dextromethadone produces CNS plasticity effects and behavioral effects of potential clinical relevance, especially in light of the recent discoveries on the neurobiology and neuropathology of neuropsychiatric diseases, disorders, symptoms and conditions, including depression, anxiety, pseudobulbar affect, fatigue, and obsessive compulsive disorder; self-injurious behaviors chosen from trichotillomania, dermotillomania, and nail biting; depersonalization disorder; addiction to prescription drugs, illicit drugs, or alcohol; and behavioral addictions; pain including neuropathic pain; alcohol withdrawal; and cough. The neuroplasticity and behavioral experimental results disclosed in this application, taken together with the increase in plasma BDNF determined by dextromethadone administration in 100% of the tested Asian subjects (N=2) compared to placebo provides support for potential therapeutic effects for Asian and/or Japanese patients.

In summary, the new data and results disclosed above, support the safety and efficacy of dextromethadone and teach continued clinical development of dextromethadone as a therapeutic agent and/or as a neuroplasticity modulator, including for populations, such as the Asian and/or Japanese population, that exhibit differences in PK and PD parameters and characteristics compared to Caucasian populations.

### Example 10: Mechanism of Action: The Endorphin System and its Relation to NMDARs; Selective Targeting of MOR-NR1 Dual Receptor Heterodimers; NMDAR Shepherd Affinity; Ligand-Directed Signaling

This Example 10 demonstrates shepherding as providing a new mechanism of action that explains the selectivity of the NMDAR channel blocker dextromethadone for NMDARs on neurons part of mood controlling brain circuitry.

A. Premise

The endorphin system, well known for its central role in pain/analgesia (Pasternak G W, Pan Y X. Mu opioids and their receptors: evolution of a concept. Pharmacol Rev. 2013; 65(4):1257-1317. Published 2013 Sep. 27), regulates the affective component of experience (e.g., pleasure and suffering). The endorphin system is the main physiological regulator of homeostatic mood and well-being, and directs choices, social interactions, and cognitive abilities/interests. Conditions (well-being, contentedness), and functions (cognitive and motivational functions, e.g., ability and willingness to concentrate on a task; learning, memory formation) and neuropsychiatric disorders (e.g., altered moods, depressed or manic, anxiety states, addictions and compulsive behaviors), are highly regulated by the endorphin system. The endorphin system homeostasis is altered in neuropsychiatric disorders, such as MDD, GAD, OCDs, addiction disorders and related disorders (Lutz P E, Kieffer B L. Opioid receptors: distinct roles in mood disorders. Trends Neurosci. 2013; 36(3):195-206).

The clinical applications of chronic uses of opioid drugs, the drugs that led to the characterization of the receptor-ligand interaction in the endorphin system, are limited by tolerance, physical dependence, and addiction. Despite these drawbacks, because of lack of alternatives, up to the 1950s, opioids were used widely for the treatment of neuropsychiatric disorders, including mood disorders and anxiety.

The direct drug (or endogenous ligand) interaction with opioid receptors (MORs, DORs and KORs and others) is responsible for the opioid effect (Pasternak and Pan., 2013). Not all agonists to the endorphin system are mood enhancers: while activation of MORs is associated with a rewarding response (beta-endorphin and MOR agonists), the contrary is true for activation of KORs (dynorphin and KOR agonists), which is associated with dysphoria.

Experiences can be novel or repeated. Novelty, in particular, is associated with endorphin release.

B. Novelty Experience

When the novelty experience has favorable evolutionary/species preserving features (e.g., sexual activity, food intake, or even plain physical exercise), beta-endorphin is released and the mu opioid receptor (MOR) is activated with sensations of pleasure, relaxation, and even euphoria (MOR agonist like sensations).

When the novelty experience has unfavorable evolutionary/species preserving features (e.g., the experience has potential or actual damaging consequences for species preservation as in the case of pain), dynorphin is released and the kappa opioid receptor (KOR) is activated with dysphoric sensations (KOR agonist like sensations).

The receptor binding effects of endorphins that are released after a repeat experience (i.e., not a novel experience) are downregulated by NMDAR receptor activation (tolerance) and by the NMDAR-mediated neural plasticity consequential to the first, novel experience (change in synaptic framework and change in Ca²⁺ influx following the repeat stimulus). This tolerance to the effects of a repeat experience compared to the novel experience is true for each repeat experience, because each last experience becomes the “novel” experience relative to the previous experience. The same applies to repeated intake of an opioid agonist drug, e.g., for recreational purposes or for analgesic purposes: the effect of repeat doses will be different (e.g., gradually less intense) compared to the preceding “recreational fix” or “analgesic effect.” This well-known phenomenon, tolerance, is caused by activation of NMDARs and downstream consequences of a differential Ca²⁺ influx compared to the preceding experience.

Synaptic framework “virginity” to a particular experience (reversal of tolerance) can be at least partially restored if enough time is allowed between experiences [the amount of time required will depend on the individual (baseline synaptic framework) and on the type and intensity of the experience, e.g., food, sex, or opioid as a recreational “fix”, or opioid as an analgesic, “pain killer”]. Time between stimulations (i.e., time without glutamate release in that particular synaptic cleft part of a select circuit and thus the time without additional NMDAR activation) allows for a return to functional baseline (closed state of NMDAR channel) and a new structural (LTP+LTD) baseline within the specific synaptic framework expressed on the membrane of specific cells involved in the experience, i.e., select neurons part of the endorphin system.

Thus, if sufficient time has elapsed, an experience can be repeated with the same or very similar effects (intensity of emotional response) compared to a novel experience (reversal of tolerance). If the experience has a strong evolutionary species-preservation connotation, e.g., the experience of food and sex, the elapsed time between experiences necessary to allow NMDARs to return to a close state and thus again mu receptors to elicit strong response to an endorphin burst is short. This is also true for opioid addicts who allow enough time between “fixes” or, when opioids are used for post-surgical pain, when enough time separates two surgical operations and thus the two painful events treated with opioids: when sufficient time is allowed to elapse between two doses of drug, the effects of the repeat opioid drug will be close to the effects experienced after a first time use because the NMDAR associated to the opioid receptor has returned to its baseline activity.

Established experimental models of depression in mice exposed to stress are based on loss of interest for sex (FUST, female urine sniffing test) and loss of interest for novelty food (NSFT, novelty-suppressed feeding test). In data disclosed by the inventors, dextromethadone has been shown to exert antidepressant-like effects in these models. The postulated mechanism of action for these antidepressant-like effects, based on Example 2 and confirmed by the sustained therapeutic effects of dextromethadone disclosed in Example 3, signals potential neural plasticity induced disease-modifying effects.

Opioid receptors and NMDARs (but not AMPARs) co-localize in the same areas of the brain (Narita et al., 2008) and are structurally associated (MOR-NR1 form receptor heterodimers in vivo) in the post-synaptic area of select neurons). Of note activation of AMPARs is necessary for triggering voltage dependent calcium influx via GluN2A and GluN2B channels because the opening of these channels is dependent on depolarization and release of Mg²⁺ block (in the presence of Mg²⁺ block these channel subtypes are completely blocked). On the other hand, GluN2C and GluN2D allow some Ca²⁺ influx at resting membrane potential (Kuner et al., 1996; Kotermanski et al., 2009). Dextromethadone may thus preferentially act on GluN2C NMDAR subtypes and Glun2D subtypes (Examples 1, 5, and 6).

NMDAR activation is the molecular mechanism for tolerance to endorphins (this can be seen as a physiological and evolutionary species preserving mechanism, so individuals are not incentivized to indulge in futile hedonistic behaviors) and is also the molecular mechanism for the well-known phenomenon of tolerance and addiction to certain effects of opioid drugs (Trujillo K A, Akil H. Inhibition of morphine tolerance and dependence by the NMDA receptor antagonist MK-801. Science. 1991; 251(4989):85-87). Interestingly, levels of tolerance (onset and intensity) differ for different effects: tolerance to respiratory depression and euphoria are rapid and intense, while tolerance to the analgesic effects is somewhat slower and less intense. Finally, there is little or no tolerance to the constipating effects of opioids. This latter effect is mainly a peripheral effect of opioids, suggesting that neural plasticity may be the mechanism of tolerance for central effects like euphoria. This differential tolerance for the different effects of opioids also signals select MOR-NR1 heterodimer activation by opioids. In light of the present inventors' experimental findings (Examples 1-11) and other observations, tolerance to these opioid effects, physical dependence, and the addiction liability of opioids (and the dysphoria of withdrawal, including dysphoria persisting in addicts after resolution of physical dependence) and compulsive behaviors are potentially determined by preferential pathological activation of GluN2C and or GluN2D NMDAR subtypes associated with MORs. The inventors disclose that the same mechanism, hyperactivation of select MOR-NR1 heterodimers, is at the basis of MDD.

NMDAR activation regulates the physiological functioning of the endogenous opioid system by decreasing (tolerance) the effects of endorphins (or opioids) caused by repeated (not novel) stimulation-induced experience (or induced by repeated administration of opioids). By definition, there can be no tolerance to a novel experience and there can be no tolerance to the first dose of an opioid agonist drug. Tolerance, a form of learning/memory (NMDAR hyperactivity with neural plasticity consequences) develops to a repeat experience and to a repeat dose of an opioid agonist drug. The molecular mechanism of tolerance (to a repeat experience or to a repeat dose of an opioid) is PAM of the NMDAR structurally associated (physically coupled) with the opioid receptor. The increase in NMDAR channel opening (PAM effect) enhances Ca²⁺ influx (Narita et al., 2008). Excessive Ca²⁺ influx in the postsynaptic neuron expressing in its synaptic hotspot MOR-NR1 heterodimers is thus the molecular basis of tolerance (decreasing effects of repeat experiences or repeat opioid doses for analgesia or recreational purposes).

Repeat “positive” experiences will cause activation of NMDARs structurally associated with its MOR (physical coupling of NR1-MOR) and will determine tolerance to the surge of beta-endorphin with a relative or even absolute loss of interest in repeating such “positive” experience that has lost its novelty. At the same time, a repeat “positive” experience may determine a state of contentedness, especially if the “right” amount of time is allowed to elapse between repeat experiences. This “right amount of time” will vary according to the individual (and its synaptic framework) and the type of experience [generally, experiences of food and sex, necessary for survival (species preserving experiences) will have a shorter “right amount of time” i.e., the elapsed time that allows to experience pleasure with repeat experience is shorter, compared to other stimuli that are less crucial for survival).

This physiologic NMDAR activation by endorphins (“positive” experience) and its downstream effects (LTP) will decrease with time if the repeat experience is reiterated. If time is allowed to lapse between experiences, there will be a return to baseline activity of NMDAR in the absence of the PAM effects of endorphins. This elapsed time (“quietness” in between exposures) allows closure of the channel and a decrease in Ca²⁺ influx, with physiological downstream consequences, e.g., LTP and new layers of memory). When the experience is repeated after some time, the associated MOR will again be able to respond physiologically to beta-endorphin, with a return of the reward with the repetition of the experience and thus a return in interest for the experience.

As seen with experimental models disclosed by the inventors, if the NMDAR channel part of the MOR-NR1 complex is pathologically active (e.g., because of chronic stress) there is excessive Ca²⁺ entry with cell dysfunction and halting of the LTP machinery and the loss of interest in food and sex (and other activities: anhedonia) will persist over time, as was the case in the experimental models of the isolated symptom of depression. MDD in patients was successfully reversed by the low affinity NMDAR channel blocker dextromethadone in a sustained manner (Example 3).

In predisposed individuals [individuals with a “predisposed” synaptic framework, in particular a “predisposed” NMDAR framework, e.g., NMDARs prone to remain hyperactivated (pathologically hyperactive) after a stimulus] a few repeat “positive” experiences, or even a single “positive”, rewarding, novel experience, may trigger, worsen or maintain neuropsychiatric disorders, based on persistent NR1-MOR heterodimer hyperactivation (e.g., addictions, especially opioid addiction, and/or behavioral addictions, but also OCDs and maniacal states, or even depression because of inability to again achieve that once in a lifetime “blissful state,” e.g. procured by an opioid “fix”). Furthermore, fluctuating NMDAR dysregulation may be the molecular basis for the clinical manifestations of bipolar disorder.

By the same mechanism (hyperactivation of NR1-MOR), repeat doses of mu agonist opioids will cause tolerance and dependence and cause withdrawal with physical (hyperactivation of peripheral NMDARs coupled with MORs) and psychiatric symptoms (hyperactivation of peripheral NMDARs coupled with MORs) upon abrupt discontinuation of the drug or administration of an antagonist (Trujillo and Akil, 1991). The same mechanism (persistent lower level hyperactivation of NMDARs) may trigger MDD after resolution of the physical withdrawal symptomatology. When strong mu agonist opioids are used for pain or for recreational purposes, as a general rule, an analgesic effect on pain, or a euphoric “fix”, or respiratory depression, can practically always be obtained by increasing the dose (no ceiling to analgesic and euphoric effects), implying that NMDAR hyperactivation and its consequent tolerance can be surmounted by a high enough dose of a full agonist mu opioid. This general rule has exceptions at its extremes, e.g., the hyperalgesia seen in chronic pain patients treated with very high doses of mu opioid agonists, where the NMDAR hyperactivity is so accentuated by increasing chronic doses of mu agonists (or their metabolites) that it can no longer be surmounted by a higher opioid dose, and actually the hyperalgesia is worsened by escalating doses. In this situation the hyperalgesia can be resolved or improved by rotation to a different mu agonist, generally at a lower equianalgesic dose (Pasternak and Pan, 2013). Analogies to this model of intense hyperactivation of NMDARs induced by very high doses of chronic opioids can be drawn with very intense repeated traumatic experiences, e.g., PTSD in war veterans.

Repeat “negative” experiences (or dwelling on negative experiences) will cause hyperactivation of NMDARs structurally associated with the KOR, with tolerance to a new surge of dynorphin and a decrease in the height of dysphoria associated with similarly negative experiences (habituation to the effects of negative experience, higher tolerance for predicaments), but may also determine a persistent low level dysphoria (MDD, PTSD) or sensitization to mild events. Both kindling and sensitization are known to be NMDAR mediated phenomena (Trujillo and Akil, 1991; Trujillo K A. Are NMDA receptors involved in opiate-induced neural and behavioral plasticity? A review of preclinical studies. Psychopharmacology (Berl). 2000; 151(2-3):121-141). Patients with severe depression are in general less reactive not only to positive experiences (anhedonia, a known hallmark of depression), but will also be less reactive to negative experience (indifference to loss, e.g., indifference to bereavement or loss of a job; this indifference, a less emphasized manifestation of depression, is captured by question 6 of the SDQ scale). The relative “indifference” to war events seen in some experienced soldiers, while necessary for efficient (not panicky) warfare reactions, may thus be a manifestation of NMDAR hyperactivation (NR1-KOR) and a decrease response of the KOR receptor to dynorphin stimulation.

In predisposed individuals, repeat “negative” experiences or even a single “negative” novel experience (especially if particularly “strong”) may trigger, worsen, or maintain neuropsychiatric disorders (e.g., MDD related disorders, including PTSD and bereavement disorder). These persistent neuropsychological symptoms following a traumatic experience can be explained at a molecular level by NR1-KOR hyperactivation, with excessive Ca²⁺ influx causing impairment of the LTP machinery.

MDD may thus be caused by hyperactive NMDARs associated with MOR and or KOR.

As disclosed in this application, when NMDAR activation is excessive, e.g., pathologically and tonically activated GluN1-GluN2C and 2D subtypes, neuropsychiatric disorders may be triggered, maintained or worsened because of excessive Ca²⁺ influx and consequential dysregulation of the neural plasticity machinery, i.e., dysregulation of downstream signaling for transcription, synthesis, assembly and expression of synaptic proteins and transcription, synthesis and release of neurotrophic factors, including BDNF (see Example 2) and consequential alterations in LTP/LTD. The clinical manifestations of tonic hyperactivation of NMDARs depend on the affected brain region or more precisely, on the neuronal population and associated receptors and functional circuits affected. In the case of MDD and related disorders, the tonic hyperactivation of NMDARs that are physically coupled (structurally associated) with opioid receptors (e.g., NR1-MOR and/or NR1-KOR, especially of GluN2C subtypes) disrupts the physiological regulatory function of the endorphin system, causing MDD and related disorders.

As the knowledge taught by the use of safe and well tolerated NMDAR channel blockers (such as dextromethadone) advances, neuropsychiatrists will be able to understand disorders in relation to NMDAR hyperactivity (response to an NMDAR channel blocker) or NMDAR hypoactivity (worsening after administration of an NMDAR channel blocker). Disorders that are not secondary to NMDAR hyperactivity will not improve or will worsen after administration of dextromethadone.

The clinical manifestations of hyperactivation of NMDARs associated with receptors (including opioid receptors) are thus related to the affected neurons and neuronal population and circuits expressing select receptors physically coupled with said NMDARs. These clinical manifestations of NMDAR hyperactivation depend on the individual's unique NMDAR framework, which is determined genetically and is then shaped epigenetically by environmental stimulation and varies according to developmental phases (i.e. age), sociocultural variables, and even gender differences.

NMDARs are central to memory formation (learning, LTP/LTD) and are ubiquitous in the CNS (and extra CNS where they are necessary for signaling precise instructions related to the main functions of these cells, e.g., insulin production in Langerhans pancreatic cells or production of immunological memory in lymphocytes). NMDARs are structurally associated with select receptors [e.g., opioid receptors in the endorphin system and other receptors for other CNS systems and circuits (or even extra CNS receptors in other tissues)] that differ according to the functions of the particular neuronal population and circuit. When hyperactive NMDARs are structurally associated with opioid receptors, such as in the endorphin system, neuropsychiatric disorders, such as MDD and related disorders, may develop. When hyperactive NMDARs are structurally associated with other receptors, e.g., nicotinic receptors, a different neuropsychiatric disorder may develop, e.g., cognitive impairment.

Ketamine, dextromethorphan, and dextromethadone have low affinity for opioid receptors (memantine does not). These NMDAR channel uncompetitive blockers (e.g., ketamine, dextromethorphan and dextromethadone, but not memantine), by down regulating excessive Ca²⁺ influx in neurons with hyperactive NMDARs structurally associated (physically coupled) with opioid receptors, potentially restore the physiologic responses of these opioid receptors to endorphins, with remission of the neuropsychiatric disorder caused by a dysregulation of the endorphin system.

Endorphins, the physiological neuropeptides that bind opioid receptors, are involved in well-being, reward mechanisms, stress reduction and response to novelty stimuli. Disruption of endorphin pathways is associated with the isolated symptom of depression (Lutz et al., 2015) and endorphin levels have been associated with response to antidepressants (Kubryak O V, Umriukhin A E, Emeljanova I N, et al. Increased β-endorphin level in blood plasma as an indicator of positive response to depression treatment. Bull Exp Biol Med. 2012; 153(5):758-760).

The present inventors have presented evidence for the NMDAR channel uncompetitive blocking actions of dextromethadone (Example 1), including preferential actions on pathologically and tonically hyperactive NMDARs, e.g., GluN1-GluN2C subtypes (Examples 1, 5, 6), and the present inventors have presented evidence that this down-regulation of Ca²⁺ currents by dextromethadone may be therapeutic in animal models and humans (Example 3) via neural plasticity mechanisms.

Furthermore, the present inventors are disclosing that MDD and related disorders may be caused by select hyperactivation of pathologically and tonically activated NMDARs structurally associated with opioid receptors. The NR1-MOR or KOR interaction regulates the physiological effects of endorphins (the effect of endorphins or opioids on MOR and KOR is regulated by the state of the structurally associate NMDAR). NMDAR hyperactivation disrupts the physiological endorphin interaction and ultimately interferes with the NMDAR regulated neural plasticity (synaptic structure and thus synaptic function) that is manifested by real time mood states, cognitive functions and social interactions at any given time during the life of an individual.

As disclosed above, the ability of a potentially therapeutic drug to preferentially target a select NMDAR population, e.g. pathologically and tonically hyperactive GluN1-GluN2C and or GluN1-GluN2D subtypes, while sparing physiologically and phasically opening/closing of NMDARs (e.g., GluN1-GluN2A and GluN1-GluN2B subtypes, strongly gated by the Mg²⁺ block) is crucial for avoiding cognitive side effects, ranging from mild to moderate intensity dissociative symptoms (dextromethorphan and ketamine) to coma, as seen with MK-801 (Trujillo, 2000). These side effects are seen when the function of voltage gated receptors is blocked or may be seen when blockage of any NMDAR subtype is excessive, interfering with its physiological function, including excessive block of the relatively voltage independent NMDAR subtypes (e.g., NR1-NR2C physiologically and tonically open, as opposed to pathologically and tonically active). The preferential block for GluN1-GluN2C and or GluN1-GluN2D subtypes shown for all the clinically tolerated NMDAR channel blockers tested (Example 1) is accentuated several fold by the presence of physiological concentrations (1 mM) of extracellular Mg²⁺ (Kuner and Schoepfer, 1996; Kotermanski and Johnson, 2009).

NMDARs are ubiquitous in the CNS (and extra CNS) and when targeting specific disorders, such as MDD and related neuropsychiatric disorders potentially caused by a dysregulated endorphin system, it would be desirable for a drug to preferentially target pathologically and tonically hyperactive NMDARs that are also functionally and structurally associated (physically coupled) with opioid receptors (e.g., NR1-MOR). This further drug selectivity [selectivity for NMDARs structurally associated with opioid receptors, on top of the previously described selectivity (preference, Example 1) for pathologically and tonically active GluN1-GluN2C and 2D subtypes], in light of the inventors' novel observations, outlined throughout the application and below, appears to be an essential feature for effectiveness of NMDAR uncompetitive blockers for the treatment of MDD and related disorders. In the case of MDD, the targeting of the MOR-NR1 heterodimer, described by Rodriguez-Munoz et al., 2012 and anticipated by Narita et al., 2008, is useful because of the physiological role of the endorphin system in maintaining the physiological state of “well-being”, which is altered in MDD and related disorders opioid receptors and NMDARs are structurally associated in select brain areas (endorphin pathways) to form heterodimers (MOR-NR1) in the post-synaptic region of neurons (Narita et al., 2008; Rodriguez-Munoz et al., 2012).

Thus, for diseases triggered, maintained or worsened by a disruption of NMDARs on neurons part of the endorphin pathway, the select targeting of NMDARs structurally associated (physically coupled) with opioid receptors (the receptors for endorphins) is desirable.

Shepherd Affinity Hypothesis; Ligand Directed Signaling; Dual Receptor; Biased Signaling:

For effective treatment of MDD and related disorders, a drug with affinity for both opioid receptors and NMDARs may be advantageous for the purpose of selectively targeting NMDARs structurally associated (physically coupled) with opioid receptors expressed on the membrane of neurons part of the endorphin system. NMDAR channel blockers without affinity for opioid receptors (e.g., memantine) may not selectively target/reach the endorphin system (but may selectively reach another system and potentially be effective for disease triggered by dysfunction of that system, e.g., Alzheimer disease, by selectively targeting NMDARs associated with another receptor, e.g., a nicotinic receptor), and are thus ineffective for MDD and related disorders (Zarate et al., 2006; Kishi T, Matsunaga S, Iwata N. A Meta-Analysis of Memantine for Depression. J Alzheimers Dis. 2017; 57(1):113-121). Drugs that act only on opioid receptors, e.g., the mu full agonist morphine [(levomorphine, which does not have NMDAR channel blocker activity (Gorman et al., 1997)], will actually have the opposite effects on MDD: by targeting the “euphoric” MOR, levomorphine acts as a PAM at NMDARs, selectively targeting the MOR-NR1 heterodimer. Even a designer opioid combination, selectively targeting (antagonist action) the “dysphoric” KOR (e.g., the buprenorphine samidorphan combination), might selectively target the endorphin system, but is also likely to trigger NMDAR activation in the physically coupled receptor, resulting in tolerance to the KOR antagonistic effects, with reversal of the therapeutic effects on MDD produced via KOR antagonism by the buprenorphine/samidorphan combination. In fact, the designer combination drug for reversal of dysphoria via KOR antagonism showed initial effectiveness followed by loss of efficacy for the treatment of the isolated symptom of depression (Ragguett R M, Rong C, Rosenblat J D, Ho R C, McIntyre R S. Pharmacodynamic and pharmacokinetic evaluation of buprenorphine+samidorphan for the treatment of major depressive disorder. Expert Opin Drug Metab Toxicol. 2018; 14(4):475-482; Zajecka J M, Stanford A D, Memisoglu A, Martin W F, Pathak S. Buprenorphine/samidorphan combination for the adjunctive treatment of major depressive disorder: results of a phase III clinical trial (FORWARD-3). Neuropsychiatr Dis Treat. 2019; 15:795-808. Published 2019 Apr. 4). The loss of efficacy of the buprenorphine samidorphan combination is consistent with a mechanism of tolerance to the KOR antagonistic effects via activation (PAM effect) of the structurally associated NMDAR (KOR-NR1 heterodimer).

In order to be effective for MDD and related disorders, drugs that have both opioid and NMDAR actions should not be high affinity opioid drugs (strong opioids) because the opioid agonist effects of strong (high affinity) opioids prevail on NMDAR block, e.g., for racemic methadone and levomethadone or racemethorphan and levomethorphan the opioid effects prevail on the NMDAR channel blocking effects. However, the NMDAR blocking activity is able to prevent tolerance (it prevents the PAM effect of MOR activation) and there is lesser need for dose escalation and a tendency for maintaining a stable dose with methadone (MOR agonist+NMDAR channel blocker) compared with levomorphine (MOR agonist without NMDAR channel blocking activity).

Strong opioids [e.g., the full opioid agonist l-morphine, devoid of NMDAR blocking activity (Gorman et al., 1997)] thus exert opioid effects and induce tolerance (i.e., act as PAM at NMDAR causing hyperactivation and excessive Ca²⁺ influx). Tolerance can be generally surmounted by increasing the dose: this is well known in the cancer pain treatment field (Pasternak and Pan, 2013), where the medical need for pain control overcomes the downside of some narcotic side effects and high doses of opioids are routinely used for pain control. Drugs that possess both, activity as strong mu agonists and NMDAR blocking actions (e.g., racemic methadone and levomethadone or racemethorphan or levomethorphan) show less tolerance to the analgesic effects (less dose escalation compared to morphine).

On the other hand, certain dextro-isomers of some high affinity strong opioid drugs, while maintaining similar NMDAR blocking actions compared to the racemic mixture, are drugs with low affinity for opioid receptors, i.e., dextromethorphan and dextromethadone, (Codd et al., 1995). Dextromethadone has no clinically meaningful opioid effects at doses that may be therapeutic for disorders triggered or maintained by NMDAR hyperactivity, e.g., for (Example 3). The low opioid receptor affinity of these drugs does not result in clinically evident opioid effects: by increasing the dose, the dose limiting side effects of dextromethadone and dextromethorphan are not those typical of opioids (narcosis, respiratory depression), where even very high doses of dextromethadone are administered. This lack of opioid effects at high doses is seen also in rodent studies: death was preceded by narcosis and respiratory depression only in racemic methadone and l-methadone treated animals but not in dextromethadone treated animals (in these animals death was an “all or none” sudden phenomenon preceded by convulsions (Scott C C, Robbins E B, Chen K K: Pharmacologic comparison of the optical isomers of methadone. J Pharm Exp Ther. 1948; 93: 282-286).

Furthermore, opioids without NMDAR channel blocking actions, e.g., morphine (l-morphine), by acting as PAMs at the NMDAR, with no NMDAR channel blocking activity, may actually trigger, worsen or maintain neuropsychiatric symptoms and disorders, including depression, and including especially depression within the realm of addictive disorders.

D. Re-Thinking NMDAR as Therapeutic and Diagnostic Targets for Neuropsychiatric Disorders: Shepherd Affinity as a Strategy to Target NMDARs Expressed by Select Neuronal Populations Part of the Endorphin System.

From the experimental data (Examples 1-11) the present inventors are able to disclose the characteristics of a useful NMDAR channel blocker for MDD. Those characteristics include: (1) Low micromolar affinity for NMDARs with uncompetitive channel block (Example 1); (2) Similar affinity across the main receptor subtypes (2A-D) (Example 1); (3) Preferential affinity for receptor subtypes less subject to Mg²⁺ block (less subject to voltage gated phasic activation), e.g., GluN1-GluN2C and GluN1-GluN2D (Example 1) [This preferential affinity is magnified several fold in the presence of physiological concentrations of Mg²⁺ (Kuner and Schoepfer, 1996; Kotermanski and Johnson, 2009; Patch Clamp study, Example 6).]; (4) Relatively high “trapping” and substantially useful kinetics: “on” and “off” kinetics at the NMDAR (Example 6); (5) Ability to antagonize the effects of low glutamate concentrations, with or without PAMs and or agonists (Example 5); (6) No cognitive side effects in patients at MDD-effective doses (Example 3), signaling sparing of NMDARs involved in ongoing real time “cognitive” functioning necessary for awareness; (7) Low affinity for opioid receptors: shepherd affinity* for NMDARs structurally associated (physically coupled) with opioid receptors and thus tropism for the endorphin system; (8) The brain concentration of the NMDAR channel blocker (dextromethadone) should be sufficient to exert an action on pathologically and tonically hyperactive NMDAR channels while sparing physiologically working channels, both tonic and phasic. Dextromethadone reaches concentrations 3-4 times higher in the brain compared to plasma concentrations. Its unique chemical structure, its low molecular weight (345.91) and partition coefficient (log P=3.30) allow desirable CNS penetration; and (9) Phasically and physiologically working channels, already blocked by Mg²⁺, except during the depolarized state, are unlikely to be affected by dextromethadone, because of slow onset. (10) Positively charged molecule: the positive charge allows dextromethadone to exert its block during resting membrane potential (at the most negative voltage), similarly to the block exerted by Mg²⁺: when depolarization occurs in the context of external stimulations and presynaptic glutamate release, both Mg²⁺ and dextromethadone are expelled from the channel allowing physiological responses to stimuli, as confirmed by the absence of cognitive effects by dextromethadone at MDD-therapeutic doses (Example 3).

NMDAR Shepherd Affinity (MDD) is defined as follows: opioid receptor affinity resulting in negligible opioid clinical effects (e.g., very weak partial opioid agonist) unable to surmount the therapeutic effects of NMDAR blocking activity but able to direct the drug to the target cell population, e.g., cells expressing NR1-MOR structurally coupled heterodimers at the post-synaptic hotspot, e.g., cells part of the endorphin pathway.

NMDAR Shepherd affinity is defined as follows: definition: receptor affinity for select receptors (e.g., opioid receptors in the case of MDD or nAChR/NMDAR complex in the case of Alzheimer's disease or other select heterodimeric receptors in the case of other neuropsychiatric disorders), that directs an NMDAR channel blocker to the target cell population: cells expressing NMDARs-receptor structurally coupled heterodimers, e.g., nAChR/NMDAR complex (Elnagar M R, Walls A B, Helal G K, Hamada F M, Thomsen M S, Jensen A A. Probing the putative α7 nAChR/NMDAR complex in human and murine cortex and hippocampus: Different degrees of complex formation in healthy and Alzheimer brain tissue. PLoS One. 2017; 12(12):e0189513. Published 2017 Dec. 20) in the case of Alzheimer's disease and e.g., the drug memantine.

The shepherd affinity for drugs with NMDAR antagonist therapeutic activity should result in clinically tolerated or negligible shepherd effects (as is the case for the opioid affinity of dextromethorphan and dextromethadone), unable to surmount (e.g., via PAM effects) the NMDAR therapeutic blocking effects on pathologically hyperactive channels: by increasing the dose, the dose limiting side effects, if any, are NMDAR related and not related to the shepherd affinity receptor effects. Furthermore, the endogenous ligand, in virtue of its receptor affinity and its physiological concentration, should be able to displace therapeutic concentrations of the shepherd affinity drug. This displacement would allow physiological ligand-receptor mechanisms to resume (e.g., endorphins at opioid receptors), and at the same time may favor shepherding of the displaced drug molecule to the structurally associated NMDAR, determining channel closure with downregulation of excessive Ca²⁺ influx and its downstream therapeutic consequences.

The inability of opioid effects to surmount NMDAR clinical effects is a common feature for all the clinically well tolerated, FDA approved NMDAR channel blockers with effects on MDD, including dextromethorphan, ketamine and esketamine and is also true for dextromethadone.

In the case of MDD and related disorders, shepherd affinity directs the drug to hyperactive NMDARs structurally associated with opioid receptors, selectively correcting the NMDAR dysregulation in the endorphin circuitry (e.g., correcting the NR1-MOR heterodimer functional relationship). Shepherd affinity (in this case low affinity for opioid receptors) determines low affinity dextromethadone binding to the opioid receptor without clinically meaningful opioid effects. The low affinity allows displacement of dextromethadone by circulating endorphins and shepherds binding to the structurally associated NMDAR, with block of Ca²⁺ currents and downstream effects, including restoration of the physiologic opioid receptor-endorphin relationship, restoration of ongoing neural plasticity and resolution of MDD manifestations.

The ability of NMDAR channel blockers to selectively target NMDARs that form complexes (structural coupling) with other receptors on the membrane of select cell populations may be selectively therapeutic (and diagnostic) for a multiplicity of diseases, in addition to diseases caused by dysfunctional NMDAR associated with opioid receptors (disease due to impairment of the endorphin system), as disclosed above for MDD and related disorders.

E. Directing NMDAR Channel Blockers to Target Select Neuronal Populations Via Shepherd Affinity for Specific Receptors Structurally Associated with NMDARs

“NMDAR Shepherd Affinity” may thus be a tool for selective targeting of NMDARs (e.g., as is the case with dextromethadone, a low affinity NMDAR channel blocker with preference for NR1-NR2C subtypes) expressed by select cells, e.g., substantia nigra cells, for Parkinson disease, or by caudate nucleus neurons, for Huntington disease, or by motor neurons, for ALS, and so on for a multiplicity of diseases and disorders. This selective targeting of neuronal populations and or circuits is accomplished with “NMDAR shepherd affinity”: low affinity targeting of receptors selectively expressed by neurons part of circuits involved in diseases and structurally associated (physically coupled) with NMDARs (in the case of MDD, shepherd affinity is represented by selective low affinity for opioid receptors, part of the mood-regulating endorphin system).

Hyperactive NMDARs are implicated in a multiplicity of diseases and disorders, e g., diseases and disorders as have been disclosed by the inventors, underscoring the well-known ubiquity of NMDAR expression on virtually all vertebrate cells. Memantine use for Alzheimer's disease may be another example (albeit hitherto undescribed as such) of NMDAR shepherd affinity: here the shepherd affinity may be for the nAChR/NMDAR complex or the sigma 1 receptor or the imidazoline 11 receptor, all receptors for which memantine has low affinity (Elnagar et al., 2017). Amantadine, a low affinity NMDAR antagonist may have some receptor selectivity for neurons in the pars compacta of the substantia nigra, e.g., via NMDAR shepherd affinity for sigma 1 receptors (Peeters M, Romieu P, Maurice T, Su T P, Maloteaux J M, Hermans E. Involvement of the sigma 1 receptor in the modulation of dopaminergic transmission by amantadine”. The European Journal of Neuroscience 2004. 19 (8): 2212-20), or shepherd affinity for another receptor even more specific for this neuronal population. Riluzole, another low affinity NMDAR channel blocker, may benefit from a shepherd affinity for select receptors on motor neurons. An NMDAR channel blocker drug with shepherd affinity for motor neurons, e.g., via serotonin receptors (as shown by Rickli et al., 2018 for dextromethadone), may improve weakness in certain pathological states (Nardelli P, Powers R, Cope T C, Rich M M. Increasing motor neuron excitability to treat weakness in sepsis. Ann Neurol. 2017; 82(6):961-971).

The postulated shepherd affinity is thus a direct function of the select structural association (physical coupling) of NMDARs with other receptors, including opioid receptors, in the case of MDD and related disorders. The endogenous ligand, e.g., beta-endorphin in the case of MOR shepherd affinity, or dynorphin in the case of KOR shepherd affinity, displaces the low affinity dextromethadone molecule from the opioid receptor (the physiological interaction endogenous ligand-receptor is thus undisturbed by the low affinity drug) and dextromethadone is available for binding to the structurally associated hyperactive open channel of the structurally associated NMDAR. In turn, the blocking effects at the NMDAR, via a reduction of excessive Ca²⁺ influx, favor the binding of the endogenous ligand, beta-endorphin or dynorphin, thereby reversing the “tolerance like mechanism” (described by Trujillo and Akil, 1991 for opioids and analgesia), which may have been at the basis of the disruption of the endorphin circuit causing MDD.

NMDAR shepherd affinity characteristics include (1) low affinity and (2) weak or no agonistic effects. These are discussed below.

Low affinity: the affinity/concentration of the NMDAR channel blocker drug for the target shepherd receptor (the opioid receptor in the case of MDD) should be lower than the affinity/concentration of the natural ligand for the same receptor (e.g., beta-endorphin has a several fold higher affinity for the mu opioid receptor compared to dextromethadone, endorphins can therefore displace the therapeutic (MDD) concentrations of a drug like dextromethadone with low affinity for opioid receptors). The displacement of the drug by the natural ligand potentially favors its binding to the structurally associated, physically coupled, NMDAR.

Weak or no agonistic effects (and or favorable side effects): the shepherd affinity to a specific target receptor should not elicit clinically significant adverse effects (but could result in some favorable additional effects potentially clinically meaningful, which may add to the favorable clinical effects determined by the NMDAR block). Strong agonist drugs elicit clinical effects that surmount the NMDAR channel blocking effect, as occurs with racemic or levo-methadone and racemic or levo-methorphan, and therefore the NMDAR effects are less clinically useful [they may remain partially useful, e.g., for reducing tolerance in the treatment of opioid addiction and pain (e.g., racemic methadone), but have limited usefulness in MDD and related disorders].

While the ability to shepherd NMDAR channel blockers to NMDARs expressed on the postsynaptic area of select cellular populations part of a dysfunctional circuit may be important for the selective targeting of certain diseases (e.g., MDD and other disorders related to dysfunction of the endorphin system), a drug like dextromethadone, which is very well tolerated (e.g., because of preferential affinity for pathologically and tonically hyperactivated receptor subtypes less subject to Mg²⁺ block, such as GluN1-GluN2C and/or GluN1-GluN2D subtypes and thus a very well tolerated and potentially flexible drug less prone to cognitive side effects from block of phasically active GluN1-GluN2A and GluN1-GluN2B or tonically and physiologically active GluN1-GluN2C and/or GluN1-GluN2D subtypes), could also be effective (e.g., at higher doses than the doses effective for MDD) for diseases caused by hyperactive NMDARs structurally associated (physically coupled) with other (non-opioid) receptors.

The actions of dextromethadone at different receptors [nicotinic (Talka et al., 2015); sigma-1 (Maneckjee et al., 1997); SET, NET (Codd et al., 1995); serotonin receptors and their subtypes, including especially 5-HT2A and 5-HT2C receptors (Rickli et al, 2018); and histamine receptors (Codd et al., 1995; Kristensen et al., 1995)], might not only potentially determine direct receptor mediated actions, as previously assumed, but potentially may instead or also exert effects via shepherd-affinity, i.e., directing the dextromethadone molecule to select populations expressing one or more of these receptors targeted with low affinity by dextromethadone and thus selectively blocking pathologically and tonically hyperactive NMDAR associated with these receptors, with targeted downstream neural plasticity effects in select neurons part of select circuits.

The present inventors hypothesize that the efficacy of certain low affinity NMDAR channel blockers for MDD and related disorders (dextromethorphan, ketamine, dextromethadone) is dependent on their low affinity for opioid receptors (NMDAR shepherd-affinity): this low affinity for opioid receptors allows selective targeting of hyperactive NMDAR associated with the opioid receptor and thus, selective targeting of neurons part of the dysfunctional endorphin pathway. This explains why memantine, an NMDAR channel uncompetitive blocker with activity similar to dextromethorphan, ketamine and dextromethadone (Example 1), but with no affinity for opioid receptors, and thus, devoid of opioid shepherd affinity for cells expressing the NR1-MOR heterodimer complex and unable of selective targeting of NMDARs expressed by neurons part of the endorphin pathway, is ineffective for MDD (Zarate et al., 2006; Kishi et al., 2017).

Furthermore, naloxone abolishes the antidepressant effects of ketamine (Williams N R, Heifets B D, Blasey C, et al. Attenuation of Antidepressant Effects of Ketamine by Opioid Receptor Antagonism. Am J Psychiatry. 2018; 175(12):1205-1215). By binding to opioid receptors, the opioid antagonist drug naloxone interferes with the lower affinity opioid receptor binding by ketamine and therefore interferes with its shepherd affinity [naloxone high affinity (antagonist) binding to opioid receptors effectively blinds the low affinity shepherd affinity for the same receptor] for preferentially targeting NMDARs structurally associated with opioid receptors (this is yet another novel disclosure by the inventors). While it has been assumed that naloxone may interfere with the weak opioidergic effects of ketamine or may interfere with the effects of endorphins, the reversal of ketamine effectiveness in MDD by naloxone (as evidenced by Willians et al., 2018, describing the effectiveness of ketamine alone and lack of effectiveness of ketamine+naloxone) is instead likely due to the blinding (ketamine can no longer target NMDARs physically coupled with opioid receptors) of its “shepherd affinity” for opioid receptors.

The present inventors consider that the contribution of weak opioidergic effects to antidepressant actions in the case of ketamine (and dextromethadone and dextromethorphan) is unlikely: if this were the case, these weak opioid effects, even if clinically meaningful, would disappear within a few hours of ketamine (and dextromethadone and dextromethorphan) administration (as their plasma levels drop), but instead the antidepressant effects last for days or weeks. Furthermore, none of these drugs appear to have clinically meaningful opioid effects with escalating doses: as the dose is increased “dissociative” like effects, more typical of NMDAR channel blockers, tend to appear and not opioid effects. Also, if the binding to opioid receptors were important for MDD therapeutic actions, doubling the dose would increase effectiveness and this is not the case with the NMDAR channel blockers that are therapeutic for MDD (Example 3). Of note, while the dosing therapeutic window for these NMDAR related cognitive side effects is narrow for ketamine and esketamine, it is wide for dextromethorphan (an over-the-counter drug) and dextromethadone (Example 3). Aside from the showing of the lack of clinically meaningful opioid effects for dextromethadone at doses therapeutic for MDD, the same lack of clinically meaningful opioid effects has been shown for higher doses, including lack of respiratory depressant effects and lack of abuse liability (Isbell and Eisenman, 1948; Fraser and Isbell, 1962; Olsen, G. D., Wendel, H. A., Livermore, J. D., Leger, R. M., Lynn, R. K. and Gerber, N., Clinical effects and pharmacokinetics of racemic methadone and its optical isomers, Clin. Pharmacol. Ther., 21 (1976) 147-157; Scott et al., 1948) and has been recognized by the DEA in a recent publication (Drug Enforcement Administration. Diversion Control Division. Drug & Chemical Evaluation Section. Methadone. Jul. 19, 2019).

For MDD the shepherd-affinity of low affinity opioids with NMDAR blocking action strongly relies on “low affinity” for the opioid receptor, in the absence clinically meaningful opioid effects: molecules with high affinity for opioid receptors determine opioid agonist actions that not only obscure (with opioid effects) but also counteract (PAM action of strong opioids at the associated NMDAR) the low affinity NMDAR blocking actions of the same drug: e.g., racemic methadone and levomethadone are strong opioids and their NMDAR effects are obscured by their narcotic effects. The PAM at NMDARs exerted by morphine described by Trujillo and Akil, 1991 and shown by Narita et al., 2008, and is the molecular basis of morphine tolerance and addiction liability. The NMDAR mechanism for opioid tolerance is also shown by earlier work by one of the present inventors, Charles Inturrisi (in Gorman et al., 1997), and was known to be clinically relevant by the other present inventor (Manfredi et al., 1997).

On the other hand, uncoupling NMDAR channel blockers from opioid receptors, e.g., by adding an opioid antagonist, may allow the NMDAR channel blocker to target another cell population (the drug will no longer be selective for cells with opioid receptors, e.g., cells involved in the endorphin system). The combination with an opioid antagonist (e.g., dextromethadone/naloxone or another opioid antagonist, e.g., sandimorphan) by blinding the opioid shepherd effect, may no longer be effective for MDD but may be effective for another disease or disorder requiring NMDAR channel block selective (preferential) for another cell population (e.g., a cell population with an abundance of nicotinic receptors) and thus may be effective for a different disease, e.g., dementia. The unmet need for a multiplicity of NMDAR channel blockers selective for different diseases and disorders has been note by the present inventors. For example, the addition of naloxone (or another opioid antagonist) to ketamine, dextromethadone, dextromethorphan or any other NMDAR channel blocker with low (or even high affinity for opioid receptors, e.g., levorphanol), will not only antagonize any opioid effects but will blind the shepherd opioid affinity, and thus will uncouple the NMDAR actions from the opioid receptor and potentially decrease the effectiveness of these drugs for MDD, but may “allow” the NMDAR shepherd affinity to be taken over by the “next in line” low affinity shepherd receptor. In the case of dextromethadone the “next in line” low affinity shepherd receptor may potentially be: nicotinic (Talka et al., 2015); sigma-1 (Maneckjee et al., 1997); SET, NET (Codd et al., 1995); serotonin receptors and their subtypes, including especially 5-HT2A and 5-HT2C receptors (Rickli et al., 2018); and histamine receptors (Codd et al., 1995; Kristensen et al., 1995).

As learned from the present inventors' gentamicin experiments (Example 5) and the literature on gentamicin toxicity, PAMs may target select cellular populations (e.g., for the PAM gentamicin, cells in the inner ear or kidney cells) and cause selective excitotoxicity. Furthermore, certain molecules, including endogenous molecules, including quinolinic acid, may act as NMDAR agonists and select neuronal populations may be more affected by this agonist action, e.g., neurons part of the endorphin pathway, in the case of quinolinic acid. Dextromethadone is potentially effective in decreasing excessive Ca²⁺ via NMDARs pathologically hyperactive because of effects of PAMs and or agonists (Example 5).

In light of the present inventors' experimental results (Examples 1-11), MDD may be viewed as a disease of the endorphin pathway where select NMDARs structurally associated with opioid receptors have become pathologically hyper-stimulated: pathologically and tonically hyperactivated by low concentration glutamate, e.g., low levels of extracellular synaptic glutamate induced by stimuli (e.g., stress), with or without PAMs (e.g., morphine or others) and with or without a toxic agonist (e.g., quinolinic acid or others), or even chronic low level excessive glutamate caused by defective clearing mechanisms, e.g., defective EAATs or astrocytic pathology.

Endorphins can no longer bind effectively to opioid receptors when the associated NMDARs are hyperactive (this same molecular mechanism is shared by other pathological states, including opioid tolerance, substance use disorder, chronic pain disorder, other addiction disorders, impulsivity disorders, OCDs, and MDD and related disorders). NMDAR channel blockers, selective (shepherd affinity) for NMDAR structurally associated with opioid receptors (e.g., ketamine, dextromethorphan, dextromethadone), selectively block Ca²⁺ influx in neurons expressing structurally associated (physically coupled) MORs-NMDARs complexes described by Narita et al., 2008, and Rodriguez-Munoz et al., 2012. The downstream effects of reducing the excessive Ca²⁺ influx into cells with pathologically hyperactive NMDARs associated with opioid receptors will restore physiological binding of endorphins [endorphins will displace low affinity opioids (e.g., dextromethadone) from opioid receptors because of their much higher affinity and contribute to favor the binding of the displaced drug to the MOR-associated NMDAR channel binding site]. Finally, NMDAR regulated neural plasticity will resume within the endorphin pathway with production of synaptic proteins and formation of “new healthy emotional memory” and resolution of MDD.

F. Evidence of the MOR-NMDAR Shepherd Hypothesis for MDD

Dextromethadone and all three FDA-approved and tested (Example 1) NMDAR uncompetitive channel blockers have similar low micromolar activity at NMDARs, including a shared preference for GluN1-GluN2C subtypes (Example 1). Among these four drugs, memantine is the only one that has failed to show effectiveness for MDD. The ineffectiveness of memantine in MDD signals that opioid receptor affinity may be required for NMDAR channel blockers to reach select NMDARs part of heterodimeric GluN1-MOR structures expressed by the cell membrane of select neurons, e.g., neurons part of the endorphin system. Furthermore, ketamine drops its efficacy for MDD when an opioid antagonist is added (Williams et al., 2018). Taken together, these findings and observations suggest that low opioid affinity may guide (shepherd) MDD-effective NMDAR uncompetitive channel blockers, so they selectively target neurons expressing NMDARs structurally associated with opioid receptors (e.g., MOR-GluN1 complexes. NMDAR uncompetitive channel blockers are ineffective for depression if they have no affinity for opioid receptors (e.g., memantine, Zarate et al., 2006; Kishi et al., 2017) or, if they have affinity for opioid receptors, they are rendered ineffective for MDD when an opioid antagonist is added (e.g., ketamine, as shown by Williams et al., 2018).

Despite mounting evidence to the contrary, to this day, many of those skilled in the art remain concerned about the abuse liability of dextromethadone. Many skilled in the art assume that the mood lifting effects of dextromethadone may be due to opioid effects (morphine-like effects) from direct interaction with opioid receptors. Similar concerns are still in place for ketamine (Sanacora et al., 2015): the “shepherd affinity” mechanism disclosed in this application is unknown to those skilled in the art, including experts in the field.

While opioid agonists have euphoric and other receptor mediated effects, these effects are limited to the time of binding of the drug to the receptor and are known to cease and rebound upon discontinuation of the drug. The present inventors' Phase 2 results unexpectedly detected two strong signals indicating that the effects of dextromethadone are not symptomatic effects mediated by a direct agonist action at opioid receptors, but are disease-modifying effects, potentially mediated by NMDAR effects targeted selectively via shepherd affinity directing dextromethadone to select NMDARs associated with opioid receptors (part of the endorphin system), with downstream effects, including neural plasticity (Examples 1-11). The first signal is the sustained therapeutic effect for at least seven days after discontinuation of dextromethadone (Example 3) suggests an effect that goes beyond opioid receptor occupancy: receptor occupancy effects would cease after approximately 24 from drug discontinuation, as seen when racemic methadone is used for maintenance of opioid use disorder, or after 6-12 hours, as seen when racemic methadone is used for the treatment of pain. From the present inventors' experience and observations and from the literature review on the uses of racemic methadone and its isomers and from the available scientific literature on racemethorphan and its isomers, it can be inferred that effects secondary to opioid receptor occupancy (pain relieving effects or relief of symptoms and signs of opioid tolerance) require high affinity opioid agonistic action, while NMDAR mediated neural plasticity effects, e.g., therapeutic effects for MDD, require low affinity for opioid receptors (NMDAR shepherd affinity), without clinically meaningful opioid effects.

The second signal for shepherd affinity: for MDD, the 25 mg dose of dextromethadone was as effective or more effective and faster in onset compared to the 50 mg dose (Example 3). Effects mediated by occupancy of opioid receptors have little or no ceiling effect (Pasternak and Pan, 2013): doubling the dose will result in enhanced effects (e.g., when racemic methadone is administered for opioid abuse disorder or for pain, its effects clearly increase when the dose is increased, as is the case with other high affinity opioid agonists like morphine). The lack of opioid effects at doses that relieve MDD signal that the mechanism for MDD effectiveness is not related to opioid receptor occupancy but to NMDAR channel blocking actions. The NMDAR actions at select receptors part of the endorphin system are potentially directed by shepherd low affinity for structurally associated opioid receptors.

NMDAR uncompetitive channel blockers with low affinity for opioid receptors (dextromethorphan, ketamine, dextromethadone) are all effective for MDD, supporting the hypothesis that these drugs may reinstate the physiological endorphin-opioid receptor interactions by reducing NMDAR channel hyperactivity and Ca²⁺ influx in select neurons expressing NMDARs that are structurally associated with opioid receptors (endorphin system), e.g., GluN2C subunit-containing subtypes. This effect requires selective targeting (via shepherd affinity) of neurons expressing opioid receptors.

It is interesting to note that select astrocytic populations (e.g., those in CA1 hippocampal area) highly express MOR (Nam et al., 2018). These MORs are thought to play a central role memory formation (Nam et al., 2019; Zhang H, Largent-Milnes T_{M}, Vanderah T W. Glial neuroimmune signaling in opioid reward. Brain Res Bull. 2020; 155:102-111). The astrocyte role in extracellular glutamate homeostasis is well recognized, and astrocyte derived glutamate is key to NMDAR mediated potentiation of inhibitory synaptic transmission (Kang et al., 1998), as well as key to NMDAR mediated neuronal slow inward current and LTD (Fellin et al., 2004; Navarrete M, Cuartero M I, Palenzuela R, et al. Astrocytic p38α MAPK drives NMDA receptor-dependent long-term depression and modulates long-term memory. Nat Commun. 2019; 10(1):2968).

Of note, sub-anaesthetic doses of ketamine with antidepressant-like effects upregulate the expression of glutamate transporters EAAT2 and EAAT3 in rat hippocampus (Zhu X, Ye G, Wang Z, Luo J, Hao X. Sub-anesthetic doses of ketamine exert antidepressant-like effects and upregulate the expression of glutamate transporters in the hippocampus of rats. Neurosci Lett. 2017; 639:132-137), suggesting a possible role of astrocytic NMDAR in EAAT2 expression control, and therefore in tonic glutamate level control. Thus, low affinity uncompetitive NMDAR channel blockers, such as ketamine, dextromethorphan, dextromethadone and memantine (Example 1), by blocking excessive Ca²⁺ currents through the channel pore of astrocytic NMDARs may control excitotoxicity by yet another mechanism: upregulation of the expression of glutamate transporters, which in turn downregulate tonic levels of glutamate. The preferential targeting (shepherd effect) by dextromethadone of structurally associated, physically coupled, NMDAR-MOR expressed on the membrane of select astrocytic populations, might thus contribute to the antidepressant mechanisms of dextromethadone by different mechanisms, including by mediating a balanced control of extracellular glutamate levels.

Finally, the antidepressant effects of dextromethadone may also be exerted by targeting structurally associated, physically coupled, NMDAR-MOR expressed on the membrane of select glial cell populations (Zhang et al., 2020).

In conclusion, the effects of clinically tolerated NMDAR channel blockers are likely irrelevant at NR1-GluN2A and NR1-GluN2B channels in the presence of physiological Mg²⁺ block. In particular, in the hyperpolarized state, there is complete Mg²⁺ block of GluN1-GluN2A and GluN1-GluN2B subtypes (see Figure 1 of Kuner and Schoepfer, 1996), signaling no potential room for effects for uncompetitive NMDAR channel blockers on these subtypes in the hyperpolarized state. Mg²⁺ at physiological concentrations exerts a 100% effective gating over Ca²⁺ influx and therefore there is no GluN1-GluN2A and GluN2B subtype contribution to LTP from non-depolarized neurons. Without depolarizing events, these subtypes remain closed: these subtypes cannot contribute to memory formation (e.g., during sensory deprivation, in the absence of depolarizing sensory events).

On the other hand, these hyperpolarized neurons, with no Ca²⁺ influx via GluN1-GluN2A and GluN2B subtypes can instead receive Ca²⁺ influx, and thus can maintain some degree of neural plasticity (i.e., synthesis of some synaptic proteins), because, even in the hyperpolarized resting state, there is incomplete block of GluN1-GluN2C and GluN2D subtypes (see Figure 1 of Kuner and Schoepfer, 1996). Therefore, even without depolarizing events, these subtypes remain partially open to Ca²⁺ influx and able to direct cellular function related to neural plasticity, e.g., these subtypes may direct memory formation even during sensory deprivation, in the absence of depolarizing sensory events.

In case of excessive chronic (tonic) extracellular glutamate concentrations, caused by excessive presynaptic release of non-depolarizing glutamate amounts, or defective clearance, with or without PAMs or agonists (other than glutamate and glycine), and excessive (pathological) and chronic (tonic) activation of GluN1-GluN2C and GluN2D subtypes, there is potential therapeutic room for the Ca²⁺ blocking effects of uncompetitive NMDAR channel blockers, shown in the present inventors' FLIPR to have an insurmountable profile (Example 1).

All together the data supports the mechanism of action outlined above for dextromethadone in MDD: Dextromethadone is selective for tonically and pathologically hyperactive GluN1-GluN2C (and potentially GluN1-GluN2D subtypes) and, in particular, tonically and pathologically hyperactive GluN1-GluN2C and GluN1-GluN2D subtypes physically coupled with opioid receptors (part of the endorphin pathway). In summary, the evidence for disclosing the actions of dextromethadone as a disease-modifying treatment for MDD and related disorders is derived from Examples 1-11

Dextromethadone has also affinity for 5-HT2A-5-HT2C channels (Rickli et al., 2018). While this affinity is lower [Rickli et al., 2018, report that dextromethadone is a 5-HT2A agonist (Ki 520 nM) and 5-HT2C agonist (Ki 1900 nM)] compared to the low nanomolar affinity for opioid receptors (Codd et al., 1995), it could potentially serve as shepherd affinity. This affinity for 5-HT2A and 5-HT2C channels could result in a serotonin receptor shepherding effect, analogous to the shepherding opioid affinity effect described for opioid receptors. Thus, dextromethadone could be selective for NMDARs associated with both the serotonin and the opioid systems. The endorphin and the serotonin system are known to be neurotransmitter systems central to the pathophysiology of MDD and its CNS circuitry and thus the preferential targeting of NMDAR structurally associated with serotonin and or opioid receptors may be crucial for the therapeutic effectiveness of dextromethadone. Also, the affinity for nicotinic receptors potentially explains via the same shepherding mechanism, dextromethadone's positive effects on select indicators of cognitive function (Example 3 and Example 9).

### Example 11

A. Select Effects of d-Methadone in Western Diet Treated Rats

All the procedures involving animals were performed in compliance with institutional guidelines that respect national and international laws and policies (Council Directive of the European Economic Community 86/609, OJ L 358, 1, Dec. 12, 1987; NIH Guide for the Care and Use of Laboratory Animals, NIH Publication No. 85-23, 1985). The study design was approved by the Ethics Committee of the University of Padua for the care and use of laboratory animals and by the Italian Ministry of Health (authorization number 721/2017).

Male Sprague-Dawley rats (200±50 g) were housed 3 per cage at a temperature of 21° C., alternating 12 hours of light and 12 hours of dark. After a period of acclimatization, the rats were divided into two appropriately randomized groups: a control group that continued to take the standard diet and another group which was fed with a diet with a high content of fats (60% kcal from fat, High Fat Diet, HFD). This diet was enriched also with fructose in drinking water, at a concentration of 30% (w/V). The combination of HFD and fructose is a model of the so-called “Western diet”. After 26 weeks, the rats on the HFD diet were randomly divided into 2 subgroups. The animals were daily treated for 15 days by gastric gavage with respectively:

aqueous vehicle (Western Diet subgroup);
d-methadone (10 mg/kg body weight).

B. Effect of d-Methadone on Hepatic Inflammation

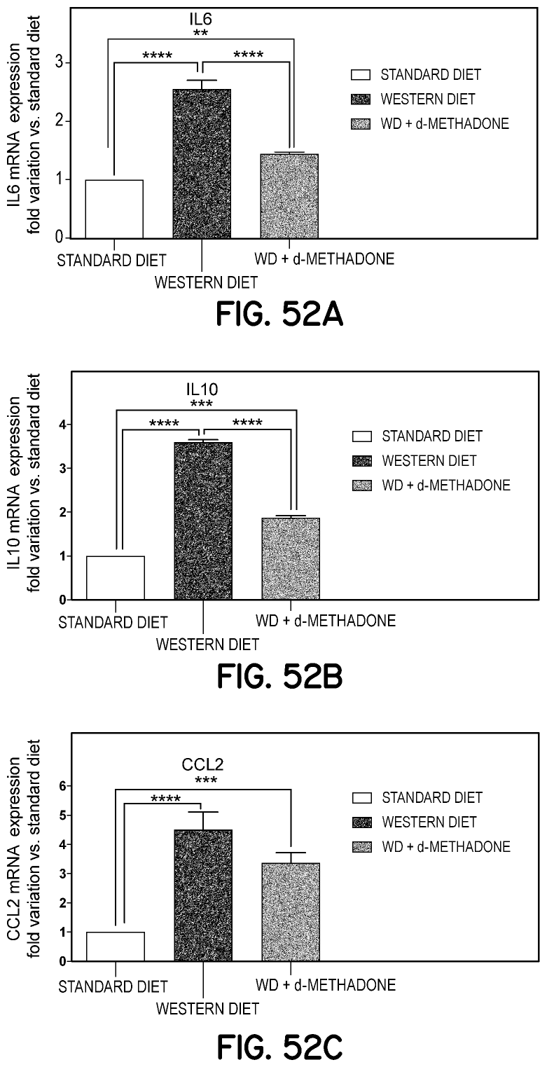

The gene expression of three cytokines involved in inflammation was measured by qRT-PCR in the rat livers. Referring to FIGS. 52A and 52B, the gene expression of the pro-inflammatory interleukin IL-6 and of the anti-inflammatory interleukin IL-10 was significantly increased by Western Diet administration, indicating an increase of hepatic inflammation, probably accompanied by hepatic efforts for regeneration. Interestingly, d-methadone treatment was able to counteract this effect, even if it didn't restore the physiological IL-6 and IL-10 expression. Furthermore, also the gene expression of CCL2, a chemokine involved in inflammation and in the recruitment of immune cells in the liver, was increased by Western Diet with respect to standard diet (see FIG. 52C). D-methadone treatment didn't affect significantly this increase, although a decreasing tendency could be observed in d-methadone-treated animals compared to untreated rats fed with Western diet.

C. Effect of d-Methadone on Liver Status and Hepatic Lipid Metabolism

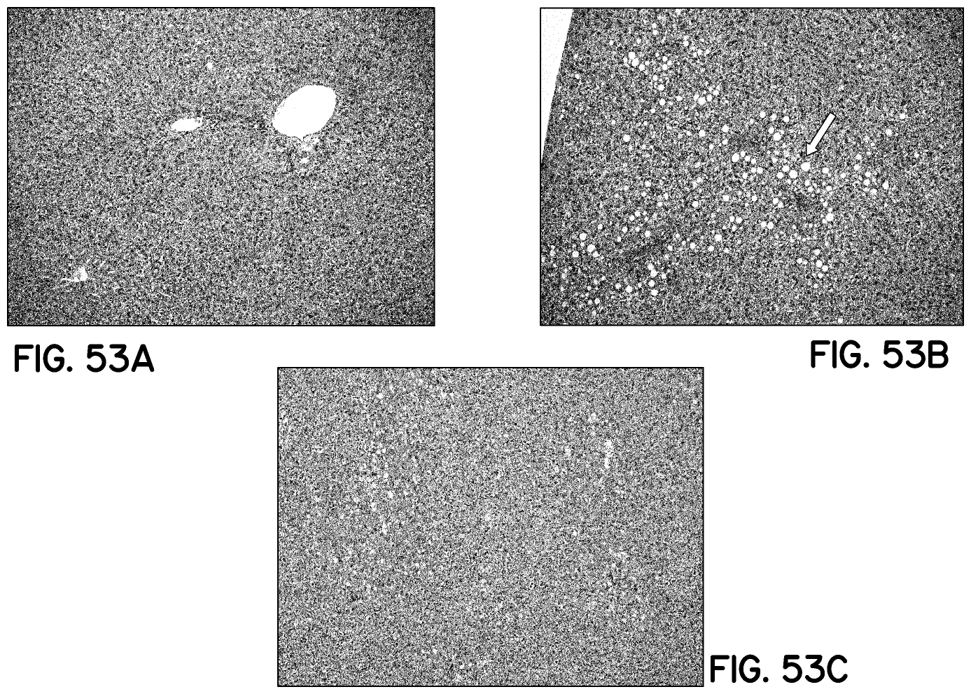

The present inventors also performed a histological analysis of liver tissue by hematoxylin-eosin staining of paraffine-embedded liver slices. At histology, rats fed with Standard diet shows a normal liver architecture (FIG. 53A), whereas lipid accumulation leading to hepatic steatosis with the typical ballooning was observed in rats fed with Western diet (FIG. 53B, arrow), while a reduction of steatosis could be observed in the rats treated with d-methadone (FIG. 53C).

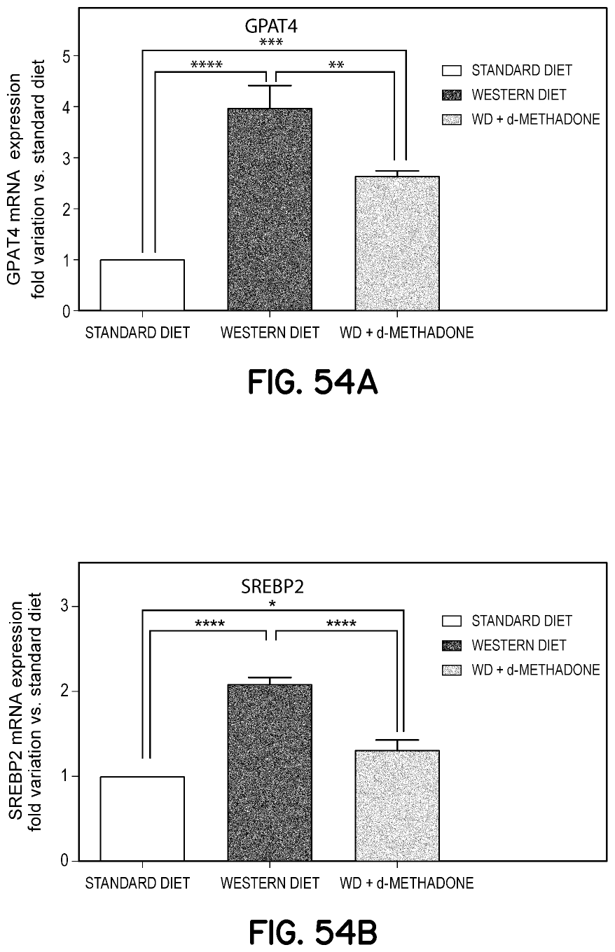

In order to support the histological data indicating the presence of hepatic steatosis, the present inventors measured the expression of two genes involved in lipid metabolism, i.e. GPAT4 and SREPB2, by qRT-PCR. As expected, the gene expression of both GPAT4 and SREPB2 was significantly increased by Western Diet administration, and d-methadone treatment was able to cause a significant drop of their expression, even if this decrease didn't restore their physiological levels (see FIGS. 54A and 54B).

Aside for adding potential indication to the therapeutic spectrum of dextromethadone (NAFLD and NASH), these data confirm that dextromethadone effects are not only symptomatic but are potentially disease-modifying: symptomatic treatments for mood disorders are not expected to exert measurable effects on inflammatory parameters. However, disease modifying treatments may potentially modulate different aspects of physiopathology, including metabolic and inflammatory states implicated and or associated with MDD.

While the present invention has been disclosed by reference to the details of preferred embodiments of the invention, it is to be understood that the disclosure is intended as an illustrative rather than in a limiting sense, as it is contemplated that modifications will readily occur to those skilled in the art, within the spirit of the invention and the scope of the amended claims.

What is claimed is:

1. A method of modifying the course and severity of a neuropsychiatric disorder comprising:
administering a composition to a subject suffering from a neuropsychiatric disorder, the neuropsychiatric disorder being selected from Major Depressive Disorder, Persistent Depressive Disorder, Disruptive Mood Dysregulation Disorder, Premenstrual Dysphoric Disorder, Postpartum Depression Disorder, Bipolar Disorder, Hypomania and Mania disorder, Generalized Anxiety Disorder, Social Anxiety Disorder, Somatic Symptom Disorder, Bereavement Depressive Disorder, Adjustment Depressive Disorder, Post-traumatic Stress Disorder, Obsessive Compulsive Disorder, Chronic Pain Disorder, Overactive Bladder Disorder, and Substance Use Disorder;
wherein the composition includes a substance selected from dextromethadone, dextromethadone metabolites, d-methadol, d-alpha-acetylmethadol, d-alpha-normethadol, l-alpha-normethadol, and pharmaceutically acceptable salts thereof.

2. The method of claim 1, wherein the substance is the sole active agent in the composition for treating said neuropsychiatric disorder.

3. The method of claim 1, wherein the substance is isolated from its enantiomer or synthesized de novo.

4. The method of claim 1, wherein the administering of the composition occurs under conditions effective for the substance to bind to an NMDA receptor of the subject and cause relief to the subject by modifying the course and severity of said neuropsychiatric disorder.

5. The method of claim 4, wherein relief is chosen from cure of said neuropsychiatric disorder, prevention of said neuropsychiatric disorder, reduction in severity of said neuropsychiatric disorder, and reduction in duration of said neuropsychiatric disorder.

6. The method of claim 1, wherein the administering of the composition occurs as monotherapy.

7. The method of claim 1, wherein the administering of the composition occurs as part of adjunctive treatment to a second substance.

8. The method of claim 1, wherein the administering of the composition occurs under conditions effective for an action at a ion channel, neurotransmitter systems, neurotransmitter pathway, or receptor selected from an ionotropic glutamate receptor, a 5-HT2A receptor, a 5-HT2C receptor, an opioid receptor, an AChR, a SERT, a NET, a sigma 1 receptor, a K channel, a Na channel, and a Ca channel.

9. The method of claim 8, wherein the administering of the composition occurs under conditions effective for an action at an ionotropic glutamate receptor, and wherein the ionotropic glutamate receptor is an NMDAR.

10. The method of claim 9, wherein the action at the ionotropic glutamate receptor includes voltage dependent channel block of NMDARs expressed by the membrane of a cell.

11. The method of claim 10, wherein the action at the ionotropic glutamate receptor includes voltage dependent channel block of NMDARs expressed by the membrane of a cell with a preferential effect on NMDAR containing NR2C and NR2D subunits.

12. The method of claim 9, wherein the action at the ionotropic glutamate receptor includes the induction of synthesis of NMDAR subunits or other synaptic proteins that contribute to neuronal plasticity and contributes to the membrane expression of said synaptic proteins.

13. The method of claim 1, wherein the subject is a vertebrate.

14. The method of claim 13, wherein the vertebrate is a human.

15. The method of claim 1, wherein the substance is dextromethadone.

16. The method of claim 15, wherein the dextromethadone is in the form of a pharmaceutically acceptable salt.

17. The method of claim 15, wherein the dextromethadone is delivered at a total daily dosage of 0.1 mg to 5,000 mg.

18. The method of claim 1, wherein the administering of the composition modifies the course and severity of said neuropsychiatric disorder in a subject, and wherein the relief begins within a period of time chosen from two weeks or less after the initial administration of the substance, seven days or less after the initial administration of the substance, four days or less after the initial administration of the substance, and two days or less after the initial administration of the substance.

19. The method of claim 15, wherein a therapeutic effect of dextromethadone resulting from administering the composition reaches an effect size greater than or equal to 0.3 in phase 2 clinical trials or an effect size greater than or equal to 0.5 in phase 2 clinical trials, or an effect size greater than or equal to 0.7 in phase 2 clinical trials.

20. The method of claim 19, wherein the therapeutic effect is sustained for at least one week after the discontinuation of treatment.

21. The method of claim 19, wherein the duration of the therapeutic effect after the discontinuation of treatment is equal to or greater than the duration of the treatment.

22. The method of claim 1, wherein the administering of the composition occurs in addition to or in combination with the administration of one or more antidepressant medications to the subject.

23. The method of claim 1, wherein the administering of the composition occurs in addition to or in combination with the administration of one or more of magnesium, zinc, or lithium to the subject.

24. The method of claim 15, wherein administering the composition results in disease-modification of said neuropsychiatric disorder.

25. The method of claim 24, wherein said subject has a body mass index equal or less than 35.

26. The method of claim 1, wherein administering the composition is used to improve cognitive function, improve social function, improve sleep, improve sexual function, improve ability to perform at work, or improve motivation for social activities.

27. The method of claim 1, wherein the administering of the composition is performed orally, buccally, sublingually, rectally, vaginally, nasally, via aerosol, transdermally, parenterally, intravenously, subcutaneously, epidurally, intrathecally, intra-auricularly, intraocularly, or topically.

28. The method of claim 1, wherein the administering of the composition occurs at a dose of 25 mg per day.

29. The method of claim 1, wherein the administration of the composition includes administering a loading dose of the composition followed by administration of a daily dose of the composition.

30. The method of claim 29, wherein the loading dose of the composition includes an amount of the substance that is greater than the amount of the substance present in each daily dose of the composition.

31. The method of claim 30, wherein plasma levels at or higher than steady state are reached on the first day of administration of the composition.

32. The method of claim 30, wherein plasma levels at or higher than steady state are reached within 4 hours of administration of the composition.

33. The method of claim 1, wherein, following administering of the composition, total plasma levels of the substance in the subject are in a range of 5 ng/ml to 3000 ng/ml.

34. The method of claim 1, wherein, following administering of the composition, unbound levels of the substance in the subject are in a range of 0.1 nM to 1,500 nM.

35. The method of claim 1, wherein the administering of the composition occurs as an intermittent treatment schedule selected from every other day, once every three days, once weekly, every other week, every other two weeks, one week per month, every other month, every other 2 months, every other three months, one week per year, and one month per year.

36. The method of claim 35, wherein the administration of the composition is alternated with a placebo in the selected intermittent treatment schedule.

37. The method of claim 36, wherein instead of or in addition to placebo the method includes one or more of magnesium, zinc, or lithium.

38. The method of claim 1, further associated with a digital application to monitor the course of the disorder including the digital monitoring of symptoms and signs and functional and disability outcomes.

39. The method of claim 8, wherein the receptor is an opioid receptor and is chosen from MOR, KOR, and DOR.

40. A method for treating a neuropsychiatric disorder, comprising:
diagnosing an individual with a neuropsychiatric disorder chosen from Major Depressive Disorder, Persistent Depressive Disorder, Disruptive Mood Dysregulation Disorder, Premenstrual Dysphoric Disorder, Postpartum Depression Disorder, Bipolar Disorder, Hypomania and Mania disorder, Generalized Anxiety Disorder, Social Anxiety Disorder, Somatic Symptom Disorder, Bereavement Depressive Disorder, Adjustment Depressive Disorder, Post-traumatic Stress Disorder, Obsessive Compulsive Disorder, Chronic Pain Disorder, and Substance Use Disorder;
developing a course of treating the neuropsychiatric disorder of said individual; and
administering a substance to said individual as at least part of said course of treating the MDD of said individual, the substance being chosen from dextromethadone, dextromethadone metabolites, d-methadol, d-alpha-acetylmethadol, d-alpha-normethadol, l-alpha-normethadol, and pharmaceutically acceptable salts thereof.

41. A method for treating MDD, comprising:
diagnosing an individual with MDD; developing a course of treating the MDD of said individual; and
administering dextromethadone to said individual as at least part of said course of treating the MDD of said individual.

42. A method of treating a neuropsychiatric disorder comprising:
inducing the transcription, the synthesis and the membrane expression in a subject of NMDAR subunits, AMPAR subunits, or other synaptic proteins that contribute to neuronal plasticity and assembled NMDAR channels;
wherein the subject suffers from a neuropsychiatric disorder, the neuropsychiatric disorder being selected from Major Depressive Disorder, Persistent Depressive Disorder, Disruptive Mood Dysregulation Disorder, Premenstrual Dysphoric Disorder, Postpartum Depression Disorder, Bipolar Disorder, Hypomania and Mania disorder, Generalized Anxiety Disorder, Social Anxiety Disorder, Somatic Symptom Disorder, Bereavement Depressive Disorder, Adjustment Depressive Disorder, Post-traumatic Stress Disorder, Obsessive Compulsive Disorder, Chronic Pain Disorder, Overactive Bladder Disorder and Substance Use Disorder; and
wherein inducing the transcription, the synthesis and the membrane expression of NMDAR subunits, AMPAR subunits, or other synaptic proteins that contribute to neuronal plasticity is accomplished by administering to the subject a substance selected from dextromethadone, dextromethadone metabolites, d-methadol, d-alpha-acetylmethadol, d-alpha-normethadol, l-alpha-normethadol, and pharmaceutically acceptable salts thereof.

43. The method of claim 42, wherein treatment of said neuropsychiatric disorder results in relief of said neuropsychiatric disorder, said relief being chosen from cure of said neuropsychiatric disorder, prevention of said neuropsychiatric disorder, reduction in severity of said neuropsychiatric disorder, and reduction in incidence of said neuropsychiatric disorder.

44. The method of claim 42, wherein the subject is a vertebrate.

45. The method of claim 42, wherein the vertebrate is a human.

46. The method of claim 42, wherein the substance is dextromethadone.

47. The method of claim 42, wherein the dextromethadone is in the form of a pharmaceutically acceptable salt.

48. The method of claim 42, wherein the dextromethadone is delivered at a total daily dosage of 0.1 mg to 5,000 mg.

49. The method of claim 42, wherein the relief of the subject from said neuropsychiatric disorder begins two weeks or less after the initial administration of the substance.

50. The method of claim 42, wherein the relief of the subject from said neuropsychiatric disorder begins 7 days or less after the initial administration of the substance.

51. The method of claim 42, wherein a therapeutic effect of dextromethadone reaches an effect size greater than or equal to 0.3 in phase 2 clinical trials or an effect size greater than or equal to 0.5 in phase 2 clinical trials, or an effect size greater than or equal to 0.7 in phase 2 clinical trials.

52. The method of claim 51, wherein the therapeutic effect is sustained for at least one week after the discontinuation of treatment.

53. The method of claim 51, wherein the duration of the therapeutic effect after the discontinuation of treatment is equal to or greater than the duration of the treatment.

54. The method of claim 42, wherein the administering of the composition occurs in combination with the administration of antidepressant medications to the subject.

55. The method of claim 42, wherein the administering of the composition occurs in combination with the administration of one or more of magnesium, zinc, or lithium to the subject.

56. The method of claim 46, wherein dextromethadone is used as a disease modifying agent or as a cure for patients with a diagnosis of MDD and related neuropsychiatric disorders and body mass index equal or less than 35.

57. The method of claim 42, wherein administering the composition is used to improve cognitive function, improve social function, improve sleep, improve sexual function, improve ability to perform at work.

58. The method of claim 42, wherein the administering of the composition is performed orally, buccally, sublingually, rectally, vaginally, nasally, via aerosol, transdermally, parenterally, intravenously, subcutaneously, epidurally, intrathecally, intra-auricularly, intraocularly, or topically.

59. The method of claim 42, wherein the administering of the composition occurs at a dose of 0.01-1000 mg per day.

60. The method of claim 42, wherein the administration of the composition includes administering a loading dose of the composition followed by administration of a daily dose of the composition.

61. The method of claim 60, wherein the loading dose of the composition includes an amount of the substance that is two times or more the amount of the substance present in each daily dose of the composition.

62. The method of claim 42, wherein steady state is reached on the first day of administration of the composition.

63. The method of claim 42, wherein steady state is reached within 4 hours of administration of the composition.

64. The method of claim 42, wherein, following administration of the composition, unbound levels of the substance in the subject are 5 ng/ml to 3000 ng/ml.

65. The method of claim 42, wherein, following administration of the composition, unbound levels of the substance in the subject are 0.5 nM to 1,500 nM.

66. The method of claim 42, wherein the administering of the composition occurs as an intermittent treatment schedule selected from once a week, every other day, once every three days, once weekly, every other week, every other two days, every other 3 days, every two weeks, and every other month.

67. The method of claim 66, wherein the administration of the composition is alternated with a placebo in the selected intermittent treatment schedule.

68. The method of claim 67, wherein instead of placebo or in addition to placebo it includes one or more of magnesium, zinc, or lithium.

69. The method of claim 42, further associated with a digital application to monitor the course of the disorder, including symptoms and signs and functional and disability outcomes.

70. A method for treating a disease or disorder characterized by a dysfunction of ion channels, comprising:
diagnosing an individual with a disease or disorder characterized by a dysfunction of ion channels;
developing a course of treating the disease or disorder of said individual, wherein the course of treating the disease or disorder involves resolution of the dysfunction of ion channels; and
administering a substance to said individual as at least part of said course of resolving the dysfunction of ion channels, the substance being chosen from dextromethadone, dextromethadone metabolites, d-methadol, d-alpha-acetylmethadol, d-alpha-normethadol, l-alpha-normethadol, and pharmaceutically acceptable salts thereof.

71. The method of claim 70, wherein the ion channels are integral to one or more NMDARs.

72. The method of claim 70, wherein the ion channels are integral to NMDARs comprising the Glun2C subunit.

73. The method of claim 70, wherein the ion channels are integral to NMDARs comprising the Glun2D subunit.

74. The method of claim 70, wherein the ion channels are integral to NMDARs comprising the Glun2B subunit.

75. The method of claim 70, wherein the ion channels are integral to NMDARs comprising the Glun2A subunit.

76. The method of claim 70, wherein the ion channels are integral to NMDARs comprising the Glun3A subunits.

77. A method for diagnosing a disorder as a disorder caused, worsened, or maintained by pathologically hyperactive NMDAR channels comprising:
administering a composition to a subject, the composition including a substance selected from dextromethadone, dextromethadone metabolites, d-methadol, d-alpha-acetylmethadol, d-alpha-normethadol, l-alpha-normethadol, and pharmaceutically acceptable salts thereof, said subject having been diagnosed with at least one disorder of unclear pathophysiology chosen from neurological disorders, neuropsychiatric disorders, ophthalmic disorders, otologic disorders, metabolic disorders, osteoporosis, urogenital disorders, renal impairment, infertility, premature ovarian failure, liver disorders, immunological disorders, oncological disorders, cardiovascular disorders;
determining the effectiveness of said composition in said at least one disorder by measuring endpoints specific for each disorder before and after the administration of the composition; and
diagnosing subjects exhibiting improvement of specific endpoints with a disorder caused, worsened, or maintained by pathologically hyperactive NMDAR channels.

78. The method of claim 70, wherein the ion channels are integral to NMDARs comprising the GluN3B subunit.

79. A method for preventing acute and chronic complications, including ARDS, DIC, and renal, GI, and nervous system complications, from infectious diseases, including COVID-19, comprising:
administering a composition to a subject, the composition including a substance selected from dextromethadone, dextromethadone metabolites, d-methadol, d-alphaacetylmethadol, d-alpha-normethadol, l-alpha-normethadol, and pharmaceutically acceptable salts thereof.

80. A method for treating and diagnosing acute and chronic complications, including ARDS, DIC, and renal, GI, and nervous system complications, from infectious diseases, including COVID-19, comprising:
administering a composition to a subject, the composition including a substance selected from dextromethadone, dextromethadone metabolites, d-methadol, d-alphaacetylmethadol, d-alpha-normethadol, l-alpha-normethadol, and pharmaceutically acceptable salts thereof.

81. A method for treating and diagnosing lung diseases, disorders and conditions caused by hyper activation of NMDAR, including NMDAR of the GluN1-GluN2D subtype, the lung diseases, disorders, and conditions including asthma, ARDS, COPD, pulmonary fibrosis and pulmonary infections, and their sequelae, the method comprising:
administering a composition to a subject, the composition including a substance selected from dextromethadone, dextromethadone metabolites, d-methadol, d-alphaacetylmethadol, d-alpha-normethadol, l-alpha-normethadol, and pharmaceutically acceptable salts thereof.

82. A method for treating GI diseases, disorders and conditions, including liver and pancreatic diseases, including ulcers, irritable bowel syndrome, inflammatory bowel disease, NAFLD, NASH and metabolic diseases comprising:
administering a composition to a subject, the composition including a substance selected from dextromethadone, dextromethadone metabolites, d-methadol, d-alphaacetylmethadol, d-alpha-normethadol, l-alpha-normethadol, and pharmaceutically acceptable salts thereof.

83. A method for treating renal and urogenital diseases, disorders and conditions, including renal failure, infertility, premature ovarian failure, premenstrual syndrome, and endometriosis comprising:
administering a composition to a subject, the composition including a substance selected from dextromethadone, dextromethadone metabolites, d-methadol, d-alphaacetylmethadol, d-alpha-normethadol, l-alpha-normethadol, and pharmaceutically acceptable salts thereof.

84. A method for treating cardiovascular disorders and conditions, including ischemic heart disease and congestive heart failure comprising:
administering a composition to a subject, the composition including a substance selected from dextromethadone, dextromethadone metabolites, d-methadol, d-alphaacetylmethadol, d-alpha-normethadol, l-alpha-normethadol, and pharmaceutically acceptable salts thereof.

85. A method for diagnosing or preventing or treating acute and chronic diseases, disorders and conditions caused by hyper-activation of NMDAR by endogenous inflammatory molecules reactive to infective agents including SARS-CoV-2 virus infection, including quinolinic acid and other inflammatory molecules, comprising:
administering a composition to a subject, the composition including a substance selected from dextromethadone, dextromethadone metabolites, d-methadol, d-alphaacetylmethadol, d-alpha-normethadol, l-alpha-normethadol, and pharmaceutically acceptable salts thereof.

86. A method diagnosing or preventing or treating acute and chronic lung diseases, including asthma, caused by hyper-activation of NMDAR by endogenous or endogenous agents, including hyper activation of NMDAR GluN1-GluN2D subtypes, comprising:
administering a composition to a subject, the composition including a substance selected from dextromethadone, dextromethadone metabolites, d-methadol, d-alphaacetylmethadol, d-alpha-normethadol, l-alpha-normethadol, and pharmaceutically acceptable salts thereof.
